(19) United States
(12) Patent Application Publication
(10) Pub. No.: US 2023/0033285 A1
(43) Pub. Date: Feb. 2, 2023

(54) Protease Inhibitors for Treatment of Coronavirus Infections

(71) Applicant: The Scripps Research Institute, La Jolla, CA (US)

(72) Inventors: Arnab K. Chatterjee, San Diego, CA (US); Jian Jeffrey Chen, San Diego, CA (US); Elshan Nakath, La Jolla, CA (US); Alireza Rahimi, La Jolla, CA (US); Anil Kumar Gupta, San Diego, CA (US); Gennadii Grabovyi, La Jolla, CA (US); Katy Wilson, La Jolla, CA (US); Sourav Ghorai, La Jolla, CA (US); Armen Nazarian, La Jolla, CA (US); James Pedroarena, La Jolla, CA (US); Wrickban Mazumdar, La Jolla, CA (US); Frank Weiss, La Jolla, CA (US); Lirui Song, San Diego, CA (US); Malina A. Bakowski, La Jolla, CA (US); Laura Riva, San Diego, CA (US); Karen Wolff, Encinitas, CA (US); Case W. McNamara, San Marcos, CA (US); Thomas F. Rogers, Del Mar, CA (US); Jacqueline Malvin, San Diego, CA (US); Shuangwei Li, San Diego, CA (US); Sean Joseph, San Diego, CA (US); Ashley Woods, San Diego, CA (US); Yuyin Liu, San Diego, CA (US); Neechi Okwor, Del Mar, CA (US)

(21) Appl. No.: 17/842,415

(22) Filed: Jun. 16, 2022

Related U.S. Application Data
(60) Provisional application No. 63/202,552, filed on Jun. 16, 2021.
(60) Provisional application No. 63/266,234, filed on Dec. 30, 2021.
(60) Provisional application No. 63/268,735, filed on Mar. 1, 2022.

(51) Int. Cl. C07D 491/048; C07D 403/12; C07D 207/26; C07D 401/12; C07D 211/76; C07D 223/10; C07D 405/14; C07D 413/14; C07D 401/14; C07D 413/12; A61P 31/14

(52) U.S. Cl. CPC C07D 491/048; C07D 403/12; C07D 207/26; C07D 401/12; C07D 211/76; C07D 223/10; C07D 405/14; C07D 413/14; C07D 401/14; C07D 413/12; A61P 31/14

(57) ABSTRACT

Provided herein are compounds of Formula (I), their pharmaceutically acceptable salts, and their pharmaceutical compositions:

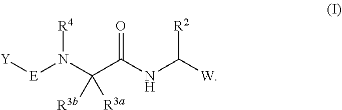

wherein R¹, R², R^{3a}, R^{3b}, R⁴, R⁵, and A are defined in the present disclosure. The compounds are potent inhibitors of the main protease (M^{pro}) of severe acute respiratory syndrome Coronavirus-2 (SARS-CoV-2), and they are useful in treating or preventing COVID-19 in a subject.

### CROSS-REFERENCE TO RELATED APPLICATIONS

This application claims the benefit of priority of U.S. Provisional Patent Application Ser. No. 63/202,552, filed Jun. 16, 2021, U.S. Provisional Patent Application Ser. No. 63/266,234, filed Dec. 30, 2021, and U.S. Provisional Patent Application Ser. No. 63/268,735, filed Mar. 1, 2022, the contents of which are incorporated by reference in their entirety.

### BACKGROUND

In early December of 2019, the severe acute respiratory syndrome coronavirus 2 (SARS-CoV-2) was identified as the cause of rapidly increasing numbers of severe pneumonia-like symptoms termed COVID-19. Since then, SARS-CoV-2 has rightfully been given its pandemic status by the World Health Organization (WHO). As of May 7, 2021, SARS-CoV-2 has spread throughout the world causing more than 155,000,000 confirmed infections and more than 3,250,000 reported deaths in 223 different countries. Development of several effective anti-SARS-CoV-2 vaccines could contribute to the control of the pandemic; however, emergence of SARS-CoV-2 strains with escape mutations that render some of the vaccines less effective and overall limited global supply of COVID-19 vaccines make a case for continued effort to identify therapeutic interventions. Yet, despite an extensive effort by the research community, antiviral treatment options for COVID-19 remain limited. These include corticosteroids such as dexamethasone and the intravenously-delivered antiviral remdesivir for treatment of patients with severe or critical COVID-19.

Remdesivir, a nucleotide analog prodrug and an RNA-dependent RNA polymerase (RdRp) inhibitor with broad antiviral activity, demonstrated positive clinical endpoints in a Phase III Adaptive COVID-19 Treatment Trial (median time to recovery shortened from 15 to 11 days) that justified its emergency use authorization by the US Food & Drug Administration for treatment of hospitalized COVID-19 patients. However, Remdesivir, together with hydroxychloroquine, lopinavir and interferon regimens, has recently failed to reduce mortality of hospitalized COVID-19 patients in a large multi-center WHO SOLIDARITY trial. Remdesivir's modest efficacy and intravenous delivery make the discovery of new or supplemental therapies that produce greater clinical improvements and can be administered outside of a hospital setting (i.e. orally) highly desirable.

Beyond RdRp, other high-value drug targets have been identified in SARS-CoV-2. Belonging to the genus betacoronavirus, this virus encodes two large overlapping polyprotein precursors (pp1a and pp1ab), four structural proteins (spike, envelope, membrane, and nucleocapsid), and several accessory proteins. The two polyproteins (pp1a/pp1ab) must be cleaved into its individual, nonstructural proteins for successful viral replication (Y. Chen et al. J Med Virol. 2020; 92(10):2249.). Two viral proteases are essential and responsible for processing the polyproteins: the main protease (M^{pro} or 3CL protease) and a papain-like protease (Hilgenfeld R. From SARS to MERS: crystallographic studies on coronaviral proteases enable antiviral drug design. FEBS J. 2014; 281(18):4085-96). Importantly, M^{pro} cleaves polypeptides after a glutamine residue in the P1 position of the substrate, which is a unique activity not observed in other human proteases and suggests that this viral protease can be specifically and selectively inhibited by a small molecule inhibitor (Zhang L et al. α-Ketoamides as Broad-Spectrum Inhibitors of Coronavirus and Enterovirus Replication: Structure-Based Design, Synthesis, and Activity Assessment. J Med Chem. 2020; 63(9):4562-4578).

Examination of the active site of M^{pro} reveals four sites (S1′, S1, S2, and S4), which, in turn, can accommodate four corresponding fragments of the substrate (P1′, P1, P2, and P3, respectively). Because polypeptides are the natural substrate, then peptidomimetic inhibitors are a rationale choice for high-affinity small molecules for proteases. Affinity of a peptidomimetic inhibitor can be further enhanced by introducing a warhead in P1 to form a covalent bond with the catalytic site Cys145, which is an essential residue for the antiviral activity (Dai W et al. Structure-based design of antiviral drug candidates targeting the SARS-CoV-2 main protease. Science. 2020; 368(6497):1331-1335). In addition, it is well established that a glutamine derivative (γ-lactam) is highly preferred to occupy the S1 site of cysteine proteases, which not only mimics the native P1 glutamine of the substrates but also increases the activity of inhibitors (Dragovich P S et al. Structure-based design, synthesis, and biological evaluation of irreversible human rhinovirus 3C protease inhibitors. 4. Incorporation of P1 lactam moieties as L-glutamine replacements. J Med Chem. 1999; 42(7):1213-24). Finally, a bicycloproline moiety, either (1R,2S,5S)-6,6-dimethyl-3-aza-bicyclo[3.1.0]hexane-2-formamide (P2 of boceprevir) or (1S,3aR,6aS)-octahydrocyclopenta[c]pyrrole-1-formamide (P2 of telaprevir), as a P2 fragment, suitably occupy the S2 pocket of M^{pro} (Qiao J et al. SARS-CoV-2 Mpro inhibitors with antiviral activity in a transgenic mouse model. Science. 2021; 371(6536):1374-1378). As previously reported, the rigid and hydrophobic bicycloproline can increase exposure of an orally administered compound (Yip Y et al. Discovery of a novel bicycloproline P2 bearing peptidyl alpha-ketoamide LY514962 as HCV protease inhibitor. Bioorg Med Chem Lett. 2004; 14(1):251-6). Thus, the modifications to the molecule representing the P3 fragment and the specific warhead, are important for imparting favorable biological activity and pharmacokinetic properties for an optimal drug candidate.

### SUMMARY

The present disclosure provides a surprisingly potent inhibitor of COVID-2 M^{pro} as a compound of Formula (I) or its pharmaceutically acceptable salt:

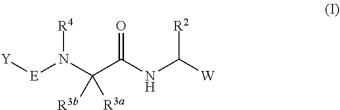

wherein
Y is

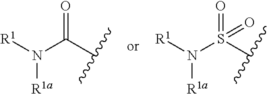

wherein
R¹ and R^{1a} are independently selected from the group consisting of H, C₁-C₈-alkyl, C₃-C₁₀-cycloalkyl, —(C₃-C₁₀-cycloalkyl)-(C₆-C₁₀-aryl), C₆-C₁₀-aryl, 5- to 10-membered heteroaryl (wherein 1-4 heteroaryl members are independently selected from N, O, and S), —(C₁-C₆-alkyl)(C₆-C₁₀-aryl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S)), and —(C₁-C₆-alkyl)(5- to 10-membered heteroaryl (wherein 1-4 heteroaryl members are independently selected from N, O, and S)); or R¹ and R^{1a} together with the nitrogen atom to which they are bound form a 4-10 membered mono or bicyclic fused, bridged, or spiro-fused ring wherein the ring members are selected from C, N, O, and S;
wherein R¹ and R^{1a} are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₁-C₆-haloalkoxy, C₃-C₈-cycloalkyl (optionally substituted with 1-3 substituents independently selected from halo and NH₂), CN, and CONR⁷R⁸; or Y is selected from the group consisting of C₁-C₆-alkyl (optionally interrupted by one or more heteroatoms selected from —NH—, O, and S), C₁-C₆-haloalkyl, —(C₁-C₆-alkyl)-(C₃-C₁₀-cycloalkyl), 5- to 10-membered heteroaryl (wherein 1-4 heteroaryl members are independently selected from N, O, and S), —C(O)—C₆-C₁₀-aryl, —C(O)-(5- to 10-membered heteroaryl (wherein 1-4 heteroaryl members are independently selected from N, O, and S)), —C(O)—O—C₆-C₁₀-aryl, —C(O)—C₆-C₁₀-aryl, —C(O)—NH—C₆-C₁₀-aryl, —C(O)—C₁-C₆-alkyl-O—C₆-C₁₀-aryl, —C(O)—O—(C₁-C₆-alkyl)(C₆-C₁₀-aryl), —(C₂-C₆-alkenyl)(C₆-C₁₀-aryl), -(5- to 10-membered heteroaryl (wherein 1-4 heteroaryl members are independently selected from N, O, and S))—(C₆-C₁₀-aryl), —C(O)—O—(C₁-C₆-alkyl), —C(O)—C₁-C₆-alkyl-(5- to 10-membered heteroaryl (wherein 1-4 heteroaryl members are independently selected from N, O, and S)), —(C₁-C₆-alkyl)(C₆-C₁₀-aryl), —C(O)-(3- to 6-membered heterocycloalkyl (wherein 1-4 ring members are independently selected from N, O, and S)), —C(O)—O-(3- to 6-membered heterocycloalkyl (wherein 1-4 ring members are independently selected from N, O, and S)), —C(O)—C₁-C₆-alkyl optionally substituted with halo, —C(O)—C₃-C₁₀-cycloalkyl, and —C(O)—NH-(5- to 10-membered heteroaryl (wherein 1-4 heteroaryl members are independently selected from N, O, and S));
   wherein Y is optionally substituted with one to three substituents selected from the group consisting of halo, C₁-C₆-alkyl, C₁-C₆-haloalkyl, C₁-C₆-alkoxy, C₃-C₈-cycloalkyl (optionally substituted with 1-3 substituents independently selected from halo and NH₂), NH₂, C₁-C₆-alkyl optionally substituted with NH₂, 3- to 6-membered heterocycloalkyl (wherein 1-4 ring members are independently selected from N, O, and S), 5- to 10-membered heteroaryl (wherein 1-4 heteroaryl members are independently selected from N, O, and S), CN, COR⁷, CONR⁷R⁸, OR⁷, SR⁷, NR⁷C(O)O—(C₁-C₆-alkyl)(C₆-C₁₀-aryl), NR⁷C(O)R⁸, and SO₂R⁷;
R⁷ and R⁸ are independently selected from H, C₁-C₆-alkyl, C₁-C₆-haloalkyl, and C₃-C₈-cycloalkyl;
E is a bond, —C(O)—, or —NHC(R^{9a})(R^{9b})C(O)—;
R^{9a} and R^{9b} are independently selected from the group consisting of H, C₁-C₆-alkyl, C₃-C₈-cycloalkyl, —C₁-C₆-alkyl-O—C₁-C₆-alkyl, —C(O)C₁-C₆-alkyl (optionally substituted with one to three of C₁-C₆-alkyl, C₃-C₈-cycloalkyl, halo, C₁-C₆-haloalkyl), 3- to 6-membered heterocycloalkyl (wherein 1-4 ring members are independently selected from N, O, and S), and —(C₁-C₆-alkyl)-(3- to 6-membered heterocycloalkyl (wherein 1-4 ring members are independently selected from N, O, and S)),
   R^{9a} and R^{9b} are optionally and independently substituted with 1 to 5 substituents independently selected from the group consisting of halo, OH, NH₂, C₁-C₆-alkyl, C₁-C₆-haloalkyl, C₃-C₈-cycloalkyl (optionally substituted with 1-3 substituents independently selected from halo and NH₂);
W is selected from the group consisting of CN, C(O)H, —C(O)CH₂OH, —C(O)CH₂OC(O)R⁵, and —C(O)CH₂OC(O)C(O)R⁵,
   wherein R⁵ is selected from the group consisting of H, C₁-C₂₀-alkyl, C₆-C₁₀-aryl optionally substituted with CN, —(C₁-C₆-alkyl)-(C₆-C₁₀-aryl), C₁-C₆-alkoxy, —(C₃-C₈-cycloalkyl)-(3- to 6-membered heterocycloalkyl (wherein 1-4 ring members are independently selected from N, O, and S)), and -(3- to 6-membered heterocycloalkyl (wherein 1-4 ring members are independently selected from N, O, and S))—(C₃-C₈-cycloalkyl);
R² is selected from the group consisting of

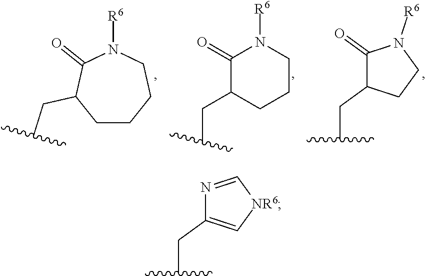

R⁶ is H or C₁-C₆-alkyl;
R^{3a}, R^{3b}, and R⁴ are selected independently from the group consisting of H, C₁-C₆-alkyl, C₃-C₁₀-cycloalkyl, —(C₁-C₆-alkyl)(C₃-C₁₀-cycloalkyl), —(C₁-C₆-alkyl)(C₆-C₁₀-aryl), —(C₁-C₆-alkyl)(3- to 6-membered heterocycloalkyl (wherein 1-4 ring members are independently selected from N, O, and S), C₂-C₈-alkenyl, and —(C₁-C₆-alkyl)(5- to 10-membered heteroaryl) (wherein 1-4 heteroaryl members are independently selected from N, O, and S),
   wherein R^{3a}, R^{3b}, and R⁴ are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₃-C₈-cycloalkyl (optionally substituted with 1-3 substituents independently selected from halo and NH₂), CN, and CONR⁷R⁸;
or R^{3a} and R⁴, R^{3b} and R⁴, or R^{3a} and R^{3b} together with the atoms to which they are bound form a 3-10 membered mono- or bicyclic ring that, if bicyclic, is optionally fused, bridged, or spiro-fused, wherein the mono- or bicyclic ring is optionally substituted with one to three substituents selected from halo, OH, C₂-C₆-alkenyl, C₁-C₆-haloalkyl, C₁-C₆-alkoxy, C₃-C₈-cycloalkyl (optionally and independently substituted with 1-3 substituents selected from halo and NH₂), C₁-C₆-alkyl optionally substituted with NH₂, 3- to 6-membered heterocycloalkyl (wherein 1-4 ring members are independently selected from N, O, and S), CN, and CONR⁷R⁸;
   or a pharmaceutically acceptable salt thereof.

In Formula (I), Y is

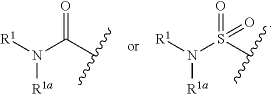

R¹ and R^{1a} are independently selected from the group consisting of H, C₁-C₈-alkyl, C₃-C₁₀-cycloalkyl, C₆-C₁₀-aryl, 5- to 10-membered heteroaryl (wherein 1-4 heteroaryl members are independently selected from N, O, and S), —(C₁-C₆-alkyl)(C₆-C₁₀-aryl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S)), and —(C₁-C₆-alkyl)(5- to 10-membered heteroaryl (wherein 1-4 heteroaryl members are independently selected from N, O, and S)).

In some embodiments, R¹ and R^{1a} together with the nitrogen atom to which they are bound form a 4-10 membered mono or bicyclic fused, bridged, or spiro-fused ring wherein the ring members are selected from C, N, O, and S.

R¹ and R^{1a} are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₁-C₆-haloalkoxy, C₃-C₈-cycloalkyl (optionally substituted with 1-3 substituents independently selected from halo and NH₂), CN, and CONR⁷R⁸.

In other embodiments, Y is selected from the group consisting of 5- to 10-membered heteroaryl (wherein 1-4 heteroaryl members are independently selected from N, O, and S), —C(O)—C₆-C₁₀-aryl, —C(O)-(5- to 10-membered heteroaryl (wherein 1-4 heteroaryl members are independently selected from N, O, and S)), —C(O)—O—C₆-C₁₀-aryl, —C(O)—NH—C₆-C₁₀-aryl, —C(O)—C₁-C₆-alkyl-O—C₆-C₁₀-aryl, —C(O)—C₁-C₆-alkyl-(5- to 10-membered heteroaryl (wherein 1-4 heteroaryl members are independently selected from N, O, and S)), —(C₁-C₆-alkyl)(C₆-C₁₀-aryl), —C(O)-(3- to 6-membered heterocycloalkyl (wherein 1-4 ring members are independently selected from N, O, and S)), —C(O)—O-(3- to 6-membered heterocycloalkyl (wherein 1-4 ring members are independently selected from N, O, and S)), —C(O)—C₁-C₆-alkyl optionally substituted with halo, —C(O)—C₃-C₁₀-cycloalkyl, and —C(O)—NH-(5- to 10-membered heteroaryl (wherein 1-4 heteroaryl members are independently selected from N, O, and S));
wherein Y is optionally substituted with one to three substituents selected from the group consisting of halo, C₁-C₆-alkyl, C₁-C₆-haloalkyl, C₁-C₆-alkoxy, C₃-C₈-cycloalkyl (optionally substituted with 1-3 substituents independently selected from halo and NH₂), NH₂, C₁-C₆-alkyl optionally substituted with NH₂, 3- to 6-membered heterocycloalkyl (wherein 1-4 ring members are independently selected from N, O, and S), CN, COR⁷, CONR⁷R⁸, OR⁷, SR⁷, and SO₂R⁷.

R⁷ and R⁸ are independently selected from H, C₁-C₆-alkyl, and C₃-C₈-cycloalkyl.

E is a bond, —C(O)—, or —NHC(R^{9a})(R^{9b})C(O)—.

R^{9a} and R^{9b} are independently selected from the group consisting of H, C₁-C₆-alkyl, C₃-C₈-cycloalkyl, —C₁-C₆-alkyl-O—C₁-C₆-alkyl, —C(O)C₁-C₆-alkyl (optionally substituted with one to three of C₁-C₆-alkyl, C₃-C₈-cycloalkyl, halo, and C₁-C₆-haloalkyl), 3- to 6-membered heterocycloalkyl (wherein 1-4 ring members are independently selected from N, O, and S).

R^{9a} and R^{9b} are optionally and independently substituted with 1 to 5 substituents independently selected from the group consisting of halo, OH, NH₂, C₁-C₆-alkyl, C₁-C₆-haloalkyl, C₃-C₈-cycloalkyl (optionally substituted with 1-3 substituents independently selected from halo and NH₂).

W is selected from the group consisting of CN, C(O)H, —C(O)CH₂OH, —C(O)CH₂OC(O)R⁵, and —C(O)CH₂OC(O)C(O)R⁵.

R⁵ is selected from the group consisting of H, C₁-C₂₀-alkyl, C₆-C₁₀-aryl, —(C₃-C₅-cycloalkyl)-(3- to 6-membered heterocycloalkyl (wherein 1-4 ring members are independently selected from N, O, and S)), and -(3- to 6-membered heterocycloalkyl (wherein 1-4 ring members are independently selected from N, O, and S))—(C₃-C₈-cycloalkyl).

R² is selected from the group consisting of

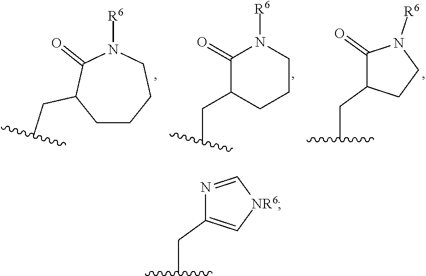

R⁶ is H or C₁-C₆-alkyl.

R^{3a}, R^{3b}, and R⁴ are selected independently from the group consisting of H, C₁-C₆-alkyl, C₃-C₁₀-cycloalkyl, —(C₁-C₆-alkyl)(C₃-C₁₀-cycloalkyl), —(C₁-C₆-alkyl)(C₆-C₁₀-aryl), —(C₁-C₆-alkyl)(3- to 6-membered heterocycloalkyl (wherein 1-4 ring members are independently selected from N, O, and S), and —(C₁-C₆-alkyl)(5- to 10-membered heteroaryl) (wherein 1-4 heteroaryl members are independently selected from N, O, and S).

R^{3a}, R^{3b}, and R⁴ are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₃-C₈-cycloalkyl (optionally substituted with 1-3 substituents independently selected from halo and NH₂), CN, and CONR⁷R⁸.

In some embodiments, R^{3a} and R⁴, R^{3b} and R⁴, or R^{3a} and R^{3b} together with the atoms to which they are bound form a 3-10 membered mono- or bicyclic ring that, if bicyclic, is optionally fused, bridged, or spiro-fused, wherein the mono- or bicyclic ring is optionally substituted with one to three substituents selected from halo, OH, C₂-C₆-alkenyl, C₁-C₆-haloalkyl, C₁-C₆-alkoxy, C₃-C₈-cycloalkyl (optionally and independently substituted with 1-3 substituents selected from halo and NH₂), C₁-C₆-alkyl optionally substituted with NH₂, 3- to 6-membered heterocycloalkyl (wherein 1-4 ring members are independently selected from N, O, and S), CN, and CONR⁷R⁸.

The present disclosure also provides in embodiments a pharmaceutical composition comprising a compound or a pharmaceutically acceptable salt thereof as described herein and a pharmaceutically acceptable carrier.

In another embodiment, the present disclosure provides a method for inhibiting the main protease (M^{pro}) of severe acute respiratory syndrome Coronavirus-2 (SARS-CoV-2). The method comprises contacting M^{pro} with a compound or pharmaceutically acceptable thereof as described herein.

Another embodiment is a method for treating COVID-19 in a subject suffering therefrom, or for preventing COVID-19 in a subject. The method comprises contacting M^{pro} with a compound or pharmaceutically acceptable thereof as described herein.

The present disclosure also provides, in an embodiment, a compound or pharmaceutically acceptable salt thereof as described herein for inhibiting the main protease (M^{pro}) of severe acute respiratory syndrome Coronavirus-2 (SARS-CoV-2) in a subject.

The present disclosure provides in another embodiment a compound or pharmaceutically acceptable salt thereof as described herein for treating COVID-19 in a subject suffering therefrom, or for preventing COVID-19 in a subject.

### DETAILED DESCRIPTION

Compounds of the present disclosure are potent inhibitors of M^{pro}, exhibit significant metabolic stability, and are useful in oral dosing to patients for treatment of COVID-19 and for prophylaxis against COVID-19.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O— hexyl, —O-isohexyl, and —O-neohexyl.

The term “haloalkoxy” or “haloalkoxyl” refers to an alkoxy group having the number of indicated carbon atoms and is substituted with 1 to 6 halides as defined herein and combinations thereof. Exemplary haloalkoxy groups include —OCHF₂ and —OCF₃.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. Polycyclic cycloalkyl includes rings that can be fused, bridged, and/or spiro-fused. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. Polycyclic heterocycloalkyl includes rings that can be fused, bridged, and/or spiro-fused. In addition, a heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula (I) includes a pharmaceutically acceptable salt of a tautomer of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The present disclosure includes all pharmaceutically acceptable isotopically-labelled compounds of Formula (I), wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, F, ¹⁵O and ¹³N, can be useful in Positron Emission Topography (PET) studies for examining substrate receptor occupancy. Isotopically-labeled compounds of Formula (I) may generally be prepared by conventional techniques known to those skilled in the art or by processes analogous to those described in the accompanying Examples using an appropriate isotopically-labeled reagents in place of the non-labeled reagent previously employed.

The present disclosure includes all pharmaceutically acceptable isotopically-labelled compounds of Formula (I), wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, F, ¹⁵O and ¹³N, can be useful in Positron Emission Topography (PET) studies for examining substrate receptor occupancy. Isotopically-labeled compounds of Formula (I) may generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the invention encompasses any tautomeric, geometric, or stereoisomeric form, and mixtures thereof, and is not to be limited merely to any one tautomeric, geometric, or stereoisomeric form utilized within the formula drawings.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### COMPOUNDS

The M^{pro} inhibitor compound of the present disclosure conforms in various embodiments to Formula (I):

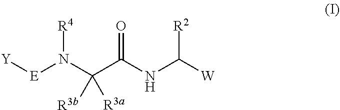

wherein R², R^{3a}, R^{3b}, R⁴, E, W, and Y are defined in summary hereinabove.

In some embodiments, W is C(O)H. In other embodiments, W is CN. In still other embodiments, W is selected from the group consisting of C(O)CH₂OH, —C(O)CH₂OC(O)R⁵, and —C(O)CH₂OC(O)C(O)R⁵. In an exemplary embodiment, W is C(O)CH₂OH. In some embodiments, W is —C(O)CH₂OC(O)R⁵, and R⁵ is C₁-C₂₀-alkyl, C₆-C₁₀-aryl optionally substituted with CN, —(C₁-C₆-alkyl)-(C₆-C₁₀-aryl), or C₁-C₆-alkoxy.

The present disclosure also provides a Formula (I) compound, per various embodiments, wherein R² is

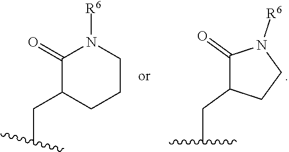

In some embodiments, R² is

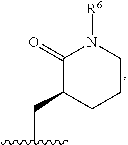

and in other embodiments, R² is

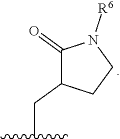

R⁶ is H in various illustrative embodiments.

Further embodiments of the present disclosure provide a compound wherein R^{3a} is H and R^{3b} is selected from the group consisting of optionally substituted C₁-C₆-alkyl and —(C₁-C₆-alkyl)(C₃-C₁₀-cycloalkyl). In some embodiments, R^{3a} is H and R^{3b} is optionally substituted C₁-C₆-alkyl. Examples of R^{3b} include and

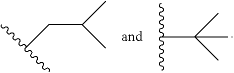

In various embodiments, R^{3a} is H and R^{3b} is

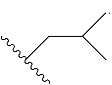 [SMILES: *CC(C)C]

In other embodiments, the present disclosure provides a Formula (I) compound where in the moiety

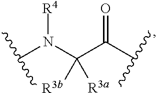

R^{3a} is H, and R^{3b} and R⁴ together with the atoms to which they are bound form an optionally substituted 3-10 membered mono- or bicyclic ring. If the ring is bicyclic, then it can be optionally fused, bridged, or spiro-fused. Examples of the moiety

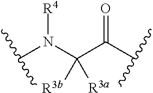

include those in the group consisting of

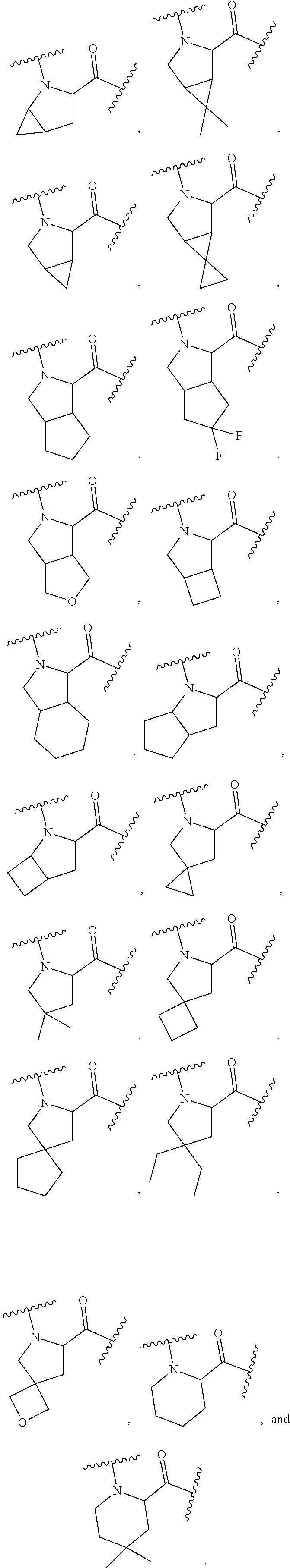

In some embodiments, E is a bond. In other embodiments, E is —C(O)—. In some embodiments, E is —NHC(R^{9a})(R^{9b})C(O)—.

In various embodiments, Y is

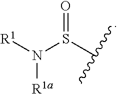

In exemplary embodiments, R¹ is H. Illustrative examples of Y include those selected from the group consisting of.

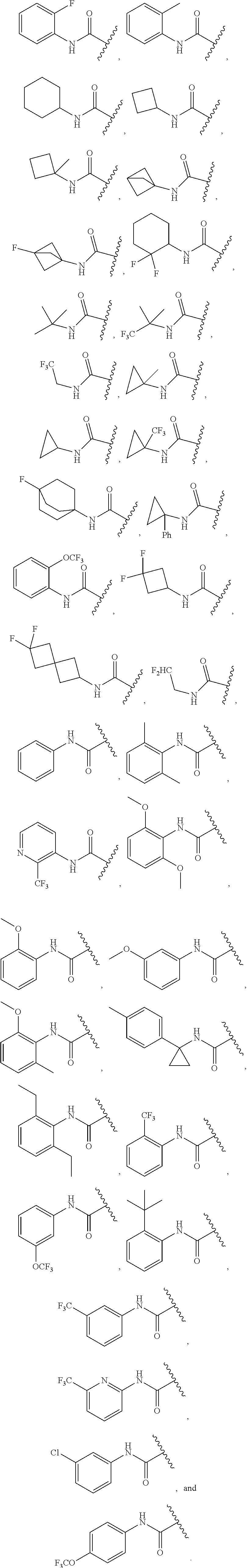

In some embodiments, disclosed is a compound of Formula (I), or a pharmaceutically acceptable salt thereof, wherein
Y is

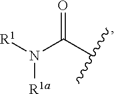

wherein R¹ is H and R^{1a} is C₁-C₈-alkyl, C₃-C₁₀-cycloalkyl, or C₆-C₁₀-aryl, wherein R^{1a} is optionally substituted with 1 to 5 substituents independently selected from the group consisting of halo, C₁-C₆-alkyl, C₁-C₆-haloalkyl, and C₁-C₆-haloalkoxy;
or Y is C₁-C₆-alkyl, —C(O)—C₁-C₆-alkyl, —C(O)—C₁-C₆-alkyl-O—C₆-C₁₀-aryl, or -(5- to 10-membered heteroaryl (wherein 1-4 heteroaryl members are independently selected from N, O, and S))—(C₆-C₁₀-aryl), wherein Y is optionally substituted with one to three substituents selected from the group consisting of halo, C₁-C₆-alkyl, and C₁-C₆-haloalkyl;
E is a bond, —C(O)—, or —NHCH(C₁-C₆-alkyl)C(O)—;
W is CN, C(O)H, —C(O)CH₂OH, or —C(O)CH₂OC(O)(C₁-C₂₀-alkyl); R² is

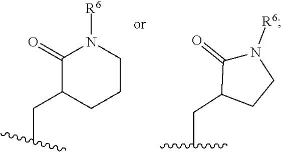

R^{3a}, R^{3b}, and R⁴ are selected independently from the group consisting of H, C₁-C₆-alkyl, and —(C₁-C₆-alkyl)(C₃-C₁₀-cycloalkyl), wherein the cycloalkyl is optionally substituted with one to three C₁-C₆-alkyl;
or the moiety

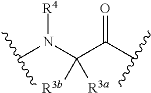

is selected from the group consisting of

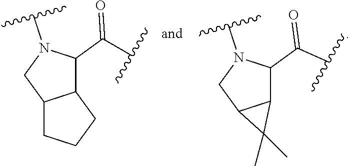 [SMILES: *C(=O)C1C2CCCC2CN1* ; *C(=O)C1C2C(CN1*)C2(C)C]

In some embodiments, disclosed is a compound of Formula (I), or a pharmaceutically acceptable salt thereof, wherein
Y is

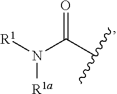

wherein R¹ is H and R^{1a} is C₁-C₈-alkyl or C₆-C₁₀-aryl, wherein R^{1a} is optionally substituted with 1 to 5 substituents independently selected from the group consisting of halo, C₁-C₆-haloalkyl, and C₁-C₆-haloalkoxy;
E is —C(O)—;
W is —C(O)CH₂OH;
R² is

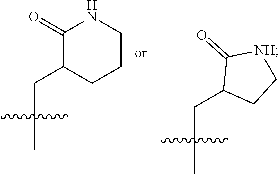 [SMILES: *CC1CCCNC1=O ; *CC1CCNC1=O]

R^{3a} is H;
R^{3b} is C₁-C₆-alkyl or —(C₁-C₆-alkyl)(C₃-C₁₀-cycloalkyl); and
R⁴ is H.

In some embodiments, disclosed is a compound of Formula (I), or a pharmaceutically acceptable salt thereof, wherein
Y is

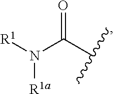

wherein R¹ is H and R^{1a} is tert-butyl or phenyl, wherein R^{1a} is substituted with 1 to 5 substituents independently selected from the group consisting of fluoro, trifluoromethyl, and trifluoromethoxy;
E is —C(O)—;
W is —C(O)CH₂OH;
R² is

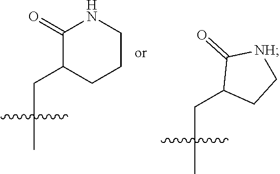 [SMILES: *CC1CCCNC1=O ; *CC1CCNC1=O]

R^{3a} is H;
R^{3b} is iso-butyl, neo-pentyl, or —CH₂-cyclopropyl; and
R⁴ is H.

Specific examples of Formula (I) compounds or their pharmaceutically acceptable salts constitute additional embodiments of the disclosure. Some of these are illustrated throughout the examples and shown in Table 1 below. In some embodiments, the compound of Formula (I) is selected from the group consisting of:
N—((S)-1-(((S)-4-hydroxy-3-oxo-1-((S)-2-oxopyrrolidin-3-yl)butan-2-yl)amino)-4-methyl-1-oxopentan-2-yl)-4-methoxy-1H-indole-2-carboxamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-phenyloxalamide;
N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl pentadecanoate;
(S)-3-((S)-2-(2-((2-fluorophenyl)amino)-2-oxoacetamido)-4-methylpentanamido)-2-oxo-4-((S)-2-oxopyrrolidin-3-yl)butyl 4-(piperidin-1-yl)cyclohexane-1-carboxylate;
(S)-3-((S)-2-(2-((2-fluorophenyl)amino)-2-oxoacetamido)-4-methylpentanamido)-2-oxo-4-((S)-2-oxopyrrolidin-3-yl)butyl 1-cyclohexylpiperidine-4-carboxylate;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2-methoxyphenyl)oxalamide;
N¹—((S)-3-cyclohexyl-1-(((S)-4-hydroxy-3-oxo-1-((S)-2-oxopyrrolidin-3-yl)butan-2-yl)amino)-1-oxopropan-2-yl)-N²-(o-tolyl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(3-methoxyphenyl)oxalamide;
(S)-2-(3-(2-fluorophenyl)ureido)-N—((S)-4-hydroxy-3-oxo-1-((S)-2-oxopyrrolidin-3-yl)butan-2-yl)-4-methylpentanamide;
(S)-3-((S)-2-(2-((2,6-dimethylphenyl)amino)-2-oxoacetamido)-4-methylpentanamido)-2-oxo-4-((S)-2-oxopyrrolidin-3-yl)butyl 2-oxo-2-phenylacetate;
N¹-(2,6-dimethylphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2,6-dimethylphenyl)-N²-((2S)-1-((4-hydroxy-3-oxo-1-((S)-2-oxopyrrolidin-3-yl)butan-2-yl)amino)-4-methyl-1-oxopentan-2-yl)oxalamide;
N¹-cyclohexyl-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2,6-dimethoxyphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2-methoxy-6-methylphenyl)oxalamide;
N¹-(2,6-diethylphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
(S)-3-((S)-2-(2-(cyclohexylamino)-2-oxoacetamido)-4-methylpentanamido)-2-oxo-4-((S)-2-oxopyrrolidin-3-yl)butyl heptanoate;
(S)-3-((S)-3-cyclohexyl-2-(2-(cyclohexylamino)-2-oxoacetamido)propanamido)-2-oxo-4-((S)-2-oxopyrrolidin-3-yl)butyl heptanoate;
N¹—((S)-3-(2-fluorophenyl)-1-(((S)-4-hydroxy-3-oxo-1-((S)-2-oxopyrrolidin-3-yl)butan-2-yl)amino)-1-oxopropan-2-yl)-N²-(o-tolyl)oxalamide;
N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹-(3-fluorobicyclo[1.1.1]pentan-1-yl)-N²—((S)-1-(((S)-4-hydroxy-3-oxo-1-((S)-2-oxopyrrolidin-3-yl)butan-2-yl)amino)-4,4-dimethyl-1-oxopentan-2-yl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(1-(p-tolyl)cyclopropyl)oxalamide;
N¹—((S)-3-cyclohexyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-3-cyclohexyl-1-(((R)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2,2,2-trifluoroethyl)oxalamide;
N¹—((S)-1-(((S)-4-hydroxy-3-oxo-1-((S)-2-oxopiperidin-3-yl)butan-2-yl)amino)-4-methyl-1-oxopentan-2-yl)-N²-(o-tolyl)oxalamide;
N¹-(2,6-diethylphenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-1-(((S)-4-hydroxy-3-oxo-1-((S)-2-oxopiperidin-3-yl)butan-2-yl)amino)-4-methyl-1-oxopentan-2-yl)-N²-(1-methylcyclopropyl)oxalamide;
(1S,3aR,6aS)-2-(2-((2-fluorophenyl)amino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide;
(1S,3aR,6aS)-2-(2-(tert-butylamino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide;
(1S,3aR,6aS)-2-(2-(tert-butylamino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
(1S,3aR,6aS)-2-(2-((2-fluorophenyl)amino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
N¹-(2-fluorophenyl)-N²—((S)-1-(((S)-4-hydroxy-3-oxo-1-((S)-2-oxoazepan-3-yl)butan-2-yl)amino)-4-methyl-1-oxopentan-2-yl)oxalamide;
4-methoxy-N—((S)-4-methyl-1-oxo-1-(((S)-1-oxo-3-((S)-2-oxopyrrolidin-3-yl)propan-2-yl)amino)pentan-2-yl)-1H-indole-2-carboxamide;
(1R,2S,5S)-6,6-dimethyl-N—((S)-1-oxo-3-((S)-2-oxopyrrolidin-3-yl)propan-2-yl)-3-(2-(4-(trifluoromethoxy)phenoxy)acetyl)-3-azabicyclo[3.1.0]hexane-2-carboxamide;
N¹—((S)-4-methyl-1-oxo-1-(((S)-1-oxo-3-((S)-2-oxopyrrolidin-3-yl)propan-2-yl)amino)pentan-2-yl)-N²-phenyloxalamide;
N¹—((S)-4-methyl-1-oxo-1-(((S)-1-oxo-3-((S)-2-oxopyrrolidin-3-yl)propan-2-yl)amino)pentan-2-yl)-N²-(o-tolyl)oxalamide;
N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide;
N¹—((S)-3-cyclohexyl-1-oxo-1-(((S)-1-oxo-3-((S)-2-oxopyrrolidin-3-yl)propan-2-yl)amino)propan-2-yl)-N²-(2-fluorophenyl)oxalamide;
N¹-(2-fluorophenyl)-N²—((S)-4-methyl-1-oxo-1-(((S)-1-oxo-3-((S)-2-oxopiperidin-3-yl)propan-2-yl)amino)pentan-2-yl)oxalamide;
N¹-cyclohexyl-N²—((S)-4-methyl-1-oxo-1-(((S)-1-oxo-3-((S)-2-oxopyrrolidin-3-yl)propan-2-yl)amino)pentan-2-yl)oxalamide;
N¹—((S)-4-methyl-1-oxo-1-(((S)-1-oxo-3-((S)-2-oxopyrrolidin-3-yl)propan-2-yl)amino)pentan-2-yl)-N²-(1-methylcyclopropyl)oxalamide;
(1S,3aR,6aS)-2-(2-((2-fluorophenyl)amino)-2-oxoacetyl)-N—((S)-1-oxo-3-((S)-2-oxopyrrolidin-3-yl)propan-2-yl)octahydrocyclopenta[c]pyrrole-1-carboxamide;
(1S,3aR,6aS)-2-(2-(2,4-dichlorophenoxy)acetyl)-N—((S)-1-oxo-3-((S)-2-oxopiperidin-3-yl)propan-2-yl)octahydrocyclopenta[c]pyrrole-1-carboxamide;
(1R,2S,5S)-3-(2-(tert-butylamino)-2-oxoacetyl)-6,6-dimethyl-N—((S)-1-oxo-3-((S)-2-oxopiperidin-3-yl)propan-2-yl)-3-azabicyclo[3.1.0]hexane-2-carboxamide;
(1S,3aR,6aS)-2-(2-oxo-2-((1,1,1-trifluoro-2-methylpropan-2-yl)amino)acetyl)-N—((S)-1-oxo-3-((S)-2-oxopiperidin-3-yl)propan-2-yl)octahydrocyclopenta[c]pyrrole-1-carboxamide;
(1R,2S,5S)-3-(2-(cyclohexylamino)-2-oxoacetyl)-6,6-dimethyl-N—((S)-1-oxo-3-((S)-2-oxopiperidin-3-yl)propan-2-yl)-3-azabicyclo[3.1.0]hexane-2-carboxamide;
(1S,3aR,6aS)-2-((S)-3,3-dimethyl-2-(2,2,2-trifluoroacetamido)butanoyl)-N—((S)-1-oxo-3-((S)-2-oxopiperidin-3-yl)propan-2-yl)octahydrocyclopenta[c]pyrrole-1-carboxamide;
(1S,3aR,6aS)-2-((R)-3-fluoro-3-methyl-2-(2,2,2-trifluoroacetamido)butanoyl)-N—((S)-1-oxo-3-((S)-2-oxopiperidin-3-yl)propan-2-yl)octahydrocyclopenta[c]pyrrole-1-carboxamide;
(1S,3aR,6aS)-2-((S)-2-(3,3-difluorocyclobutyl)-2-(2,2,2-trifluoroacetamido)acetyl)-N—((S)-1-oxo-3-((S)-2-oxopiperidin-3-yl)propan-2-yl)octahydrocyclopenta[c]pyrrole-1-carboxamide;
N—((S)-3,3-dimethyl-1-oxo-1-((1S,3aR,6aS)-1-(((S)-1-oxo-3-((S)-2-oxopiperidin-3-yl)propan-2-yl)carbamoyl)hexahydrocyclopenta[c]pyrrol-2(1H)-yl)butan-2-yl)-5-(trifluoromethyl)isoxazole-3-carboxamide;
(1S,3aR,6aS)-2-(O-(difluoromethyl)-N-(2,2,2-trifluoroacetyl)-L-threonyl)-N—((S)-1-oxo-3-((S)-2-oxopiperidin-3-yl)propan-2-yl)octahydrocyclopenta[c]pyrrole-1-carboxamide;
(1S,3aR,6aS)-2-((S)-2-(1-methylcyclopropyl)-2-(2,2,2-trifluoroacetamido)acetyl)-N—((S)-1-oxo-3-((S)-2-oxopiperidin-3-yl)propan-2-yl)octahydrocyclopenta[c]pyrrole-1-carboxamide;
(1R,2S,5S)-3-((S)-3-methoxy-3-methyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide;
(1R,2S,5S)-3-((R)-3-fluoro-3-methyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide;
(1R,2S,5S)—N—((S)-1-cyano-2-((S)-2-oxopyrrolidin-3-yl)ethyl)-3-((S)-4,4-dimethyl-2-(2,2,2-trifluoroacetamido)pentanoyl)-6,6-dimethyl-3-azabicyclo[3.1.0]hexane-2-carboxamide;
N¹—((S)-1-(((S)-1-cyano-2-((S)-2-oxopyrrolidin-3-yl)ethyl)amino)-4-methyl-1-oxopentan-2-yl)-N²-(2-fluorophenyl)oxalamide;
(1R,2S,5S)—N—((S)-1-cyano-2-((S)-2-oxopyrrolidin-3-yl)ethyl)-3-(2-((2-fluorophenyl)amino)-2-oxoacetyl)-6,6-dimethyl-3-azabicyclo[3.1.0]hexane-2-carboxamide;
(1S,3aR,6aS)—N—((S)-1-cyano-2-((S)-2-oxopiperidin-3-yl)ethyl)-2-(2-((2-fluorophenyl)amino)-2-oxoacetyl)octahydrocyclopenta[c]pyrrole-1-carboxamide;
(1S,3aR,6aS)—N—((S)-1-cyano-2-((S)-2-oxopiperidin-3-yl)ethyl)-2-(2-(2,4-dichlorophenoxy)acetyl)octahydrocyclopenta[c]pyrrole-1-carboxamide;
(1S,3aR,6aS)—N—((S)-1-cyano-2-((S)-2-oxopyrrolidin-3-yl)ethyl)-2-(2-((2-fluorophenyl)amino)-2-oxoacetyl)octahydrocyclopenta[c]pyrrole-1-carboxamide;
(1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide;
(S)—N—((S)-1-cyano-2-((S)-2-oxopyrrolidin-3-yl)ethyl)-5-(2-((2-fluorophenyl)amino)-2-oxoacetyl)-5-azaspiro[2.4]heptane-6-carboxamide; (1R,2S,5S)—N—((S)-1-cyano-2-((S)-2-oxopyrrolidin-3-yl)ethyl)-6,6-dimethyl-3-(2-oxo-2-((1-(trifluoromethyl)cyclopropyl)amino)acetyl)-3-azabicyclo[3.1.0]hexane-2-carboxamide;
(1R,2S,5S)—N—((S)-1-cyano-2-((S)-2-oxopiperidin-3-yl)ethyl)-3-((S)-3-methoxy-3-methyl-2-(2,2,2-trifluoroacetamido)butanoyl)-6,6-dimethyl-3-azabicyclo[3.1.0]hexane-2-carboxamide;
(1R,2S,5S)—N—((S)-1-cyano-2-((S)-2-oxopiperidin-3-yl)ethyl)-3-((R)-3-fluoro-3-methyl-2-(2,2,2-trifluoroacetamido)butanoyl)-6,6-dimethyl-3-azabicyclo[3.1.0]hexane-2-carboxamide;
(1R,2S,5S)—N—((S)-1-cyano-2-((S)-2-oxopiperidin-3-yl)ethyl)-6,6-dimethyl-3-((S)-2-(tetrahydro-2H-pyran-4-yl)-2-(2,2,2-trifluoroacetamido)acetyl)-3-azabicyclo[3.1.0]hexane-2-carboxamide;
(1R,2S,5S)—N—((S)-1-cyano-2-((S)-2-oxopiperidin-3-yl)ethyl)-3-((S)-2-(4,4-difluorocyclohexyl)-2-(2,2,2-trifluoroacetamido)acetyl)-6,6-dimethyl-3-azabicyclo[3.1.0]hexane-2-carboxamide;
(1S,3aR,6aS)—N—((S)-1-cyano-2-((S)-2-oxopiperidin-3-yl)ethyl)-2-((S)-3,3-dimethyl-2-(2,2,2-trifluoroacetamido)butanoyl)octahydrocyclopenta[c]pyrrole-1-carboxamide;
(1S,3aR,6aS)—N—((S)-1-cyano-2-((S)-2-oxopiperidin-3-yl)ethyl)-2-((R)-3-fluoro-3-methyl-2-(2,2,2-trifluoroacetamido)butanoyl)octahydrocyclopenta[c]pyrrole-1-carboxamide;
(3aS,4S,6aR)—N—((S)-1-cyano-2-((S)-2-oxopiperidin-3-yl)ethyl)-5-((S)-3,3-dimethyl-2-(2,2,2-trifluoroacetamido)butanoyl)hexahydro-1H-furo[3,4-c]pyrrole-4-carboxamide;
(1S,3aR,6aS)—N—((S)-1-cyano-2-((S)-2-oxopiperidin-3-yl)ethyl)-2-((S)-3,3-dimethyl-2-(2,2,2-trifluoroacetamido)butanoyl)-5,5-difluorooctahydrocyclopenta[c]pyrrole-1-carboxamide;
(1S,3aR,6aS)—N—((S)-1-cyano-2-((S)-2-oxopiperidin-3-yl)ethyl)-2-((2S)-3,3-dimethyl-2-(tetrahydrofuran-2-carboxamido)butanoyl)octahydrocyclopenta[c]pyrrole-1-carboxamide;
N—((S)-1-((1S,3aR,6aS)-1-(((S)-1-cyano-2-((S)-2-oxopiperidin-3-yl)ethyl)carbamoyl)hexahydrocyclopenta[c]pyrrol-2(1H)-yl)-3,3-dimethyl-1-oxobutan-2-yl)-5-(trifluoromethyl)isoxazole-3-carboxamide;
(1S,3aR,6aS)—N—((S)-1-cyano-2-((S)-2-oxopiperidin-3-yl)ethyl)-2-(O-(difluoromethyl)-N-(2,2,2-trifluoroacetyl)-L-threonyl)octahydrocyclopenta[c]pyrrole-1-carboxamide;
(1S,3aR,6aS)—N—((S)-1-cyano-2-((S)-2-oxopiperidin-3-yl)ethyl)-2-(N-(2,2,2-trifluoroacetyl)-O-(trifluoromethyl)-L-threonyl)octahydrocyclopenta[c]pyrrole-1-carboxamide;
(1S,3aR,6aS)—N—((S)-1-cyano-2-((S)-2-oxopiperidin-3-yl)ethyl)-2-(N-(2,2,2-trifluoroacetyl)-O-(trifluoromethyl)-L-seryl)octahydrocyclopenta[c]pyrrole-1-carboxamide;
(1S,3aR,6aS)—N—((S)-1-cyano-2-((S)-2-oxopiperidin-3-yl)ethyl)-2-(O-(difluoromethyl)-N-(2,2,2-trifluoroacetyl)-L-seryl)octahydrocyclopenta[c]pyrrole-1-carboxamide;
(1S,3aR,6aS)—N—((S)-1-cyano-2-((S)-2-oxopiperidin-3-yl)ethyl)-2-((S)-4,4,4-trifluoro-2-(2,2,2-trifluoroacetamido)-3-(trifluoromethyl)butanoyl)octahydrocyclopenta[c]pyrrole-1-carboxamide;
(1S,3aR,6aS)-2-((S)-2-acetamido-4,4,4-trifluoro-3-(trifluoromethyl)butanoyl)-N—((S)-1-cyano-2-((S)-2-oxopiperidin-3-yl)ethyl)octahydrocyclopenta[c]pyrrole-1-carboxamide;
(1S,3aR,6aS)—N—((S)-1-cyano-2-((S)-2-oxopiperidin-3-yl)ethyl)-2-((S)-3-(3,3-difluoroazetidin-1-yl)-2-(2,2,2-trifluoroacetamido)propanoyl)octahydrocyclopenta[c]pyrrole-1-carboxamide;
(1S,3aR,6aS)—N-((1S)-1-cyano-2-(2-oxopiperidin-3-yl)ethyl)-2-((1S,2R)-2-(2,2,2-trifluoroacetamido)-[1,1′-bi(cyclopropane)]-2-carbonyl)octahydrocyclopenta[c]pyrrole-1-carboxamide;
(1S,3aR,6aS)—N—((S)-1-cyano-2-((S)-2-oxopiperidin-3-yl)ethyl)-2-((S)-2-(1-methylcyclopropyl)-2-(2,2,2-trifluoroacetamido)acetyl)octahydrocyclopenta[c]pyrrole-1-carboxamide;
1,1,1-trifluoro-2-methylpropan-2-yl ((S)-1-((1S,3aR,6aS)-1-(((S)-1-cyano-2-((S)-2-oxopiperidin-3-yl)ethyl)carbamoyl)hexahydrocyclopenta[c]pyrrol-2(1H)-yl)-3,3-dimethyl-1-oxobutan-2-yl)carbamate;
(1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-3-azaspiro[bicyclo[3.1.0]hexane-6,1′-cyclopropane]-2-carboxamide;
N¹-(2-fluorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(o-tolyl)oxalamide;
N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide;
(1S,3aR,6aS)—N—((S)-4-hydroxy-3-oxo-1-((S)-2-oxopiperidin-3-yl)butan-2-yl)-2-(2-oxo-2-(o-tolylamino)acetyl)octahydrocyclopenta[c]pyrrole-1-carboxamide;
(1S,3aR,6aS)—N—((R)-4-hydroxy-3-oxo-1-((S)-2-oxopiperidin-3-yl)butan-2-yl)-2-(2-oxo-2-(o-tolylamino)acetyl)octahydrocyclopenta[c]pyrrole-1-carboxamide;
(S)-5-(2-((2-fluorophenyl)amino)-2-oxoacetyl)-N—((S)-4-hydroxy-3-oxo-1-((S)-2-oxopyrrolidin-3-yl)butan-2-yl)-5-azaspiro[2.4]heptane-6-carboxamide;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(3-(trifluoromethoxy)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
N¹-(3-fluorobicyclo[1.1.1]pentan-1-yl)-N²—((S)-1-(((S)-4-hydroxy-3-oxo-1-((S)-2-oxopyrrolidin-3-yl)butan-2-yl)amino)-3-(1-methylcyclobutyl)-1-oxopropan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethoxy)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(3-(trifluoromethyl)phenyl)oxalamide;
N¹-(2-(tert-butyl)phenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(4-(trifluoromethoxy)phenyl)oxalamide;
N¹-(3-chlorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl 3-methylbutanoate;
(S)-3-((S)-2-(2-((2-fluorophenyl)amino)-2-oxoacetamido)-4-methylpentanamido)-2-oxo-4-((S)-2-oxopiperidin-3-yl)butyl 3-methylbutanoate;
(S)-3-((S)-2-(2-((2-fluorophenyl)amino)-2-oxoacetamido)-4-methylpentanamido)-2-oxo-4-((S)-2-oxopiperidin-3-yl)butyl acetate;
(S)-3-((S)-2-(2-((2-fluorophenyl)amino)-2-oxoacetamido)-4-methylpentanamido)-2-oxo-4-((S)-2-oxopyrrolidin-3-yl)butyl acetate;
(S)-3-((S)-2-(2-((2-fluorophenyl)amino)-2-oxoacetamido)-4-methylpentanamido)-2-oxo-4-((S)-2-oxopiperidin-3-yl)butyl isopropyl carbonate;
(S)-3-((S)-2-(2-((2-fluorophenyl)amino)-2-oxoacetamido)-4-methylpentanamido)-2-oxo-4-((S)-2-oxopyrrolidin-3-yl)butyl isopropyl carbonate;
(S)-2-((S)-2-(2-((4-fluorobicyclo[2.2.2]octan-1-yl)amino)-2-oxoacetamido)-4-methylpentanamido)-3-((S)-2-oxopiperidin-3-yl)propanoic acid;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(4-fluorobicyclo[2.2.2]octan-1-yl)oxalamide;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(6-(trifluoromethyl)pyridin-2-yl)oxalamide;
N¹-(tert-butyl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
N¹-(tert-butyl)-N²—((S)-3-cyclopropyl-1-(((S)-4-hydroxy-3-oxo-1-((S)-2-oxopiperidin-3-yl)butan-2-yl)amino)-1-oxopropan-2-yl)oxalamide;
N¹-(tert-butyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)pyridin-3-yl)oxalamide;
(S)—N—((S)-1-cyano-2-((S)-2-oxopyrrolidin-3-yl)ethyl)-5-(4-methylpentanoyl)-5-azaspiro[2.4]heptane-6-carboxamide;
(S)-3-((S)-2-(1H-indole-2-carboxamido)-4-methylpentanamido)-2-oxo-4-((S)-2-oxopyrrolidin-3-yl)butyl heptanoate;
(S)-3-((S)-2-(4-methoxy-1H-indole-2-carboxamido)-4-methylpentanamido)-2-oxo-4-((S)-2-oxopyrrolidin-3-yl)butyl heptanoate;
(S)-3-((S)-2-((((3-chlorobenzyl)oxy)carbonyl)amino)-4-methylpentanamido)-2-oxo-4-((S)-2-oxopyrrolidin-3-yl)butyl isobutyrate;
(S)-3-((S)-2-((((3-chlorobenzyl)oxy)carbonyl)amino)-4-methylpentanamido)-2-oxo-4-((S)-2-oxopyrrolidin-3-yl)butyl 2-phenylacetate;
3-chlorobenzyl ((S)-1-(((S)-4-hydroxy-3-oxo-1-((S)-2-oxopyrrolidin-3-yl)butan-2-yl)amino)-4-methyl-1-oxopentan-2-yl)carbamate;
(S)-3-((S)-2-(4-methoxy-1H-indole-2-carboxamido)-4-methylpentanamido)-2-oxo-4-((S)-2-oxopyrrolidin-3-yl)butyl 1-cyclohexylpiperidine-4-carboxylate;
(S)-3-((S)-2-(4-methoxy-1H-indole-2-carboxamido)-4-methylpentanamido)-2-oxo-4-((S)-2-oxopyrrolidin-3-yl)butyl 4-(piperidin-1-yl)cyclohexane-1-carboxylate;
N—((S)-3-cyclopropyl-1-oxo-1-(((S)-1-oxo-3-((S)-2-oxopyrrolidin-3-yl)propan-2-yl)amino)propan-2-yl)-5-(2-fluorophenyl)isoxazole-3-carboxamide;
5-(2-fluorophenyl)-N—((S)-4-methyl-1-oxo-1-(((S)-1-oxo-3-((S)-2-oxopiperidin-3-yl)propan-2-yl)amino)pentan-2-yl)isoxazole-3-carboxamide;
N—((S)-4-methyl-1-oxo-1-(((S)-1-oxo-3-((S)-2-oxopiperidin-3-yl)propan-2-yl)amino)pentan-2-yl)-5-(pyridin-2-yl)isoxazole-3-carboxamide;
N—((S)-4-methyl-1-oxo-1-(((S)-1-oxo-3-((S)-2-oxopyrrolidin-3-yl)propan-2-yl)amino)pentan-2-yl)-5-(pyridin-2-yl)isoxazole-3-carboxamide;
N—((S)-1-(((S)-4-hydroxy-3-oxo-1-((S)-2-oxopiperidin-3-yl)butan-2-yl)amino)-4-methyl-1-oxopentan-2-yl)-4-methoxy-1H-indole-2-carboxamide;
(1R,2S,5S)—N—((S)-1-cyano-2-((S)-2-oxopiperidin-3-yl)ethyl)-6,6-dimethyl-3-(2-(trifluoromethyl)isonicotinoyl)-3-azabicyclo[3.1.0]hexane-2-carboxamide;
(1R,2S,5S)-3-(2-acetamido-5,5,5-trifluoro-4-methylpentanoyl)-N—((S)-1-cyano-2-((S)-2-oxopiperidin-3-yl)ethyl)-6,6-dimethyl-3-azabicyclo[3.1.0]hexane-2-carboxamide;
(1R,2S,5S)-3-((S)-3,3-dimethyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide;
N¹—((S)-1-(((R)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
(R)—N—((S)-3-(3-fluorophenyl)-1-(((S)-4-hydroxy-3-oxo-1-((S)-2-oxopiperidin-3-yl)butan-2-yl)amino)-1-oxopropan-2-yl)-2-hydroxy-4-methylpentanamide;
N¹-(4-fluorobicyclo[2.2.2]octan-1-yl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
(S)—N—((S)-1-cyano-2-((S)-2-oxopiperidin-3-yl)ethyl)-4,4-dimethyl-2-(4-methylpentanamido)pentanamide;
(S)-3-((S)-2-(4-methoxy-1H-indole-2-carboxamido)-4-methylpentanamido)-2-oxo-4-((S)-2-oxopyrrolidin-3-yl)butyl pentadecanoate; and
(S)-3-((S)-2-(2-((2-fluorophenyl)amino)-2-oxoacetamido)-4-methylpentanamido)-2-oxo-4-((S)-2-oxopiperidin-3-yl)butyl 2-cyanobenzoate, or a pharmaceutically acceptable salt thereof.

In some embodiments, the compound or pharmaceutically acceptable salt thereof may demonstrate an EC50 value (e.g., in Hela cells) of less than 0.05 μM. For example, the compound may be selected from the group consisting of:
N¹-(2,6-diethylphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2,6-diethylphenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
(1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide;
N¹-(2-fluorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(o-tolyl)oxalamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(3-(trifluoromethoxy)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethoxy)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(3-(trifluoromethyl)phenyl)oxalamide;
N¹-(2-(tert-butyl)phenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹-(3-chlorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopiperidin-3-yl)butyl isopropyl carbonate;
(S)-3-((S)-2-(1H-indole-2-carboxamido)-4-methylpentanamido)-2-oxo-4-((S)-2-oxopyrrolidin-3-yl)butyl heptanoate; and
(S)-3-((S)-2-(4-methoxy-1H-indole-2-carboxamido)-4-methylpentanamido)-2-oxo-4-((S)-2-oxopyrrolidin-3-yl)butyl heptanoate, or a pharmaceutically acceptable salt thereof.

In some embodiments, the compound or pharmaceutically acceptable salt thereof may demonstrate an EC50 value (e.g., in Hela cells) of from 0.05 μM to less than 0.2 μM. For example, the compound may be selected from the group consisting of:
N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl 1-cyclohexylpiperidine-4-carboxylate;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(3-methoxyphenyl)oxalamide;
(S)-3-((S)-2-(2-((2,6-dimethylphenyl)amino)-2-oxoacetamido)-4-methylpentanamido)-2-oxo-4-((S)-2-oxopyrrolidin-3-yl)butyl 2-oxo-2-phenylacetate;
(S)-3-((S)-2-(2-(cyclohexylamino)-2-oxoacetamido)-4-methylpentanamido)-2-oxo-4-((S)-2-oxopyrrolidin-3-yl)butyl heptanoate;
N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-3-cyclohexyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-3-cyclohexyl-1-(((R)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-1-(((S)-4-hydroxy-3-oxo-1-((S)-2-oxopiperidin-3-yl)butan-2-yl)amino)-4-methyl-1-oxopentan-2-yl)-N²-(o-tolyl)oxalamide;
(1S,3aR,6aS)-2-(2-((2-fluorophenyl)amino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide;
(1S,3aR,6aS)-2-(2-((2-fluorophenyl)amino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
N¹—((S)-4-methyl-1-oxo-1-(((S)-1-oxo-3-((S)-2-oxopyrrolidin-3-yl)propan-2-yl)amino)pentan-2-yl)-N²-(o-tolyl)oxalamide;
N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide;
N¹—((S)-3-cyclohexyl-1-oxo-1-(((S)-1-oxo-3-((S)-2-oxopyrrolidin-3-yl)propan-2-yl)amino)propan-2-yl)-N²-(2-fluorophenyl)oxalamide;
N¹-(2-fluorophenyl)-N²—((S)-4-methyl-1-oxo-1-(((S)-1-oxo-3-((S)-2-oxopiperidin-3-yl)propan-2-yl)amino)pentan-2-yl)oxalamide;
(1S,3aR,6aS)-2-(2-(2,4-dichlorophenoxy)acetyl)-N—((S)-1-oxo-3-((S)-2-oxopiperidin-3-yl)propan-2-yl)octahydrocyclopenta[c]pyrrole-1-carboxamide;
(1R,2S,5S)—N—((S)-1-cyano-2-((S)-2-oxopyrrolidin-3-yl)ethyl)-3-((S)-4,4-dimethyl-2-(2,2,2-trifluoroacetamido)pentanoyl)-6,6-dimethyl-3-azabicyclo[3.1.0]hexane-2-carboxamide;
N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
(1S,3aR,6aS)—N—((S)-4-hydroxy-3-oxo-1-((S)-2-oxopiperidin-3-yl)butan-2-yl)-2-(2-oxo-2-(o-tolylamino)acetyl)octahydrocyclopenta[c]pyrrole-1-carboxamide;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹-(3-fluorobicyclo[1.1.1]pentan-1-yl)-N²—((S)-1-(((S)-4-hydroxy-3-oxo-1-((S)-2-oxopyrrolidin-3-yl)butan-2-yl)amino)-3-(1-methylcyclobutyl)-1-oxopropan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(4-(trifluoromethoxy)phenyl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl 3-methylbutanoate;
(S)-3-((S)-2-(2-((2-fluorophenyl)amino)-2-oxoacetamido)-4-methylpentanamido)-2-oxo-4-((S)-2-oxopiperidin-3-yl)butyl 3-methylbutanoate;
(S)-3-((S)-2-(2-((2-fluorophenyl)amino)-2-oxoacetamido)-4-methylpentanamido)-2-oxo-4-((S)-2-oxopiperidin-3-yl)butyl acetate;
(S)-3-((S)-2-(2-((2-fluorophenyl)amino)-2-oxoacetamido)-4-methylpentanamido)-2-oxo-4-((S)-2-oxopyrrolidin-3-yl)butyl acetate;
(S)-3-((S)-2-(2-((2-fluorophenyl)amino)-2-oxoacetamido)-4-methylpentanamido)-2-oxo-4-((S)-2-oxopyrrolidin-3-yl)butyl isopropyl carbonate;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
N¹-(tert-butyl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
N—((S)-3-cyclopropyl-1-oxo-1-(((S)-1-oxo-3-((S)-2-oxopyrrolidin-3-yl)propan-2-yl)amino)propan-2-yl)-5-(2-fluorophenyl)isoxazole-3-carboxamide;
5-(2-fluorophenyl)-N—((S)-4-methyl-1-oxo-1-(((S)-1-oxo-3-((S)-2-oxopiperidin-3-yl)propan-2-yl)amino)pentan-2-yl)isoxazole-3-carboxamide;
(1R,2S,5S)-3-(2-acetamido-5,5,5-trifluoro-4-methylpentanoyl)-N—((S)-1-cyano-2-((S)-2-oxopiperidin-3-yl)ethyl)-6,6-dimethyl-3-azabicyclo[3.1.0]hexane-2-carboxamide;
(S)-3-((S)-2-(4-methoxy-1H-indole-2-carboxamido)-4-methylpentanamido)-2-oxo-4-((S)-2-oxopyrrolidin-3-yl)butyl pentadecanoate; and
(S)-3-((S)-2-(2-((2-fluorophenyl)amino)-2-oxoacetamido)-4-methylpentanamido)-2-oxo-4-((S)-2-oxopiperidin-3-yl)butyl 2-cyanobenzoate,
   or a pharmaceutically acceptable salt thereof.

In some embodiments, the compound or pharmaceutically acceptable salt thereof may demonstrate an EC50 value (e.g., in Hela cells) of from 0.2 μM to less than 0.5 μM. For example, the compound may be selected from the group consisting of:
N—((S)-1-(((S)-4-hydroxy-3-oxo-1-((S)-2-oxopyrrolidin-3-yl)butan-2-yl)amino)-4-methyl-1-oxopentan-2-yl)-4-methoxy-1H-indole-2-carboxamide;
(S)-3-((S)-2-(2-((2-fluorophenyl)amino)-2-oxoacetamido)-4-methylpentanamido)-2-oxo-4-((S)-2-oxopyrrolidin-3-yl)butyl pentadecanoate;
(S)-3-((S)-2-(2-((2-fluorophenyl)amino)-2-oxoacetamido)-4-methylpentanamido)-2-oxo-4-((S)-2-oxopyrrolidin-3-yl)butyl 4-(piperidin-1-yl)cyclohexane-1-carboxylate;
N¹—((S)-3-cyclohexyl-1-(((S)-4-hydroxy-3-oxo-1-((S)-2-oxopyrrolidin-3-yl)butan-2-yl)amino)-1-oxopropan-2-yl)-N²-(o-tolyl)oxalamide;
N¹-(2,6-dimethylphenyl)-N²-((2S)-1-((4-hydroxy-3-oxo-1-((S)-2-oxopyrrolidin-3-yl)butan-2-yl)amino)-4-methyl-1-oxopentan-2-yl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2-methoxy-6-methylphenyl)oxalamide;
(S)-3-((S)-3-cyclohexyl-2-(2-(cyclohexylamino)-2-oxoacetamido)propanamido)-2-oxo-4-((S)-2-oxopyrrolidin-3-yl)butyl heptanoate;
N¹-(3-fluorobicyclo[1.1.1]pentan-1-yl)-N²—((S)-1-(((S)-4-hydroxy-3-oxo-1-((S)-2-oxopyrrolidin-3-yl)butan-2-yl)amino)-4,4-dimethyl-1-oxopentan-2-yl)oxalamide;
(1R,2S,5S)-6,6-dimethyl-N—((S)-1-oxo-3-((S)-2-oxopyrrolidin-3-yl)propan-2-yl)-3-(2-(4-(trifluoromethoxy)phenoxy)acetyl)-3-azabicyclo[3.1.0]hexane-2-carboxamide;
(S)-5-(2-((2-fluorophenyl)amino)-2-oxoacetyl)-N—((S)-4-hydroxy-3-oxo-1-((S)-2-oxopyrrolidin-3-yl)butan-2-yl)-5-azaspiro[2.4]heptane-6-carboxamide;
(S)-3-((S)-2-(4-methoxy-1H-indole-2-carboxamido)-4-methylpentanamido)-2-oxo-4-((S)-2-oxopyrrolidin-3-yl)butyl 1-cyclohexylpiperidine-4-carboxylate;
N—((S)-1-(((S)-4-hydroxy-3-oxo-1-((S)-2-oxopiperidin-3-yl)butan-2-yl)amino)-4-methyl-1-oxopentan-2-yl)-4-methoxy-1H-indole-2-carboxamide; and
(1R,2S,5S)-3-((S)-3,3-dimethyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide,
   or a pharmaceutically acceptable salt thereof.

In some embodiments, the compound or pharmaceutically acceptable salt thereof may demonstrate an EC50 value (e.g., in Hela cells) of from 0.5 μM to 1 μM. For example, the compound may be selected from the group consisting of:
N¹—((S)-1-(((S)-4-hydroxy-3-oxo-1-((S)-2-oxopyrrolidin-3-yl)butan-2-yl)amino)-4-methyl-1-oxopentan-2-yl)-N²-(1-(p-tolyl)cyclopropyl)oxalamide;
(1S,3aR,6aS)-2-(2-(tert-butylamino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
N¹-cyclohexyl-N²—((S)-4-methyl-1-oxo-1-(((S)-1-oxo-3-((S)-2-oxopyrrolidin-3-yl)propan-2-yl)amino)pentan-2-yl)oxalamide;
(1S,3aR,6aS)-2-(2-oxo-2-((1,1,1-trifluoro-2-methylpropan-2-yl)amino)acetyl)-N—((S)-1-oxo-3-((S)-2-oxopiperidin-3-yl)propan-2-yl)octahydrocyclopenta[c]pyrrole-1-carboxamide;
(1R,2S,5S)-3-(2-(cyclohexylamino)-2-oxoacetyl)-6,6-dimethyl-N—((S)-1-oxo-3-((S)-2-oxopiperidin-3-yl)propan-2-yl)-3-azabicyclo[3.1.0]hexane-2-carboxamide;
N¹—((S)-1-(((S)-1-cyano-2-((S)-2-oxopyrrolidin-3-yl)ethyl)amino)-4-methyl-1-oxopentan-2-yl)-N²-(2-fluorophenyl)oxalamide;
(1S,3aR,6aS)—N—((S)-1-cyano-2-((S)-2-oxopiperidin-3-yl)ethyl)-2-(2-((2-fluorophenyl)amino)-2-oxoacetyl)octahydrocyclopenta[c]pyrrole-1-carboxamide;
(1S,3aR,6aS)—N—((S)-1-cyano-2-((S)-2-oxopiperidin-3-yl)ethyl)-2-(2-(2,4-dichlorophenoxy)acetyl)octahydrocyclopenta[c]pyrrole-1-carboxamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(4-fluorobicyclo[2.2.2]octan-1-yl)oxalamide;
(S)-3-((S)-2-((((3-chlorobenzyl)oxy)carbonyl)amino)-4-methylpentanamido)-2-oxo-4-((S)-2-oxopyrrolidin-3-yl)butyl isobutyrate;
(S)-3-((S)-2-((((3-chlorobenzyl)oxy)carbonyl)amino)-4-methylpentanamido)-2-oxo-4-((S)-2-oxopyrrolidin-3-yl)butyl 2-phenylacetate;
3-chlorobenzyl ((S)-1-(((S)-4-hydroxy-3-oxo-1-((S)-2-oxopyrrolidin-3-yl)butan-2-yl)amino)-4-methyl-1-oxopentan-2-yl)carbamate;
(S)-3-((S)-2-(4-methoxy-1H-indole-2-carboxamido)-4-methylpentanamido)-2-oxo-4-((S)-2-oxopyrrolidin-3-yl)butyl 4-(piperidin-1-yl)cyclohexane-1-carboxylate;
N—((S)-4-methyl-1-oxo-1-(((S)-1-oxo-3-((S)-2-oxopiperidin-3-yl)propan-2-yl)amino)pentan-2-yl)-5-(pyridin-2-yl)isoxazole-3-carboxamide;
N—((S)-4-methyl-1-oxo-1-(((S)-1-oxo-3-((S)-2-oxopyrrolidin-3-yl)propan-2-yl)amino)pentan-2-yl)-5-(pyridin-2-yl)isoxazole-3-carboxamide;
(1R,2S,5S)—N—((S)-1-cyano-2-((S)-2-oxopiperidin-3-yl)ethyl)-6,6-dimethyl-3-(2-(trifluoromethyl)isonicotinoyl)-3-azabicyclo[3.1.0]hexane-2-carboxamide;
N¹—((S)-1-(((R)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
(R)—N—((S)-3-(3-fluorophenyl)-1-(((S)-4-hydroxy-3-oxo-1-((S)-2-oxopiperidin-3-yl)butan-2-yl)amino)-1-oxopropan-2-yl)-2-hydroxy-4-methylpentanamide; and
N¹-(4-fluorobicyclo[2.2.2]octan-1-yl)-N²—((S)-1-(((S)-4-hydroxy-3-oxo-1-((S)-2-oxopiperidin-3-yl)butan-2-yl)amino)-4-methyl-1-oxopentan-2-yl)oxalamide,
   or a pharmaceutically acceptable salt thereof.

### Pharmaceutical Composition and Utility Thereof

The disclosure also provides a pharmaceutical composition comprising a therapeutically effective amount of one or more compounds according to Formula (I) or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The present disclosure, in providing Formula (I) compounds exhibiting high antiviral potencies, also provides a method for inhibiting the main protease (M^{pro}) of severe acute respiratory syndrome Coronavirus-2 (SARS-CoV-2), comprising contacting M^{pro} with a Formula (I) compound or pharmaceutically acceptable thereof. The contacting can occur in vivo, such as a in a host organism, or it can occur in vitro or ex vivo. In an additional embodiment, a compound or pharmaceutically acceptable salt as described herein is useful in a method for treating COVID-19 in a subject by administering to the subject the compound or salt by any administration route described herein. In an embodiment, the administration is by oral dosing. The method also is useful in a prophylaxis regimen for preventing a subject from developing COVID-19, such as in compromised subject populations, where viral loads are high, or a combination thereof.

The following non-limiting examples illustrate additional embodiments of the present disclosure.

### EXAMPLES

Abbreviations: ACN for acetonitrile; aq. for aqueous; Boc for tert-butoxycarbonyl; BOP for (benzotriazol-1-yloxy)tris(dimethylamino)phosphonium hexafluorophosphate; Bu for butyl; calcd. for calculated; conc. or con. for concentrated; DAST for diethylaminosulfur trifluoride; DBU for 2,3,4,6,7,8,9,10-octahydropyrimido[1,2-a]azepine; DCC for N,N′-dicyclohexylcarbodiimide; DCM for dichloromethane or methylene chloride; DIPEA for diisopropylethylamine; DMF for N,N-dimethylformamide; DMSO for dimethyl sulfoxide; DMF for N,N-dimethylformamide; DMP for Dess-Martin periodinane reagent; EDCI for N-(3-dimethylaminopropyl)-N′-ethylcarbodiimide hydrochloride; eq. or equiv. for equivalent or equivalents; Et for ethyl; Et₂O for diethyl ether; EtOAc for ethyl acetate; h for hour or hours; HATU for (1-[bis(dimethylamino)methylene]-1H-1,2,3-triazolo[4,5-b]pyridinium 3-oxide hexafluorophosphate; HBTU for N,N,N′,N′-tetramethyl-O-(1H-benzotriazol-1-yl)uronium hexafluorophosphate; IPA for isopropyl alcohol; LCMS for liquid chromatography-mass spectrometry; MeOH for methanol; MS for mass spectrometry; NMM for N-methylmorpholine; NMR for nuclear magnetic resonance; min for minute or minutes; PyBOP for benzotriazole-1-yl-oxy-tris-pyrrolidino-phosphonium hexafluorophosphate; R_{f} for retention factor; rt or RT for room temperature (ambient temperature); sat. for saturated; SFC for supercritical fluid chromatography; soln. for solution; t-Bu for tert-butyl; TEA for triethylamine; TLC for thin-layer chromatography; THE for tetrahydrofuran, UPLC for ultra-performance liquid chromatography; and V for volume.

General Synthetic Schemes

Compounds of Formula (I) are synthesized according to the following general schemes or its variations familiar to those skilled in the art, or by adaptation of, and as exemplified in the specific synthesis examples that follow:

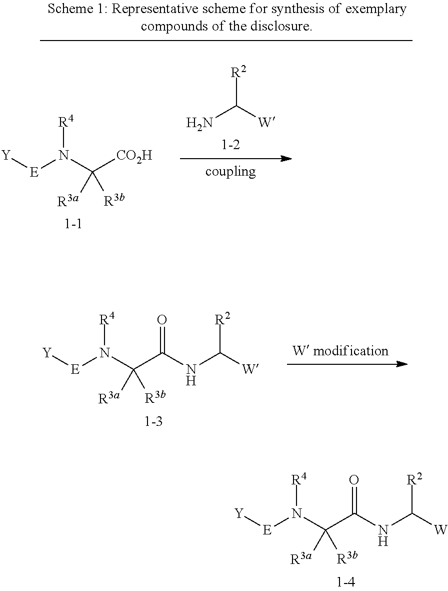

As shown in Scheme 1, compounds of formula (1-4) can be prepared from compounds of formula (1-1). Carboxylic acid (1-1) or its corresponding acid chloride or active ester prepared from readily available starting materials according to steps familiar to those skilled in the art, can be coupled with amine (1-2) or a corresponding salt with standard peptide coupling agents such as HATU, EDCI, DCC, BOP, HBTU, and PyBOP, wherein Y, E, R², R^{3a}, R^{3b} and R⁴ are as described in the Summary and W′ is either as W is described in the Summary or a moiety that can be transformed to W using procedures known to one of skill in the art to give compound of formula (1-3). Modification of W′ in compounds of formula (1-3) using synthetic methodologies know to one of skill in the art to convert W′ to W provides compounds of formula (1-4).

Compound of formula (1-4) and compounds of formula (1-3), wherein W′ is W, are representative of compounds of Formula (I).

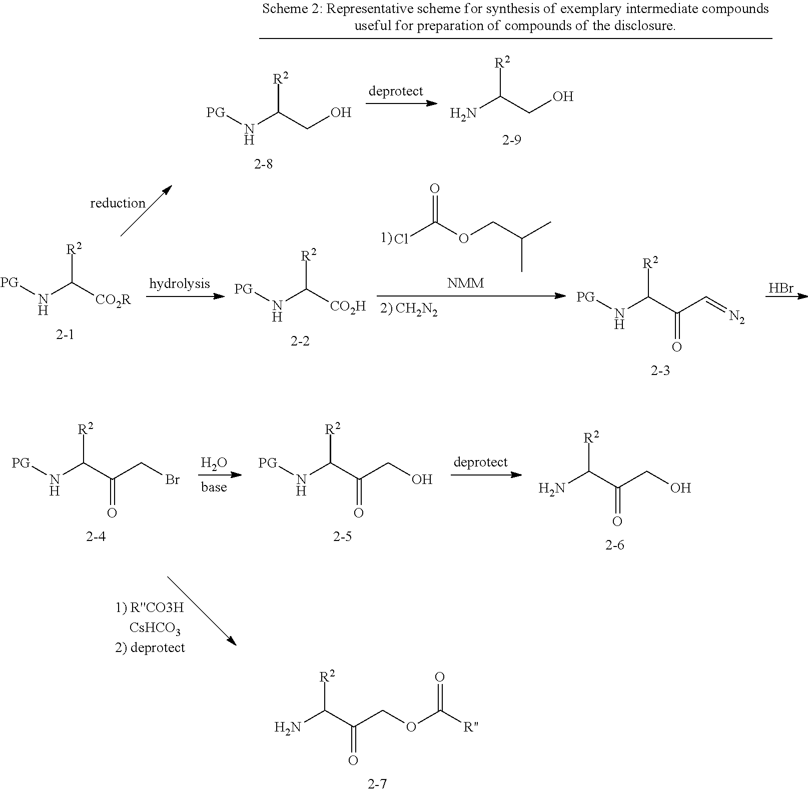

As shown in Scheme 2, compounds of formula (2-6), compounds of formula (2-7), and compounds of formula (2-9) can be prepared from compounds of formula (2-1). Compounds of formula (2-1), wherein R is C₁-C₄-alkyl and PG is an amine protecting group, can be hydrolyzed to give compounds of formula (2-2). Compounds of formula (2-2) can be reacted with isobutyl chloroformate in the presence of a tertiary amine base followed in a second reaction by treatment with diazomethane to give compounds of formula (2-3). Compounds of formula (2-3) can be treated with an acid, such as hydrobromic acid, to give compounds of formula (2-4). Compounds of formula (2-4) can be reacted with water in the presence of a base such as sodium bicarbonate or cesium bicarbonate to give compounds of formula (2-5). Compounds of formula (2-5) can be deprotected using methodologies know to one of skill in the art and dependent upon the amine protecting group to give compounds of formula (2-6). Compounds of formula (2-4) can also be reacted with R″CO₂H in a first reaction, wherein R″ is R⁵ or —C(O)R⁵, wherein R⁵ is as described in the Summary, in the presence of a base such as cesium bicarbonate and then deprotected in a second reaction to give compounds of formula (2-7).

Compounds of formula (2-1) can be reduced with a reductant such as lithium borohydride to give compounds of formula (2-8). Compounds of formula (2-8) can be deprotected to give compounds of formula (2-9).

Compounds of formula (2-5), compounds of formula (2-6), compounds of formula (2-7), compounds of formula (2-8), and compounds of formula (2-9) are intermediates useful in the preparation of compounds of Formula (I).

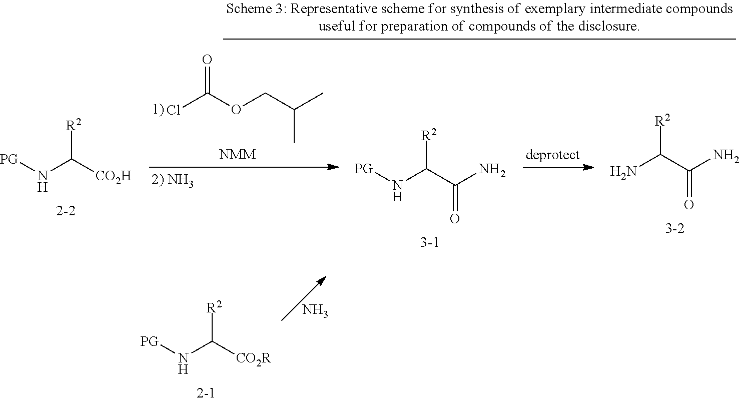

As shown in Scheme 3, compounds of formula (3-1) and compounds of formula (3-2) can be prepared from compounds of formula (2-2). Compounds of formula (2-2) can first be reacted with isobutyl chloroformate in the presence of a tertiary amine base, such as 4-methylmorpholine and then in a second reaction with ammonia to give compounds of formula (3-1). Compounds of formula (3-1) can be deprotected using methods known to one of skill in the art and dependent upon the particular amine protecting group to give compounds of formula (3-2).

Alternatively, compounds of formula (2-1), wherein R is a C₁-C₄-alkyl group, can be reacted with ammonia to give compounds of formula (3-1). As described above, compounds of formula (3-1) can be deprotected to give compounds of formula (3-2).

Compounds of formula (3-1) and formula (3-2) are intermediates useful in the preparation of compounds of Formula (I).

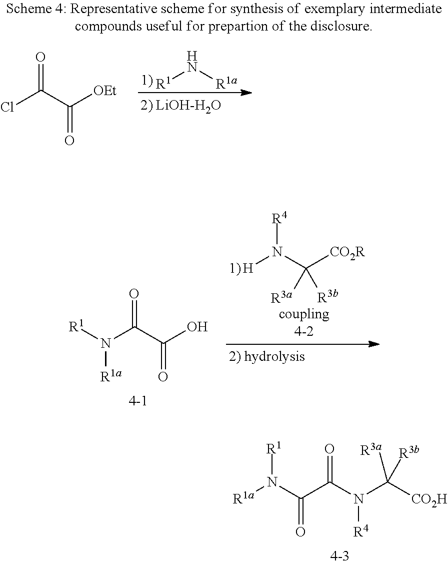

As shown in Scheme 4, compounds of formula (4-3) can be prepared from methyl or ethyl 2-chloro-2-oxoacetate. Accordingly, methyl or ethyl 2-chloro-2-oxoacetate can be reacted first with an amine, (R¹)(R²)NH, and then in a second reaction hydrolyzed with an alkoxide base such as lithium hydroxide to give compounds of formula (4-1). Compounds of formula (4-1) can be coupled with amino esters of formula (4-2) or a corresponding salt using standard peptide coupling reagents described in Scheme 1. Subsequent ester hydrolysis provides compounds of formula (4-3).

Compounds of formula (4-3) are intermediates useful in the preparation of compounds of Formula (I).

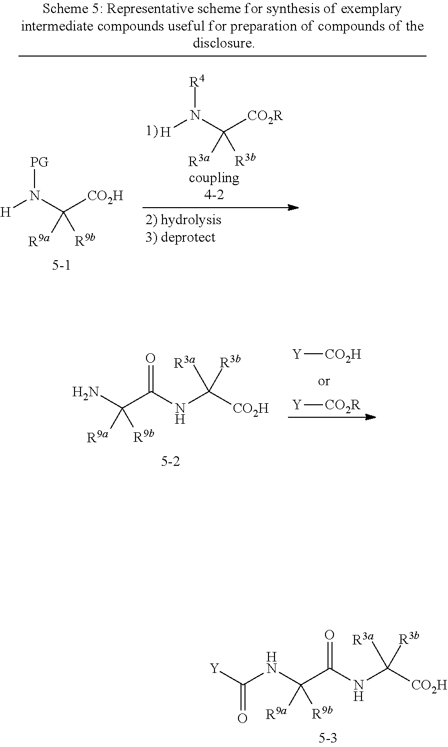

As shown in Scheme 5, compounds of formula (5-3) can be prepared beginning from compounds of formula (5-1). Compounds of formula (5-1) can be coupled using standard peptide coupling reagents with compounds of formula (4-2) or a corresponding salt. Subsequent ester hydrolysis and amine protecting group removal supplies compounds of formula (5-2). Compounds of formula (5-2) or a corresponding salt can be coupled with carboxylic acids, Y—CO₂H, using standard amide bond coupling reagents or reacted with amino esters, Y—CO₂R, in the presence of a base to give compound of formula (5-3).

Compounds of formula (5-3) are intermediates useful in the preparation of compounds of Formula (I).

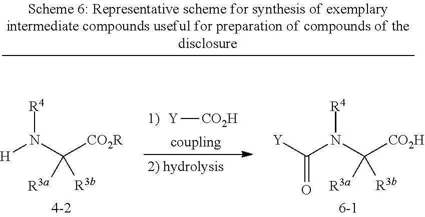

As shown in Scheme 6, compound of formula (6-1) can be prepared from compound of formula (4-2). Amino esters of formula (4-2) or a corresponding salt can be coupled with carboxylic acids, Y—CO₂H, using standard amide bond coupling reagents followed by ester hydrolysis to give compounds of formula (6-1).

Compounds of formula (6-1) are intermediates useful in the preparation of compounds of Formula (I).

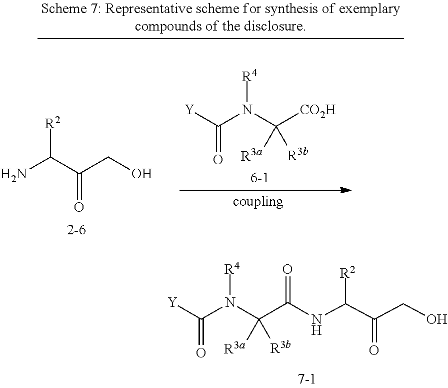

As shown in Scheme 7, compounds of formula (7-1) can be prepared from compounds of formula (2-6). Compound of formula (2-6) or a corresponding salt can be coupled with compounds of formula (6-1) using standard peptide coupling procedures to give compounds of formula (7-1).

Compounds of formula (7-1) are representative of compounds of Formula (I).

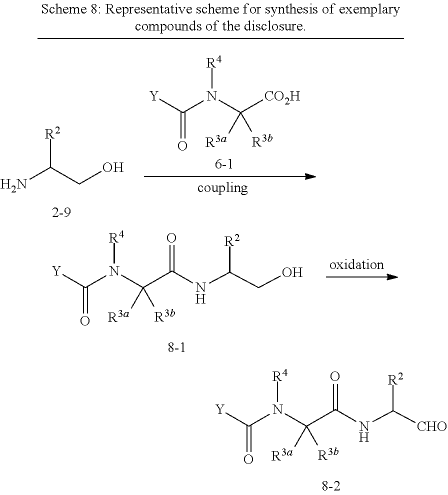

As shown in Scheme 8, compounds of formula (8-1) and compounds of formula (8-2) can be prepared from compounds of formula (2-9) or a corresponding salt. Compounds of formula (8-1) can be prepared from the coupling of compounds of formula (2-9) with compounds of formula (6-1) using standard amide bond coupling reagents and reaction conditions. Compound of formula (81) can be converted to compounds of formula (8-2) by treatment with a suitable oxidant such as Dess-Martin periodinane.

Compounds of formula (8-2) are representative of compounds of Formula (I).

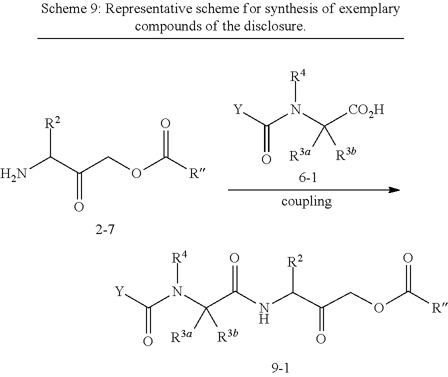

As shown in Scheme 9, compounds of formula (9-1) can be prepared from compounds of formula (2-7) or a corresponding salt. Compounds of formula (9-1) can be prepared from the coupling of compounds of formula (2-7) with compounds of formula (6-1) using standard amide bond coupling reagents and reaction conditions.

Compounds of formula (9-1) are representative of compounds of Formula (I).

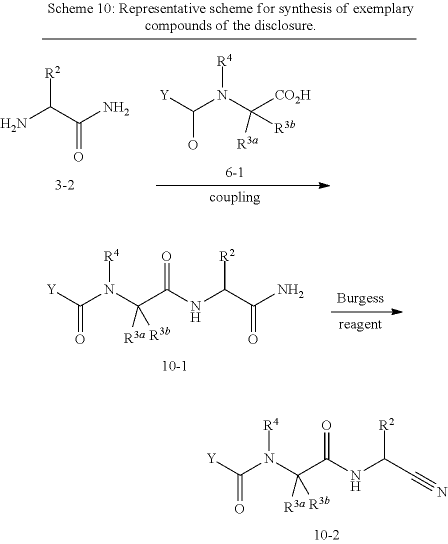

As shown in Scheme 10, compounds of formula (10-2) can be prepared from compounds of formula (3-2). Compound of formula (3-2) or a corresponding salt can be coupled with compounds of formula (6-1) using standard peptide coupling procedures to give compounds of formula (10-1). Compounds of formula (10-1) can be dehydrated using a reagent such as Burgess reagent to give compounds of formula (10-2).

Compounds of formula (10-2) are representative of compounds of Formula (I).

### Preparation of Intermediate Compounds

### Methyl (S)-2-((tert-butoxycarbonyl)amino)-3-((S)-2-oxopyrrolidin-3-yl)propanoate (1)

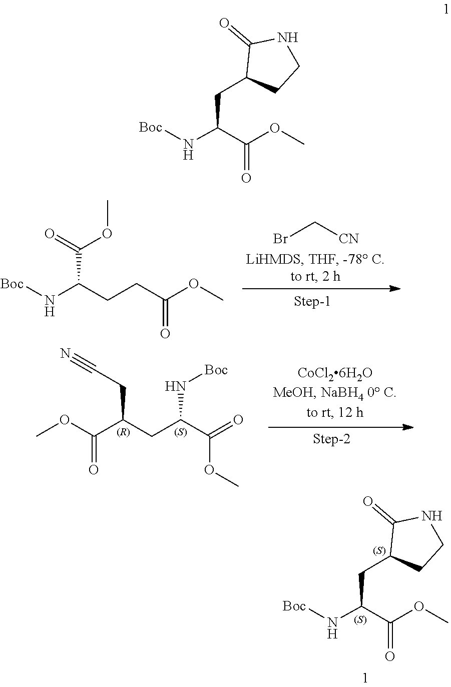

### Step 1: Dimethyl (2S,4R)-2-((tert-butoxycarbonyl)amino)-4-(cyanomethyl)pentanedioate

To the stirred solution of dimethyl (tert-butoxycarbonyl)-L-glutamate (100 g, 363.2 mol) in tetrahydrofuran (1000 mL), molecular sieves 4A (25 g) were added, and the resulting mixture was stirred at room temperature for 10 min. The reaction mixture was cooled to −78° C., lithium bis(trimethylsilyl)amide solution 1 M in THF (810 mL, 799 mol) was added, and the resulting mixture was stirred at −78° C. for 1.5 h. Bromoacetonitrile (65.36 g, 544.09 mol) was added to the above solution at −78° C. dropwise over 1 h, and the reaction mixture was stirred at −78° C. for 2 h. After completion of the reaction, the reaction mixture was quenched with methanol (50 mL) and stirred for 10 min at −78° C. The resulting solution was quenched with acetic acid (44 mL) in tetrahydrofuran (500 mL) and stirred for 10 min at −78° C. The cooling bath was removed and replaced with an ice cold water bath, and the reaction mixture was warmed up to 0-5° C. Brine solution (50 g NaCl in 500 mL water) was added. The organic layer was separated, and the aqueous part was extracted with tetrahydrofuran (2×500 mL). The combined organic layers were dried over anhydrous sodium sulfate and concentrated. The crude material was purified by silica-gel column chromatography eluted with 15% ethyl acetate in hexane to afford dimethyl (2S,4R)-2-((tert-butoxycarbonyl)amino)-4-(cyanomethyl)pentanedioate (80 g) as a pale yellow oil. [TLC system: EtOAc:petroleum ether (4:6); Rf: 0.4].

### Step 2: methyl (S)-2-((tert-butoxycarbonyl)amino)-3-((S)-2-oxopyrrolidin-3-yl)propanoate (1)

To a stirred solution dimethyl (2S,4R)-2-((tert-butoxycarbonyl)amino)-4-(cyanomethyl)pentanedioate (80.0 g, 254.5 mmol) in methanol (2000 mL), cobalt chloride (19.83 g, 152.7 mmol) was added at 0° C. Sodium borohydride (57.77 g, 1527 mmol) was added portionwise at 0° C. and the reaction mixture was stirred at room temperature for 24 h. It was quenched with saturated ammonium chloride solution (800 mL), filtered through a bed of diatomaceous earth that was washed with methanol (500 mL). The filtrate was concentrated under reduced pressure and the resulting aqueous layer was extracted with ethyl acetate (3×500 mL). The combined organic layers were dried over anhydrous sodium sulfate, concentrated under reduced pressure and purified by silica gel column chromatography eluted with 20% ethyl acetate in hexane to afford methyl (S)-2-((tert-butoxycarbonyl)amino)-3-((S)-2-oxopyrrolidin-3-yl)propanoate (1) (45 g) as a pale yellow semi solid. [TLC system: EtOAc:petroleum ether (7:3); Rf: 0.2]. LCMS m/z 287.42 (M+1); ¹H NMR (400 MHz, DMSO-d₆) δ ppm 5.86 (s, 1H), 5.49 (d, J=7.6 Hz, 1H), 4.32 (t, J=7.6 Hz, 1H), 3.74 (s, 3H), 3.37-3.33 (m, 2H), 2.47-2.46 (m, 2H), 2.17-2.11 (m, 1H), 1.87-1.68 (m, 2H). 1.44 (s, 9H).

### Methyl (S)-2-((tert-butoxycarbonyl)amino)-3-((S)-2-oxopiperidin-3-yl)propanoate (2)

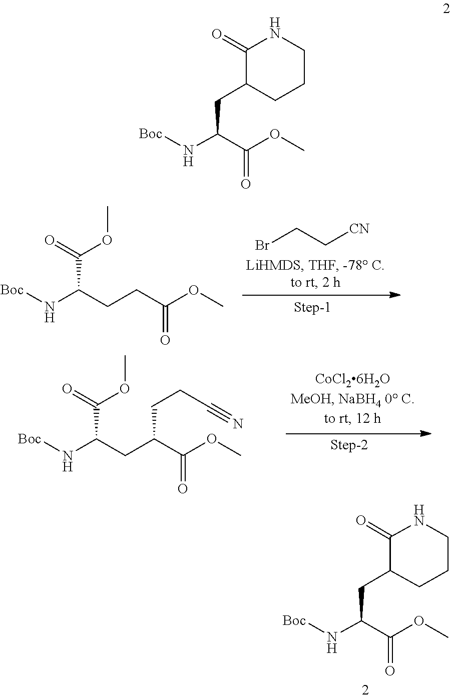

### Step 1. Dimethyl (2S,4S)-2-((tert-butoxycarbonyl)amino)-4-(2-cyanoethyl)pentanedioate

To the stirred solution of dimethyl (tert-butoxycarbonyl)-L-glutamate (25 g, 90.8 mol) in tetrahydrofuran (250 mL), molecular sieves 4A (10 g) were added, and the resulting mixture was stirred at room temperature for 10 min. The reaction mixture was cooled to −78° C. Lithium bis(trimethylsilyl)amide solution 1 M in THF (180 mL, 181.6 mol) was added, and the resulting mixture was stirred at −78° C. for 1.5 h. 3-Bromopropanenitrile (14.59 g, 108.9 mol) was added to the above solution at −78° C. dropwise over 1 h, and the reaction mixture was stirred at −78° C. for 2 h. After completion of the reaction, the reaction mixture was quenched with methanol (12.5 mL) and stirred for 10 min at −78° C. The resulting solution was quenched with acetic acid (11 mL) in tetrahydrofuran (125 mL) and stirred for 10 min at −78° C. The cooling bath was removed and replaced with ice cold water bath, and the reaction mixture was warmed to 0-5° C. Brine solution (12.5 g NaCl in 125 mL water) was added. The resulting mixture was filtered. The organic layer was separated, and the aqueous layer was extracted with tetrahydrofuran (3×125 mL). The combined organic layers were dried over anhydrous sodium sulfate and concentrated. The crude material was purified by silica gel column chromatography eluted with 20% ethyl acetate in hexane to afford dimethyl (2S,4S)-2-((tert-butoxycarbonyl)amino)-4-(2-cyanoethyl)pentanedioate (14.0 g) as a pale yellow oil. [TLC system: EtOAc:petroleum ether (4:6); R_{f}: 0.4].

### Step 2. Methyl (S)-2-((tert-butoxycarbonyl)amino)-3-((S)-2-oxopiperidin-3-yl)propanoate (2)

To a stirred solution dimethyl (2S,4S)-2-((tert-butoxycarbonyl)amino)-4-(2-cyanoethyl)pentanedioate (14.0 g, 42.6 mmol) in methanol (350 mL), cobalt chloride (3.32 g, 25.6 mmol) was added at 0° C. Sodium borohydride (9.67 g, 255.6 mmol) was added portionwise at 0° C., and the reaction mixture was stirred at room temperature for 24 h. After completion of the reaction, the reaction mixture was quenched with saturated ammonium chloride solution (140 mL), filtered through a bed of diatomaceous earth that was subsequently washed with methanol (200 mL). The filtrate was concentrated under reduced pressure to remove methanol. The resulting aqueous layer was extracted with ethyl acetate (3×500 mL). The combined organic layers were dried over anhydrous sodium sulfate, concentrated, and purified by silica gel column chromatography eluted with 20% ethyl acetate in hexane to afford methyl (S)-2-((tert-butoxycarbonyl)amino)-3-((S)-2-oxopiperidin-3-yl)propanoate (2) (11.0 g) as a pale yellow semi solid. [TLC system: EtOAc:petroleum ether (7:3); R_{f}: 0.2]. LCMS m/z 245.27 (M-t-Bu); ¹H NMR (400 MHz, DMSO-d₆) δ ppm 5.73 (s, 1H), 5.54 (d, J=8.4 Hz, 1H), 4.34-4.33 (m, 1H), 3.73 (s, 3H), 3.33-3.30 (m, 2H), 2.38-2.30 (m, 2H), 2.29-2.25 (m, 1H), 2.00-1.70 (m, 3H), 1.55-1.51 (m, 1H), 1.44 (s, 9H).

### tert-Butyl ((S)-4-hydroxy-3-oxo-1-((S)-2-oxopyrrolidin-3-yl)butan-2-yl)carbamate (5)

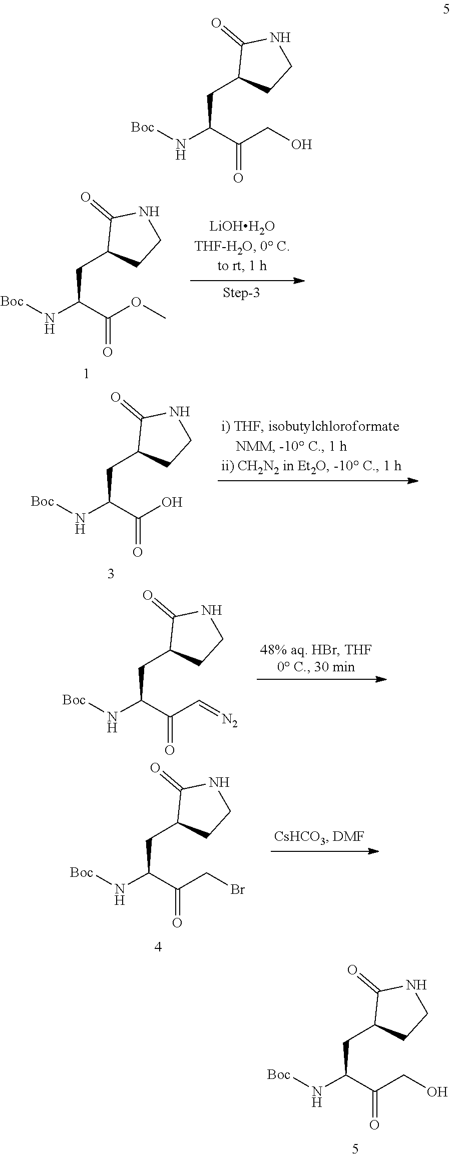

### Step 1. (S)-2-((tert-Butoxycarbonyl)amino)-3-((S)-2-oxopyrrolidin-3-yl)propanoic Acid (3)

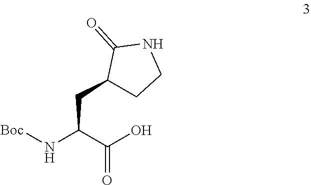 [SMILES: CC(C)(C)OC(=O)N[C@@H](C[C@@H]1CCNC1=O)C(=O)O]

To a stirred solution of methyl (S)-2-((tert-butoxycarbonyl)amino)-3-((S)-2-oxopyrrolidin-3-yl)propanoate (1) (14.5 g, 50.64 mmol) in tetrahydrofuran (50 mL), lithium hydroxide monohydrate (2.55 g, 60.77 mmol) in water (10 mL) was added at 0° C. and the reaction mixture was stirred at 0° C. for 1 h. After completion, the reaction mixture was acidified with 2 N hydrochloric acid to pH-5 and extracted with ethyl acetate (3×300 mL). The combined organic layers were dried over anhydrous sodium sulphate and concentrated under reduced pressure to afford (S)-2-((tert-butoxycarbonyl)amino)-3-((S)-2-oxopyrrolidin-3-yl)propanoic acid (11.0 g) as a pale yellow gum. TLC system: EtOAc:petroleum ether (7:3); R_{f}: 0.1.

### Step 2. tert-Butyl ((S)-4-diazo-3-oxo-1-((S)-2-oxopyrrolidin-3-yl)butan-2-yl)carbamate

To a stirred solution of (S)-2-((tert-butoxycarbonyl)amino)-3-((S)-2-oxopyrrolidin-3-yl)propanoic acid (3) (11.0 g, 36.72 mmol) in tetrahydrofuran (300 mL), N-methylmorpholine (4.83 g, 47.74 mmol) was added, then isobutyl chloroformate (10.18 g, 73.45 mmol) was added at −10° C., and the reaction mixture was stirred at −10° C. for 1 h. After completion, the reaction mixture was filtered and washed with tetrahydrofuran (50 mL). Freshly prepared diazomethane in diethyl ether (prepared from 5.0 mole equivalent of Diazald®) was added to the filtrate at −10° C. and the reaction mixture was stirred at room temperature for 1 h. After completion, the reaction mixture was diluted with water (100 mL) and extracted with ethyl acetate (3×300 mL). The combined organic layers were dried over anhydrous sodium sulphate and concentrated under reduced pressure to afford tert-butyl ((S)-4-diazo-3-oxo-1-((S)-2-oxopyrrolidin-3-yl)butan-2-yl)carbamate (10.8 g, crude) as a yellow liquid. TLC system: EtOAc:petroleum ether (7:3); R_{f}: 0.1.

### Step 3. tert-Butyl ((S)-4-bromo-3-oxo-1-((S)-2-oxopyrrolidin-3-yl)butan-2-yl)carbamate (4)

To a stirred solution of tert-butyl ((S)-4-diazo-3-oxo-1-((S)-2-oxopyrrolidin-3-yl)butan-2-yl)carbamate (10.8 g, 36.44 mmol) in tetrahydrofuran (300 mL) was added 48% aqueous hydrobromic acid (6.87 g, 43.73 mmol) dropwise at 0° C. and the mixture was stirred at 0° C. for 30 min. After completion, the reaction mixture was basified with saturated sodium bicarbonate and extracted with ethyl acetate (3×150 mL). The combined organic layers were dried over anhydrous sodium sulphate and concentrated under reduced pressure to afford tert-butyl ((S)-4-bromo-3-oxo-1-((S)-2-oxopyrrolidin-3-yl)butan-2-yl)carbamate (12.5 g, crude) as a yellow liquid. TLC system: EtOAc:petroleum ether (7:3); R_{f}: 0.4.

### Step 4. Synthesis of tert-butyl ((S)-4-hydroxy-3-oxo-1-((S)-2-oxopyrrolidin-3-yl)butan-2-yl)carbamate (5)

To the stirred solution of tert-butyl ((S)-4-bromo-3-oxo-1-((S)-2-oxopyrrolidin-3-yl)butan-2-yl)carbamate (4) (4.8 g, 13.8 mmol) and benzyl alcohol (2.97 g, 27.5 mmol) in dimethylformamide (50 mL), cesium bicarbonate (6.47 g, 41.4 mmol) was added and the reaction mixture was stirred at room temperature for 24 h. After completion, the reaction mixture was diluted with water (100 mL) and extracted with ethyl acetate (3×300 mL). The combined organic layers were dried over anhydrous sodium sulfate and concentrated under reduced pressure. The crude residue was then purified by silica gel (230-400 mesh) column chromatography using 5-7% MeOH in DCM as a gradient to afford tert-butyl ((S)-4-hydroxy-3-oxo-1-((S)-2-oxopyrrolidin-3-yl)butan-2-yl)carbamate (2.3 g) as an off-white solid. TLC system: EtOAc:petroleum ether (7:3); Rf: 0.5. Analytical Data: LCMS m/z 309.35 (M+Na); ¹H NMR (400 MHz, DMSO-d₆) δ ppm 7.63 (s, 1H), 7.36 (d, J=7.8 Hz, 1H), 5.07 (t, J=5.9 Hz, 1H), 4.27-4.08 (m, 3H), 3.14 (t, J=7.9 Hz, 2H), 2.24-2.08 (m, 2H), 1.81 (t, J=5.4 Hz, 1H), 1.68-1.57 (m, 2H), 1.38 (s, 9H).

The above transformation was carryout using tert-butyl ((S)-4-bromo-3-oxo-1-((S)-2-oxopyrrolidin-3-yl)butan-2-yl)carbamate (4) (200 mg) and cesium bicarbonate (3 eq.) in DMF (2 mL) in the absence of benzyl alcohol under similar reaction condition provided the same product (100 mg) of after purification.

### (S)-3-((S)-2-Amino-4-hydroxy-3-oxobutyl)pyrrolidin-2-one hydrochloride (6)

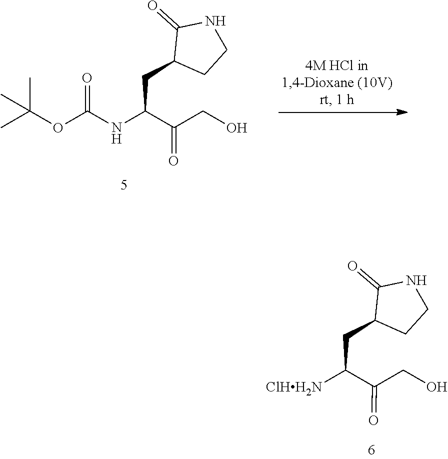

To a stirred solution of tert-butyl ((S)-4-hydroxy-3-oxo-1-((S)-2-oxopyrrolidin-3-yl)butan-2-yl)carbamate (5) (0.3 g, 1.048 mmol) in 1,4-dioxane (1.5 mL) was added 4 M HCl in 1,4-dioxane (0.42 mL, 4.19 mmol) at 0° C., and the resultant mixture was stirred at room temperature for 2 h. After completion, volatiles were removed under reduced pressure and the resulting crude product was washed with diethyl ether (2×5 mL) to afford ((S)-3-((S)-2-amino-4-hydroxy-3-oxobutyl)pyrrolidin-2-one hydrochloride (6) (230 mg) as a yellow solid. TLC system: MeOH:DCM (1:9); R_{f}. 0.1. LCMS m/z 187.28 (M+1).

### Synthesis of tert-butyl ((S)-4-hydroxy-3-oxo-1-((S)-2-oxopiperidin-3-yl)butan-2-yl)carbamate (8)

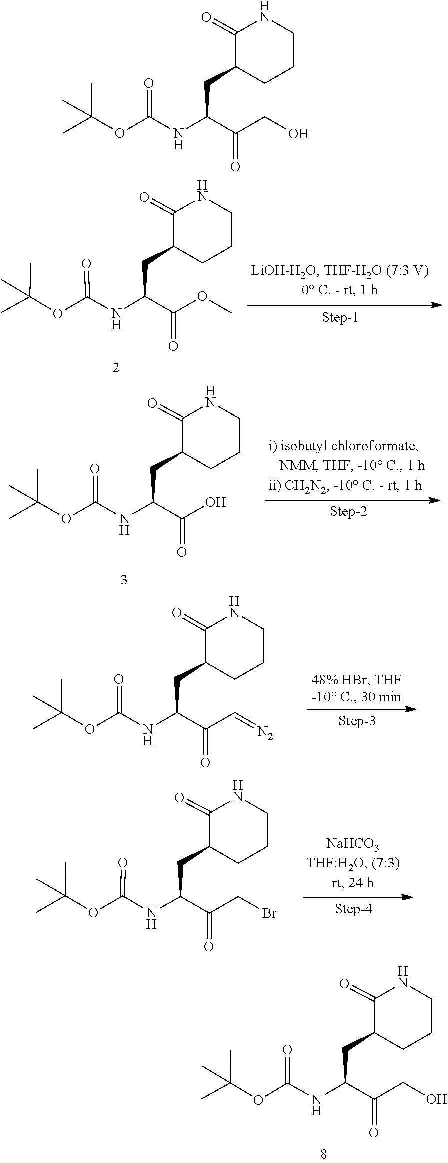

### Step 1. ((S)-2-((tert-butoxycarbonyl)amino)-3-((S)-2-oxopiperidin-3-yl)propanoic Acid

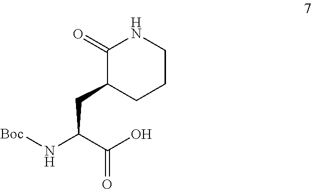 [SMILES: CC(C)(C)OC(=O)N[C@@H](C[C@@H]1CCCNC1=O)C(=O)O]

To a stirred solution of methyl (S)-2-((tert-butoxycarbonyl)amino)-3-((S)-2-oxopiperidin-3-yl)propanoate (2) (10 g, 33.33 mmol) in tetrahydrofuran (70 mL), lithium hydroxide monohydrate (1.68 g, 39.99 mmol) in water (30 mL) was added at 0° C. and the mixture was stirred for 1 h. After completion, the reaction mixture was cooled 0° C., acidified with 2 N hydrochloric acid (pH-5) and extracted with ethyl acetate (3×300 mL). The combined organic layers were dried over anhydrous sodium sulphate and concentrated under reduced pressure to afford ((S)-2-((tert-butoxycarbonyl)amino)-3-((S)-2-oxopiperidin-3-yl)propanoic acid (7) (8.0 g) as a pale yellow gum. TLC system: MeOH:DCM (1:9); R_{f}: 0.1.

### Step 2. Synthesis of tert-butyl ((S)-4-diazo-3-oxo-1-((S)-2-oxopiperidin-3-yl)butan-2-yl)carbamate

To a stirred solution of ((S)-2-((tert-butoxycarbonyl)amino)-3-((S)-2-oxopiperidin-3-yl)propanoic acid (7) (8.0 g, 29.96 mmol) in tetrahydrofuran (100 mL), N-methylmorpholine (6.58 mL, 59.93 mmol) was added, then isobutyl chloroformate (6.13 mL, 44.9 mmol) was added at −10° C., and the reaction mixture was stirred at −10° C. for 1 h. After completion, the reaction mixture was filtered and washed with tetrahydrofuran (50 mL). Freshly prepared diazomethane in diethyl ether (prepared from 4.0 eq. of Diazald®) was added to the filtrate at −10° C. and the reaction mixture was stirred at room temperature for 1 h. After completion, the reaction mixture was diluted with water (100 mL) and extracted with ethyl acetate (3×300 mL). The combined organic layers were dried over anhydrous sodium sulphate and concentrated under reduced pressure to afford tert-butyl ((S)-4-diazo-3-oxo-1-((S)-2-oxopiperidin-3-yl)butan-2-yl)carbamate (9.8 g, crude) as a yellow liquid. TLC system: EtOAc:petroleum ether (7:3); R_{f}: 0.2.

### Step 3. Synthesis of tert-butyl ((S)-4-bromo-3-oxo-1-((S)-2-oxopiperidin-3-yl)butan-2-yl)carbamate

To a stirred solution tert-butyl ((S)-4-diazo-3-oxo-1-((S)-2-oxopiperidin-3-yl)butan-2-yl)carbamate (9.8 g, 31.6 mmol) in tetrahydrofuran (100 mL) was added 48% aqueous hydrobromic acid (6.4 mL, 37.94 mmol) dropwise at −10° C. and the mixture was stirred at −10° C. for 30 min. After completion, the reaction mixture was basified with saturated sodium bicarbonate and extracted with ethyl acetate (3×150 mL). The combined organic layers were dried over anhydrous sodium sulphate and concentrated under reduced pressure. The crude residue was then purified by column chromatography over silica gel (230-400 mesh) using 50-70% ethyl acetate in petroleum ether as a gradient to afford tert-butyl ((S)-4-bromo-3-oxo-1-((S)-2-oxopiperidin-3-yl)butan-2-yl)carbamate (5.4 g) as a yellow liquid. TLC system: EtOAc: petroleum ether (7:3); R_{f}: 0.4.

### Step 4. Synthesis of tert-butyl ((S)-4-hydroxy-3-oxo-1-((S)-2-oxopiperidin-3-yl)butan-2-yl)carbamate

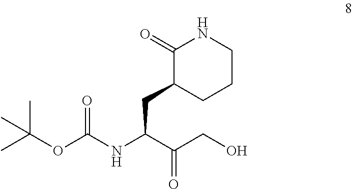 [SMILES: CC(C)(C)OC(=O)N[C@@H](C[C@@H]1CCCNC1=O)C(=O)CO]

To the stirred solution of tert-butyl ((S)-4-bromo-3-oxo-1-((S)-2-oxopiperidin-3-yl)butan-2-yl)carbamate (4.8 g, 14.92 mmol) in THF:H₂O (35:15 mL), sodium bicarbonate (6.26 g, 74.58 mmol) was added and the reaction mixture was stirred at room temperature for 24 h. After completion, the reaction mixture was diluted with water (100 mL) and extracted with ethyl acetate (3×150 mL). The combined organic layers were dried over anhydrous sodium sulfate and concentrated under reduced pressure. The crude residue was then purified by silica gel (230-400 mesh) column chromatography using 5-7% MeOH in DCM as a gradient to afford tert-butyl ((S)-4-hydroxy-3-oxo-1-((S)-2-oxopiperidin-3-yl)butan-2-yl)carbamate (8) (2.3 g) as an off-white solid. TLC system: EtOAc:petroleum ether (7:3); R_{f}. 0.5. Analytical Data: LCMS m/z 323.38 (M+Na); ¹H NMR (400 MHz, DMSO-d₆) δ ppm 7.63 (s, 1H), 7.36 (d, J=7.8 Hz, 1H), 5.07 (t, J=5.9 Hz, 1H), 4.22-4.13 (m, 3H), 3.14 (t, J=7.9 Hz, 2H), 2.19-2.08 (m, 1H), 2.05-1.86 (m, 2H), 1.77-1.74 (m, 1H), 1.64-1.58 (m, 2H), 1.38 (s, 10H).

### tert-Butyl ((S)-1-hydroxy-3-((S)-2-oxopyrrolidin-3-yl)propan-2-yl)carbamate (9)

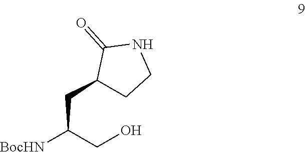 [SMILES: CC(C)(C)OC(=O)N[C@H](CO)C[C@@H]1CCNC1=O]

To a solution of methyl (S)-2-((tert-butoxycarbonyl)amino)-3-((S)-2-oxopyrrolidin-3-yl)propanoate (1) (6.0 g, 20.95 mmol) in THE (42 mL) was added LiBH₄ (2 M in THF, 26.18 mL, 52.38 mmol) dropwise at 0° C. followed by MeOH (18 mL). The reaction mixture was stirred at ambient temperature for 3 h. After completion of reaction, the reaction mixture was quenched with 1 N HCl solution (50 mL) and extracted with EtOAc (100 mL×2). The combined organic layers were washed with brine (50 mL) and dried over Na₂SO₄, filtered and concentrated under reduced pressure. The crude compound was purified by column chromatography over silica gel (100% EtOAc as an eluent) to afford 2.2 g of tert-butyl ((S)-1-hydroxy-3-((S)-2-oxopyrrolidin-3-yl)propan-2-yl)carbamate (9) as an off white solid. [TLC system: MeOH:DCM (1:9); R_{f} value: 0.1]. Analytical Data: LCMS m/z=281.30 [M+Na]; ¹H NMR (400 MHz, CDCl₃) δ ppm 5.61 (s, 1H), 5.41 (s, 1H), 3.75 (s, 1H), 3.66-3.63 (m, 2H), 3.60-3.57 (m, 2H), 3.02 (s, 1H), 2.53-2.41 (m, 2H), 1.97-1.89 (m, 2H), 1.86-1.85 (m, 1H), 1.44 (s, 9H).

### tert-Butyl ((S)-1-oxo-3-((S)-2-oxopyrrolidin-3-yl)propan-2-yl)carbamate (10)

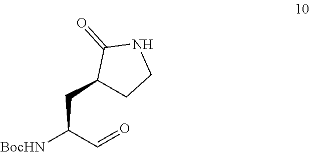 [SMILES: CC(C)(C)OC(=O)N[C@H](C=O)C[C@@H]1CCNC1=O]

To a stirred solution of tert-butyl ((S)-1-hydroxy-3-((S)-2-oxopyrrolidin-3-yl)propan-2-yl)carbamate (9) (9.0 g, 34.84 mmol) in DCM (180 mL), was added Dess-Martin reagent (22.1 g, 52.26 mmol) at −10° C. The reaction mixture was stirred at room temperature for 2 h. After completion of the reaction, the reaction mixture was quenched with saturated NaHSO₃ solution and extracted with DCM (2×250 mL). The combined organic fractions were washed with saturated NaHCO₃ solution and brine (50 mL), and dried over anhydrous Na₂SO₄, filtered and concentrated under reduced pressure. The crude compound was purified by column chromatography over silica gel (100% EtOAc as an eluent) to afford 2.65 g of tert-butyl ((S)-1-oxo-3-((S)-2-oxopyrrolidin-3-yl)propan-2-yl)carbamate (10) as an off white solid. [TLC system: 100% EtOAc; R_{f}value: 0.3]. Analytical Data: LCMS m/z=157.18 [M-Boc]; ¹H NMR (400 MHz, CDCl₃) δ ppm 9.57 (s, 1H), 6.09 (s, 1H), 5.55 (s, 1H), 4.19 (t, J=4.4 Hz, 1H), 3.41-3.34 (m, 2H), 2.50-2.44 (m, 2H), 2.03-1.86 (m, 3H), 1.44 (s, 9H).

### (S)-3-((S)-2-Amino-3-hydroxypropyl)pyrrolidin-2-one hydrochloride (11)

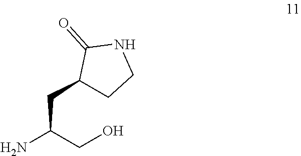 [SMILES: N[C@H](CO)C[C@@H]1CCNC1=O]

To a solution of tert-butyl ((S)-1-hydroxy-3-((S)-2-oxopyrrolidin-3-yl)propan-2-yl)carbamate (9) (0.45 g, 1.66 mmol) in dioxane (5 mL) was added 4 M HCl in dioxane (5 mL) at 0° C. and the resultant mixture was stirred at room temperature for 2 h. After completion, the volatiles were removed under reduced pressure to afford (S)-3-((S)-2-amino-3-hydroxypropyl)pyrrolidin-2-one hydrochloride (11) (0.4 g) as a brown gummy solid. TLC system: MeOH:hexane (1:9); R_{f}: 0.1. LCMS m/z=159.09 (M+1).

### tert-Butyl ((S)-1-amino-1-oxo-3-((S)-2-oxopyrrolidin-3-yl)propan-2-yl)carbamate (12)

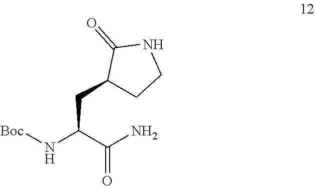 [SMILES: CC(C)(C)OC(=O)N[C@@H](C[C@@H]1CCNC1=O)C(N)=O]

To a stirred solution of (S)-2-((tert-butoxycarbonyl)amino)-3-((S)-2-oxopyrrolidin-3-yl)propanoic acid (1.0 g, 3.67 mmol) in tetrahydrofuran (10 mL), N-methylmorpholine (0.56 g, 5.51 mmol) was added followed by addition of isobutyl chloroformate (1.02 g, 7.34 mmol) at −10° C. and the reaction mixture was stirred at 10° C. for 1 h. After completion of the reaction, the reaction mixture was filtered and washed with tetrahydrofuran (10 mL). 25% Aqueous ammonia solution was added to the filtrate at 0° C. and the reaction mixture was stirred at room temperature for 2 h. After completion of the reaction, the reaction mixture was diluted with water (30 mL) and extracted with ethyl acetate (3×30 mL). The combined organic layers were dried over anhydrous sodium sulphate and concentrated under reduced pressure. The obtained crude residue was purified by Davisil® grade silica gel column chromatography using 8% methanol in dichloromethane to afford tert-butyl ((S)-1-amino-1-oxo-3-((S)-2-oxopyrrolidin-3-yl)propan-2-yl)carbamate (0.6 g, 60%) as an off-white solid. TLC system: MeOH:DCM (1:9); R_{f}: 0.2.

### (S)-2-Amino-3-((S)-2-oxopiperidin-3-yl)propanamide hydrochloride (13)

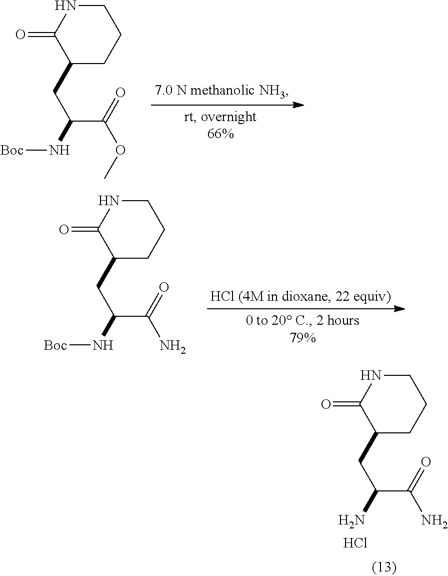

### Step 1. tert-Butyl ((S)-1-amino-1-oxo-3-((S)-2-oxopiperidin-3-yl)propan-2-yl)carbamate

Methyl (S)-2-((tert-butoxycarbonyl)amino)-3-((S)-2-oxopiperidin-3-yl)propanoate (1 equiv, 3.3 mmol, 1.0 g) was dissolved in 7.0 M methanolic ammonia (42 equiv, 140 mmol, 20 mL). The reaction was stirred overnight at room temperature after which the volatiles were removed under vacuum. The residue was purified by flash chromatography (SiO₂, graduate elution in CH₂Cl₂:CH₃OH 0→25%) to afford tert-butyl ((S)-1-amino-1-oxo-3-((S)-2-oxopiperidin-3-yl)propan-2-yl)carbamate as a white solid (650 mg, 66% yield). Analytical Data: LCMS m/z=186.28 (M-Boc)*; ¹H NMR (400 MHz, DMSO-d₆) δ ppm 7.45 (s, 1H), 7.23 (s, 1H), 6.95-6.89 (m, 2H), 3.94-3.89 (m, 1H), 3.11 (s, 2H), 2.10-2.02 (m, 2H), 1.92-1.88 (m, 1H), 1.75-1.74 (m, 1H), 1.55-1.37 (m, 12H).

### Step 2. (S)-2-Amino-3-((S)-2-oxopiperidin-3-yl)propanamide hydrochloride (13)

A solution of tert-butyl ((S)-1-amino-1-oxo-3-((S)-2-oxopiperidin-3-yl)propan-2-yl)carbamate (1 equiv. 2.2 mmol, 0.65 g) in dioxane (12 mL) was cooled to 0° C. and 4.0 M HCl in dioxane (22 equiv, 48.4 mmol, 12.0 mL) was added to the frozen reaction mixture. The reaction mixture was then warmed up to room temperature and was stirred for 2 hours after which the volatiles were removed under vacuum. The white residue was then washed with DCM (2×20 mL) and dried overnight under vacuum to afford of (S)-2-amino-3-((S)-2-oxopiperidin-3-yl)propanamide hydrochloride as a white solid (0.375 g, 79% yield.)

### Synthesis of 2-((2-fluorophenyl)amino)-2-oxoacetic Acid (14)

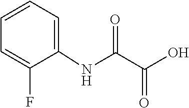 [SMILES: O=C(O)C(=O)Nc1ccccc1F]

### Step 1. Synthesis of ethyl 2-((2-fluorophenyl)amino)-2-oxoacetate

To a stirred solution of 2-fluoroaniline (3.0 g, 26.98 mmol) in DCM (30 mL) at 0° C. was added ethyl 2-chloro-2-oxoacetate (3.7 g, 28.33 mmol), then TEA (4.02 mL, 28.33 mmol) was added dropwise over 5 min, and the mixture was stirred at room temperature for 2 h. After completion, volatiles were removed through vacuum at 25° C. The obtained crude product was dissolved in diethyl ether (100 mL) and filtered through a pad of diatomaceous earth washed with diethyl ether (5×50 mL). The collected filtrate was dried over Na₂SO₄ and concentrated under reduced pressure to afford ethyl 2-((2-fluorophenyl)amino)-2-oxoacetate (4.0 g) as a colorless gummy liquid. TLC system: EtOAc:petroleum ether (3:7); R_{f}: 0.6.

### Step 2. Synthesis of 2-((2-fluorophenyl)amino)-2-oxoacetic Acid (14)

To a solution of ethyl 2-((2-fluorophenyl)amino)-2-oxoacetate (4.0 g, 20.29 mmol) in THE (30.0 mL) at 0° C., was added LiOH—H₂O (1.0 g, 24.35 mmol) in water (10 mL). The resultant mixture was stirred at room temperature for 1 h. After completion, the reaction mixture was acidified to pH ˜4 using 5% aq. HCl (40 mL) and the aqueous layer was extracted with ethyl acetate (2×70 mL). The combined organic layers were dried over anhydrous sodium sulfate, filtered and concentrated to yield 2-((2-fluorophenyl)amino)-2-oxoacetic acid (14) (3.2 g) as a colorless gummy liquid. TLC system: EtOAc:petroleum ether (7:3); R_{f}: 0.1.

### Synthesis of methyl (2-((2-fluorophenyl)amino)-2-oxoacetyl)-L-leucinate (15)

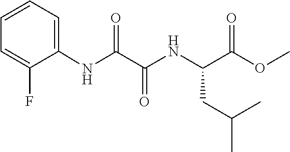 [SMILES: COC(=O)[C@H](CC(C)C)NC(=O)C(=O)Nc1ccccc1F]

To a solution of 2-((2-fluorophenyl)amino)-2-oxoacetic acid (14) (5.0 g, 27.2 mmol) in anhydrous DMF (40 mL) was added methyl L-leucinate hydrochloride (4.0 g, 27.2 mmol), HATU (13.47 g, 35.3 mmol) and DIPEA (14.2 mL, 81.6 mmol) at 0° C. The resultant mixture was stirred at room temperature for 2 h. After completion, the reaction mixture diluted with ethyl acetate (100 mL), washed with saturated aqueous NaHCO₃ (70 mL) followed by water (70 mL) and brine (40 mL). The organic layer was dried over anhydrous sodium sulfate, filtered, and concentrated under reduce pressure. The crude material was then purified by column chromatography over silica gel (230-400 mesh) (using 20-30% EtOAc in petroleum ether as an eluent) to afford methyl (2-((2-fluorophenyl)amino)-2-oxoacetyl)-L-leucinate (15) (4.0 g) as an off-white solid. TLC system: EtOAc:petroleum ether (3:7); R_{f}: 0.5.

### Synthesis of (2-((2-fluorophenyl)amino)-2-oxoacetyl)-L-leucine (16)

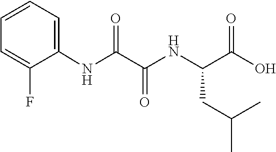 [SMILES: CC(C)C[C@H](NC(=O)C(=O)Nc1ccccc1F)C(=O)O]

To a solution of methyl (2-((2-fluorophenyl)amino)-2-oxoacetyl)-L-leucinate (15) (4.0 g, 12.9 mmol) in THE (30.0 mL) at 0° C., was added LiOH—H₂O (0.65 g, 15.48 mmol) in water (7.0 mL). The resultant mixture was stirred at room temperature for 1 h. After completion, the reaction mixture was acidified to pH ˜4 using 5% aq. HCl (20 mL) and the aqueous layer was extracted with ethyl acetate (2×70 mL). The combined organic layers were dried over anhydrous sodium sulfate, filtered and concentrated to yield (2-((2-fluorophenyl)amino)-2-oxoacetyl)-L-leucine (16) (3.2 g) as a colorless gummy liquid. TLC system: EtOAc:petroleum ether (7:3); R_{f}: 0.1. LCMS m/z=295.24 (M−H)⁻; ¹H NMR (400 MHz, DMSO-d₆) δ ppm 12.81 (s, 1H), 10.30 (s, 1H), 9.09 (d, J=8.3 Hz, 1H), 7.69-7.65 (m, 1H), 7.34-7.25 (m, 3H), 4.34-4.29 (m, 1H), 1.83 (t, J=10.5 Hz, 1H), 1.59-1.56 (m, 2H), 0.91-0.87 (m, 6H).

### Synthesis of 2-((4,4-difluorocyclohexyl)amino)-2-oxoacetic Acid (17)

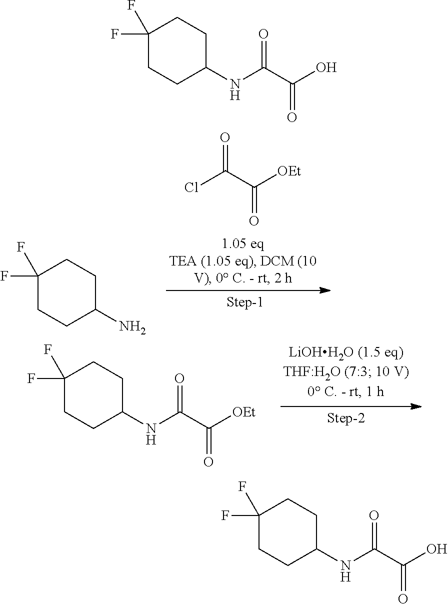

### Step 1. Synthesis of ethyl 2-((4,4-difluorocyclohexyl)amino)-2-oxoacetate

To stirred solution of 4,4-difluorocyclohexan-1-amine (1.0 g, 5.85 mmol) in dichloromethane (10 mL) was added triethylamine (1.6 mL, 11.69 mmol) followed by ethyl 2-chloro-2-oxoacetate (0.7 mL, 5.85 mmol) at 0° C. and the resultant mixture was stirred at room temperature for 2 h. After completion, the reaction mixture was concentrated under reduce pressure (bath temperature <25° C.) to give crude residue. Diethyl ether was added to the residue, the obtained solids were removed by filtration, and the filtrate was dried over anhydrous sodium sulphate and concentrate under reduce pressure to give ethyl 2-((4,4-difluorocyclohexyl)amino)-2-oxoacetate (1.6 g) as a brown liquid. TLC system: EtOAc in petroleum ether (3:7); R_{f}: 0.3.

### Step 2. Synthesis of 2-((4,4-difluorocyclohexyl)amino)-2-oxoacetic Acid

To a solution of ethyl 2-((4,4-difluorocyclohexyl)amino)-2-oxoacetate (1.7 g, 7.23 mmol) in THE (10 mL) and water (5 mL) was added an aqueous solution of LiOH—H₂O (0.36 g, 8.68 mmol) in water (4 mL) dropwise over 5 min at 0° C. and the reaction mixture was stirred at ambient temperature for 1 h. After completion, the reaction mixture was washed with ethyl acetate (2×50 mL). The aqueous layer was acidified with 1 N HCl and extracted with 10% MeOH in DCM (4×50 mL), and the combined organic layers were dried over anhydrous sodium sulphate and concentrated under reduced pressure to afford 2-((4,4-difluorocyclohexyl)amino)-2-oxoacetic acid (17) (0.8 g) as a brown solid. TLC system: MeOH in DCM (1:9); R_{f}: 0.2.

### 5-(2-Fluoropropan-2-yl)isoxazole-3-carboxylic Acid (18)

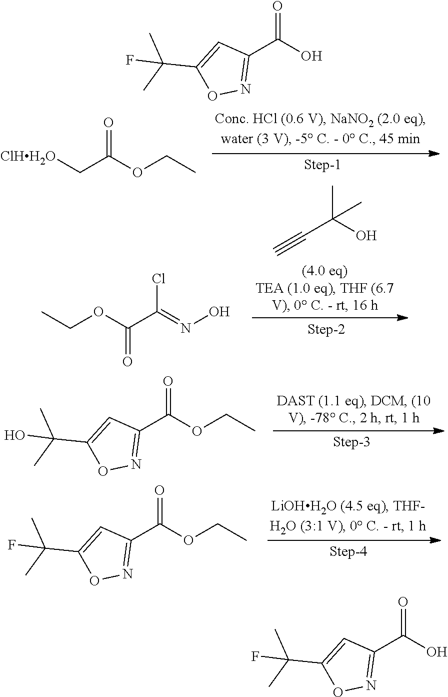

### Step 1. Ethyl (Z)-2-chloro-2-(hydroxyimino)acetate

To a stirred solution of ethyl glycinate hydrochloride (5.0 g, 35.82 mmol) in water (7.5 mL), concentrated hydrochloric acid (3.0 mL) was added at 0° C. The resulting mixture was cooled to −5° C., then sodium nitrite (4.94 g, 71.64 mmol) in water (7.5 mL) was added dropwise at −5° C., and the reaction mixture was stirred at 0° C. for 45 min. After completion of the reaction, the reaction mixture was diluted with brine solution (50 mL) and extracted with ethyl acetate (3×50 mL). The combined organic layers were dried over anhydrous sodium sulphate and concentrated under reduced pressure to give ethyl (Z)-2-chloro-2-(hydroxyimino)acetate (4.5 g, crude) as a yellow liquid. The compound was used in the next step without additional purification. TLC system: EtOAc:hexane (1:9); R_{f}: 0.5

### Step 2. Ethyl 5-(2-hydroxypropan-2-yl)isoxazole-3-carboxylate

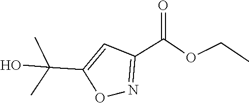

To a stirred solution of ethyl (Z)-2-chloro-2-(hydroxyimino)acetate (4.5 g, 29.69 mmol) and 2-methylbut-3-yn-2-ol (9.99 g, 118.77 mmol) in tetrahydrofuran (20 mL), triethylamine (3.0 g, 29.69 mmol) in tetrahydrofuran (10 mL) was added dropwise at 0° C. for 1 h and the reaction mixture was stirred at room temperature for 16 h. After completion, the reaction mixture was diluted with brine solution (30 mL) and extracted with ethyl acetate (3×50 mL). The combined organic layers were dried over anhydrous sodium sulfate and concentrated under reduced pressure. The obtained crude residue was purified by silica gel column chromatography using 15% ethyl acetate in hexane to afford ethyl 5-(2-hydroxypropan-2-yl)isoxazole-3-carboxylate (1.7 g) as colourless liquid. TLC system: EtOAc:hexane (2:8); R_{f}: 0.25. LCMS m/z=200.30 (M+1).

### Step 3. Ethyl 5-(2-fluoropropan-2-yl)isoxazole-3-carboxylate

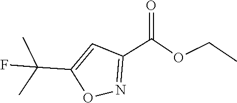

To a stirred solution of ethyl 5-(2-hydroxypropan-2-yl)isoxazole-3-carboxylate (1.5 g, 7.52 mmol) in dichloromethane (10 mL), diethylaminosulfur trifluoride (1.33 g, 8.28 mmol) in dichloromethane (5 mL) was added at −78° C. The reaction mixture was stirred at −78° C. for 2 h and room temperature for 1 h. After completion of the reaction, the reaction mixture was quenched with saturated sodium bicarbonate solution (20 mL) and extracted with dichloromethane (3×40 mL). The combined organic layers were dried over anhydrous sodium sulfate and concentrated under reduced pressure. The obtained crude residue was purified by silica gel column chromatography using 6% ethyl acetate in hexane to afford ethyl 5-(2-fluoropropan-2-yl)isoxazole-3-carboxylate (1.0 g) as a colourless liquid. TLC system: EtOAc:hexane (2:8); R_{f}: 0.6. LCMS m/z=202.19 (M+1).

### Step 4. 5-(2-Fluoropropan-2-yl)isoxazole-3-carboxylic Acid (18)

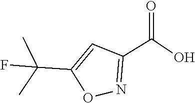

To a stirred solution ethyl 5-(2-fluoropropan-2-yl)isoxazole-3-carboxylate (1.0 g, 4.97 mmol) in tetrahydrofuran (15 mL), lithium hydroxide monohydrate (0.94 g, 22.37 mmol) in water (5 mL) was added at 0° C. and the reaction mixture was stirred at room temperature for 2 h. After completion of the reaction, the mixture was concentrated, and the residue was diluted with water (20 mL) and acidified with 2 N hydrochloric acid (pH-3) at 0° C. The resulting mixture was extracted with ethyl acetate (3×30 mL). The combined organic layers were dried over anhydrous sodium sulfate and concentrated under reduced pressure to afford 5-(2-fluoropropan-2-yl)isoxazole-3-carboxylic acid (0.84 g) as pale yellow liquid. TLC system: EtOAc:hexane (2:8); R_{f}: 0.05. LCMS m/z=174.14 (M+1).

### (5-(2-Fluoropropan-2-yl)isoxazole-3-carbonyl)-L-leucine (19)

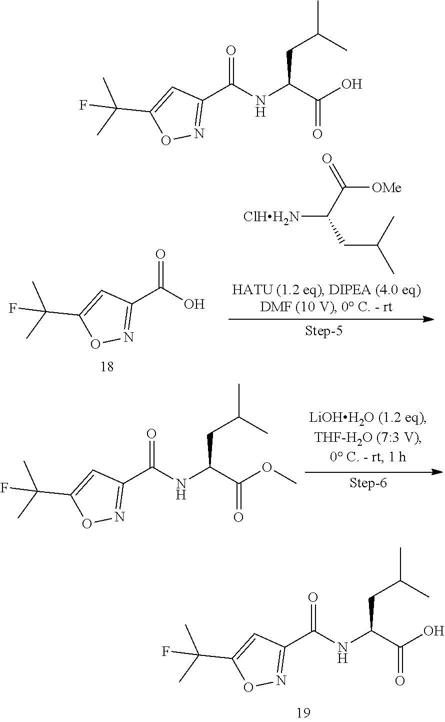

### Step 1. Methyl (5-(2-fluoropropan-2-yl)isoxazole-3-carbonyl)-L-leucinate

To a stirred solution of 5-(2-fluoropropan-2-yl)isoxazole-3-carboxylic acid (0.2 g, 1.16 mmol), methyl L-leucinate hydrochloride (0.21 g, 1.16 mmol) and (1-[bis(dimethylamino)methylene]-1H-1,2,3-triazolo[4,5-b]pyridinium 3-oxide hexafluorophosphate (0.53 g, 1.39 mmol) in DMF (5 mL), N,N-diisopropylethylamine (0.6 g, 4.64 mmol) was added at 0° C. and the reaction mixture was stirred at room temperature for 2 h. After completion of the reaction, the reaction mixture was diluted with water (30 mL) and extracted with ethyl acetate (3×30 mL). The combined organic layers were dried over anhydrous sodium sulphate and concentrated under reduced pressure. The obtained crude residue was purified by Davisil® grade silica gel column chromatography using 10% ethyl acetate in hexane to afford methyl (5-(2-fluoropropan-2-yl)isoxazole-3-carbonyl)-L-leucinate (0.27 g) as an off-white solid. TLC system: EtOAc:hexane (2:8); R_{f}: 0.5. LCMS m/z=301.23 (M+1).

### Step 2. (5-(2-Fluoropropan-2-yl)isoxazole-3-carbonyl)-L-leucine (19)

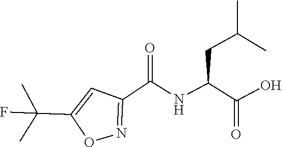 [SMILES: CC(C)C[C@H](NC(=O)c1cc(C(C)(C)F)on1)C(=O)O]

To a stirred solution methyl (5-(2-fluoropropan-2-yl)isoxazole-3-carbonyl)-L-leucinate (0.15 g, 0.5 mmol) in tetrahydrofuran (3 mL), lithium hydroxide monohydrate (0.025 g, 0.6 mmol) in water (1 mL) was added at 0° C. and the reaction mixture was stirred at room temperature for 2 h. After completion, the reaction mixture was concentrated. The residue was diluted with water (10 mL) and acidified with 2 N hydrochloric acid (pH-3) at 0° C. The resulting mixture was extracted with ethyl acetate (3×30 mL). The combined organic layers were dried over anhydrous sodium sulfate and concentrated under reduced pressure to afford (5-(2-fluoropropan-2-yl)isoxazole-3-carbonyl)-L-leucine (0.13 g) as an off-white solid. TLC system: EtOAc:hexane (2:8); R_{f}: 0.05. LCMS m/z=287.31 (M+1).

### (1S,3aR,6aS)-2-(2-(tert-Butylamino)-2-oxoacetyl)octahydrocyclopenta[c]pyrrole-1-carboxylic Acid (20)

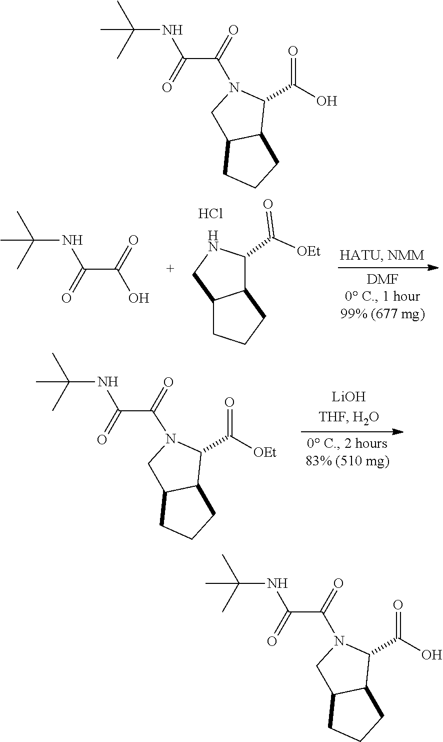

### Step 1. Ethyl (1S,3aR,6aS)-2-(2-(tert-butylamino)-2-oxoacetyl)octahydrocyclopenta[c]pyrrole-1-carboxylate

To a mixture of ethyl (1S,3aR,6aS)-octahydrocyclopenta[c]pyrrole-1-carboxylate (1 equiv, 2.18 mmol, 479 mg) and 2-(tert-butylamino)-2-oxoacetic acid (1.1 equiv, 2.40 mmol, 348 mg) in pre-cooled to 0° C. DMF (10.8 mL), HATU (1.25 equiv, 2.73 mmol, 1.027 g) was added. This mixture was stirred at 0° C. for 10 min, after which NMM (2.56 equiv, 5.58 mmol, 610 μL) was added dropwise over a 1 min period. The reaction was continued at 0° C. for 1 hour, after which it was quenched with deionized water (50 mL), NaCl (saturated aqueous solution, 20 mL), and EtOAc (20 mL). The aqueous layer was separated and extracted with EtOAc (3×20 mL). The combined organic phases were dried over Na₂SO₄ and concentrated in vacuo. Purification by flash column chromatography (SiO₂, graduate elution in CH₂Cl₂:CH₃OH 0→1.5%) yielded ethyl (1S,3aR,6aS)-2-(2-(tert-butylamino)-2-oxoacetyl)octahydrocyclopenta[c]pyrrole-1-carboxylate as a white solid (677 mg, 99% yield).

### Step 2. Synthesis of (1S,3aR,6aS)-2-(2-(tert-butylamino)-2-oxoacetyl)octahydrocyclopenta[c]pyrrole-1-carboxylic Acid

A solution of ethyl (1S,3aR,6aS)-2-(2-(tert-butylamino)-2-oxoacetyl)octahydrocyclopenta[c]pyrrole-1-carboxylate (1 equiv, 2.18 mmol, 677 mg) in THE (4.4 mL) was stirred at 0° C. for 10 min, and a solution of LiOH (2 equiv, 4.36 mmol, 105 mg) in H₂O (4.4 mL) was added dropwise over a 1 min period. The reaction mixture was then stirred at 0° C. for 2 hours, followed by treatment with HCl (1 M soln. in H₂O, 10 mL) and EtOAc (10 mL). The aqueous layer was separated and extracted with EtOAc (3×10 mL). The combined organic phases were dried over Na₂SO₄, and concentrated to yield (1S,3aR,6aS)-2-(2-(tert-butylamino)-2-oxoacetyl)octahydrocyclopenta[c]pyrrole-1-carboxylic acid as a white solid (510 mg, 83% yield).

### (1S,3aR,6aS)-2-(2-((2-Fluorophenyl)amino)-2-oxoacetyl)octahydrocyclopenta[c]pyrrole-1-carboxylic Acid (21)

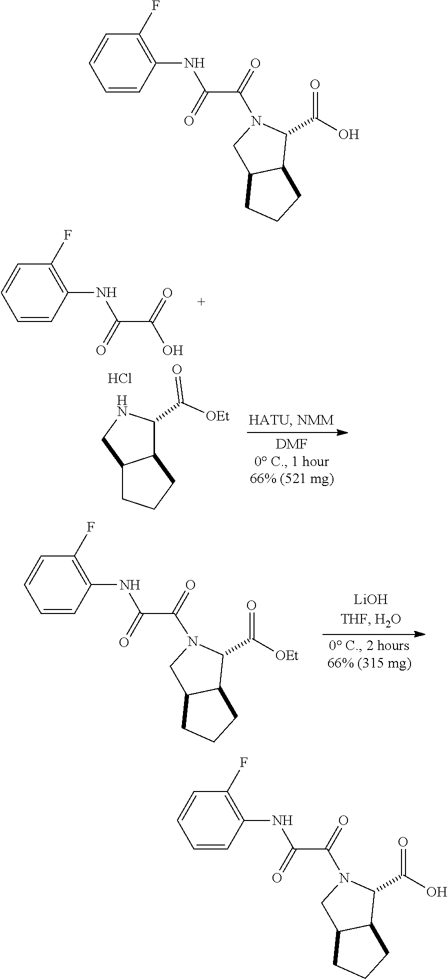

### Step 1. Ethyl (1S,3aR,6aS)-2-(2-((2-fluorophenyl)amino)-2-oxoacetyl)octahydrocyclopenta[c]pyrrole-1-carboxylate

To a mixture of ethyl (1S,3aR,6aS)-octahydrocyclopenta[c]pyrrole-1-carboxylate hydrochloride (1 equiv, 2.28 mmol, 500 mg) and 2-((2-fluorophenyl)amino)-2-oxoacetic acid (1.25 equiv, 2.85 mmol, 522 mg) in pre-cooled to 0° C. DMF (11.4 mL), HATU (1.25 equiv, 2.85 mmol, 1.084 g) was added. This mixture was stirred at 0° C. for 10 min, after which NMM (2.56 equiv, 5.84 mmol, 642 μL) was added dropwise over a 1 min period. The reaction was continued at 0° C. for 1 hour, after which it was quenched with deionized water (50 mL), saturated aqueous NaCl (20 mL), and EtOAc (20 mL). The aqueous layer was separated and extracted with EtOAc (3×20 mL). The combined organic phases were dried over Na₂SO₄ and concentrated in vacuo. Purification by flash column chromatography (SiO₂, graduate elution in CH₂Cl₂:CH₃OH 0→1.5%) yielded ethyl (1S,3aR,6aS)-2-(2-((2-fluorophenyl)amino)-2-oxoacetyl)octahydrocyclopenta[c]pyrrole-1-carboxylate as a white solid (521 mg, 66% yield).

### Step 2. (1S,3aR,6aS)-2-(2-((2-Fluorophenyl)amino)-2-oxoacetyl)octahydrocyclopenta[c]pyrrole-1-carboxylic Acid

A solution of ethyl (1S,3aR,6aS)-2-(2-((2-fluorophenyl)amino)-2-oxoacetyl)octahydrocyclopenta[c]pyrrole-1-carboxylate (1 equiv, 1.5 mmol, 521 mg) in THE (3 mL) was stirred at 0° C. for 10 min, after which a solution of LiGH (2 equiv; 3 mmol, 72 mg) in H₂O (3 mL) was added dropwise over a 1 min period. The reaction mixture was then stirred at 0° C. for 2 hours, followed by treatment with HCl (1 M soln. in H₂O, 10 mL) and EtOAc (10 mL). The aqueous layer was separated and extracted with EtOAc (3×10 mL). The combined organic phases were dried over Na₂SO₄ and concentrated in vacuo, thus yielding (1S,3aR,6aS)-2-(2-((2-fluorophenyl)amino)-2-oxoacetyl)octahydrocyclopenta[c]pyrrole-1-carboxylic acid as a white solid (315 mg, 66% yield).

### (1R,2S,5S)-3-((S)-3,3-Dimethyl-2-(2,2,2-trifluoroacetamido)butanoyl)-6,6-dimethyl-3-azabicyclo[3.1.0]hexane-2-carboxylic Acid (22)

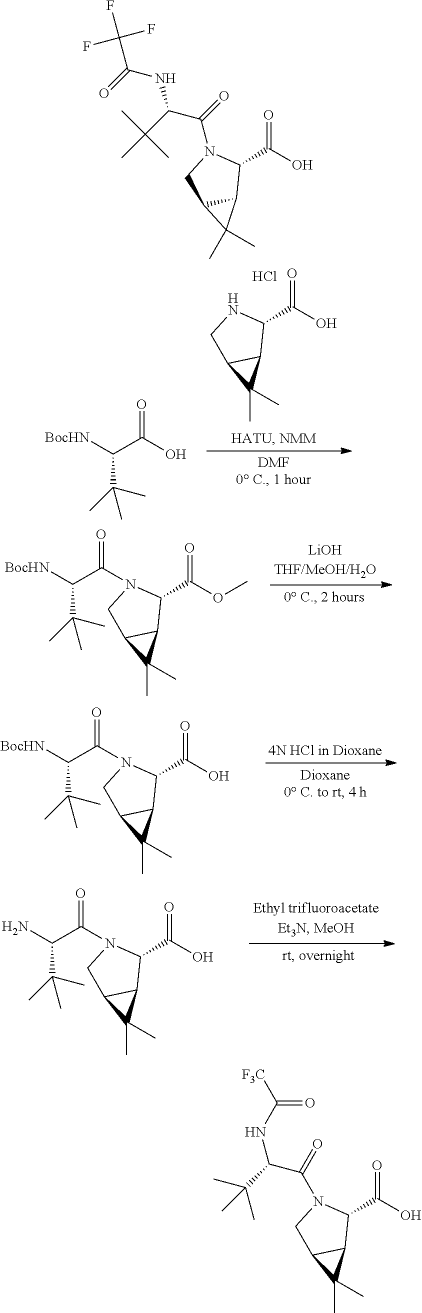

### Step 1. Synthesis of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate

A solution of (S)-2-((tert-butoxycarbonyl)amino)-3,3-dimethylbutanoic acid (2.16 mmol, 500 mg) in DMF (5 mL) was stirred at 0° C. for 5 min. HATU (1.2 equiv, 2.59 mmol, 986 mg) and NMM (3 equiv, 6.49 mmol, 713 μL) were added dropwise, and after stirring for another 5 min, methyl (1R,2S,5S)-6,6-dimethyl-3-azabicyclo[3.1.0]hexane-2-carboxylate (2.59 mmol, 534 mg) was added. The reaction was continued at 0° C. for 1 hour and was quenched with H₂O (50 mL), NaCl (sat. aq. soln., 20 mL), and EtOAc (20 mL). The aqueous layer was separated and extracted with EtOAc (3×20 mL). The combined organic phases were dried over Na₂SO₄, and concentrated in vacuo. Purification by flash column chromatography (SiO₂, graduate elution in CH₂Cl₂:CH₃OH 0→1.5%) yielded methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate as a white solid (750 mg, 91% yield).

### Step 2. Synthesis of (1R,2S,5S)-3-((S)-2-((tert-butoxycarbonyl)amino)-3,3-dimethylbutanoyl)-6,6-dimethyl-3-azabicyclo[3.1.0]hexane-2-carboxylic Acid

A solution of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate (369 μmol, 141 mg) in THE (1 mL) was stirred at 0° C. for 10 min, and a solution of LiOH (2 equiv; 737.25 μmol, 18 mg) in H₂O (500 mL) was added dropwise. The reaction mixture was then stirred at 0° C. for 2 hours, followed by treatment with HCl (1 M in H₂O, 3 mL) and EtOAc (10 mL). The aqueous layer was separated and extracted with EtOAc (3×10 mL). The combined organic phases were dried over Na₂SO₄ and concentrated in vacuo, thus yielding (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid as a white solid (120 mg, 88% yield).

### Step 3. Synthesis of (1R,2S,5S)-3-((S)-2-amino-3,3-dimethylbutanoyl)-6,6-dimethyl-3-azabicyclo[3.1.0]hexane-2-carboxylic acid hydrochloride

A solution of (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid (325.67 μmol, 120 mg) in dioxane (1 mL) was stirred at 0° C. for 10 min, and a solution of HCl (22 equiv; 7.16 mmol, 1.79 mL) in dioxane (4 M) was added dropwise. The reaction mixture was gradually warmed to room temperature and stirred for another 4 hours. After reaction was complete, the mixture was concentrated in vacuo to give (1R,2S,5S)-3-((S)-2-amino-3,3-dimethylbutanoyl)-6,6-dimethyl-3-azabicyclo[3.1.0]hexane-2-carboxylic acid hydrochloride. It was used for the next step without further purification.

### Step 4. Synthesis of (1R,2S,5S)-3-((S)-3,3-dimethyl-2-(2,2,2-trifluoroacetamido)butanoyl)-6,6-dimethyl-3-azabicyclo[3.1.0]hexane-2-carboxylic Acid

Triethylamine (4 equiv, 1.49 mmol, 207.19 μL.) was added to a solution of (1R,2S,5S)-3-((S)-2-amino-3,3-dimethylbutanoyl)-6,6-dimethyl-3-azabicyclo[3.1.0]hexane-2-carboxylic acid hydrochloride (372.64 μmol, 100 mg) in MeOH (1 mL). Then ethyl trifluoroacetate (1.3 equiv, 484.43 μmol, 69 mg.) was added, and the reaction mixture was stirred at room temperature for 12 h. The volatiles were removed by rotary evaporation, and the residue was dissolved in H₂O (5 mL) and extracted with EtOAc (3×10 mL). The combined organic phases were washed with brine, dried over Na₂SO₄, and concentrated in vacuo to give (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid which was used for the next step without further purification.

### Synthesis of Compounds of Formula (I)

Illustrative compounds of the present disclosure are synthesized according to, or by adaptation of, the following procedures.

### Example 1. Synthesis of N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide (39)

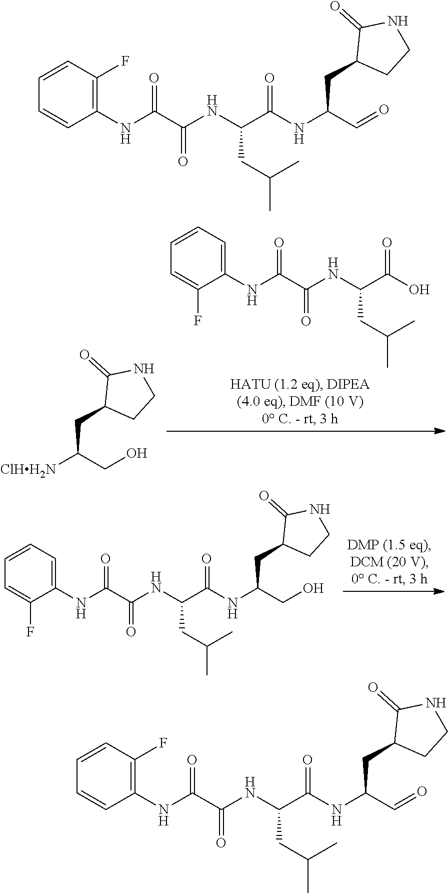

### Step 1. N¹-(2-fluorophenyl)-N²—((S)-1-(((S)-1-hydroxy-3-((S)-2-oxopyrrolidin-3-yl)propan-2-yl)amino)-4-methyl-1-oxopentan-2-yl)oxalamide

To a solution of (2-((2-fluorophenyl)amino)-2-oxoacetyl)-L-leucine (0.46 g, 1.57 mmol) and (S)-3-((S)-2-amino-3-hydroxypropyl)pyrrolidin-2-one hydrochloride (0.4 g, 1.57 mmol) in DMF (3 mL) at 0° C. was added HATU (0.78 g, 2.04 mmol) and DIPEA (1.09 mL, 6.27 mmol), and the resultant mixture was stirred at room temperature for 3 h. After completion, cold water (40 mL) was added to the reaction mixture and extracted with EtOAc (3×50 mL). The combined organic layers were dried over anhydrous sodium sulfate, filtered and concentrated under reduced pressure. The crude material was then purified by column chromatography over silica gel (230-400 mesh) using 90-95% ethyl acetate in petroleum ether as a gradient to afford N¹-(2-fluorophenyl)-N²—((S)-1-(((S)-1-hydroxy-3-((S)-2-oxopyrrolidin-3-yl)propan-2-yl)amino)-4-methyl-1-oxopentan-2-yl)oxalamide (0.3 g) as an off-white solid. TLC system: EtOAc:petroleum ether (10:0); R_{f}: 0.3. LCMS m/z=437.52 (M+1)

### Step 2. Synthesis of N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide (39)

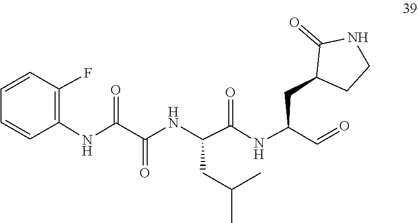 [SMILES: CC(C)C[C@H](NC(=O)C(=O)Nc1ccccc1F)C(=O)N[C@H](C=O)C[C@@H]1CCNC1=O]

To a solution of N¹-(2-fluorophenyl)-N²—((S)-1-(((S)-1-hydroxy-3-((S)-2-oxopyrrolidin-3-yl)propan-2-yl)amino)-4-methyl-1-oxopentan-2-yl)oxalamide (0.3 g, 0.69 mmol) in anhydrous EtOAc (10 mL) at 0° C. under a nitrogen atmosphere was added Dess-Martin periodinane reagent (0.44 g, 1.03 mmol). The ice bath was removed, and the reaction mixture was stirred at room temperature for 3 h. A solution of 10% aqueous sodium thiosulfate (20 mL) was added, and the solution was stirred for additional 15 min. The aqueous layer was removed, and the organic layer was washed with 10% aqueous sodium thiosulfate (20 mL), followed by saturated aqueous sodium bicarbonate (2×20 mL), water (2×20 mL), and brine (1×20 mL). The organic layer was dried over anhydrous sodium sulfate, filtered, and concentrated. The obtained residue was then purified by column chromatography over silica gel (230-400 mesh) using 98-100% EtOAc in petroleum ether as an eluent to afford N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide (39) (100 mg) as an off-white solid. TLC system: 100% EtOAc; R_{f}: 0.4. Analytical Data: LCMS m/z 435.22 (M+1); ¹H NMR (400 MHz, CDCl₃) δ ppm 9.50 (d, J=1.1 Hz, 1H), 9.44 (s, 1H), 8.71 (d, J=5.0 Hz, 1H), 8.34 (q, J=5.6 Hz, 1H), 7.90 (d, J=8.7 Hz, 1H), 7.18-7.11 (m, 1H), 5.70 (s, 1H), 4.60 (t, J=4.5 Hz, 1H), 4.29 (t, J=4.3 Hz, 1H), 3.38 (q, J=4.5 Hz, 1H), 2.51-2.39 (m, 1H), 1.97-1.72 (m, 6H), 1.00 (q, J=2.7 Hz, 1H).

### Example 2. Synthesis of (S)-3-((S)-2-(2-(cyclohexylamino)-2-oxoacetamido)-4-methylpentanamido)-2-oxo-4-((S)-2-oxopyrrolidin-3-yl)butyl heptanoate (18)

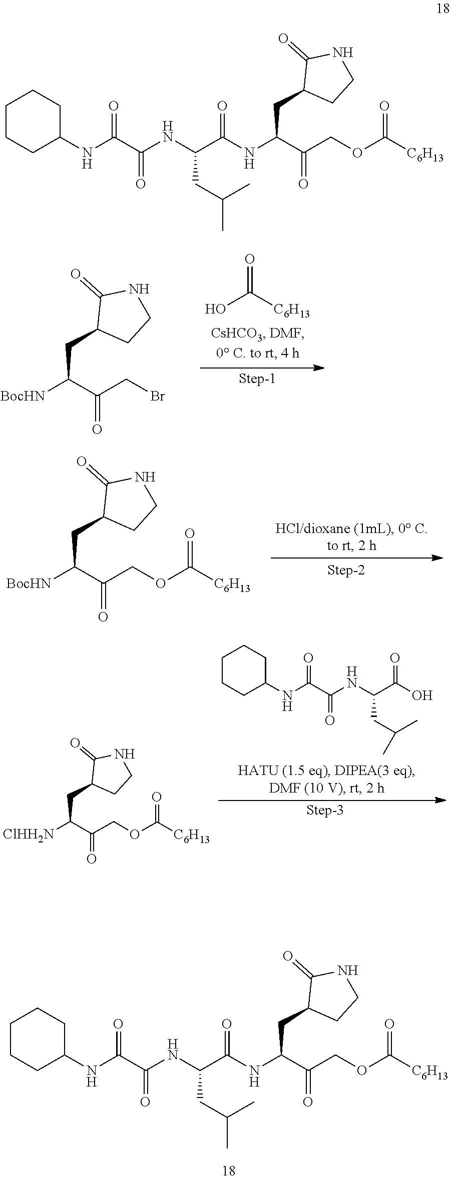

### Step 1. Synthesis of (S)-3-((tert-butoxycarbonyl)amino)-2-oxo-4-((S)-2-oxopyrrolidin-3-yl)butyl heptanoate

To a solution of tert-butyl ((S)-4-bromo-3-oxo-1-((S)-2-oxopyrrolidin-3-yl)butan-2-yl)carbamate (0.5 g, 1.43 mmol) in DMF (5 mL) was added heptanoic acid (0.23 g, 1.57 mmol) followed by CsHCO₃ (0.56 g, 2.86 mmol). The reaction mixture was stirred at ambient temperature for 4 h. After completion of reaction, the reaction mixture was quenched with cold water and extracted with EtOAc (50 mL×2). The combined organic layers were washed with cold water (50 mL) and brine (50 mL), and dried over Na₂SO₄, filtered, and concentrated under reduced pressure. The crude compound was purified by column chromatography over silica gel (Davisil®) (using EtOAc as eluent) to afford 0.51 g of (S)-3-((tert-butoxycarbonyl)amino)-2-oxo-4-((S)-2-oxopyrrolidin-3-yl)butyl heptanoate as a pale brown gum. [TLC system: MeOH:DCM (1:9); R_{f}value: 0.4].

### Step 2. Synthesis of (S)-3-amino-2-oxo-4-((S)-2-oxopyrrolidin-3-yl)butyl heptanoate hydrochloride

To a stirred solution of (S)-3-((tert-butoxycarbonyl)amino)-2-oxo-4-((S)-2-oxopyrrolidin-3-yl)butyl heptanoate (0.51 g, 1.28 mmol) in dioxane (5 mL) was added 4 M HCl in dioxane (5 mL) at 0° C. The reaction mixture was stirred at ambient temperature for 2 h. After completion of the reaction, the reaction mixture was concentrated under reduced pressure to afford 0.42 g of (S)-3-amino-2-oxo-4-((S)-2-oxopyrrolidin-3-yl)butyl heptanoate hydrochloride as an off white solid. [TLC system: MeOH:DCM (1:9); R_{f}value: 0.2].

### Step 3. Synthesis of (S)-3-((S)-2-(2-(cyclohexylamino)-2-oxoacetamido)-4-methylpentanamido)-2-oxo-4-((S)-2-oxopyrrolidin-3-yl)butyl heptanoate (18)

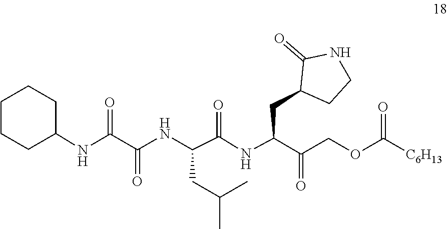

To a solution of (2-(cyclohexylamino)-2-oxoacetyl)-L-leucine (0.2 g, 0.70 mmol) in DMF (2 mL) was added HATU (0.40 g, 0.55 mmol) and DIPEA (0.37 mL, 2.11 mmol) at 0° C. (2-(Cyclohexylamino)-2-oxoacetyl)-L-leucine was prepared using the procedure described for the preparation of 2-((2-fluorophenyl)amino)-2-oxoacetic acid (14) substituting cyclohexylamine for 2-fluoroaniline in Step 1. Then, (S)-3-amino-2-oxo-4-((S)-2-oxopyrrolidin-3-yl)butyl heptanoate hydrochloride (0.24 g, 0.70 mmol) was added, and the mixture was stirred at room temperature for 2 h. After completion of reaction, the reaction mixture was quenched with cold water and extracted with EtOAc (50 mL×2). The combined organic layers were washed with cold water (50 mL) and brine (50 mL) and dried over Na₂SO₄ and filtered and concentrated under reduced pressure. The crude material was purified by column chromatography over silica gel (Davisil®) (using EtOAc as eluent) to afford 0.052 g of (S)-3-((S)-2-(2-(cyclohexylamino)-2-oxoacetamido)-4-methylpentanamido)-2-oxo-4-((S)-2-oxopyrrolidin-3-yl)butyl heptanoate (18) as an off white solid. [TLC system: MeOH:DCM (1:9); R_{f}value: 0.4]. Analytical Data: LCMS m/z=563.39 (M−1); ¹H NMR (400 MHz, DMSO-d₆) δ ppm 8.57-8.52 (m, 3H), 7.65 (s, 1H), 4.83 (s, 2H), 4.51-4.30 (m, 2H), 3.55 (d, J=8.6 Hz, 1H), 3.21-3.05 (m, 2H), 2.36 (t, J=7.3 Hz, 2H), 2.29-2.26 (m, 1H), 2.16-2.08 (m, 1H), 2.05-1.95 (m, 1H), 1.72-1.45 (m, 11H), 1.38-1.26 (m, 11H), 1.12-1.02 (s, 1H), 0.89-0.85 (m, 9H).

### Example 3. Synthesis of N—((S)-1-(((S)-4-hydroxy-3-oxo-1-((S)-2-oxopyrrolidin-3-yl)butan-2-yl)amino)-4-methyl-1-oxopentan-2-yl)-4-methoxy-1H-indole-2-carboxamide-(1)

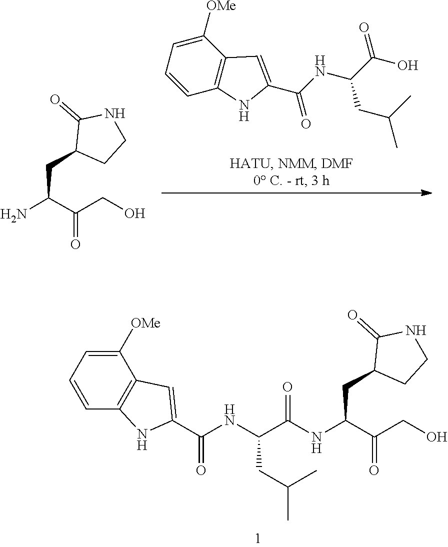 [SMILES: N[C@@H](C[C@@H]1CCNC1=O)C(=O)CO ; COc1cccc2[nH]c(C(=O)N[C@@H](CC(C)C)C(=O)O)cc12 ; COc1cccc2[nH]c(C(=O)N[C@@H](CC(C)C)C(=O)N[C@@H](C[C@@H]3CCNC3=O)C(=O)CO)cc12]

To a solution of (4-methoxy-1H-indole-2-carbonyl)-L-leucine (0.315 g, 1.03 mmol) in dry DMF (4 mL) at 0° C., was added ((S)-3-((S)-2-amino-4-hydroxy-3-oxobutyl)pyrrolidin-2-one hydrochloride (0.23 g, 1.03 mmol), and HATU (0.508 g, 1.339 mmol) followed by NMM (0.45 mL, 4.12 mmol), and the resultant mixture was stirred at room temperature for 2 h. After completion, the reaction mixture was diluted with ethyl acetate (30 mL), washed with saturated aqueous NaHCO₃ (1×20 mL), water (1×20 mL) and brine solution (1×15 mL). The organic layer was dried over anhydrous sodium sulfate, filtered, and concentrated under reduced pressure. The crude residue was then purified by column chromatography over silica gel (230-400 mesh) using 3-4% MeOH in DCM as a gradient to afford N—((S)-1-(((S)-4-hydroxy-3-oxo-1-((S)-2-oxopyrrolidin-3-yl)butan-2-yl)amino)-4-methyl-1-oxopentan-2-yl)-4-methoxy-1H-indole-2-carboxamide (1) (0.14 g) as an off-white solid along with by-product (250 mg). TLC system: MeOH: DCM (1:9); R_{f}: 0.5. Analytical Data: LCMS m/z=471.48 (M−1); ¹H NMR (400 MHz, DMSO-d₆) δ ppm 11.57 (s, 1H), 8.45 (d, J=8.0 Hz, 2H), 8.40 (d, J=7.6 Hz, 2H), 7.62 (s, 1H), 7.36 (d, J=1.5 Hz, 1H), 7.09 (t, J=7.9 Hz, 1H), 7.00 (d, J=8.2 Hz, 1H), 6.51 (d, J=7.6 Hz, 1H), 5.05 (t, J=5.9 Hz, 1H), 4.49-4.44 (m, 2H), 4.25 (dq, J=8.3, 4.6 Hz, 1H), 3.88 (s, 3H), 3.13-3.06 (m, 2H), 2.28 (d, J=8.5 Hz, 1H), 2.10 (t, J=8.5 Hz, 1H), 1.73-1.53 (m, 5H), 0.94 (d, J=6.0 Hz, 3H), 0.94 (d, J=6.0 Hz, 3H).

### Example 4. (1S,3aR,6aS)-2-(2-((2-fluorophenyl)amino)-2-oxoacetyl)-N—((S)-1-oxo-3-((S)-2-oxopyrrolidin-3-yl)propan-2-yl)octahydrocyclopenta[c]pyrrole-1-carboxamide (44)

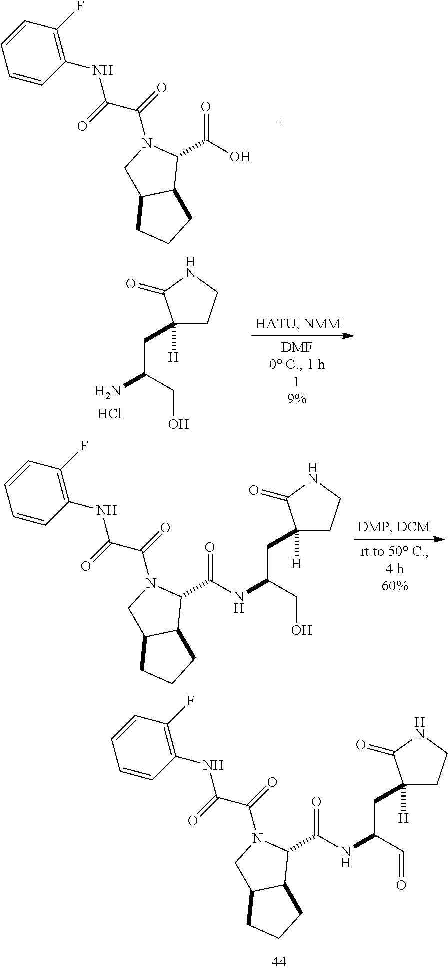

### Step 1. (1S,3aR,6aS)-2-(2-((2-fluorophenyl)amino)-2-oxoacetyl)-N—((S)-1-hydroxy-3-((S)-2-oxopyrrolidin-3-yl)propan-2-yl)octahydrocyclopenta[c]pyrrole-1-carboxamide

To a mixture of (S)-3-((S)-2-amino-3-hydroxypropyl)pyrrolidin-2-one hydrochloride (1.25 equiv, 0.156 mmol, 30 mg) and (1S,3aR,6aS)-2-(2-((2-fluorophenyl)amino)-2-oxoacetyl)octahydrocyclopenta[c]pyrrole-1-carboxylic acid (1 equiv, 0.125 mmol, 40 mg) in DMF (1.0 mL), HATU (1.25 equiv, 0.16 mmol, 60 mg) was added. This mixture was stirred at 0° C. for 10 min, after which NMM (2.5 equiv, 0.325 mmol, 36 μL) was added dropwise over a 1 min period. The reaction was continued at 0° C. for 2 hours, after which it was quenched with deionized water (10 mL), NaCl (sat. aq. soln., 5 mL), and EtOAc (5 mL). The aqueous layer was separated and extracted with EtOAc (3×5 mL). The combined organic phases were dried over Na₂SO₄ and concentrated in vacuo. Purification by flash column chromatography (SiO₂, graduate elution in CH₂Cl₂:CH₃OH 0→10%) yielded (1S,3aR,6aS)-2-(2-((2-fluorophenyl)amino)-2-oxoacetyl)-N—((S)-1-hydroxy-3-((S)-2-oxopyrrolidin-3-yl)propan-2-yl)octahydrocyclopenta[c]pyrrole-1-carboxamide as a white solid (10 mg, 11% yield, MS m/z=461.7 (M+1)).

### Step 2. (1S,3aR,6aS)-2-(2-((2-fluorophenyl)amino)-2-oxoacetyl)-N—((S)-1-oxo-3-((S)-2-oxopyrrolidin-3-yl)propan-2-yl)octahydrocyclopenta[c]pyrrole-1-carboxamide

To a mixture of (1S,3aR,6aS)-2-(2-((2-fluorophenyl)amino)-2-oxoacetyl)-N—((S)-1-hydroxy-3-((S)-2-oxopyrrolidin-3-yl)propan-2-yl)octahydrocyclopenta[c]pyrrole-1-carboxamide (1 equiv, 0.021 mmol, 10 mg) in dry DCM (1.0 mL), Dess-Martin periodinane (DMP; 1.5 equiv, 0.032 mmol, 15 mg) was added at 0° C. The reaction mixture was then warmed up to room temperature and stirred for 2 hours. After UPLC monitoring showed no progress, additional DMP (1.5 equiv, 0.032 mmol, 15 mg) was added to the reaction mixture and the temperature was raised to 50° C. The reaction was stirred at 50° C. for four hours, then extra DCM (5 mL) was added, and the reaction was quenched with 10% Na₂SO₃ (10 mL) aqueous solution. The aqueous layer was separated and extracted with DCM (2×5 mL). The combined organic layers were washed with NaHCO₃ (sat. aq. soln., 10 mL) and brine (10 mL), then were dried over Na₂SO₄, and concentrated in vacuo. Purification by flash column chromatography (SiO₂, graduate elution in CH₂Cl₂:CH₃OH 0→15%) yielded (1S,3aR,6aS)-2-(2-((2-fluorophenyl)amino)-2-oxoacetyl)-N—((S)-1-oxo-3-((S)-2-oxopyrrolidin-3-yl)propan-2-yl)octahydrocyclopenta[c]pyrrole-1-carboxamide as a white solid (44, 6 mg, 60% yield). Analytical data: LCMS m/z=459.3283 (M+1); ¹H NMR (500 MHz, methanol-d₄) δ ppm 8.14-8.09 (m, 1H), 7.25-7.11 (m, 4H), 5.04 (t, J=3.2 Hz, 0.7H), 4.45 (dd, J=4.6, 3.1 Hz, 0.7H), 3.97-3.88 (m, 2H), 3.61-3.56 (m, 0.8H), 3.41-3.36 (m, 4.8H), 3.11 (td, J=9.4, 2.3 Hz, 0.7H), 2.93-2.63 (m, 4H), 2.44-2.34 (m, 0.6H), 2.16-1.48 (m, 9H)

### Example 5. (1S,3aR,6aS)-2-(2-((2-fluorophenyl)amino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide (30)

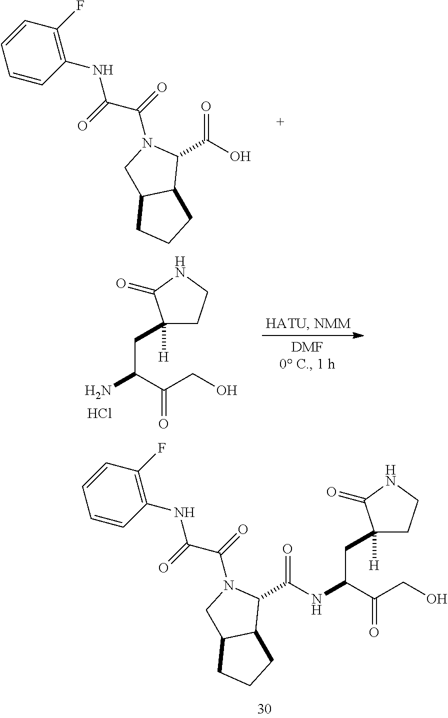

To a mixture of (S)-3-((S)-2-amino-4-hydroxy-3-oxobutyl)pyrrolidin-2-one hydrochloride (1.3 equiv, 0.18 mmol, 40 mg) and (1S,3aR,6aS)-2-(2-((2-fluorophenyl)amino)-2-oxoacetyl)octahydrocyclopenta[c]pyrrole-1-carboxylic acid (1 equiv, 0.14 mmol, 45 mg), HATU (1.2 equiv, 0.17 mmol, 64 mg) was added in pre-cooled to 0° C. DMF (1.2 mL). This mixture was stirred at 0° C. for 10 min, after which NMM (2.5 equiv, 0.35 mmol, 38 μL) was added dropwise over a 1 min period. The reaction was continued at 0° C. for 1 hour, after which it was quenched with water (10 mL), NaCl (sat. aq. soln., 5 mL), and EtOAc (5 mL). The aqueous layer was separated and extracted with EtOAc (3×5 mL). The combined organic phases were dried over Na₂SO₄ and concentrated in vacuo. Purification by flash column chromatography (SiO₂, graduate elution in CH₂Cl₂:CH₃OH 0→7%) yielded (1S,3aR,6aS)-2-(2-((2-fluorophenyl)amino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide as a white solid (10 mg, 11% yield), and was obtained as a mixture of stereoisomers (as judged by ¹H NMR, approximate ratio is 1.5:1). Analytical data: LCMS m/z=489.3654 (M+1); ¹H NMR (499 MHz, DMSO-d₆) δ ppm 10.30 (s, 0.4H), 10.19 (s, 0.6H), 8.58 (d, J=7.9 Hz, 0.4H), 8.46 (d, J=8.0 Hz, 0.6H), 7.78-7.59 (m, 1.4H), 7.52 (s, 0.6H), 7.36-7.07 (m, 3H), 5.07 (dt, J=13.7, 6.0 Hz, 1H), 4.84 (d, J=2.8 Hz, 0.4H), 4.50-4.32 (m, 1H), 4.31-4.08 (m, 2H), 3.99 (dd, J=11.8, 7.9 Hz, 0.4H), 3.84-3.64 (m, 1.2H), 3.46 (dd, J=12.7, 4.5 Hz, 0.6H), 3.14 (dq, J=16.9, 9.1 Hz, 0.8H), 2.99 (t, J=9.1 Hz, 0.6H), 2.81-2.57 (m, 3H), 2.22-2.09 (m, 0.4H), 2.06-1.38 (m, 10.6H).

### Example 6. (1S,3aR,6aS)-2-(2-((2-fluorophenyl)amino)-2-oxoacetyl)-N—((S)-4-hydroxy-3-oxo-1-((S)-2-oxopiperidin-3-yl)butan-2-yl)octahydrocyclopenta[c]pyrrole-1-carboxamide (33)

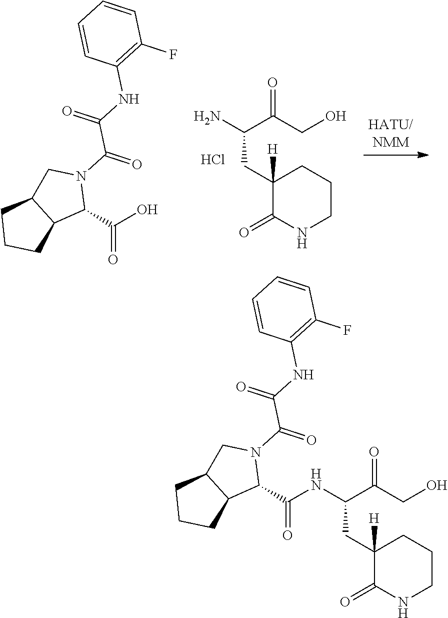 [SMILES: O=C(Nc1ccccc1F)C(=O)N1C[C@@H]2CCC[C@@H]2[C@H]1C(=O)O ; Cl ; N[C@@H](C[C@@H]1CCCNC1=O)C(=O)CO ; O=C(Nc1ccccc1F)C(=O)N1C[C@@H]2CCC[C@@H]2[C@H]1C(=O)N[C@@H](C[C@@H]1CCCNC1=O)C(=O)CO]

To a mixture of (S)-3-((S)-2-amino-4-hydroxy-3-oxobutyl)piperidin-2-one hydrochloride (1.3 equiv, 0.18 mmol, 43 mg) and (1S,3aR,6aS)-2-(2-((2-fluorophenyl)amino)-2-oxoacetyl)octahydrocyclopenta[c]pyrrole-1-carboxylic acid (1 equiv, 0.14 mmol, 45 mg), HATU (1.2 equiv, 0.17 mmol, 64 mg) was added in pre-cooled to 0° C. DMF (1.2 mL). This mixture was stirred at 0° C. for 10 min, after which NMM (2.5 equiv, 0.35 mmol, 38 μL) was added dropwise over a 1 min period. The reaction was continued at 0° C. for 1 hour, after which it was quenched with water (10 mL), NaCl (sat. aq. soln., 5 mL), and EtOAc (5 mL). The aqueous layer was separated and extracted with EtOAc (3×5 mL). The combined organic phases were dried over Na₂SO₄ and concentrated in vacuo. Purification by flash column chromatography (SiO₂, graduate elution in CH₂Cl₂:CH₃OH 0→7%) yielded desired adduct amide as a white solid (15 mg, 17% yield), and was obtained as a mixture of stereoisomers (as judged by ¹H NMR, approximate ratio is 1.5:1). Analytical data: LCMS m/z=503.6661 (M+1); ¹H NMR (499 MHz, DMSO-d₆) δ ppm 10.27 (s, 0.4H), 10.18 (s, 0.6H), 8.59 (d, J=7.8 Hz, 0.4H), 8.48 (d, J=8.0 Hz, 0.6H), 7.78-7.68 (m, 1H), 7.46-7.11 (m, 4H), 5.04 (dt, J=11.8, 6.0 Hz, 0.6H), 4.85 (d, J=2.6 Hz, 0.4H), 4.57-4.37 (m, 1H), 4.29-4.10 (m, 2.4H), 4.03-3.95 (m, 0.6H), 3.81-3.67 (m, 1.2H), 3.46 (dd, J=12.8, 4.7 Hz, 0.8H), 3.17-2.88 (m, 3H), 2.30-2.05 (m, 2H), 2.01-1.39 (m, 12H).

### Example 7. (1S,3aR,6aS)—N—((S)-1-cyano-2-((S)-2-oxopiperidin-3-yl)ethyl)-2-(2-((2-fluorophenyl)amino)-2-oxoacetyl)octahydrocyclopenta[c]pyrrole-1-carboxamide (60)

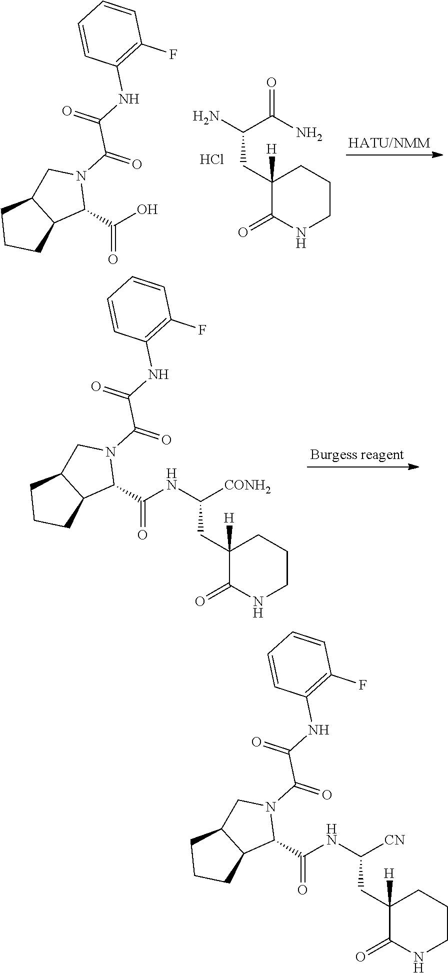

### Step 1. (1S,3aR,6aS)—N—((S)-1-amino-1-oxo-3-((S)-2-oxopiperidin-3-yl)propan-2-yl)-2-(2-((2-fluorophenyl)amino)-2-oxoacetyl)octahydrocyclopenta[c]pyrrole-1-carboxamide

To a mixture of (S)-2-amino-3-((S)-2-oxopiperidin-3-yl)propanamide hydrochloride (0.35 mmol, 78 mg) and (1S,3aR,6aS)-2-(2-((2-fluorophenyl)amino)-2-oxoacetyl)octahydrocyclopenta[c]pyrrole-1-carboxylic acid (0.42 mmol, 135 mg), HATU (0.44 mmol, 166 mg) was added in pre-cooled to 0° C. DMF (2.3 mL). This mixture was stirred at 0° C. for 10 min, after which NMM (0.9 mmol, 99 μL) was added dropwise over 1 min period. The reaction was continued at 0° C. for 1 hour, after which it was quenched with water (20 mL), NaCl (sat. aq. soln., 10 mL), and EtOAc (10 mL). The aqueous layer was separated and extracted with EtOAc (3×10 mL). The combined organic phases were dried over Na₂SO₄ and concentrated in vacuo. Purification by flash column chromatography (SiO₂, graduate elution in CH₂Cl₂:CH₃OH 0→7%) yielded (1S,3aR,6aS)—N—((S)-1-amino-1-oxo-3-((S)-2-oxopiperidin-3-yl)propan-2-yl)-2-(2-((2-fluorophenyl)amino)-2-oxoacetyl)octahydrocyclopenta[c]pyrrole-1-carboxamide as a white solid (52 mg, 31% yield).

### Step 2. (1S,3aR,6aS)—N—((S)-1-cyano-2-((S)-2-oxopiperidin-3-yl)ethyl)-2-(2-((2-fluorophenyl)amino)-2-oxoacetyl)octahydrocyclopenta[c]pyrrole-1-carboxamide

A mixture of (1S,3aR,6aS)—N—((S)-1-amino-1-oxo-3-((S)-2-oxopiperidin-3-yl)propan-2-yl)-2-(2-((2-fluorophenyl)amino)-2-oxoacetyl)octahydrocyclopenta[c]pyrrole-1-carboxamide (0.107 mmol, 52 mg) in DCM (0.53 mL) was pre-cooled to 0° C. over 10 min period, after which Burgess reagent (0.214 mmol, 51 mg) was added at once at 0° C. The reaction mixture was allowed to warm up to 23° C. over a 2-hour period, after which it was directly purified by flash column chromatography (SiO₂, graduate elution in DCM:MeOH (0→5%), thus yielding (1S,3aR,6aS)—N—((S)-1-cyano-2-((S)-2-oxopiperidin-3-yl)ethyl)-2-(2-((2-fluorophenyl)amino)-2-oxoacetyl)octahydrocyclopenta[c]pyrrole-1-carboxamide as a white solid (40 mg, 80% yield). It was obtained as a mixture of stereoisomers (as judged by ¹H NMR, approximate ratio is 1.5:1). Analytical data: LCMS m/z=470.3214 (M+1); ¹H NMR (499 MHz, DMSO-d₆) δ ppm 10.30 (s, 0.4H), 10.24 (s, 0.6H), 8.97 (d, J=8.0 Hz, 0.4H), 8.89 (d, J=8.1 Hz, 0.6H), 7.72 (td, J=7.9, 1.9 Hz, 0.4H), 7.66 (td, J=7.9, 1.8 Hz, 0.6H), 7.54 (s, 0.4H), 7.46 (s, 0.6H), 7.26 (ddddd, J=18.0, 13.1, 11.3, 8.4, 6.7 Hz, 2.4H), 7.16 (td, J=7.6, 1.6 Hz, 0.6H), 5.10-4.99 (m, 0.4H), 4.98-4.89 (m, 0.6H), 4.77 (d, J=2.0 Hz, 0.6H), 4.20 (d, J=3.8 Hz, 0.4H), 4.01 (dd, J=11.8, 7.9 Hz, 0.4H), 3.81-3.68 (m, 1.2H), 3.48 (dd, J=12.8, 3.8 Hz, 0.8H), 3.10 (d, J=11.4 Hz, 0.8H), 3.03-2.87 (m, 1.2H), 2.75 (dt, J=8.6, 4.5 Hz, 0.6H), 2.34-2.16 (m, 2H), 2.01-1.36 (m, 10H), 1.35-1.18 (m, 2H).

### Example 8. N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide (3)

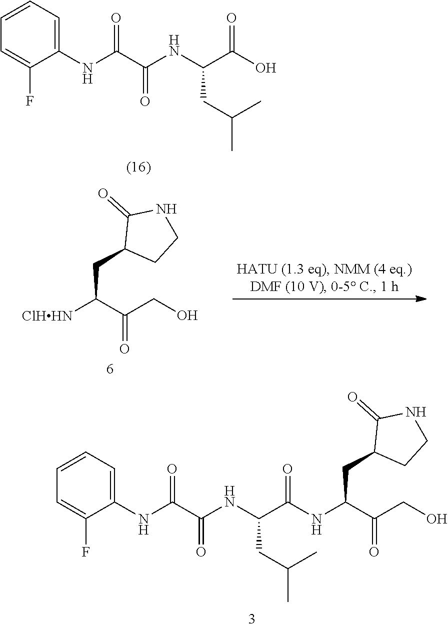

To a solution of (2-((2-fluorophenyl)amino)-2-oxoacetyl)-L-leucine (1 g, 3.45 mmol) in dry DMF (4 mL) at 0° C., was added ((S)-3-((S)-2-amino-4-hydroxy-3-oxobutyl)pyrrolidin-2-one hydrochloride (6) (1 g, 4.49 mmol) and HATU (1.5 g, 3.80 mmol) followed by NMM (1.5 mL, 13.80 mmol), and the resultant mixture was stirred at 0° C. for 30 min. After completion, the reaction mixture was diluted with ethyl acetate (30 mL) and then washed with saturated aqueous NaHCO₃ (1×20 mL), water (1×20 mL) and brine solution (1×15 mL). The organic layer was dried over anhydrous sodium sulphate, filtered, and concentrated under reduced pressure. The crude residue was then purified by column chromatography over silica gel (230-400 mesh) using 3-4% MeOH in DCM as a gradient to afford N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide (1.37 g) as an off-white solid. TLC system: MeOH: DCM (1:9); R_{f}: 0.3. Analytical Data: LCMS m/z 465.59 (M+H)⁺; ¹H NMR (400 MHz, DMSO-d₆) δ ppm 10.28 (s, 1H), 8.88 (d, J=8.8 Hz, 1H), 8.48 (d, J=8 Hz, 1H), 7.72-7.65 (m, 2H), 7.34-7.21 (m, 2H), 5.11 (t, J=5.6 Hz, 1H), 4.46-4.16 (m, 4H), 3.16-3.09 (m, 2H), 2.76 (s, 1H), 2.12-2.09 (m, 2H), 2.95-2.85 (m, 1H), 1.73-1.53 (m, 4H), 0.89-0.92 (m, 6H).

### Example 9. (1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (63)

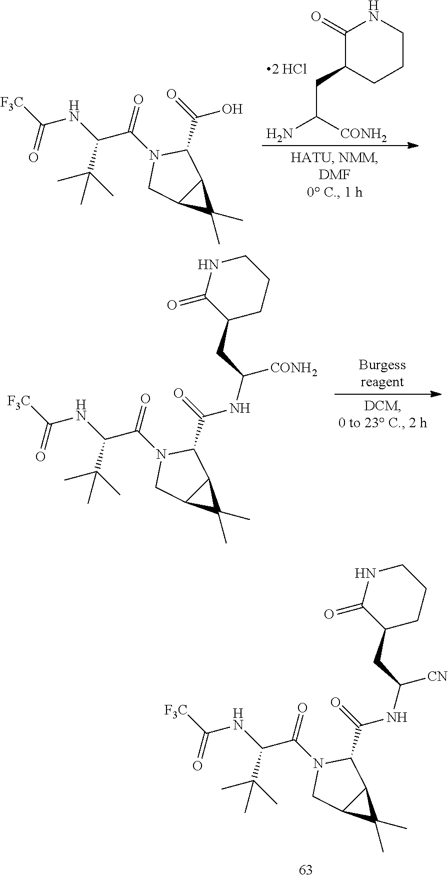

### Step 1. (1R,2S,5S)—N—((S)-1-amino-1-oxo-3-((S)-2-oxopiperidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide

(1R,2S,5S)-3-((S)-3,3-Dimethyl-2-(2,2,2-trifluoroacetamido)butanoyl)-6,6-dimethyl-3-azabicyclo[3.1.0]hexane-2-carboxylic acid (75 mg, 0.21 mmol, 1.0 eq) and HATU (87.5 mg, 0.23 mmol, 1.1 eq) were dissolved in to DMF (1.5 mL) and stirred for 10 min at 0° C. To this was added (S)-2-amino-3-((S)-2-oxopiperidin-3-yl)propanamide hydrochloride (63.5 mg. 0.23 mmol, 1.1 eq) as a solution in DMF (0.5 mL), followed by N-methylmorpholine (93.3 μL, 0.84 mmol, 4.0 eq). The reaction was continued at 0° C. for 1 hour, after which it was partitioned between H₂O/brine (1:4, 15 mL) and EtOAc (5 mL). The aqueous layer was separated and further extracted with EtOAc (3×5 mL). The combined organic layers were dried over MgSO₄, filtered, and concentrated in vacuo. The crude material was purified by flash column chromatography using a mobile phase gradient of 0→10% methanol in dichloromethane to yield (1R,2S,5S)—N—((S)-1-amino-1-oxo-3-((S)-2-oxopiperidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide as a white solid (79 mg, 0.15 mmol, 71%).

### Step 2. (1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide

(1R,2S,5S)—N—((S)-1-Amino-1-oxo-3-((S)-2-oxopiperidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (79.0 mg, 0.15 mmol, 1 eq) was dissolved in dichloromethane (1 mL) and cooled to 0° C., after which Burgess reagent (88.5 mg, 0.37 mmol, 2 eq) was added. The resultant mixture was stirred at 23° C. for 2 h. After reaction completion by LCMS, the material was directly purified by normal phase flash column chromatography using a mobile phase gradient of 0→5% methanol in dichloromethane. The resultant material was further purified on reversed-phase chromatography (C18) using a mobile phase gradient of 0→90% acetonitrile in water, yielding (1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide as a white solid (20.2 mg, 39.3 μmol, 26%). ¹H NMR (400 MHz, DMSO-d₆) δ ppm 9.39 (s, 1H), 8.99 (d, J=8.2 Hz, 1H), 7.50 (s, 1H), 5.01 (ddd, J=10.4, 8.2, 5.8 Hz, 1H), 4.41 (s, 1H), 4.16 (s, 1H), 3.90 (dd, J=10.4, 5.5 Hz, 1H), 3.68 (d, J=10.4 Hz, 1H), 3.13-3.02 (m, 2H), 2.38-2.19 (m, 2H), 1.90-1.80 (m, 1H), 1.79-1.65 (m, 2H), 1.62-1.50 (m, 2H), 1.43-1.33 (m, 1H), 1.29 (d, J=7.6 Hz, 1H), 1.02 (s, 3H), 0.98 (s, 9H), 0.84 (s, 3H); HRMS m/z calcd. for C₂₄H₃₅F₃N₅O₄⁺ (M+H)⁺ 514.2636, found 514.4242.

### Example 10. N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide (89)

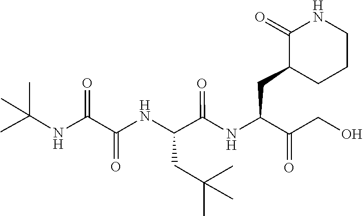 [SMILES: CC(C)(C)C[C@H](NC(=O)C(=O)NC(C)(C)C)C(=O)N[C@@H](C[C@@H]1CCCNC1=O)C(=O)CO]

### Step 1. methyl (S)-2-(2-(tert-butylamino)-2-oxoacetamido)-4,4-dimethylpentanoate

To a mixture of methyl (S)-2-amino-4,4-dimethylpentanoate (0.63 mmol, 146 mg), 2-(tert-butylamino)-2-oxoacetic acid (0.75 mmol, 109 mg), and HATU (1 eq, 0.63 mmol, 240 mg) was added dry to a vial and then dissolved in DMF (3 mL) and chilled to 0° C. This mixture was stirred at 0° C. for 10 min, after which NMM (2.3 eq, 1.4 mmol, 0.16 mL) was added dropwise over a 1 min period. The reaction was continued at 0° C. for 1 hour, after which it was quenched with deionized water (6 mL) and diluted with ethyl acetate (3 mL). This mixture was extracted with ethyl acetate (3 mL) three times, then the combined organic fractions were washed with brine (3 mL) and dried over Na₂SO₄. The crude material was then purified via normal-phase flash column chromatography (SiO₂, graduate elution in EtOAc:hexane, 0→100%), thus yielding methyl (S)-2-(2-(tert-butylamino)-2-oxoacetamido)-4,4-dimethylpentanoate as yellow oil (179 mg).

### Step 2. (S)-2-(2-(tert-butylamino)-2-oxoacetamido)-4,4-dimethylpentanoic acid

A solution of methyl (S)-2-(2-(tert-butylamino)-2-oxoacetamido)-4,4-dimethylpentanoate (1 eq, 0.63 mmol, 179 mg) in THE (1.5 mL) was stirred at 0° C. for 10 min. A solution of LiOH (2 eq, 1.2 mmol, 30. mg) in H₂O (1.5 mL) was made and added to the reaction dropwise. The reaction mixture was then stirred at 0° C. for 1 hour, followed by concentration and drying, thus yielding desired crude carboxylic acid as a white solid.

### Step 3. N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide

(S)-2-(2-(tert-Butylamino)-2-oxoacetamido)-4,4-dimethylpentanoic acid (1.2 eq, 0.41 mmol, 58 mg), (S)-3-((S)-2-amino-4-hydroxy-3-oxobutyl)piperidin-2-one hydrochloride (1 eq, 0.34 mmol, 48 mg), and HATU (1 eq, 0.34 mmol, 67 mg) were added to vial dry and dissolved in DMF (1 mL), then chilled to 0° C. This mixture was stirred at 0° C. for 10 min, after which NMM (2.3 eq, 0.79 mmol, 45 μL) was added. The reaction was continued at 0° C. for 1 hour, after which it was quenched with deionized water (15 mL). The mixture was then extracted with EtOAc (3×5 mL). Then the combined organic layers were washed with brine (5 mL) and dried over Na₂SO₄, and concentrated. The residue was purified through normal-phase flash column chromatography (silica, gradient elution in CH₂Cl₂:CH₃OH 0→5%.), and then by reverse-phase flash column chromatography (C18, gradient elution in CH₃CN:H₂O, 0→80%) to give compound 89 (26. mg; 16.7% yield).). LCMS m/z=455.4780 (M+1); ¹H NMR (500 MHz, DMSO-d₆) δ ppm 8.60 (d, J=9.0 Hz, 1H), 8.51 (d, J=8.7 Hz, 0.2H), 8.41 (d, J=7.4 Hz, 0.2H), 8.39 (d, J=7.9 Hz, 1H), 7.86 (s, 0.1H), 7.83 (s, 1H), 7.44 (s, 1H), 7.37 (s, 0.1H), 5.07 (t, J=6.0 Hz, 1H), 5.01 (t, J=6.0 Hz, 0.1H), 4.50-4.41 (m, 1H), 4.33 (td, J=9.1, 3.4 Hz, 1H), 4.24-4.07 (m, 2.5H), 3.12-3.07 (m, 2.5H), 2.18-2.05 (m, 2.5H), 1.84-1.58 (m, 5H), 1.56-1.43 (m, 0.6H), 1.32 (s, 12H), 0.88 (s, 12H).

### Example 11. (S)-3-((S)-2-(2-((2-fluorophenyl)amino)-2-oxoacetamido)-4-methylpentanamido)-2-oxo-4-((S)-2-oxopiperidin-3-yl)butyl 3-methylbutanoate (104)

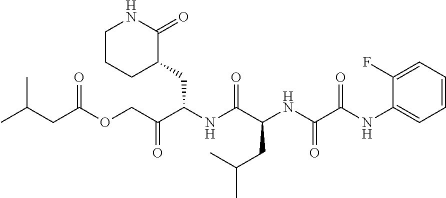 [SMILES: CC(C)CC(=O)OCC(=O)[C@H](C[C@@H]1CCCNC1=O)NC(=O)[C@H](CC(C)C)NC(=O)C(=O)Nc1ccccc1F]

To a stirred solution of N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide (1 g, 2.1 mmol, Example 12) in DCM (10 mL), TEA (0.425, 4.2 mmol) and 3-methylbutanoyl chloride (0.378 g, 3.1 mmol) were added at −70° C., and the resultant mixture was stirred at −70° C. for 3 h. After completion, the reaction mixture was diluted with water (50 mL) and extracted with ethyl acetate (2×60 mL). The combined organic layers were washed with brine solution (1×40 mL) dried over anhydrous sodium sulphate and concentrated under reduced pressure. The crude residue was then purified by flash column chromatography over silica gel (230-400 mesh) using 60% EtOAc in petroleum ether to afford (S)-3-((S)-2-(2-((2-fluorophenyl)amino)-2-oxoacetamido)-4-methylpentanamido)-2-oxo-4-((S)-2-oxopiperidin-3-yl)butyl 3-methylbutanoate (0.566 g) as a white solid. LCMS m/z 563.68 (M+1); ¹H NMR (400 MHz, DMSO-d₆) δ ppm 10.21 (s, 1H), 8.96 (d, J=8.4 Hz, 1H), 8.56 (d, J=8 Hz, 1H), 7.72-7.71 (m, 1H), 7.46 (s, 1H), 7.31-7.22 (m, 3H), 4.85-4.79 (m, 2H), 4.46-4.36 (m, 2H), 3.10 (s, 2H), 2.25-2.11 (m, 4H), 2.02-2.00 (m, 1H), 1.91-1.53 (m, 7H), 1.35-1.32 (m, 1H), 0.93-0.87 (m, 12H).

Compounds 103 and 105-108, for instance, were prepared in a manner analogous to that described in Example 11.

### Example 12. N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide (21)

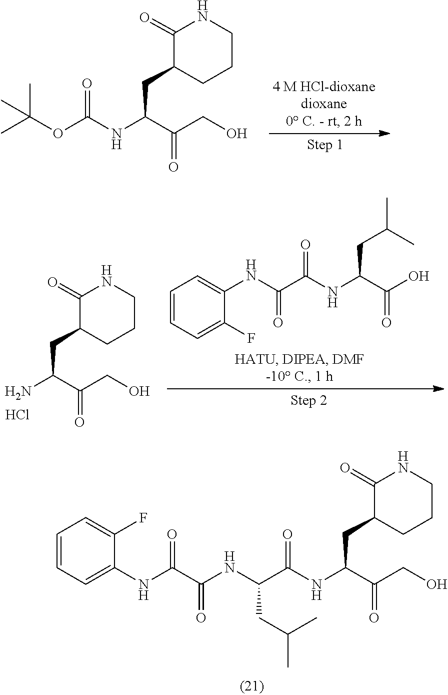

### Step 1. (S)-3-((S)-2-amino-4-hydroxy-3-oxobutyl)piperidin-2-one hydrochloride

To a stirred solution of tert-butyl ((S)-4-hydroxy-3-oxo-1-((S)-2-oxopiperidin-3-yl)butan-2-yl)carbamate (2 g, 6.65 mmol) in 1,4-dioxane (10 mL) was added 4 M HCl in 1,4-dioxane (20 mL) at 0° C., and the resultant mixture was stirred at room temperature for 2 h. After completion, the solvent was removed under reduced pressure, and the residue was then washed with diethyl ether (2×100 mL) to afford (S)-3-((S)-2-amino-4-hydroxy-3-oxobutyl)piperidin-2-one hydrochloride (1.58 g). TLC system: MeOH: DCM (1:9); R_{f}: 0.1.

### Step 2. N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide

To a stirred solution of (S)-3-((S)-2-amino-4-hydroxy-3-oxobutyl)piperidin-2-one hydrochloride (1.58 g, 6.6 mmol) and (2-((2-fluorophenyl)amino)-2-oxoacetyl)-L-leucine (1.52 g, 5.13 mmol) in DMF (15 mL) was added HATU (2.34 g, 6.15 mmol) followed by DIPEA (2.7 mL, 15.39 mmol) at −10° C., and the resultant mixture was stirred at 10° C. for 3 h. After completion, the reaction mixture was diluted with water (50 mL) and extracted with ethyl acetate (2×100 mL). The combined organic layers were washed with brine solution (3×100 mL), dried over anhydrous sodium sulfate, and concentrated under reduced pressure. The crude material was then purified by flash column chromatography over silica gel (230-400 mesh) using 7% MeOH:DCM to afford N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide (0.622 g). TLC system: 10% MeOH:DCM; R_{f}: 0.3. LCMS m/z 479.55 (M+1); ¹H NMR (400 MHz, DMSO-d₆) δ ppm 10.28 (d, J=6.7 Hz, 1H), 8.82 (t, J=8.4 Hz, 1H), 8.59 (d, J=8.0 Hz, 1H), 7.70 (t, J=7.1 Hz, 1H), 7.45 (s, 1H), 7.28 (m, J=7.1 Hz, 1H), 5.08 (t, J=5.9 Hz, 1H), 4.45 (m, J=8.4 Hz, 1H), 4.18 (m, J=9.6 Hz, 1H), 3.11 (s, 1H), 2.14 (m, J=8.6 Hz, 1H), 1.83 (s, 1H), 1.69 (m, J=8.8 Hz, 1H), 1.56 (d, J=8.5 Hz, 1H), 1.31 (m, J=11.4 Hz, 1H), 0.90 (q, J=4.9 Hz, 1H).

### Example 13. N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide (96)

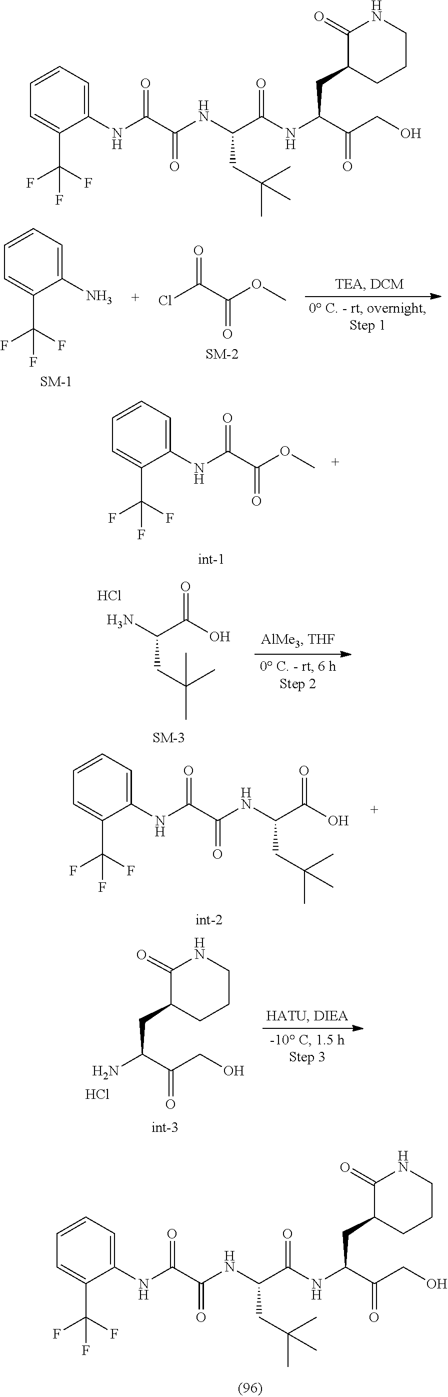

### Step 1: methyl 2-oxo-2-((2-(trifluoromethyl)phenyl)amino)acetate (int-1)

To a stirred solution of 2-(trifluoromethyl) aniline (SM-1, 9.00 g, 55.86 mmol, 1 equiv) and TEA (5.65 g, 55.86 mmol, 1.0 equiv) in DCM (90.00 ml) was added methyl 2-chloro-2-oxoacetate (SM-2, 8.21 g, 67.03 mmol, 1.2 equiv) dropwise at 0° C. under a nitrogen atmosphere. The resulting mixture was allowed to warm to ambient temperature and stirred overnight. The resulting mixture was concentrated under reduced pressure. The residue was purified by silica gel column chromatography eluted with petroleum ether/THF (9:1) to afford the title compound (int-1; 7.80 g, 56.50%). LCMS m/z=248 (M+1).

### Step 2. (S)-4,4-dimethyl-2-(2-oxo-2-((2-(trifluoromethyl)phenyl)amino)acetamido)pentanoic Acid (int-2)

To a stirred solution of (2S)-2-amino-4,4-dimethylpentanoic acid hydrochloride (SM-3, 1.45 g, 9.97 mmol, 1.0 equiv) and int-1 (2.47 g, 9.99 mmol, 1.0 equiv) in THE (30.00 ml) was added AlMe₃ (2.16 g, 29.96 mmol, 3.0 equiv) dropwise at 0° C. under a nitrogen atmosphere. The resulting mixture was stirred for additional 6 h at room temperature. The resulting mixture was concentrated under reduced pressure. The residue was purified by silica gel column chromatography eluted with THF/petroleum ether (9:1) to afford the title compound (int-2; 1.80 g, 50.02%). LCMS m/z=361 (M+1).

### Step 3. N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide

A solution of (S)-3-((S)-2-amino-4-hydroxy-3-oxobutyl)piperidin-2-one hydrochloride (int-3, 1.38 g, 5.83 mmol, 1.5 equiv) and int-2 (1.4 g, 3.885 mmol, 1.0 equiv) in DCM (13.00 ml) was treated with HATU (1.77 g, 4.66 mmol, 1.2 equiv) for 5 min at 10° C. under a nitrogen atmosphere followed by the addition of DIPEA (1.26 g, 9.71 mmol, 2.5 equiv) dropwise at −10° C. The resulting mixture was stirred for an additional 1.5 h at −10° C. The reaction was quenched with MeOH at −10° C. The resulting mixture was concentrated under reduced pressure. The residue was purified by silica gel column chromatography eluted with petroleum ether/THF (2:3) to afford 1.1 g crude product. The crude product was purified by SFC with the following conditions (Column: CHIRALPAK® IH, 3×25 cm, 5 m; Mobile Phase A: CO₂, Mobile Phase B: IPA:ACN=1:1; Back Pressure: 99 bar; Flow rate: 80 mL/min; Gradient: isocratic 35% B; Wave Length: 220 nm; Retention Time1 (min): 2.4; Retention Time 2(min): 3.8 (desired); Sample Solvent: IPA:ACN=1:1; Injection Volume: 5 mL; Number of Runs: 15) to afford the title compound (900.00 mg, 42.69%). A portion of the chromatographed title compound (700 mg) was dissolved in MTBE (21 mL) at 0° C. for 2 h. Then the mixture solution was left to stir at ambient temperature for 2 h and the suspended solids were collected by filtration and washed with MTBE (2 mL×2). The collected filter cake afforded the title compound (492 mg). LCMS m/z=543(M+1); ¹H NMR (400 MHz, DMSO-d₆) δ ppm 10.25 (s, 1H), 9.00 (d, J=8.8 Hz, 1H), 8.41 (d, J=8.0 Hz, 1H), 7.86 (d, J=8.1 Hz, 1H), 7.78-7.75 (m, 2H), 7.49 (t, J=7.7 Hz, 1H), 7.43 (s, 1H), 5.06 (t, J=5.9 Hz, 1H), 4.65-4.50 (m, 1H), 4.50-4.45 (m, 1H), 4.23-4.13 (m, 2H), 3.13-3.06 (m, 2H), 2.25-2.04 (m, 2H), 1.90-1.80 (m, 2H), 1.80-1.61 (m, 3H), 1.60-1.45 (m, 1H), 1.40-1.25 (m, 1H), 0.91 (s, 9H).

### Example 14. N¹—((S)-1-(((S)-4-hydroxy-3-oxo-1-((S)-2-oxopiperidin-3-yl) butan-2-yl) amino)-4,4-dimethyl-1-oxopentan-2-yl)-N²-(2-(trifluoromethoxy) phenyl) oxalamide (98)

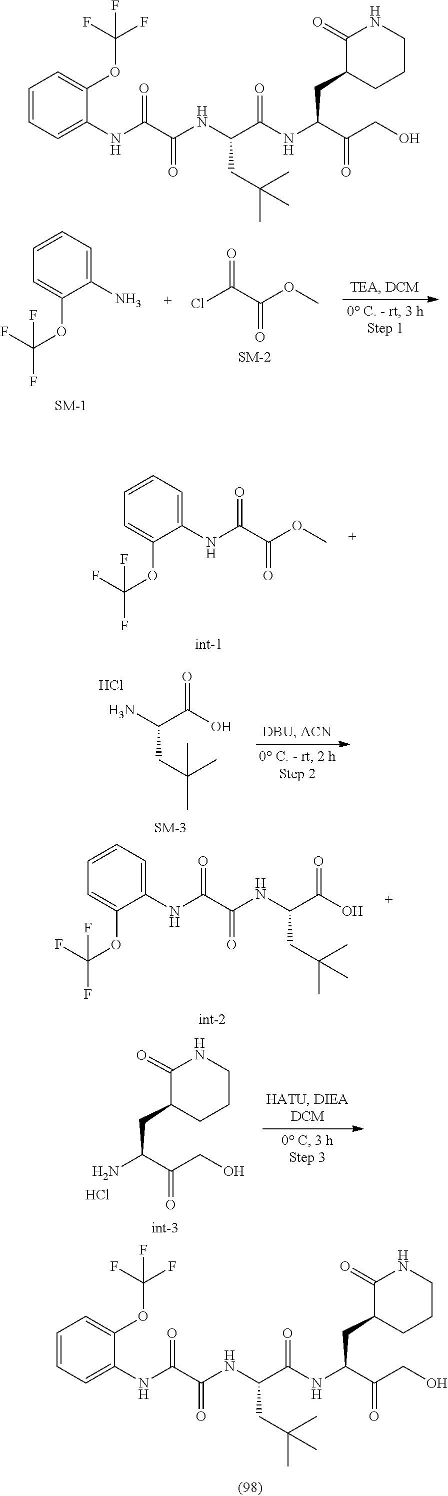

### Step 1. methyl 2-oxo-2-((2-(trifluoromethoxy)phenyl)amino)acetate (int-1)

To a stirred solution of 2-(trifluoromethoxy) aniline (SM-1, 5.0 g, 28.23 mmol, 1.0 eq) and triethylamine (8.57 mg, 84.69 mmol, 3.0 eq) in DCM (100 mL) was slowly added methyl 2-chloro-2-oxoacetate (SM-2, 3.46 g, 28.23 mmol, 1.0 eq) dropwise at 0° C. under a nitrogen atmosphere. The resulting mixture was stirred for 3 h at room temperature. After the reaction was completed, the mixture was concentrated under reduced pressure to afford methyl 2-oxo-2-((2-(trifluoromethoxy)phenyl)amino)acetate (int-1; 4.5 g, 60.57%, yield). LCMS m/z=264.1 (M+1).

### Step 2. (S)-4,4-dimethyl-2-(2-oxo-2-((2-(trifluoromethoxy)phenyl)amino)acetamido)pentanoic Acid (int-2)

To a stirred mixture of methyl 2-oxo-2-((2-(trifluoromethoxy)phenyl)amino)acetate (int-1, 4.5 g, 17.10 mmol, 1.0 eq) and (S)-2-amino-4,4-dimethylpentanoic acid (SM-3, 2.48 g, 17.10 mmol, 1.0 eq) in acetonitrile (70 mL) were slowly added 2,3,4,6,7,8,9,10-octahydropyrimido[1,2-a]azepine (3.12 g, 20.52 mmol, 3.0 eq) dropwise at 0° C. The resulting mixture was stirred for 2 h at room temperature. The reaction was monitored by LCMS. After the reaction was completed, the reaction mixture was filtered. The filtrate was concentrated under reduced pressure. The residue was purified by silica gel column chromatography eluted with petroleum ether:THF=1:1 to afford (S)-4,4-dimethyl-2-(2-oxo-2-((2-(trifluoromethoxy)phenyl)amino)acetamido)pentanoic acid (int-2; 3.70 g, 57.50%, yield). LCMS m/z=377.2 (M+1).

### Step 3. N¹—((S)-1-(((S)-4-hydroxy-3-oxo-1-((S)-2-oxopiperidin-3-yl) butan-2-yl) amino)-4,4-dimethyl-1-oxopentan-2-yl)-N²-(2-(trifluoromethoxy) phenyl) oxalamide

To a stirred solution of (S)-4,4-dimethyl-2-(2-oxo-2-((2-(trifluoromethoxy)phenyl)amino)acetamido)pentanoic acid (int-2, 3.0 g, 7.97 mmol, 1.0 eq), (S)-3-((S)-2-amino-4-hydroxy-3-oxobutyl)piperidin-2-one hydrochloride (int-3, 2.08 g, 8.77 mmol, 1.1 eq) and HATU (3.64 g, 9.57 mmol, 1.2 eq) in DCM (1.2 L) were added DIPEA (3.09 g, 23.92 mmol, 3.0 eq) dropwise at 0° C. The resulting mixture was stirred for 2 h at room temperature. After the reaction was completed, the resulting mixture was diluted with water (50 mL). The resulting mixture was extracted with EtOAc (2×150 mL). The combined organic layers were washed with brine (100 mL) and then dried over 30 g of anhydrous Na₂SO₄. The filtrate was concentrated under reduced pressure. The residue was purified by silica gel column chromatography eluted with petroleum ether:THF=1:1 to afford 1.5 g of crude product which was further purified by SFC (Column: CHIRAL ART Cellulose-SC, 3×25 cm, 5 m; Mobile Phase A: CO₂, Mobile Phase B: IPA:ACN=1:1; Flow rate: 80 mL/min; Gradient: isocratic 30% B; Column Temperature (° C.): 35; Back Pressure(bar): 100; Wave Length: 220 nm; Retention Time 1(min): 3.38 (desired); Retention Time 2(min): 4.27; Sample Solvent: IPA:ACN=1:1; Injection Volume: 4 mL; Number of Runs: 8) to give the title compound (630 mg).

The chromatographed material (200 mg) was dissolved in MTBE (6 mL) at ambient temperature. Then the mixture solution was stirred at 0° C. for 2 h and the suspended white solids were collected by filtration and washed with MTBE (1 mL×2). The collected filter cake afforded the title compound (130 mg). LCMS m/z=559.3 (M+1); ¹H NMR (400 MHz, DMSO-d₆) δ ppm 10.25 (s, 1H), 9.04 (d, J=8.8 Hz, 1H), 8.40 (d, J=8.0 Hz, 1H), 7.90 (d, J=8.0 Hz, 1H), 7.52-7.41 (m, 3H), 7.37 (d, J=7.6 Hz, 1H), 5.06 (d, J=6.0 Hz, 1H), 4.45 (d, J=8.4 Hz, 2H), 4.32-4.06 (m, 2H), 3.09 (d, J=6.6 Hz, 3H), 2.28-2.04 (m, 2H), 1.83 (d, J=9.6 Hz, 2H), 1.77-1.62 (m, 3H), 1.55 (d, J=10.8 Hz, 1H), 1.42-1.22 (m, 1H), 0.91 (s, 9H).

### Example 15. N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide (90)

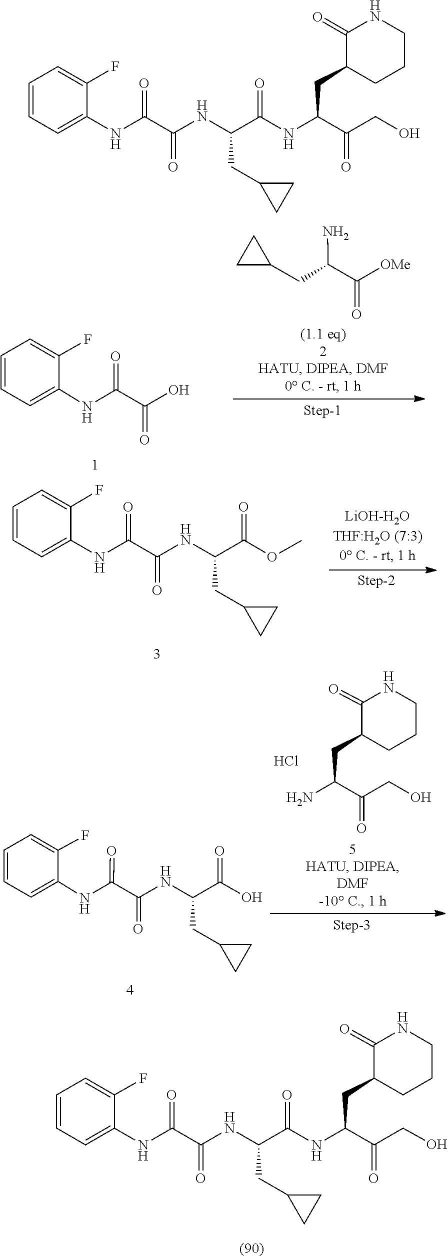

### Step 1. methyl (S)-3-cyclopropyl-2-(2-((2-fluorophenyl)amino)-2-oxoacetamido)propanoate

To a stirred solution of 2-((2-fluorophenyl)amino)-2-oxoacetic acid (1, 0.8 g, 4.36 mmol) and methyl (S)-2-amino-3-cyclopropylpropanoate (2, 0.62 g, 4.36 mmol) in DMF (10 mL) was added HATU (1.98 g, 17.44 mmol) followed by DIPEA (3 mL, 27.74 mmol) at 0° C. The resultant mixture was stirred at ambient temperature for 2 h. After completion, the reaction mixture was diluted with water (50 mL) and extracted with ethyl acetate (2×100 mL). The combined organic layers were washed with brine solution (3×100 mL), dried over anhydrous sodium sulphate, and concentrated under reduced pressure. The residue was then purified by flash column chromatography over silica gel (230-400 mesh) using 20-30% ethyl acetate in petroleum ether as the eluent to afford methyl (S)-3-cyclopropyl-2-(2-((2-fluorophenyl)amino)-2-oxoacetamido)propanoate (3, 0.56 g). TLC system: 30% EtOAc in petroleum ether; R_{f}: 0.4. LCMS m/z=309.36 (M+H).

### Step 2. (S)-3-cyclopropyl-2-(2-((2-fluorophenyl)amino)-2-oxoacetamido)propanoic Acid

To a solution of methyl (S)-3-cyclopropyl-2-(2-((2-fluorophenyl)amino)-2-oxoacetamido)propanoate (3, 0.56 g, 1.81 mmol) in THE (4 mL) and water (2 mL) was added an aqueous solution of LiOH—H₂O (0.1 g, 2.36 mmol) dropwise at 0° C., and the reaction mixture was stirred at ambient temperature for 1 h. After completion, the reaction mixture was extracted with ethyl acetate (50 mL). The aqueous layer was acidified with 10% citric acid and extracted with ethyl acetate (3×50 mL). The combined organic layers were dried over anhydrous sodium sulphate and concentrated to give (S)-3-cyclopropyl-2-(2-((2-fluorophenyl)amino)-2-oxoacetamido)propanoic acid (4, 0.35 g). TLC system: 100% EtOAc; (R_{f}: 0.1). LCMS m/z=295.20 (M+H).

### Step 3. N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide

To a stirred solution of (S)-3-cyclopropyl-2-(2-((2-fluorophenyl)amino)-2-oxoacetamido)propanoic acid (4, 0.37 g, 1.26 mmol) and (S)-3-((S)-2-amino-4-hydroxy-3-oxobutyl)piperidin-2-one hydrochloride (5, 0.3 g, 1.26 mmol) in DMF (10 mL) was added HATU (0.57 g, 1.51 mmol) followed by DIPEA (0.87 mL, 5.04 mmol) at 0° C. The resultant mixture was stirred at −10° C. for 1 h. After completion, the reaction mixture was diluted with water (50 mL) and extracted with ethyl acetate (2×100 mL). The combined organic layers were washed with brine solution (1×100 mL), dried over anhydrous sodium sulphate, and concentrated under reduced pressure. The residue was then purified by flash column chromatography over silica gel (230-400 mesh) using 100% ethyl acetate as eluent to afford N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide ((90), 0.1 g). TLC system: 10% MeOH in DCM; R_{f}: 0.4. LCMS m/z=477.2 (M+1); ¹H NMR (400 MHz, DMSO-d₆) δ ppm 10.3 (s, 1H), 8.86 (d, J=8 Hz, 1H), 8.48 (d, J=8 Hz, 1H), 7.72-7.68 (m, 1H), 7.44 (s, 1H), 7.33-7.21 (m, 3H), 5.09-5.07 (m, 1H), 4.52-4.41 (m, 2H), 4.21-4.15 (m, 2H), 3.10 (s, 2H), 2.21-2.10 (m, 2H), 1.84-1.67 (m, 4H), 1.57-1.52 (m, 2H), 1.40-1.30 (m, 1H), 0.88-0.65 (m, 1H), 0.41-0.37 (m, 2H), 0.14-0.09 (m, 2H).

### BIOLOGICAL EXAMPLES

Abbreviations: ALI for air-liquid interface; BSL3 for Biosafety Level 3; DAPI for antifade-46-diamidino-2-phenylindole; DMEM for Dulbecco's Modified Eagle Medium; DMSO for dimethyl sulfoxide; DNA for deoxyribonucleic acid; DPBS for Dulbecco's phosphate buffered saline; FBS for fetal bovine serum; LDH for lactate dehydrogenase; MTBE for methyl tert-butyl ether; MEM for minimum essential medium; MOI for multiplicity of infection; PBS for phosphate buffered saline; PET for polyethylene terephthalate; PFU for plaque-forming unit; RNA for ribonucleic acid; RT for room temperature (ambient temperature); and RT-qPCR for reverse transcription quantitative real-time polymerase chain reaction.

Virus generation. Vero E6 cells (ATCC CRL-1586) were plated in a T225 flask with complete DMEM (Corning 15-013-CV) containing 10% FBS, 1× PenStrep (Corning 20-002-CL), 2 mM L-Glutamine (Corning 25-005-CL) overnight at 37° C. and 5% CO₂. The media in the flask was removed and 2 mL of SARS-CoV-2 strain USA-WA1/2020 (BEI Resources NR-52281) in complete DMEM was added to the flask at an MOI of 0.5 and was allowed to incubate for 30 minutes at 34° C. and 5% CO₂. After incubation, 30 mL of complete DMEM was added to the flask. The flask was then placed in a 34° C. incubator at 5% CO₂ for 5 days. On day 5 post infection the supernatant was harvested and centrifuged at 1,000×g for 5 minutes. The supernatant was filtered through a 0.22 μM filter and stored at −80° C.

HeLa-ACE2 stable cell line. HeLa-ACE2 cells were generated through transduction of human ACE2 lentivirus. The lentivirus was created by co-transfection of HEK293T cells with pBOB-hACE2 construct and lentiviral packaging plasmids pMDL, pREV, and pVSV-G (Addgene) using Lipofectamine 2000 (Thermo Fisher Scientific, 11668019). Supernatant was collected 48 h after transfection then used to transduce pre-seeded HeLa cells. 12 h after transduction, stable cell lines were collected, scaled up and stored. Cells were maintained in DMEM (Gibco, 11965-092) with 10% FBS (Gibco, 10438026) and 1× sodium pyruvate (Gibco, 11360070) at 37° C. and 5% CO₂.

SARS-CoV-2/HeLa-ACE2 high-content screening assay. Compounds were acoustically transferred into 384-well μclear-bottom plates (Greiner, Part. No. 781090-2B). HeLa-ACE2 cells were seeded in 13 μL DMEM with 2% FBS at a density of 1.0×10³ cells per well. Plated cells were transported to the BSL3 facility where 13 μL of SARS-CoV-2 diluted in assay media was added to achieve ˜30-50% infected cells. Plates were incubated for 24 h at 34° C. and 5% CO₂, and then fixed with final concentration of 4% formaldehyde for 1 h at 34° C. and 5% CO₂. Plates were washed with 1×PBS 0.05% Tween 20 in between fixation and subsequent primary and secondary antibody staining. Human polyclonal plasma diluted 1:500 in Perm/Wash buffer (BD Biosciences 554723) was added to the plate and incubated at RT for 2 h. Six μg/mL of goat anti-human H+L conjugated Alexa 488 (Thermo Fisher Scientific A11013) together with 8 μM of antifade-46-diamidino-2-phenylindole (DAPI; Thermo Fisher Scientific D1306) in SuperBlock T20 (PBS) buffer (Thermo Fisher Scientific 37515) was added to the plate and incubated at RT for 1.5-2 h in the dark. Plates were imaged using the ImageXpress Micro Confocal High-Content Imaging System (Molecular Devices) with a 10× objective, with 4 fields imaged per well. Images were analyzed using the Multi-Wavelength Cell Scoring Application Module (MetaXpress), with DAPI staining identifying the host-cell nuclei (the total number of cells in the images) and the SARS-CoV-2 immunofluorescence signal leading to identification of infected cells.

Calu-3 high-content screening assay. The assay is carried out as outlined for the HeLa-ACE2 assay, with the following exceptions. Calu-3 cells (ATCC HTB-55), a kind gift from Dr. Catherine Chen at NCATS/NIH and Dr. Juan Carlos de la Torre at Scripps Research, were seeded at a density of 5,000 cells per 20 μL per well in assay media (MEM with 2% FBS) before SARS-CoV-2 diluted in assay media was added to achieve ˜30-60% infected cells. Plates were incubated for 48 h at 34° C. 5% CO₂, and then fixed with a final concentration of 4% formaldehyde. Fixed cells were stained and imaged as in the HeLa-ACE2 assay.

Uninfected host cell cytotoxicity counter screens. For both the HeLa-ACE2 and Calu3 cells, compounds were acoustically transferred into 1,536-well μclear plates (Greiner Part. No. 789091). HeLa-ACE2 cells were seeded in the assay-ready plates at 400 cells/well in DMEM with 2% FBS and plates were incubated for 24 h at 37° C. and 5% CO₂. Calu-3 cells were seeded in MEM with 2% FBS at a density of 600 cells per 5 μL per well and plates were incubated for 48 h at 37° C. and 5% CO₂. To assess cell viability, 2 μL of 50% Cell-Titer Glo (Promega No G7573) diluted in water was added to the cells and luminescence measured on an EnVision Plate Reader (Perkin Elmer).

SARS-CoV-2 primary ALI HBEC model. Normal primary human bronchial epithelial cells (HBECs) (Lonza) were cultured in Millicell-96 cell culture insert plates with 1 μm PET filters (Sigma) at an air liquid interface for at least 4 weeks using PneumaCult™-ALI Medium (Stemcell Technologies). Briefly, the HBECs were first expanded in cell culture flasks before seeding 10,000 cells per well submerged in PneumaCult™-Ex Plus Medium. After 1 week, the cells were switched into PneumaCult™-ALI Medium and medium was removed from the apical surface. The air liquid interface was maintained, and the medium exchanged every 2-3 days for at least 4 weeks to allow for differentiation of the cells. Prior to infection, the apical surface was rinsed once with DPBS and compounds were added to the basolateral chamber. 20,000 PFU SARS-CoV-2 strain USA-WA1/2020 were added to the apical surface in 50 μL PBS and allowed to incubate for 2 h. The inoculum was then removed, and the cells rinsed once with DPBS. The medium was exchanged, and fresh compound added at 24 and 48 h post-infection. Apical washes were collected at 72 h post-infection by adding 100 μL DPBS to the apical surface for 15 minutes. RNA was isolated from the apical washes using the PureLink™ Pro 96 Viral RNA/DNA Purification Kit (Thermo Fisher) and analyzed for viral RNA levels by RT-qPCR using the SuperScript™ III Platinum™ One-Step qRT-PCR Kit (Thermo Fisher) and the 2019-nCoV N1 CDC Primers and Probe set (Integrated DNA Technologies). A standard curve was generated by isolating RNA from serial dilutions of the stock virus and used to determine the PFU equivalents/mL for each sample. The viral load reductions were then determined for each experimental compound treatment compared to the neutral DMSO control and plotted in log scale. Cytotoxicity was assessed by measuring LDH activity in the basolateral media using a Cytotoxicity Detection kit (LDH) (Sigma) following the manufacturer's instructions. Averages were taken for the experimental samples and presented as a percentage of the positive control puromycin. Technical triplicates were run for both antiviral and cytotoxicity readouts.

Example 16. Results from the assays and characterizing data on exemplary compounds are presented in Table 1 and Table 2 below.

TABLE 1

| | | | LCMS | EC₅₀, μM |
|---|---|---|---|---|
| Compound | Structure | IUPAC | [M + 1] | (Hela) |
| 1 | 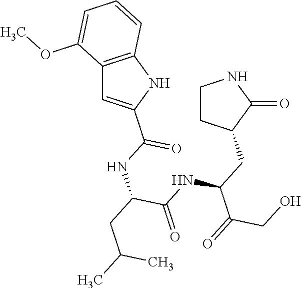 [SMILES: COc1cccc2[nH]c(C(=O)N[C@@H](CC(C)C)C(=O)N[C@@H](C[C@@H]3CCNC3=O)C(=O)CO)cc12] | N-((S)-1-(((S)-4-hydroxy-3-oxo-1- ((S)-2-oxopyrrolidin-3-yl)butan-2- yl)amino)-4-methyl-1-oxopentan-2- yl)-4-methoxy-1H-indole-2- carboxamide | 473.4713 | ** |
| 2 | 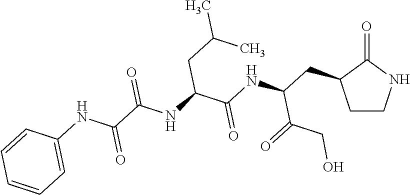 [SMILES: CC(C)C[C@H](NC(=O)C(=O)Nc1ccccc1)C(=O)N[C@@H](C[C@@H]1CCNC1=O)C(=O)CO] | N¹-((S)-1-(((S)-4-hydroxy-3-oxo-1- ((S)-2-oxopyrrolidin-3-yl)butan-2- yl)amino)-4-methyl-1-oxopentan-2- yl)-N²-phenyloxalamide | 447.22 | >1 μM |
| 3 | 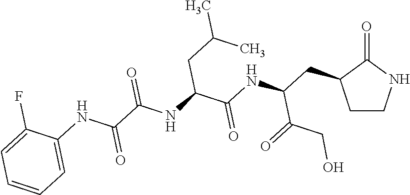 [SMILES: CC(C)C[C@H](NC(=O)C(=O)Nc1ccccc1F)C(=O)N[C@@H](C[C@@H]1CCNC1=O)C(=O)CO] | N¹-(2-fluorophenyl)-N²-((S)-1-(((S)-4- hydroxy-3-oxo-1-((S)-2- oxopyrrolidin-3-yl)butan-2-yl)amino)- 4-methyl-1-oxopentan-2-yl)oxalamide | 465.3305 | *** |
| 4 | 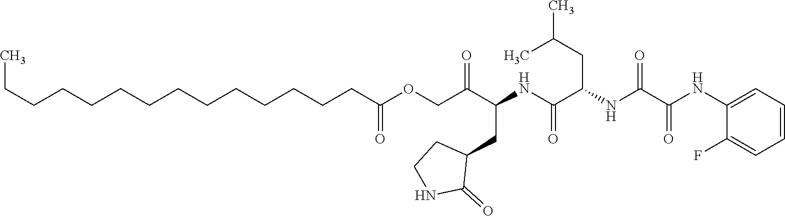 [SMILES: CCCCCCCCCCCCCCC(=O)OCC(=O)[C@H](C[C@@H]1CCNC1=O)NC(=O)[C@H](CC(C)C)NC(=O)C(=O)Nc1ccccc1F] | (S)-3-((S)-2-(2-((2- fluorophenyl)amino)-2- oxoacetamido)-4- methylpentanamido)-2-oxo-4-((S)-2- oxopyrrolidin-3-yl)butyl pentadecanoate | 688.1000 | ** |
| 5 | 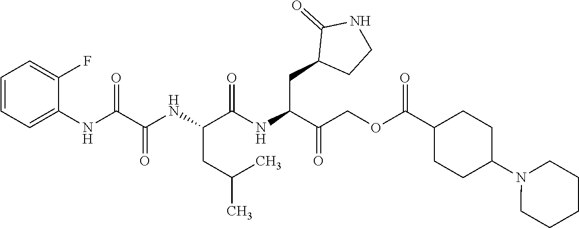 [SMILES: CC(C)C[C@H](NC(=O)C(=O)Nc1ccccc1F)C(=O)N[C@@H](C[C@@H]1CCNC1=O)C(=O)COC(=O)C1CCC(N2CCCCC2)CC1] | (S)-3-((S)-2-(2-((2- fluorophenyl)amino)-2- oxoacetamido)-4- methylpentanamido)-2-oxo-4-((S)-2- oxopyrrolidin-3-yl)butyl 4-(piperidin- 1-yl)cyclohexane-1-carboxylate | 658.2000 | ** |
| 6 | 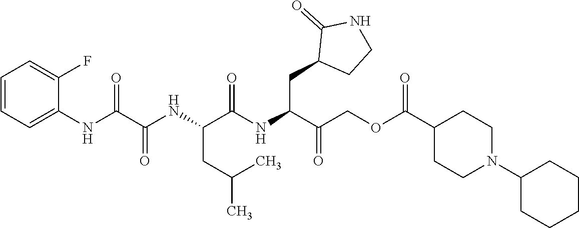 [SMILES: CC(C)C[C@H](NC(=O)C(=O)Nc1ccccc1F)C(=O)N[C@@H](C[C@@H]1CCNC1=O)C(=O)COC(=O)C1CCN(C2CCCCC2)CC1] | (S)-3-((S)-2-(2-((2- fluorophenyl)amino)-2- oxoacetamido)-4- methylpentanamido)-2-oxo-4-((S)-2- oxopyrrolidin-3-yl)butyl 1- cyclohexylpiperidine-4-carboxylate | 658.9000 | *** |
| 7 | 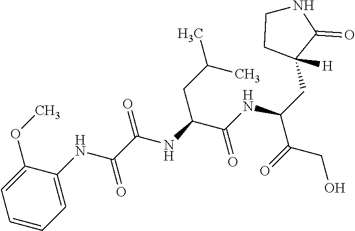 [SMILES: COc1ccccc1NC(=O)C(=O)N[C@@H](CC(C)C)C(=O)N[C@@H](C[C@@H]1CCNC1=O)C(=O)CO] | N¹-(((S)-4-hydroxy-3-oxo-1- ((S)-2-oxopyrrolidin-3-yl)butan-2- yl)amino)-4-methyl-1-oxopentan-2- yl)-N²-(2-methoxyphenyl)oxalamide | 477.3815 | >1 μM |
| 8 | 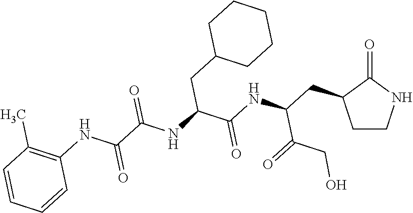 [SMILES: Cc1ccccc1NC(=O)C(=O)N[C@@H](CC1CCCCC1)C(=O)N[C@@H](C[C@@H]1CCNC1=O)C(=O)CO] | N¹-((S)-3-cyclohexyl-1-(((S)-4- hydroxy-3-oxo-1-((S)-2- oxopyrrolidin-3-yl)butan-2-yl)amino)- 1-oxopropan-2-yl)-N²-(o- tolyl)oxalamide | 502.3200 | ** |
| 9 | 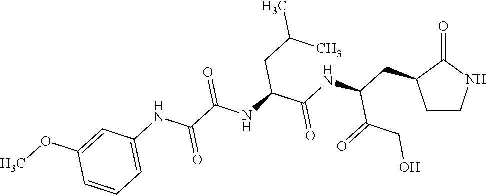 [SMILES: COc1cccc(NC(=O)C(=O)N[C@@H](CC(C)C)C(=O)N[C@@H](C[C@@H]2CCNC2=O)C(=O)CO)c1] | N¹-(((S)-4-hydroxy-3-oxo-1- ((S)-2-oxopyrrolidin-3-yl)butan-2- yl)amino)-4-methyl-1-oxopentan-2- yl)-N²-(3-methoxyphenyl)oxalamide | 477.3000 | *** |
| 10 | 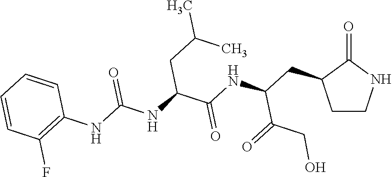 [SMILES: CC(C)C[C@H](NC(=O)Nc1ccccc1F)C(=O)N[C@@H](C[C@@H]1CCNC1=O)C(=O)CO] | (S)-2-(3-(2-fluorophenyl)ureido)-N- ((S)-4-hydroxy-3-oxo-1-((S)-2- oxopyrrolidin-3-yl)butan-2-yl)-4- methylpentanamide | 437.2600 | >1 μM |
| 11 | 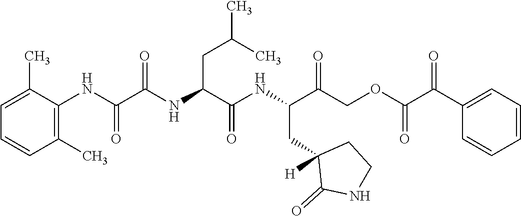 [SMILES: Cc1cccc(C)c1NC(=O)C(=O)N[C@@H](CC(C)C)C(=O)N[C@@H](C[C@@H]1CCNC1=O)C(=O)COC(=O)C(=O)c1ccccc1] | (S)-3-((S)-2-(2-((2,6- dimethylphenyl)amino)-2- oxoacetamido)-4- methylpentanamido)-2-oxo-4-((S)-2- oxopyrrolidin-3-yl)butyl 2-oxo-2- phenylacetate | 607.4182 | *** |
| 12 | 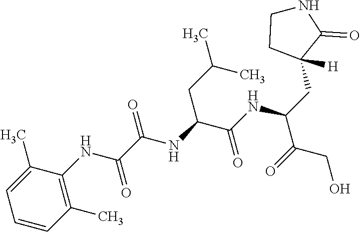 [SMILES: Cc1cccc(C)c1NC(=O)C(=O)N[C@@H](CC(C)C)C(=O)N[C@@H](C[C@@H]1CCNC1=O)C(=O)CO] | N¹-(2,6-dimethylphenyl)-N²-((S)-1- (((S)-4-hydroxy-3-oxo-1-((S)-2- oxopyrrolidin-3-yl)butan-2-yl)amino)- 4-methyl-1-oxopentan-2-yl)oxalamide | 475.3178 | >1 μM |
| 13 | 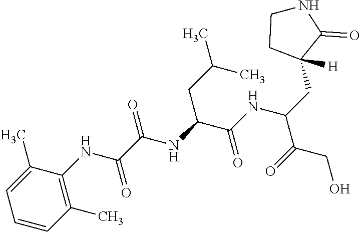 [SMILES: Cc1cccc(C)c1NC(=O)C(=O)N[C@@H](CC(C)C)C(=O)NC(C[C@@H]1CCNC1=O)C(=O)CO] | N¹-(2,6-dimethylphenyl)-N²-((2S)-1- ((4-hydroxy-3-oxo-1-((S)-2- oxopyrrolidin-3-yl)butan-2-yl)amino)- 4-methyl-1-oxopentan-2-yl)oxalamide | 475.3540 | ** |
| 14 | 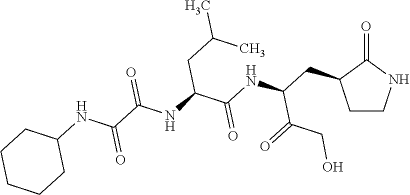 [SMILES: CC(C)C[C@H](NC(=O)C(=O)NC1CCCCC1)C(=O)N[C@@H](C[C@@H]1CCNC1=O)C(=O)CO] | N¹-cyclohexyl-N²-((S)-1-(((S)-4- hydroxy-3-oxo-1-((S)-2- oxopyrrolidin-3-yl)butan-2-yl)amino)- 4-methyl-1-oxopentan-2-yl)oxalamide | 453.3837 | >1 μM |
| 15 | 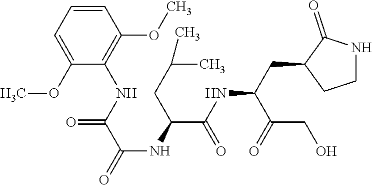 [SMILES: COc1cccc(OC)c1NC(=O)C(=O)N[C@@H](CC(C)C)C(=O)N[C@@H](C[C@@H]1CCNC1=O)C(=O)CO] | N¹-(2,6-dimethoxyphenyl)-N²-((S)-1- (((S)-4-hydroxy-3-oxo-1-((S)-2- oxopyrrolidin-3-yl)butan-2-yl)amino)- 4-methyl-1-oxopentan-2-yl)oxalamide | 507.3500 | >1 μM |
| 16 | 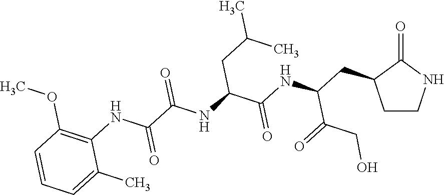 [SMILES: COc1cccc(C)c1NC(=O)C(=O)N[C@@H](CC(C)C)C(=O)N[C@@H](C[C@@H]1CCNC1=O)C(=O)CO] | N¹-((S)-1-(((S)-4-hydroxy-3-oxo-1- ((S)-2-oxopyrrolidin-3-yl)butan-2- yl)amino)-4-methyl-1-oxopentan-2- yl)-N²-(2-methoxy-6- methylphenyl)oxalamide | 492.3700 | ** |
| 17 | 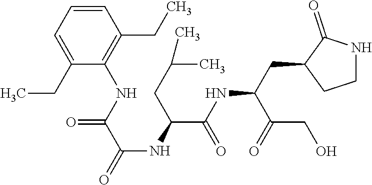 [SMILES: CCc1cccc(CC)c1NC(=O)C(=O)N[C@@H](CC(C)C)C(=O)N[C@@H](C[C@@H]1CCNC1=O)C(=O)CO] | N¹-(2,6-diethylphenyl)-N²-((S)-1-(((S)- 4-hydroxy-3-oxo-1-((S)-2- oxopyrrolidin-3-yl)butan-2-yl)amino)- 4-methyl-1-oxopentan-2-yl)oxalamide | 503.28 | **** |
| 18 | 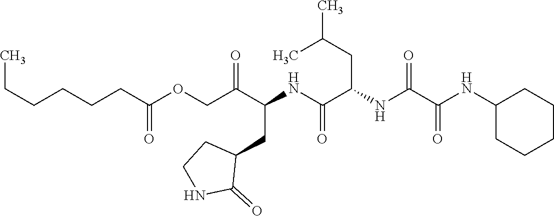 [SMILES: CCCCCCC(=O)OCC(=O)[C@H](C[C@@H]1CCNC1=O)NC(=O)[C@H](CC(C)C)NC(=O)C(=O)NC1CCCCC1] | (S)-3-((S)-2-(2-(cyclohexylamino)-2- oxoacetamido)-4- methylpentanamido)-2-oxo-4-((S)-2- oxopyrrolidin-3-yl)butyl heptanoate | 565.1735 | *** |
| 19 | 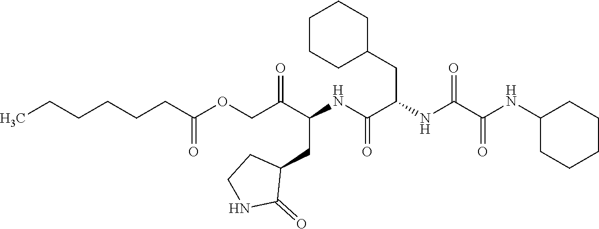 [SMILES: CCCCCCC(=O)OCC(=O)[C@H](C[C@@H]1CCNC1=O)NC(=O)[C@H](CC1CCCCC1)NC(=O)C(=O)NC1CCCCC1] | (S)-3-((S)-3-cyclohexyl-2-(2- (cyclohexylamino)-2- oxoacetamido)propanamido)-2-oxo-4- ((S)-2-oxopyrrolidin-3-yl)butyl heptanoate | 605.4634 | ** |
| 20 | 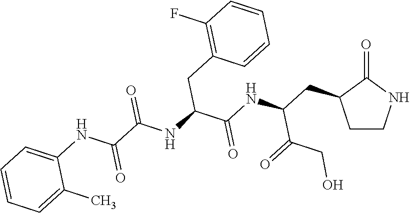 [SMILES: Cc1ccccc1NC(=O)C(=O)N[C@@H](Cc1ccccc1F)C(=O)N[C@@H](C[C@@H]1CCNC1=O)C(=O)CO] | N¹-((S)-3-(2-fluorophenyl)-1-(((S)-4- hydroxy-3-oxo-1-((S)-2- oxopyrrolidin-3-yl)butan-2-yl)amino)- 1-oxopropan-2-yl)-N²-(o- tolyl)oxalamide | 513.4500 | >1 μM |
| 21 | 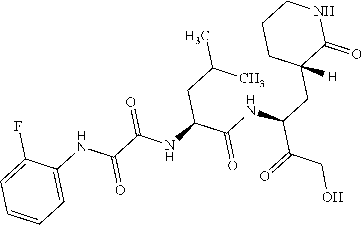 [SMILES: CC(C)C[C@H](NC(=O)C(=O)Nc1ccccc1F)C(=O)N[C@@H](C[C@@H]1CCCNC1=O)C(=O)CO] | N¹-(2-fluorophenyl)-N²-((S)-1-(((S)-4- hydroxy-3-oxo-1-((S)-2-oxopiperidin- 3-yl)butan-2-yl)amino)-4-methyl-1- oxopentan-2-yl)oxalamide | 479.0470 | *** |
| 22 | 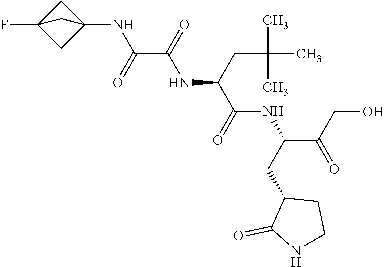 [SMILES: CC(C)(C)C[C@H](NC(=O)C(=O)NC12CC(F)(C1)C2)C(=O)N[C@@H](C[C@@H]1CCNC1=O)C(=O)CO] | N¹-(3-fluorobicyclo[1.1.1]pentan-1- yl)-N²-((S)-1-(((S)-4-hydroxy-3-oxo- 1-((S)-2-oxopyrrolidin-3-yl)butan-2- yl)amino)-4,4-dimethyl-1-oxopentan- 2-yl)oxalamide | 469.2400 | ** |
| 23 | 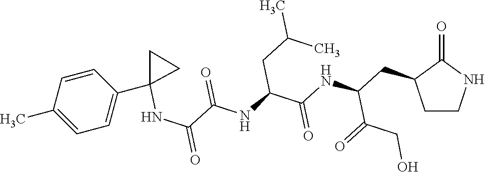 [SMILES: Cc1ccc(C2(NC(=O)C(=O)N[C@@H](CC(C)C)C(=O)N[C@@H](C[C@@H]3CCNC3=O)C(=O)CO)CC2)cc1] | N¹-(((S)-4-hydroxy-3-oxo-1- ((S)-2-oxopyrrolidin-3-yl)butan-2- yl)amino)-4-methyl-1-oxopentan-2- yl)-N²-(1-(p- tolyl)cyclopropyl)oxalamide | 501.66 | * |
| 24 | 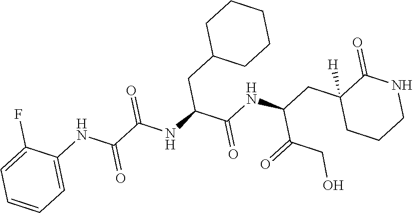 [SMILES: O=C(Nc1ccccc1F)C(=O)N[C@@H](CC1CCCCC1)C(=O)N[C@@H](C[C@@H]1CCCNC1=O)C(=O)CO] | N¹-((S)-3-cyclohexyl-1-(((S)-4- hydroxy-3-oxo-1-((S)-2-oxopiperidin- 3-yl)butan-2-yl)amino)-1-oxopropan- 2-yl)-N²-(2-fluorophenyl)oxalamide | 519.6680 | *** |
| 25 | 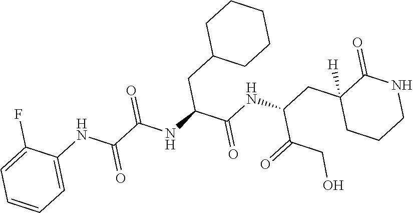 [SMILES: O=C(Nc1ccccc1F)C(=O)N[C@@H](CC1CCCCC1)C(=O)N[C@H](C[C@@H]1CCCNC1=O)C(=O)CO] | N¹-((S)-3-cyclohexyl-1-(((R)-4- hydroxy-3-oxo-1-((S)-2-oxopiperidin- 3-yl)butan-2-yl)amino)-1-oxopropan- 2-yl)-N²-(2-fluorophenyl)oxalamide | 519.6318 | *** |
| 26 | 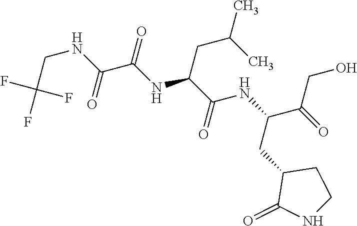 [SMILES: CC(C)C[C@H](NC(=O)C(=O)NCC(F)(F)F)C(=O)N[C@@H](C[C@@H]1CCNC1=O)C(=O)CO] | N¹-(((S)-4-hydroxy-3-oxo-1- ((S)-2-oxopyrrolidin-3-yl)butan-2- yl)amino)-4-methyl-1-oxopentan-2- yl)-N²-(2,2,2-trifluoroethyl)oxalamide | 453.1900 | >1 μM |
| 27 | 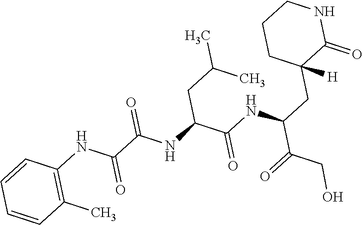 [SMILES: Cc1ccccc1NC(=O)C(=O)N[C@@H](CC(C)C)C(=O)N[C@@H](C[C@@H]1CCCNC1=O)C(=O)CO] | N¹-((S)-1-(((S)-4-hydroxy-3-oxo-1- ((S)-2-oxopiperidin-3-yl)butan-2- yl)amino)-4-methyl-1-oxopentan-2- yl)-N²-(o-tolyl)oxalamide | 475.6075 | *** |
| 28 | 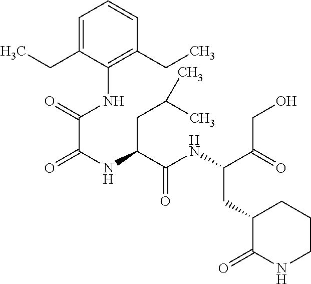 [SMILES: CCc1cccc(CC)c1NC(=O)C(=O)N[C@@H](CC(C)C)C(=O)N[C@@H](C[C@@H]1CCCNC1=O)C(=O)CO] | N¹-(2,6-diethylphenyl)-N²-((S)-1-(((S)- 4-hydroxy-3-oxo-1-((S)-2- oxopiperidin-3-yl)butan-2-yl)amino)- 4-methyl-1-oxopentan-2-yl)oxalamide | 517.2900 | **** |
| 29 | 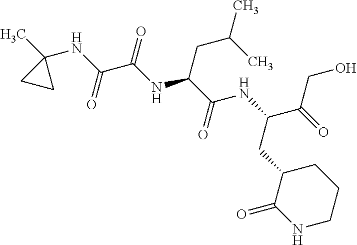 [SMILES: CC(C)C[C@H](NC(=O)C(=O)NC1(C)CC1)C(=O)N[C@@H](C[C@@H]1CCCNC1=O)C(=O)CO] | N¹-((S)-1-(((S)-4-hydroxy-3-oxo-1- ((S)-2-oxopiperidin-3-yl)butan-2- yl)amino)-4-methyl-1-oxopentan-2- yl)-N²-(1- methylcyclopropyl)oxalamide | 439.4016 | >1 μM |
| 30 | 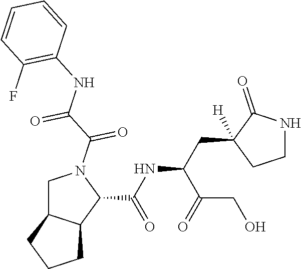 [SMILES: O=C(Nc1ccccc1F)C(=O)N1C[C@@H]2CCC[C@@H]2[C@H]1C(=O)N[C@@H](C[C@@H]1CCNC1=O)C(=O)CO] | (1S,3aR,6aS)-2-(2-((2- fluorophenyl)amino)-2-oxoacetyl)-N- ((S)-4-hydroxy-3-oxo-1-((S)-2- oxopyrrolidin-3-yl)butan-2- yl)octahydrocyclopenta[c]pyrrole-1- carboxamide | 489.3654 | *** |
| 31 | 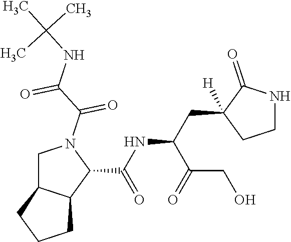 [SMILES: CC(C)(C)NC(=O)C(=O)N1C[C@@H]2CCC[C@@H]2[C@H]1C(=O)N[C@@H](C[C@@H]1CCNC1=O)C(=O)CO] | (1S,3aR,6aS)-2-(2-(tert-butylamino)- 2-oxoacetyl)-N-((S)-4-hydroxy-3-oxo- 1-((S)-2-oxopyrrolidin-3-yl)butan-2- yl)octahydrocyclopenta[c]pyrrole-1- carboxamide | 451.6749 | >1 μM |
| 32 | 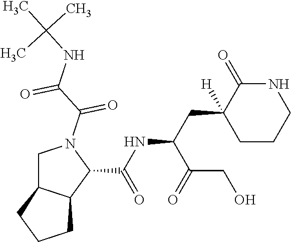 [SMILES: CC(C)(C)NC(=O)C(=O)N1C[C@@H]2CCC[C@@H]2[C@H]1C(=O)N[C@@H](C[C@@H]1CCCNC1=O)C(=O)CO] | (1S,3aR,6aS)-2-(2-(tert-butylamino)- 2-oxoacetyl)-N-((S)-4-hydroxy-3-oxo- 1-((S)-2-oxopiperidin-3-yl)butan-2- yl)octahydrocyclopenta[c]pyrrole-1- carboxamide | 465.7957 | * |
| 33 | 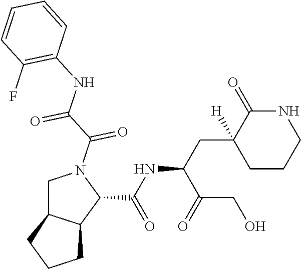 [SMILES: O=C(Nc1ccccc1F)C(=O)N1C[C@@H]2CCC[C@@H]2[C@H]1C(=O)N[C@@H](C[C@@H]1CCCNC1=O)C(=O)CO] | (1S,3aR,6aS)-2-(2-((2- fluorophenyl)amino)-2-oxoacetyl)-N- ((S)-4-hydroxy-3-oxo-1-((S)-2- oxopiperidin-3-yl)butan-2- yl)octahydrocyclopenta[c]pyrrole-1- carboxamide | 503.6661 | *** |
| 34 | 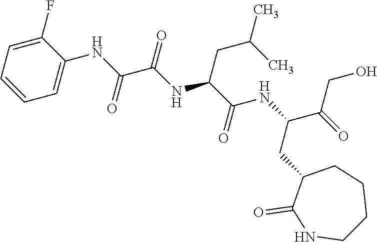 [SMILES: CC(C)C[C@H](NC(=O)C(=O)Nc1ccccc1F)C(=O)N[C@@H](C[C@@H]1CCCCNC1=O)C(=O)CO] | N¹-(2-fluorophenyl)-N²-((S)-1-(((S)-4- hydroxy-3-oxo-1-((S)-2-oxoazepan-3- yl)butan-2-yl)amino)-4-methyl-1- oxopentan-2-yl)oxalamide | 493.3500 | >1 μM |
| 35 | 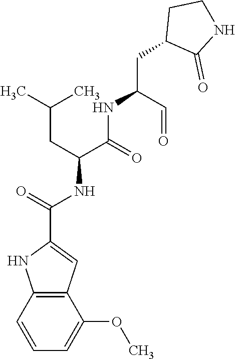 [SMILES: COc1cccc2[nH]c(C(=O)N[C@@H](CC(C)C)C(=O)N[C@H](C=O)C[C@@H]3CCNC3=O)cc12] | 4-methoxy-N-((S)-4-methyl-1-oxo-1- (((S)-1-oxo-3-((S)-2-oxopyrrolidin-3- yl)propan-2-yl)amino)pentan-2-yl)- 1H-indole-2-carboxamide | 443.5200 | >1 μM |
| 36 | 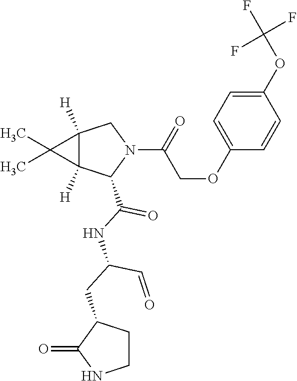 [SMILES: CC1(C)[C@@H]2[C@@H](C(=O)N[C@H](C=O)C[C@@H]3CCNC3=O)N(C(=O)COc3ccc(OC(F)(F)F)cc3)C[C@@H]21] | (1R,2S,5S)-6,6-dimethyl-N-((S)-1-oxo- 3-((S)-2-oxopyrrolidin-3-yl)propan-2- yl)-3-(2-(4- (trifluoromethoxy)phenoxy)acetyl)-3- azabicyclo[3.1.0]hexane-2- carboxamide | 512.3000 | ** |
| 37 | 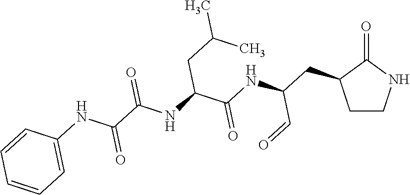 [SMILES: CC(C)C[C@H](NC(=O)C(=O)Nc1ccccc1)C(=O)N[C@H](C=O)C[C@@H]1CCNC1=O] | N¹-((S)-4-methyl-1-oxo-1-(((S)-1-oxo- 3-((S)-2-oxopyrrolidin-3-yl)propan-2- yl)amino)pentan-2-yl)-N²- phenyloxalamide | 417.3842 | >1 μM |
| 38 | 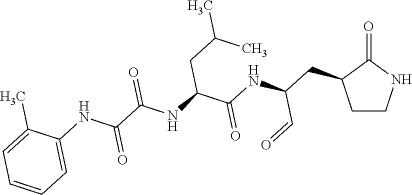 [SMILES: Cc1ccccc1NC(=O)C(=O)N[C@@H](CC(C)C)C(=O)N[C@H](C=O)C[C@@H]1CCNC1=O] | N¹-((S)-4-methyl-1-oxo-1-(((S)-1-oxo- 3-((S)-2-oxopyrrolidin-3-yl)propan-2- yl)amino)pentan-2-yl)-N²-(o- tolyl)oxalamide | 431.3282 | *** |
| 39 | 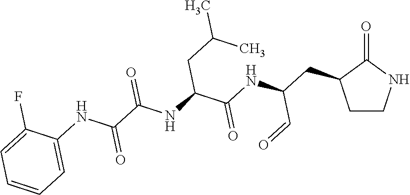 [SMILES: CC(C)C[C@H](NC(=O)C(=O)Nc1ccccc1F)C(=O)N[C@H](C=O)C[C@@H]1CCNC1=O] | N¹-(2-fluorophenyl)-N²-((S)-4-methyl- 1-oxo-1-(((S)-1-oxo-3-((S)-2- oxopyrrolidin-3-yl)propan-2- yl)amino)pentan-2-yl)oxalamide | 435.2986 | *** |
| 40 | 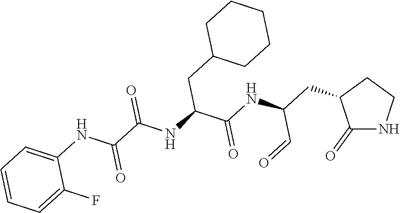 [SMILES: O=C[C@H](C[C@@H]1CCNC1=O)NC(=O)[C@H](CC1CCCCC1)NC(=O)C(=O)Nc1ccccc1F] | N¹-((S)-3-cyclohexyl-1-oxo-1-(((S)-1- oxo-3-((S)-2-oxopyrrolidin-3- yl)propan-2-yl)amino)propan-2-yl)- N²-(2-fluorophenyl)oxalamide | 475.2800 | *** |
| 41 | 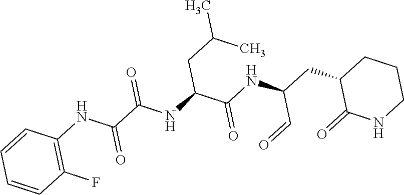 [SMILES: CC(C)C[C@H](NC(=O)C(=O)Nc1ccccc1F)C(=O)N[C@H](C=O)C[C@@H]1CCCNC1=O] | N¹-(2-fluorophenyl)-N²-((S)-4-methyl- 1-oxo-1-(((S)-1-oxo-3-((S)-2- oxopiperidin-3-yl)propan-2- yl)amino)pentan-2-yl)oxalamide | 449.2642 | *** |
| 42 | 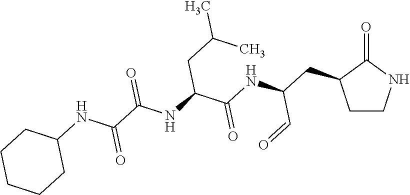 [SMILES: CC(C)C[C@H](NC(=O)C(=O)NC1CCCCC1)C(=O)N[C@H](C=O)C[C@@H]1CCNC1=O] | N¹-cyclohexyl-N²-((S)-4-methyl-1- oxo-1-(((S)-1-oxo-3-((S)-2- oxopyrrolidin-3-yl)propan-2- yl)amino)pentan-2-yl)oxalamide | 423.5700 | * |
| 43 | 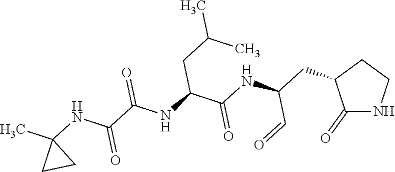 [SMILES: CC(C)C[C@H](NC(=O)C(=O)NC1(C)CC1)C(=O)N[C@H](C=O)C[C@@H]1CCNC1=O] | N¹-((S)-4-methyl-1-oxo-1-(((S)-1-oxo- 3-((S)-2-oxopyrrolidin-3-yl)propan-2- yl)amino)pentan-2-yl)-N²-(1- methylcyclopropyl)oxalamide | 395.3328 | >1 μM |
| 44 | 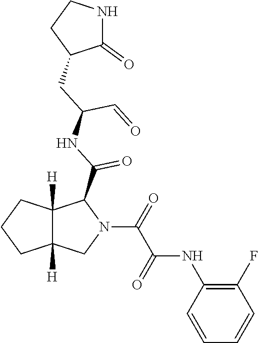 [SMILES: O=C[C@H](C[C@@H]1CCNC1=O)NC(=O)[C@@H]1[C@H]2CCC[C@H]2CN1C(=O)C(=O)Nc1ccccc1F] | (1S,3aR,6aS)-2-(2-((2- fluorophenyl)amino)-2-oxoacetyl)-N- ((S)-1-oxo-3-((S)-2-oxopyrrolidin-3- yl)propan-2- yl)octahydrocyclopenta[c]pyrrole-1- carboxamide | 459.2000 | >1 μM |
| 45 | 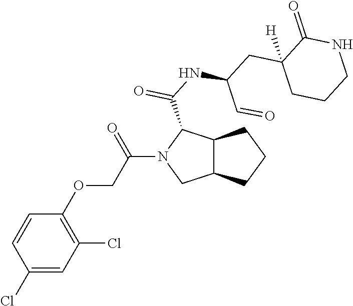 [SMILES: O=C[C@H](C[C@@H]1CCCNC1=O)NC(=O)[C@@H]1[C@H]2CCC[C@H]2CN1C(=O)COc1ccc(Cl)cc1Cl] | (1S,3aR,6aS)-2-(2-(2,4- dichlorophenoxy)acetyl)-N-((S)-1- oxo-3-((S)-2-oxopiperidin-3- yl)propan-2- yl)octahydrocyclopenta[c]pyrrole-1- carboxamide | 510.2914 | *** |
| 46 | 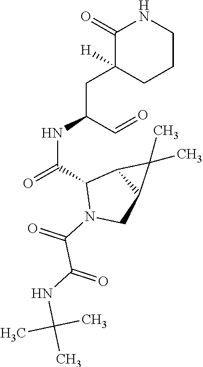 [SMILES: CC(C)(C)NC(=O)C(=O)N1C[C@H]2[C@@H]([C@H]1C(=O)N[C@H](C=O)C[C@@H]1CCCNC1=O)C2(C)C] | (1R,2S,5S)-3-(2-(tert-butylamino)-2- oxoacetyl)-6,6-dimethyl-N-((S)-1-oxo- 3-((S)-2-oxopiperidin-3-yl)propan-2- yl)-3-azabicyclo[3.1.0]hexane-2- carboxamide | 435.3590 | >1 μM |
| 47 | 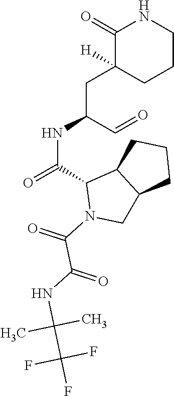 [SMILES: CC(C)(NC(=O)C(=O)N1C[C@@H]2CCC[C@@H]2[C@H]1C(=O)N[C@H](C=O)C[C@@H]1CCCNC1=O)C(F)(F)F] | (1S,3aR,6aS)-2-(2-oxo-2-((1,1,1- trifluoro-2-methylpropan-2- yl)amino)acetyl)-N-((S)-1-oxo-3-((S)- 2-oxopiperidin-3-yl)propan-2- yl)octahydrocyclopenta[c]pyrrole-1- carboxamide | 489.6189 | * |
| 48 | 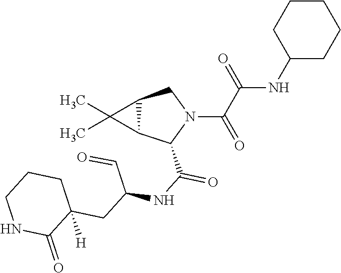 [SMILES: CC1(C)[C@@H]2[C@@H](C(=O)N[C@H](C=O)C[C@@H]3CCCNC3=O)N(C(=O)C(=O)NC3CCCCC3)C[C@@H]21] | (1R,2S,5S)-3-(2-(cyclohexylamino)-2- oxoacetyl)-6,6-dimethyl-N-((S)-1-oxo- 3-((S)-2-oxopiperidin-3-yl)propan-2- yl)-3-azabicyclo[3.1.0]hexane-2- carboxamide | 461.3000 | * |
| 49 | 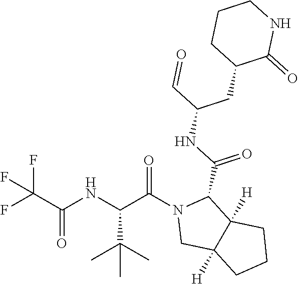 [SMILES: CC(C)(C)[C@H](NC(=O)C(F)(F)F)C(=O)N1C[C@@H]2CCC[C@@H]2[C@H]1C(=O)N[C@H](C=O)C[C@@H]1CCCNC1=O] | (1S,3aR,6aS)-2-((S)-3,3-dimethyl-2- (2,2,2-trifluoroacetamido)butanoyl)- N-((S)-1-oxo-3-((S)-2-oxopiperidin-3- yl)propan-2- yl)octahydrocyclopenta[c]pyrrole-1- carboxamide | | |
| 50 | 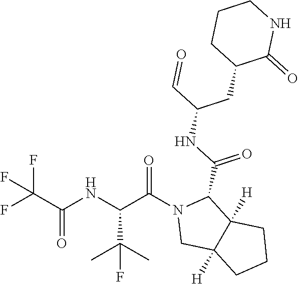 [SMILES: CC(C)(F)[C@H](NC(=O)C(F)(F)F)C(=O)N1C[C@@H]2CCC[C@@H]2[C@H]1C(=O)N[C@H](C=O)C[C@@H]1CCCNC1=O] | (1S,3aR,6aS)-2-((R)-3-fluoro-3- methyl-2-(2,2,2- trifluoroacetamido)butanoyl)-N-((S)- 1-oxo-3-((S)-2-oxopiperidin-3- yl)propan-2- yl)octahydrocyclopenta[c]pyrrole-1- carboxamide | | |
| 51 | 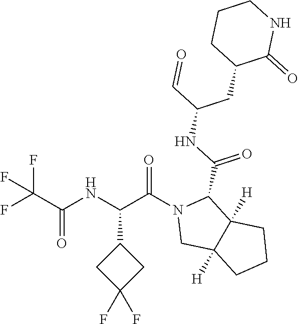 [SMILES: O=C[C@H](C[C@@H]1CCCNC1=O)NC(=O)[C@@H]1[C@H]2CCC[C@H]2CN1C(=O)[C@@H](NC(=O)C(F)(F)F)C1CC(F)(F)C1] | (1S,3aR,6aS)-2-((S)-2-(3,3- difluorocyclobutyl)-2-(2,2,2- trifluoroacetamido)acetyl)-N-((S)-1- oxo-3-((S)-2-oxopiperidin-3- yl)propan-2- yl)octahydrocyclopenta[c]pyrrole-1- carboxamide | | |
| 52 | 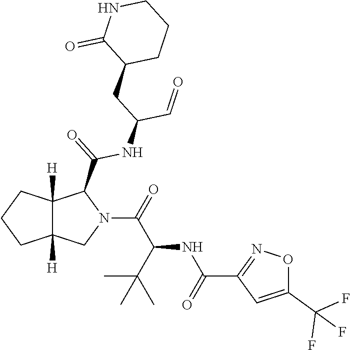 [SMILES: CC(C)(C)[C@H](NC(=O)c1cc(C(F)(F)F)on1)C(=O)N1C[C@@H]2CCC[C@@H]2[C@H]1C(=O)N[C@H](C=O)C[C@@H]1CCCNC1=O] | N-((S)-3,3-dimethyl-1-oxo-1- ((1S,3aR,6aS)-1-(((S)-1-oxo-3-((S)-2- oxopiperidin-3-yl)propan-2- yl)carbamoyl)hexahydrocyclopenta[c] pyrrol-2(1H)-yl)butan-2-yl)-5- (trifluoromethyl)isoxazole-3- carboxamide | | |
| 53 | 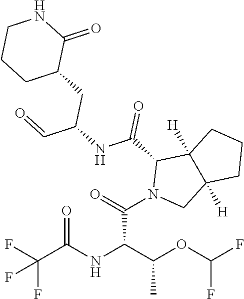 [SMILES: C[C@@H](OC(F)F)[C@H](NC(=O)C(F)(F)F)C(=O)N1C[C@@H]2CCC[C@@H]2[C@H]1C(=O)N[C@H](C=O)C[C@@H]1CCCNC1=O] | (1S,3aR,6aS)-2-(O-(difluoromethyl)- N-(2,2,2-trifluoroacetyl)-L-threonyl)- N-((S)-1-oxo-3-((S)-2-oxopiperidin-3- yl)propan-2- yl)octahydrocyclopenta[c]pyrrole-1- carboxamide | | |
| 54 | 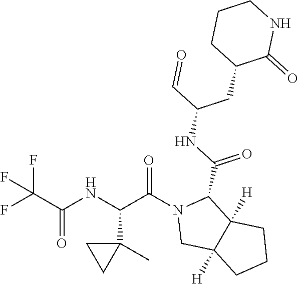 [SMILES: CC1([C@H](NC(=O)C(F)(F)F)C(=O)N2C[C@@H]3CCC[C@@H]3[C@H]2C(=O)N[C@H](C=O)C[C@@H]2CCCNC2=O)CC1] | (1S,3aR,6aS)-2-((S)-2-(1- methylcyclopropyl)-2-(2,2,2- trifluoroacetamido)acetyl)-N-((S)-1- oxo-3-((S)-2-oxopiperidin-3- yl)propan-2- yl)octahydrocyclopenta[c]pyrrole-1- carboxamide | | |
| 55 | 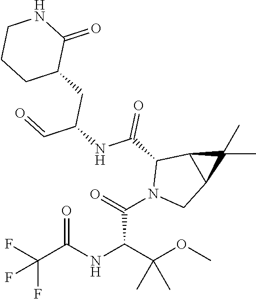 [SMILES: COC(C)(C)[C@H](NC(=O)C(F)(F)F)C(=O)N1C[C@H]2[C@@H]([C@H]1C(=O)N[C@H](C=O)C[C@@H]1CCCNC1=O)C2(C)C] | (1R,2S,5S)-3-methoxy-3- methyl-2-(2,2,2- trifluoroacetamido)butanoyl)-6,6- dimethyl-N-((S)-1-oxo-3-((S)-2- oxopiperidin-3-yl)propan-2-yl)-3- azabicyclo[3.1.0]hexane-2- carboxamide | | |
| 56 | 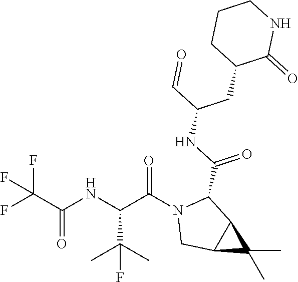 [SMILES: CC(C)(F)[C@H](NC(=O)C(F)(F)F)C(=O)N1C[C@H]2[C@@H]([C@H]1C(=O)N[C@H](C=O)C[C@@H]1CCCNC1=O)C2(C)C] | (1R,2S,5S)-3-((R)-3-fluoro-3-methyl- 2-(2,2,2-trifluoroacetamido)butanoyl)- 6,6-dimethyl-N-((S)-1-oxo-3-((S)-2- oxopiperidin-3-yl)propan-2-yl)-3- azabicyclo[3.1.0]hexane-2- carboxamide | | |
| 57 | 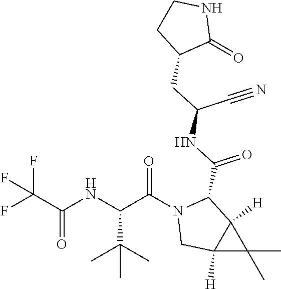 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C] | (1R,2S,5S)-N-((S)-1-cyano-2-((S)-2- oxopyrrolidin-3-yl)ethyl)-3-((S)-4,4- dimethyl-2-(2,2,2- trifluoroacetamido)pentanoyl)-6,6- dimethyl-3-azabicyclo[3.1.0]hexane- 2-carboxamide | 500.4111 | *** |
| 58 | 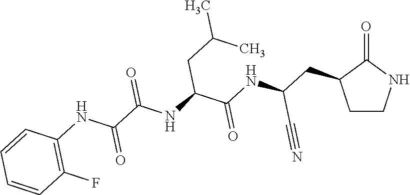 [SMILES: CC(C)C[C@H](NC(=O)C(=O)Nc1ccccc1F)C(=O)N[C@H](C#N)C[C@@H]1CCNC1=O] | N¹-(((S)-1-cyano-2-((S)-2- oxopyrrolidin-3-yl)ethyl)amino)-4- methyl-1-oxopentan-2-yl)-N²-(2- fluorophenyl)oxalamide | 432.4100 | * |
| 59 | 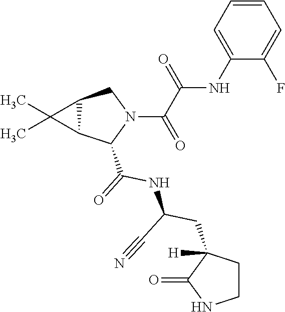 [SMILES: CC1(C)[C@@H]2[C@@H](C(=O)N[C@H](C#N)C[C@@H]3CCNC3=O)N(C(=O)C(=O)Nc3ccccc3F)C[C@@H]21] | (1R,2S,5S)-N-((S)-1-cyano-2-((S)-2- oxopyrrolidin-3-yl)ethyl)-3-(2-((2- fluorophenyl)amino)-2-oxoacetyl)- 6,6-dimethyl-3- azabicyclo[3.1.0]hexane-2- carboxamide | 456.5629 | >1 μM |
| 60 | 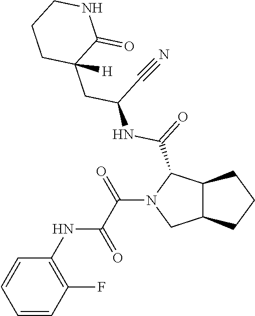 [SMILES: N#C[C@H](C[C@@H]1CCCNC1=O)NC(=O)[C@@H]1[C@H]2CCC[C@H]2CN1C(=O)C(=O)Nc1ccccc1F] | (1S,3aR,6aS)-N-((S)-1-cyano-2-((S)-2- oxopiperidin-3-yl)ethyl)-2-(2-((2- fluorophenyl)amino)-2- oxoacetyl)octahydrocyclopenta[c] pyrrole-1-carboxamide | 470.3214 | * |
| 61 | 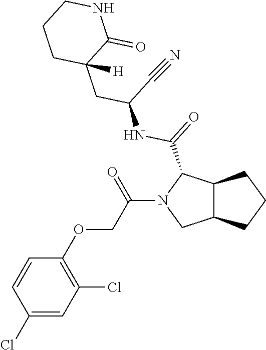 [SMILES: N#C[C@H](C[C@@H]1CCCNC1=O)NC(=O)[C@@H]1[C@H]2CCC[C@H]2CN1C(=O)COc1ccc(Cl)cc1Cl] | (1S,3aR,6aS)-N-((S)-1-cyano-2-((S)-2- oxopiperidin-3-yl)ethyl)-2-(2-(2,4- dichlorophenoxy)acetyl)octahydrocyclo- penta[c]pyrrole-1-carboxamide | 507.2865 | * |
| 62 | 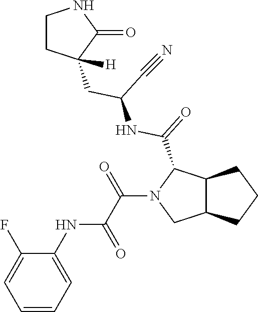 [SMILES: N#C[C@H](C[C@@H]1CCNC1=O)NC(=O)[C@@H]1[C@H]2CCC[C@H]2CN1C(=O)C(=O)Nc1ccccc1F] | (1S,3aR,6aS)-N-((S)-1-cyano-2-((S)-2- oxopyrrolidin-3-yl)ethyl)-2-(2-((2- fluorophenyl)amino)-2- oxoacetyl)octahydrocyclopenta[c] pyrrole-1-carboxamide | 456.3095 | >1 μM |
| 63 | 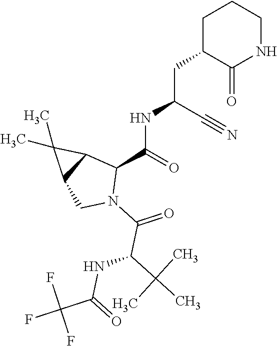 [SMILES: CC(C)(C)[C@H](NC(=O)C(F)(F)F)C(=O)N1C[C@H]2[C@@H]([C@H]1C(=O)N[C@H](C#N)C[C@@H]1CCCNC1=O)C2(C)C] | (1R,2S,5S)-N-((S)-1-cyano-2-((S)-2- oxopiperidin-3-yl)ethyl)-3-((S)-3,3- dimethyl-2-(2,2,2- trifluoroacetamido)butanoyl)-6,6- dimethyl-3-azabicyclo[3.1.0]hexane- 2-carboxamide | 514.4242 | **** |
| 64 | 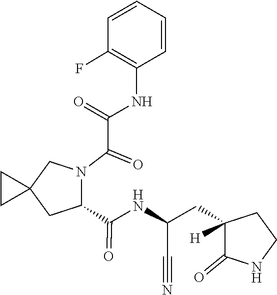 [SMILES: N#C[C@H](C[C@@H]1CCNC1=O)NC(=O)[C@@H]1CC2(CC2)CN1C(=O)C(=O)Nc1ccccc1F] | (S)-N-((S)-1-cyano-2-((S)-2- oxopyrrolidin-3-yl)ethyl)-5-(2-((2- fluorophenyl)amino)-2-oxoacetyl)-5- azaspiro[2.4]heptane-6-carboxamide | 442.18 | >1 μM |
| 65 | 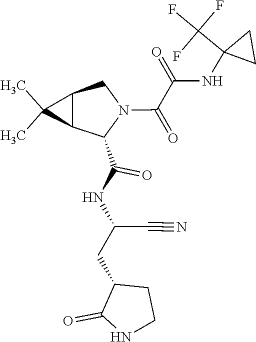 | (1R,2S,5S)-N-((S)-1-cyano-2-((S)-2- oxopyrrolidin-3-yl)ethyl)-6,6- dimethyl-3-(2-oxo-2-((1- (trifluoromethyl)cyclopropyl)amino) acetyl)-3-azabicyclo[3.1.0]hexane-2- carboxamide | 470.6000 | >1 μM |
| 66 | 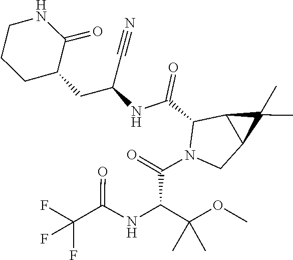 [SMILES: COC(C)(C)[C@H](NC(=O)C(F)(F)F)C(=O)N1C[C@H]2[C@@H]([C@H]1C(=O)N[C@H](C#N)C[C@@H]1CCCNC1=O)C2(C)C] | (1R,2S,5S)-N-((S)-1-cyano-2-((S)-2- oxopiperidin-3-yl)ethyl)-3-((S)-3- methoxy-3-methyl-2-(2,2,2- trifluoroacetamido)butanoyl)-6,6- dimethyl-3-azabicyclo[3.1.0]hexane- 2-carboxamide | | |
| 67 | 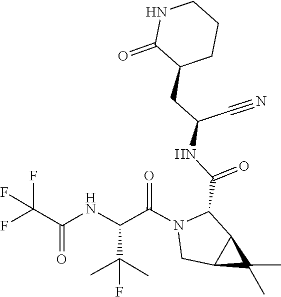 | (1R,2S,5S)-N-((S)-1-cyano-2-((S)-2- oxopiperidin-3-yl)ethyl)-3-((R)-3- fluoro-3-methyl-2-(2,2,2- trifluoroacetamido)butanoyl)-6,6- dimethyl-3-azabicyclo[3.1.0]hexane- 2-carboxamide | | |
| 68 | 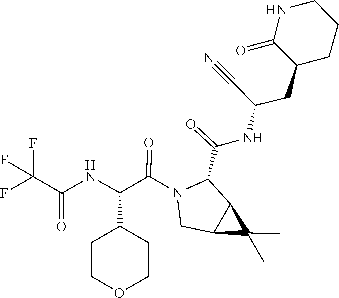 [SMILES: CC1(C)[C@@H]2[C@@H](C(=O)N[C@H](C#N)C[C@@H]3CCCNC3=O)N(C(=O)[C@@H](NC(=O)C(F)(F)F)C3CCOCC3)C[C@@H]21] | (1R,2S,5S)-N-((S)-1-cyano-2-((S)-2- oxopiperidin-3-yl)ethyl)-6,6-dimethyl- 3-((S)-2-(tetrahydro-2H-pyran-4-yl)-2- (2,2,2-trifluoroacetamido)acetyl)-3- azabicyclo[3.1.0]hexane-2- carboxamide | 542.4392 | >1 μM |
| 69 | 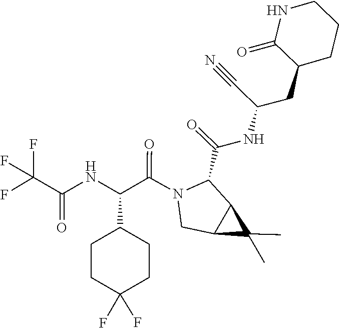 [SMILES: CC1(C)[C@@H]2[C@@H](C(=O)N[C@H](C#N)C[C@@H]3CCCNC3=O)N(C(=O)[C@@H](NC(=O)C(F)(F)F)C3CCC(F)(F)CC3)C[C@@H]21] | (1R,2S,5S)-N-((S)-1-cyano-2-((S)-2- oxopiperidin-3-yl)ethyl)-3-((S)-2-(4,4- difluorocyclohexyl)-2-(2,2,2- trifluoroacetamido)acetyl)-6,6- dimethyl-3-azabicyclo[3.1.0]hexane- 2-carboxamide | | |
| 70 | 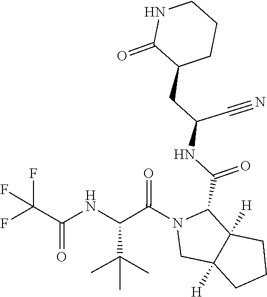 [SMILES: CC(C)(C)[C@H](NC(=O)C(F)(F)F)C(=O)N1C[C@@H]2CCC[C@@H]2[C@H]1C(=O)N[C@H](C#N)C[C@@H]1CCCNC1=O] | (1S,3aR,6aS)-N-((S)-1-cyano-2-((S)-2- oxopiperidin-3-yl)ethyl)-2-((S)-3,3- dimethyl-2-(2,2,2- trifluoroacetamido)butanoyl)octahydro- cyclopenta[c]pyrrole-1-carboxamide | | |
| 71 | 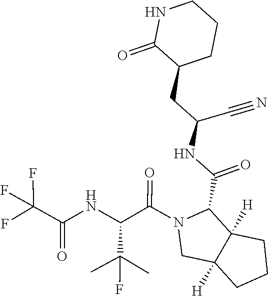 [SMILES: CC(C)(F)[C@H](NC(=O)C(F)(F)F)C(=O)N1C[C@@H]2CCC[C@@H]2[C@H]1C(=O)N[C@H](C#N)C[C@@H]1CCCNC1=O] | (1S,3aR,6aS)-N-((S)-1-cyano-2-((S)-2- oxopiperidin-3-yl)ethyl)-2-((R)-3- fluoro-3-methyl-2-(2,2,2- trifluoroacetamido)butanoyl)octahydro- cyclopenta[c]pyrrole-1-carboxamide | | |
| 72 | 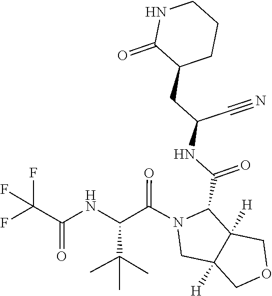 [SMILES: CC(C)(C)[C@H](NC(=O)C(F)(F)F)C(=O)N1C[C@@H]2COC[C@@H]2[C@H]1C(=O)N[C@H](C#N)C[C@@H]1CCCNC1=O] | (3aS,4S,6aR)-N-((S)-1-cyano-2-((S)-2- oxopiperidin-3-yl)ethyl)-5-((S)-3,3- dimethyl-2-(2,2,2- trifluoroacetamido)butanoyl)hexahydro- 1H-furo[3,4-c]pyrrole-4- carboxamide | | |
| 73 | 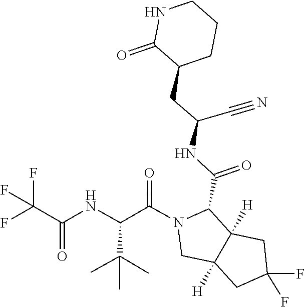 [SMILES: CC(C)(C)[C@H](NC(=O)C(F)(F)F)C(=O)N1C[C@@H]2CC(F)(F)C[C@@H]2[C@H]1C(=O)N[C@H](C#N)C[C@@H]1CCCNC1=O] | (1S,3aR,6aS)-N-((S)-1-cyano-2-((S)-2- oxopiperidin-3-yl)ethyl)-2-((S)-3,3- dimethyl-2-(2,2,2- trifluoroacetamido)butanoyl)-5,5- difluorooctahydrocyclopenta[c]pyrrole- 1-carboxamide | | |
| 74 | 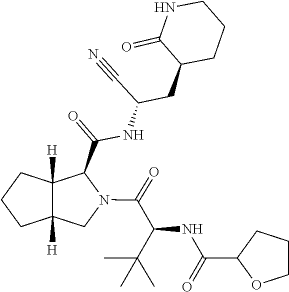 [SMILES: CC(C)(C)[C@H](NC(=O)C1CCCO1)C(=O)N1C[C@@H]2CCC[C@@H]2[C@H]1C(=O)N[C@H](C#N)C[C@@H]1CCCNC1=O] | (1S,3aR,6aS)-N-((S)-1-cyano-2-((S)-2- oxopiperidin-3-yl)ethyl)-2-((2S)-3,3- dimethyl-2-(tetrahydrofuran-2- carboxamido)butanoyl)octahydrocyclo- penta[c]pyrrole-1-carboxamide | | |
| 75 | 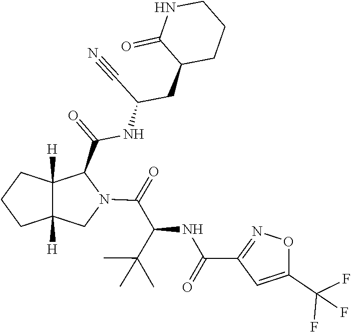 [SMILES: CC(C)(C)[C@H](NC(=O)c1cc(C(F)(F)F)on1)C(=O)N1C[C@@H]2CCC[C@@H]2[C@H]1C(=O)N[C@H](C#N)C[C@@H]1CCCNC1=O] | N-((S)-1-((1S,3aR,6aS)-1-(((S)-1- cyano-2-((S)-2-oxopiperidin-3- yl)ethyl)carbamoyl)hexahydrocyclopenta [c]pyrrol-2(1H)-yl)-3,3-dimethyl-1- oxobutan-2-yl)-5- (trifluoromethyl)isoxazole-3- carboxamide | | |
| 76 | 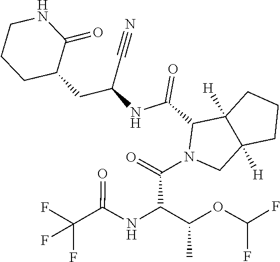 | (1S,3aR,6aS)-N-((S)-1-cyano-2-((S)-2- oxopiperidin-3-yl)ethyl)-2-(O- (difluoromethyl)-N-(2,2,2- trifluoroacetyl)-L- threonyl)octahydrocyclopenta[c]pyrroe-l 1-carboxamide | | |
| 77 | 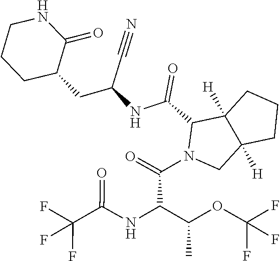 | (1S,3aR,6aS)-N-((S)-1-cyano-2-((S)-2- oxopiperidin-3-yl)ethyl)-2-(N-(2,2,2- trifluoroacetyl)-O-(trifluoromethyl)-L- threonyl)octahydrocyclopenta[c]pyrrole- 1-carboxamide | | |
| 78 | 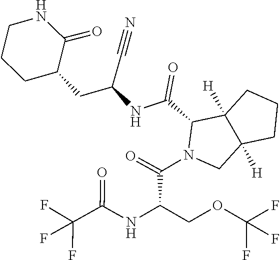 [SMILES: N#C[C@H](C[C@@H]1CCCNC1=O)NC(=O)[C@@H]1[C@H]2CCC[C@H]2CN1C(=O)[C@H](COC(F)(F)F)NC(=O)C(F)(F)F] | (1S,3aR,6aS)-N-((S)-1-cyano-2-((S)-2- oxopiperidin-3-yl)ethyl)-2-(N-(2,2,2- trifluoroacetyl)-O-(trifluoromethyl)-L- seryl)octahydrocyclopenta[c]pyrrole- 1-carboxamide | | |
| 79 | 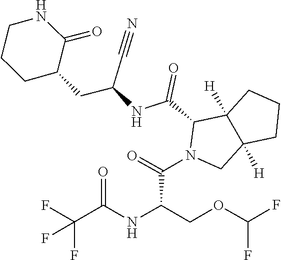 [SMILES: N#C[C@H](C[C@@H]1CCCNC1=O)NC(=O)[C@@H]1[C@H]2CCC[C@H]2CN1C(=O)[C@H](COC(F)F)NC(=O)C(F)(F)F] | (1S,3aR,6aS)-N-((S)-1-cyano-2-((S)-2- oxopiperidin-3-yl)ethyl)-2-(O- (difluoromethyl)-N-(2,2,2- trifluoroacetyl)-L- seryl)octahydrocyclopenta[c]pyrrole- 1-carboxamide | | |
| 80 | 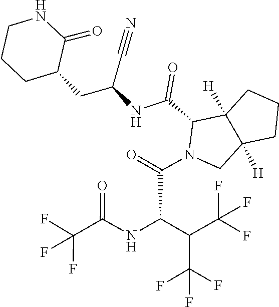 [SMILES: N#C[C@H](C[C@@H]1CCCNC1=O)NC(=O)[C@@H]1[C@H]2CCC[C@H]2CN1C(=O)[C@@H](NC(=O)C(F)(F)F)C(C(F)(F)F)C(F)(F)F] | (1S,3aR,6aS)-N-((S)-1-cyano-2-((S)-2- oxopiperidin-3-yl)ethyl)-2-((S)-4,4,4- trifluoro-2-(2,2,2-trifluoroacetamido)- 3- (trifluoromethyl)butanoyl)octahydrocyclo- penta[c]pyrrole-1-carboxamide | | |
| 81 | 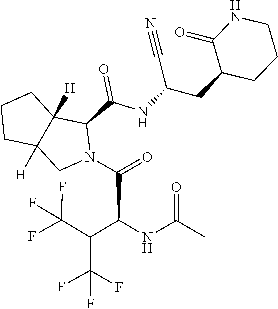 [SMILES: CC(=O)N[C@H](C(=O)N1CC2CCC[C@@H]2[C@H]1C(=O)N[C@H](C#N)C[C@@H]1CCCNC1=O)C(C(F)(F)F)C(F)(F)F] | (1S,3aR,6aS)-2-((S)-2-acetamido- 4,4,4-trifluoro-3- (trifluoromethyl)butanoyl)-N-((S)-1- cyano-2-((S)-2-oxopiperidin-3- yl)ethyl)octahydrocyclopenta[c]pyrrole- 1-carboxamide | | |
| 82 | 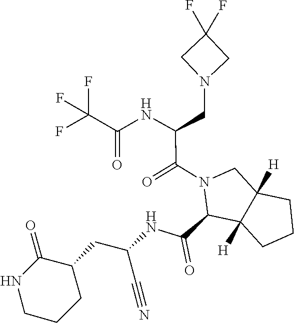 [SMILES: N#C[C@H](C[C@@H]1CCCNC1=O)NC(=O)[C@@H]1[C@H]2CCC[C@H]2CN1C(=O)[C@H](CN1CC(F)(F)C1)NC(=O)C(F)(F)F] | (1S,3aR,6aS)-N-((S)-1-cyano-2-((S)-2- oxopiperidin-3-yl)ethyl)-2-((S)-3-(3,3- difluoroazetidin-1-yl)-2-(2,2,2- trifluoroacetamido)propanoyl)octahydro- cyclopenta[c]pyrrole-1-carboxamide | | |
| 83 | 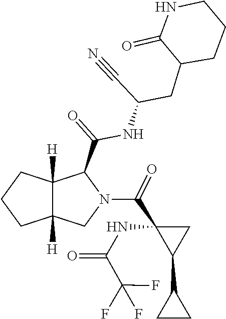 [SMILES: N#C[C@H](CC1CCCNC1=O)NC(=O)[C@@H]1[C@H]2CCC[C@H]2CN1C(=O)[C@@]1(NC(=O)C(F)(F)F)C[C@H]1C1CC1] | (1S,3aR,6aS)-N-((1S)-1-cyano-2-(2- oxopiperidin-3-yl)ethyl)-2-((1S,2R)-2- (2,2,2-trifluoroacetamido)-[1,1′- bi(cyclopropane)]-2- carbonyl)octahydrocyclopenta[c]pyrrole- 1-carboxamide | | |
| 84 | 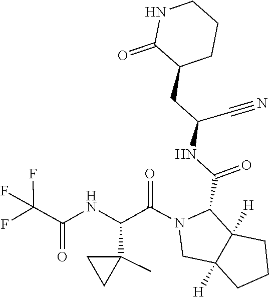 [SMILES: CC1([C@H](NC(=O)C(F)(F)F)C(=O)N2C[C@@H]3CCC[C@@H]3[C@H]2C(=O)N[C@H](C#N)C[C@@H]2CCCNC2=O)CC1] | (1S,3aR,6aS)-N-((S)-1-cyano-2-((S)-2- oxopiperidin-3-yl)ethyl)-2-((S)-2-(1- methylcyclopropyl)-2-(2,2,2- trifluoroacetamido)acetyl)octahydrocyclo- penta[c]pyrrole-1-carboxamide | | |
| 85 | 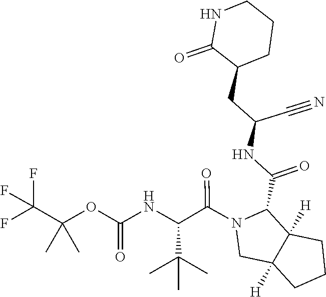 [SMILES: CC(C)(C)[C@H](NC(=O)OC(C)(C)C(F)(F)F)C(=O)N1C[C@@H]2CCC[C@@H]2[C@H]1C(=O)N[C@H](C#N)C[C@@H]1CCCNC1=O] | 1,1,1-trifluoro-2-methylpropan-2-yl ((S)-1-((1S,3aR,6aS)-1-(((S)-1-cyano- 2-((S)-2-oxopiperidin-3- yl)ethyl)carbamoyl)hexahydrocyclopenta [c]pyrrol-2(1H)-yl)-3,3-dimethyl-1- oxobutan-2-yl)carbamate | | |
| 86 | 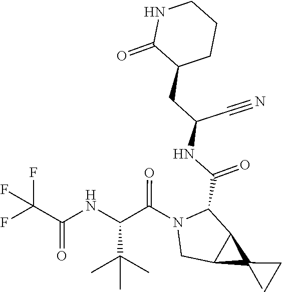 | (1R,2S,5S)-N-((S)-1-cyano-2-((S)-2- oxopiperidin-3-yl)ethyl)-3-((S)-3,3- dimethyl-2-(2,2,2- trifluoroacetamido)butanoyl)-3- azaspiro[bicyclo[3.1.0]hexane-6,1′- cyclopropane]-2-carboxamide | | |
| 87 | 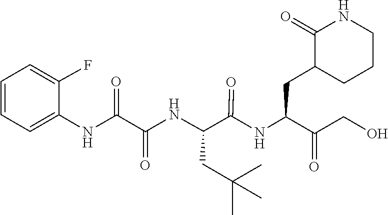 [SMILES: CC(C)(C)C[C@H](NC(=O)C(=O)Nc1ccccc1F)C(=O)N[C@@H](CC1CCCNC1=O)C(=O)CO] | N¹-(2-fluorophenyl)-N²-((S)-1-(((S)-4- hydroxy-3-oxo-1-((S)-2-oxopiperidin- 3-yl)butan-2-yl)amino)-4,4-dimethyl- 1-oxopentan-2-yl)oxalamide | 493.46 | **** |
| 88 | 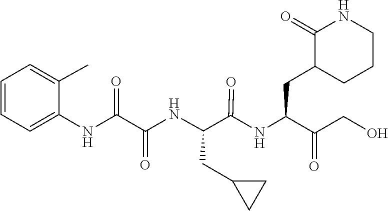 [SMILES: Cc1ccccc1NC(=O)C(=O)N[C@@H](CC1CC1)C(=O)N[C@@H](CC1CCCNC1=O)C(=O)CO] | N¹-((S)-3-cyclopropyl-1-(((S)-4- hydroxy-3-oxo-1-((S)-2-oxopiperidin- 3-yl)butan-2-yl)amino)-1-oxopropan- 2-yl)-N²-(o-tolyl)oxalamide | 473.43 | **** |
| 89 | 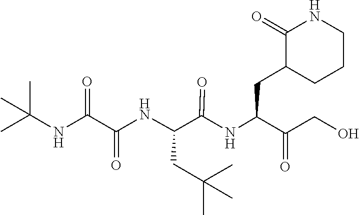 [SMILES: CC(C)(C)C[C@H](NC(=O)C(=O)NC(C)(C)C)C(=O)N[C@@H](CC1CCCNC1=O)C(=O)CO] | N¹-(tert-butyl)-N²-((S)-1-(((S)-4- hydroxy-3-oxo-1-((S)-2-oxopiperidin- 3-yl)butan-2-yl)amino)-4,4-dimethyl- 1-oxopentan-2-yl)oxalamide | 455.5 | *** |
| 90 | 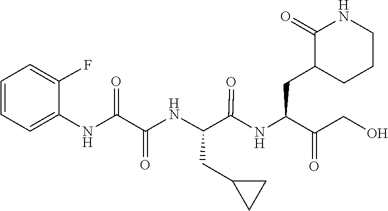 [SMILES: O=C(Nc1ccccc1F)C(=O)N[C@@H](CC1CC1)C(=O)N[C@@H](CC1CCCNC1=O)C(=O)CO] | N¹-((S)-3-cyclopropyl-1-(((S)-4- hydroxy-3-oxo-1-((S)-2-oxopiperidin- 3-yl)butan-2-yl)amino)-1-oxopropan- 2-yl)-N²-(2-fluorophenyl)oxalamide | 477.4 | **** |
| 91 | 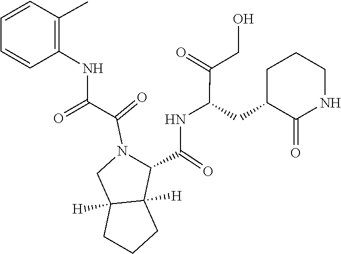 [SMILES: Cc1ccccc1NC(=O)C(=O)N1C[C@@H]2CCC[C@@H]2[C@H]1C(=O)N[C@@H](C[C@@H]1CCCNC1=O)C(=O)CO] | (1S,3aR,6aS)-N-((S)-4-hydroxy-3-oxo- 1-((S)-2-oxopiperidin-3-yl)butan-2- yl)-2-(2-oxo-2-(o- tolylamino)acetyl)octahydrocyclopenta [c]pyrrole-1-carboxamide | 499.72 | *** |
| 92 | 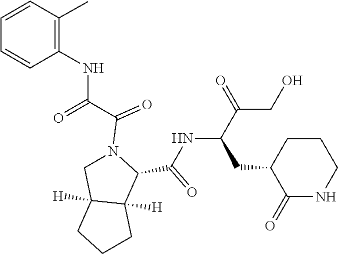 [SMILES: Cc1ccccc1NC(=O)C(=O)N1C[C@@H]2CCC[C@@H]2[C@H]1C(=O)N[C@H](C[C@@H]1CCCNC1=O)C(=O)CO] | (1S,3aR,6aS)-N-((R)-4-hydroxy-3- oxo-1-((S)-2-oxopiperidin-3-yl)butan- 2-yl)-2-(2-oxo-2-(o- tolylamino)acetyl)octahydrocyclopenta [c]pyrrole-1-carboxamide | 499.43 | >1 μM |
| 93 | 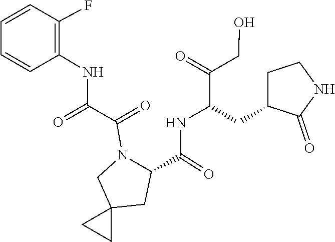 [SMILES: O=C(Nc1ccccc1F)C(=O)N1CC2(CC2)C[C@H]1C(=O)N[C@@H](C[C@@H]1CCNC1=O)C(=O)CO] | (S)-5-(2-((2-fluorophenyl)amino)-2- oxoacetyl)-N-((S)-4-hydroxy-3-oxo-1- ((S)-2-oxopyrrolidin-3-yl)butan-2-yl)- 5-azaspiro[2.4]heptane-6-carboxamide | 475.4 | ** |
| 94 | 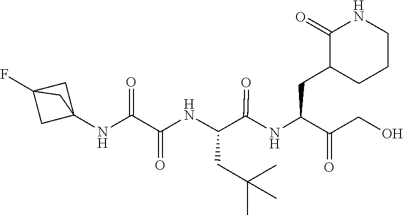 [SMILES: CC(C)(C)C[C@H](NC(=O)C(=O)NC12CC(F)(C1)C2)C(=O)N[C@@H](CC1CCCNC1=O)C(=O)CO] | N¹-(3-fluorobicyclo[1.1.1]pentan-1- yl)-N²-((S)-1-(((S)-4-hydroxy-3-oxo- 1-((S)-2-oxopiperidin-3-yl)butan-2- yl)amino)-4,4-dimethyl-1-oxopentan- 2-yl)oxalamide | 483.5 | *** |
| 95 | 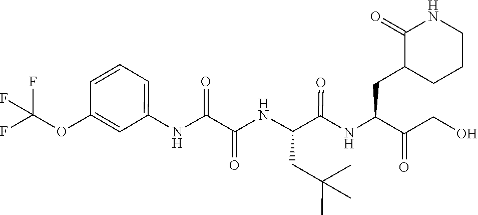 [SMILES: CC(C)(C)C[C@H](NC(=O)C(=O)Nc1cccc(OC(F)(F)F)c1)C(=O)N[C@@H](CC1CCCNC1=O)C(=O)CO] | N¹-(((S)-4-hydroxy-3-oxo-1- ((S)-2-oxopiperidin-3-yl)butan-2- yl)amino)-4,4-dimethyl-1-oxopentan- 2-yl)-N²-(3- (trifluoromethoxy)phenyl)oxalamide | 559.45 | **** |
| 96 | 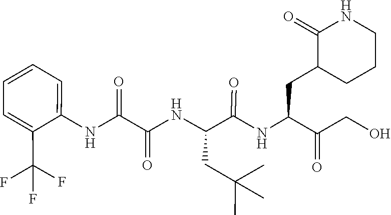 [SMILES: CC(C)(C)C[C@H](NC(=O)C(=O)Nc1ccccc1C(F)(F)F)C(=O)N[C@@H](CC1CCCNC1=O)C(=O)CO] | N¹-(((S)-4-hydroxy-3-oxo-1- ((S)-2-oxopiperidin-3-yl)butan-2- yl)amino)-4,4-dimethyl-1-oxopentan- 2-yl)-N²-(2- (trifluoromethyl)phenyl)oxalamide | 543.2 | **** |
| 97 | 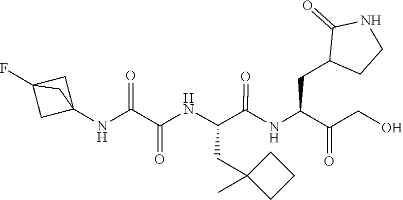 [SMILES: CC1(C[C@H](NC(=O)C(=O)NC23CC(F)(C2)C3)C(=O)N[C@@H](CC2CCNC2=O)C(=O)CO)CCC1] | N¹-(3-fluorobicyclo[1.1.1]pentan-1- yl)-N²-((S)-1-(((S)-4-hydroxy-3-oxo- 1-((S)-2-oxopyrrolidin-3-yl)butan-2- yl)amino)-3-(1-methylcyclobutyl)-1- oxopropan-2-yl)oxalamide | 481.65 | *** |
| 98 | 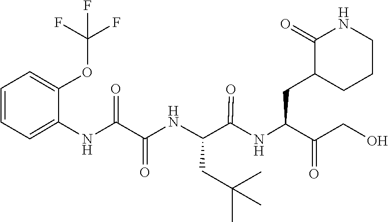 [SMILES: CC(C)(C)C[C@H](NC(=O)C(=O)Nc1ccccc1OC(F)(F)F)C(=O)N[C@@H](CC1CCCNC1=O)C(=O)CO] | N¹-(((S)-4-hydroxy-3-oxo-1- ((S)-2-oxopiperidin-3-yl)butan-2- yl)amino)-4,4-dimethyl-1-oxopentan- 2-yl)-N²-(2- (trifluoromethoxy)phenyl)oxalamide | 559.4 | **** |
| 99 | 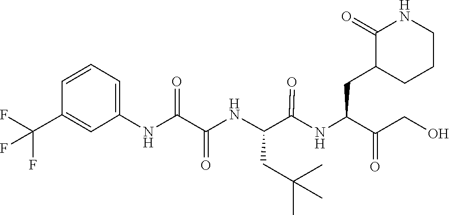 [SMILES: CC(C)(C)C[C@H](NC(=O)C(=O)Nc1cccc(C(F)(F)F)c1)C(=O)N[C@@H](CC1CCCNC1=O)C(=O)CO] | N¹-(((S)-4-hydroxy-3-oxo-1- ((S)-2-oxopiperidin-3-yl)butan-2- yl)amino)-4,4-dimethyl-1-oxopentan- 2-yl)-N²-(3- (trifluoromethyl)phenyl)oxalamide | 543.5 | **** |
| 100 | 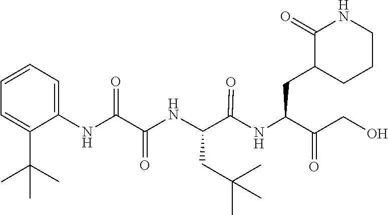 [SMILES: CC(C)(C)C[C@H](NC(=O)C(=O)Nc1ccccc1C(C)(C)C)C(=O)N[C@@H](CC1CCCNC1=O)C(=O)CO] | N¹-(2-(tert-butyl)phenyl)-N²-((S)-1- (((S)-4-hydroxy-3-oxo-1-((S)-2- oxopiperidin-3-yl)butan-2-yl)amino)- 4,4-dimethyl-1-oxopentan-2- yl)oxalamide | 531.8 | **** |
| 101 | 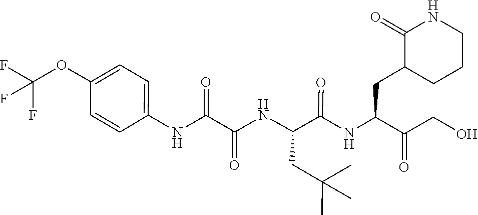 [SMILES: CC(C)(C)C[C@H](NC(=O)C(=O)Nc1ccc(OC(F)(F)F)cc1)C(=O)N[C@@H](CC1CCCNC1=O)C(=O)CO] | N¹-((S)-1-(((S)-4-hydroxy-3-oxo-1- ((S)-2-oxopiperidin-3-yl)butan-2- yl)amino)-4,4-dimethyl-1-oxopentan- 2-yl)-N²-(4- (trifluoromethoxy)phenyl)oxalamide | 559.5 | *** |
| 102 | 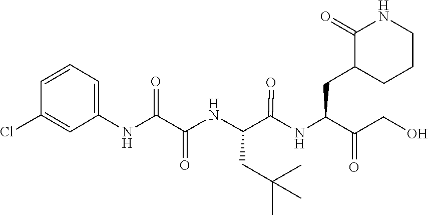 [SMILES: CC(C)(C)C[C@H](NC(=O)C(=O)Nc1cccc(Cl)c1)C(=O)N[C@@H](CC1CCCNC1=O)C(=O)CO] | N¹-(3-chlorophenyl)-N²-((S)-1-(((S)-4- hydroxy-3-oxo-1-((S)-2-oxopiperidin- 3-yl)butan-2-yl)amino)-4,4-dimethyl- l -oxopentan-2-yl)oxalamide | 509.4 | **** |
| 103 | 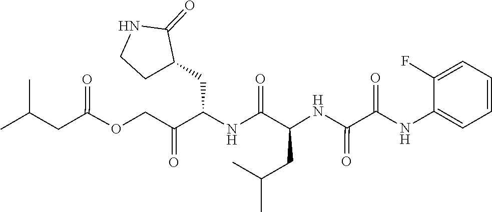 [SMILES: CC(C)CC(=O)OCC(=O)[C@H](C[C@@H]1CCNC1=O)NC(=O)[C@H](CC(C)C)NC(=O)C(=O)Nc1ccccc1F] | (S)-3-((S)-2-(2-((2- fluorophenyl)amino)-2- oxoacetamido)-4- methylpentanamido)-2-oxo-4-((S)-2- oxopyrrolidin-3-yl)butyl 3- methylbutanoate | 549.52 | *** |
| 104 | 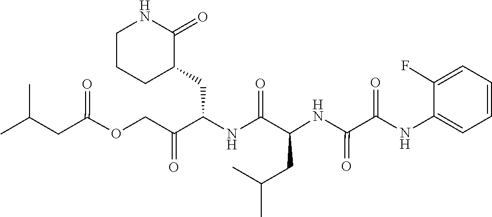 [SMILES: CC(C)CC(=O)OCC(=O)[C@H](C[C@@H]1CCCNC1=O)NC(=O)[C@H](CC(C)C)NC(=O)C(=O)Nc1ccccc1F] | (S)-3-((S)-2-(2-((2- fluorophenyl)amino)-2- oxoacetamido)-4- methylpentanamido)-2-oxo-4-((S)-2- oxopiperidin-3-yl)butyl 3- methylbutanoate | 563.68 | *** |
| 105 | 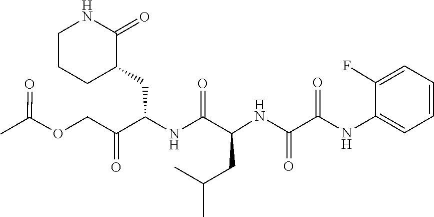 [SMILES: CC(=O)OCC(=O)[C@H](C[C@@H]1CCCNC1=O)NC(=O)[C@H](CC(C)C)NC(=O)C(=O)Nc1ccccc1F] | (S)-3-((S)-2-(2-((2- fluorophenyl)amino)-2- oxoacetamido)-4- methylpentanamido)-2-oxo-4-((S)-2- oxopiperidin-3-yl)butyl acetate | 521.3 | *** |
| 106 | 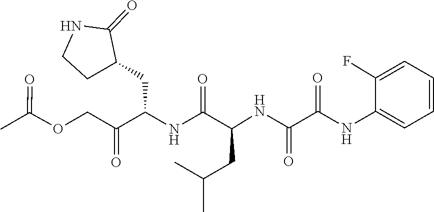 [SMILES: CC(=O)OCC(=O)[C@H](C[C@@H]1CCNC1=O)NC(=O)[C@H](CC(C)C)NC(=O)C(=O)Nc1ccccc1F] | (S)-3-((S)-2-(2-((2- fluorophenyl)amino)-2- oxoacetamido)-4- methylpentanamido)-2-oxo-4-((S)-2- oxopyrrolidin-3-yl)butyl acetate | 507.3 | *** |
| 107 | 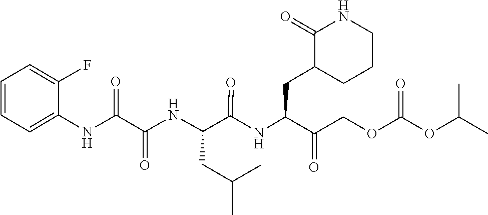 [SMILES: CC(C)C[C@H](NC(=O)C(=O)Nc1ccccc1F)C(=O)N[C@@H](CC1CCCNC1=O)C(=O)COC(=O)OC(C)C] | (S)-3-((S)-2-(2-((2- fluorophenyl)amino)-2- oxoacetamido)-4- methylpentanamido)-2-oxo-4-((S)-2- oxopiperidin-3-yl)butyl isopropyl carbonate | 565.43 | **** |
| 108 | 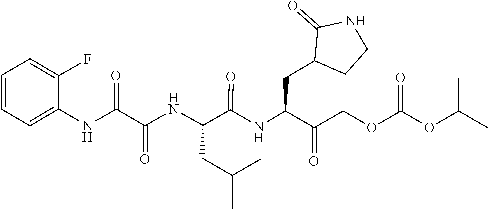 [SMILES: CC(C)C[C@H](NC(=O)C(=O)Nc1ccccc1F)C(=O)N[C@@H](CC1CCNC1=O)C(=O)COC(=O)OC(C)C] | (S)-3-((S)-2-(2-((2- fluorophenyl)amino)-2- oxoacetamido)-4- methylpentanamido)-2-oxo-4-((S)-2- oxopyrrolidin-3-yl)butyl isopropyl carbonate | 551.4798 | *** |
| 109 | 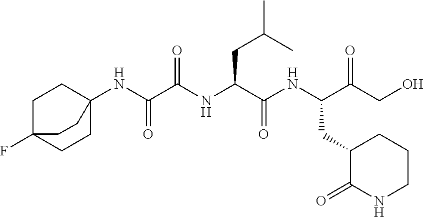 [SMILES: CC(C)C[C@H](NC(=O)C(=O)NC12CCC(F)(CC1)CC2)C(=O)N[C@@H](C[C@@H]1CCCNC1=O)C(=O)CO] | (S)-2-((S)-2-(2-((4- fluorobicyclo[2.2.2]octan-1- yl)amino)-2-oxoacetamido)-4- methylpentanamido)-3-((S)-2- oxopiperidin-3-yl)propanoic acid | | |
| 110 | 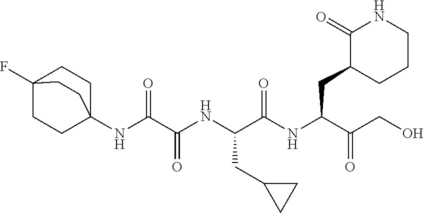 [SMILES: O=C(N[C@@H](CC1CC1)C(=O)N[C@@H](C[C@@H]1CCCNC1=O)C(=O)CO)C(=O)NC12CCC(F)(CC1)CC2] | N¹-((S)-3-cyclopropyl-1-(((S)-4- hydroxy-3-oxo-1-((S)-2-oxopiperidin- 3-yl)butan-2-yl)amino)-1-oxopropan- 2-yl)-N²-(4-fluorobicyclo[2.2.2]octan- 1-yl)oxalamide | 509.368 | >1 μM |
| 111 | 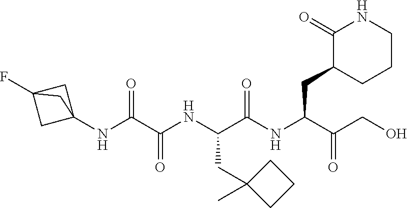 [SMILES: CC1(C[C@H](NC(=O)C(=O)NC23CC(F)(C2)C3)C(=O)N[C@@H](C[C@@H]2CCCNC2=O)C(=O)CO)CCC1] | N¹-(3-fluorobicyclo[1.1.1]pentan-1- yl)-N²-((S)-1-(((S)-4-hydroxy-3-oxo- 1-((S)-2-oxopiperidin-3-yl)butan-2- yl)amino)-3-(1-methylcyclobutyl)-1- oxopropan-2-yl)oxalamide | 495.3 | ** |
| 112 | 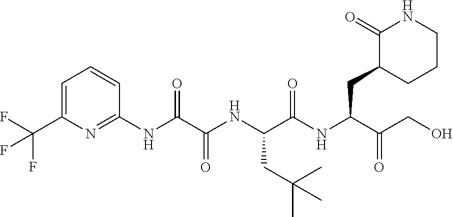 [SMILES: CC(C)(C)C[C@H](NC(=O)C(=O)Nc1cccc(C(F)(F)F)n1)C(=O)N[C@@H](C[C@@H]1CCCNC1=O)C(=O)CO] | N¹-(((S)-4-hydroxy-3-oxo-1- ((S)-2-oxopiperidin-3-yl)butan-2- yl)amino)-4,4-dimethyl-1-oxopentan- 2-yl)-N²-(6-(trifluoromethyl)pyridin- 2-yl)oxalamide | 544.36 | * |
| 113 | 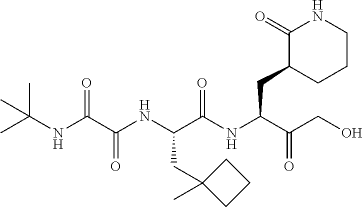 [SMILES: CC1(C[C@H](NC(=O)C(=O)NC(C)(C)C)C(=O)N[C@@H](C[C@@H]2CCCNC2=O)C(=O)CO)CCC1] | N¹-(tert-butyl)-N²-((S)-1-(((S)-4- hydroxy-3-oxo-1-((S)-2-oxopiperidin- 3-yl)butan-2-yl)amino)-3-(1- methylcyclobutyl)-1-oxopropan-2- yl)oxalamide | | *** |
| 114 | 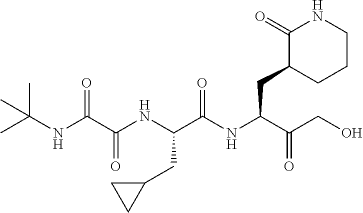 [SMILES: CC(C)(C)NC(=O)C(=O)N[C@@H](CC1CC1)C(=O)N[C@@H](C[C@@H]1CCCNC1=O)C(=O)CO] | N¹-(tert-butyl)-N²-((S)-3-cyclopropyl- 1-(((S)-4-hydroxy-3-oxo-1-((S)-2- oxopiperidin-3-yl)butan-2-yl)amino)- 1-oxopropan-2-yl)oxalamide | | |
| 115 | 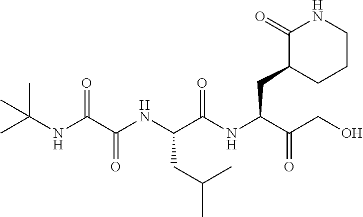 [SMILES: CC(C)C[C@H](NC(=O)C(=O)NC(C)(C)C)C(=O)N[C@@H](C[C@@H]1CCCNC1=O)C(=O)CO] | N¹-(tert-butyl)-N²-((S)-1-(((S)-4- hydroxy-3-oxo-1-((S)-2-oxopiperidin- 3-yl)butan-2-yl)amino)-4-methyl-1- oxopentan-2-yl)oxalamide | 441.39 | >1 μM |
| 116 | 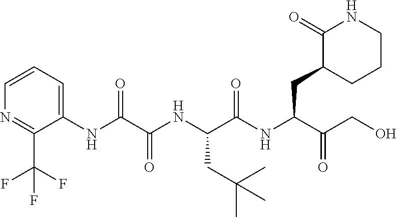 [SMILES: CC(C)(C)C[C@H](NC(=O)C(=O)Nc1cccnc1C(F)(F)F)C(=O)N[C@@H](C[C@@H]1CCCNC1=O)C(=O)CO] | N¹-((S)-1-(((S)-4-hydroxy-3-oxo-1- ((S)-2-oxopiperidin-3-yl)butan-2- yl)amino)-4,4-dimethyl-1-oxopentan- 2-yl)-N²-(2-(trifluoromethyl)pyridin- 3-yl)oxalamide | 544.3 | |
| 117 | 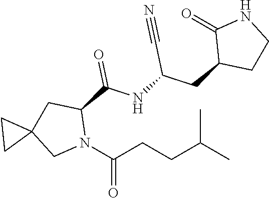 [SMILES: CC(C)CCC(=O)N1CC2(CC2)C[C@H]1C(=O)N[C@H](C#N)C[C@@H]1CCNC1=O] | (S)-N-((S)-1-cyano-2-((S)-2- oxopyrrolidin-3-yl)ethyl)-5-(4- methylpentanoyl)-5- azaspiro[2.4]heptane-6-carboxamide | 375.4 | >1 μM |
| 118 | 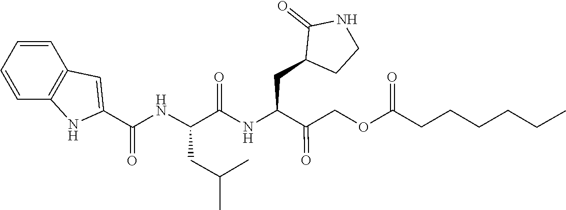 [SMILES: CCCCCCC(=O)OCC(=O)[C@H](C[C@@H]1CCNC1=O)NC(=O)[C@H](CC(C)C)NC(=O)c1cc2ccccc2[nH]1] | (S)-3-((S)-2-(1H-indole-2- carboxamido)-4-methylpentanamido)- 2-oxo-4-((S)-2-oxopyrrolidin-3- yl)butyl heptanoate | 555.69 | *** |
| 119 | 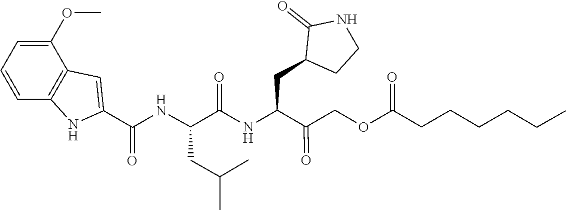 [SMILES: CCCCCCC(=O)OCC(=O)[C@H](C[C@@H]1CCNC1=O)NC(=O)[C@H](CC(C)C)NC(=O)c1cc2c(OC)cccc2[nH]1] | (S)-3-((S)-2-(4-methoxy-1H-indole-2- carboxamido)-4-methylpentanamido)- 2-oxo-4-((S)-2-oxopyrrolidin-3- yl)butyl heptanoate | 555.5 | *** |
| 120 | 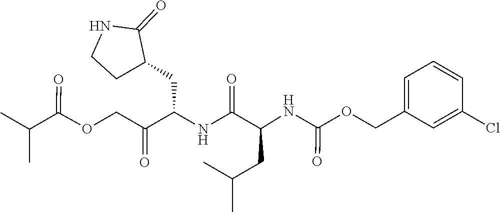 [SMILES: CC(C)C[C@H](NC(=O)OCc1cccc(Cl)c1)C(=O)N[C@@H](C[C@@H]1CCNC1=O)C(=O)COC(=O)C(C)C] | (S)-3-((S)-2-((((3- chlorobenzyl)oxy)carbonyl)amino)-4- methylpentanamido)-2-oxo-4-((S)-2- oxopyrrolidin-3-yl)butyl isobutyrate | 538.32 | >1 μM |
| 121 | 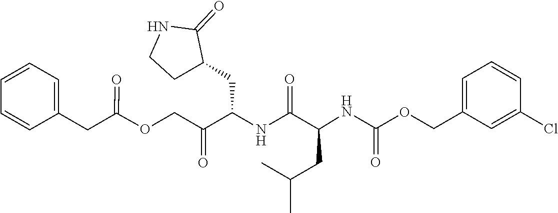 [SMILES: CC(C)C[C@H](NC(=O)OCc1cccc(Cl)c1)C(=O)N[C@@H](C[C@@H]1CCNC1=O)C(=O)COC(=O)Cc1ccccc1] | (S)-3-((S)-2-((((3- chlorobenzyl)oxy)carbonyl)amino)-4- methylpentanamido)-2-oxo-4-((S)-2- oxopyrrolidin-3-yl)butyl 2- phenylacetate | 586.17 | >1 μM |
| 122 | 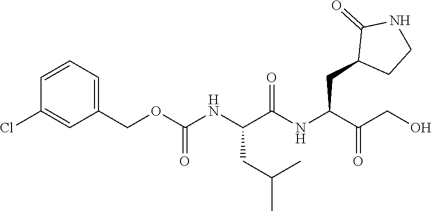 [SMILES: CC(C)C[C@H](NC(=O)OCc1cccc(Cl)c1)C(=O)N[C@@H](C[C@@H]1CCNC1=O)C(=O)CO] | 3-chlorobenzyl ((S)-1-(((S)-4- hydroxy-3-oxo-1-((S)-2- oxopyrrolidin-3-yl)butan-2-yl)amino)- 4-methyl-1-oxopentan-2-yl)carbamate | 468.23 | >1 μM |
| 123 | 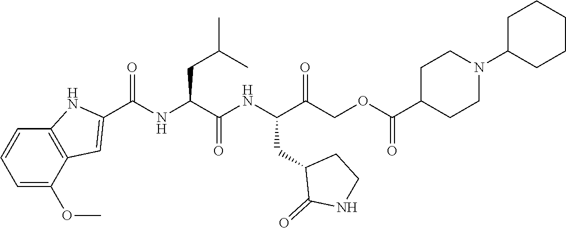 [SMILES: COc1cccc2[nH]c(C(=O)N[C@@H](CC(C)C)C(=O)N[C@@H](C[C@@H]3CCNC3=O)C(=O)COC(=O)C3CCN(C4CCCCC4)CC3)cc12] | (S)-3-((S)-2-(4-methoxy-1H-indole-2- carboxamido)-4-methylpentanamido)- 2-oxo-4-((S)-2-oxopyrrolidin-3- yl)butyl 1-cyclohexylpiperidine-4- carboxylate | 666.9629 | * |
| 124 | 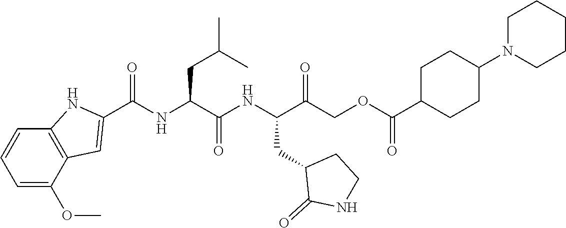 [SMILES: COc1cccc2[nH]c(C(=O)N[C@@H](CC(C)C)C(=O)N[C@@H](C[C@@H]3CCNC3=O)C(=O)COC(=O)C3CCC(N4CCCCC4)CC3)cc12] | (S)-3-((S)-2-(4-methoxy-1H-indole-2- carboxamido)-4-methylpentanamido)- 2-oxo-4-((S)-2-oxopyrrolidin-3- yl)butyl 4-(piperidin-1- yl)cyclohexane-1-carboxylate | 666.9 | >1 μM |
| 125 | 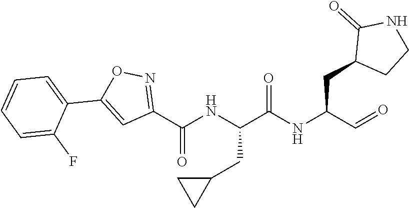 [SMILES: O=C[C@H](C[C@@H]1CCNC1=O)NC(=O)[C@H](CC1CC1)NC(=O)c1cc(-c2ccccc2F)on1] | N-((S)-3-cyclopropyl-1-oxo-1-(((S)-1- oxo-3-((S)-2-oxopyrrolidin-3- yl)propan-2-yl)amino)propan-2-yl)-5- (2-fluorophenyl)isoxazole-3- carboxamide | 457.18 | *** |
| 126 | 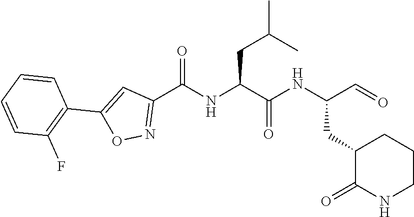 [SMILES: CC(C)C[C@H](NC(=O)c1cc(-c2ccccc2F)on1)C(=O)N[C@H](C=O)C[C@@H]1CCCNC1=O] | 5-(2-fluorophenyl)-N-((S)-4-methyl-1- oxo-1-(((S)-1-oxo-3-((S)-2- oxopiperidin-3-yl)propan-2- yl)amino)pentan-2-yl)isoxazole-3- carboxamide | 473.3004 | *** |
| 127 | 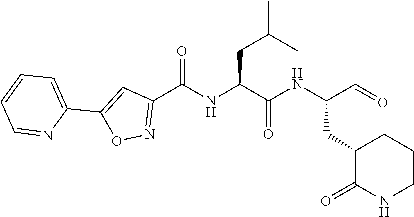 [SMILES: CC(C)C[C@H](NC(=O)c1cc(-c2ccccn2)on1)C(=O)N[C@H](C=O)C[C@@H]1CCCNC1=O] | N-((S)-4-methyl-1-oxo-1-(((S)-1-oxo- 3-((S)-2-oxopiperidin-3-yl)propan-2- yl)amino)pentan-2-yl)-5-(pyridin-2- yl)isoxazole-3-carboxamide | 456.22 | * |
| 128 | 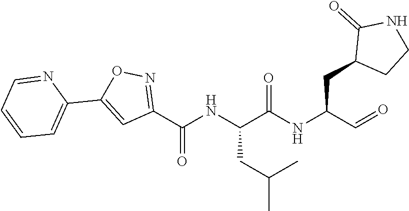 [SMILES: CC(C)C[C@H](NC(=O)c1cc(-c2ccccn2)on1)C(=O)N[C@H](C=O)C[C@@H]1CCNC1=O] | N-((S)-4-methyl-1-oxo-1-(((S)-1-oxo- 3-((S)-2-oxopyrrolidin-3-yl)propan-2- yl)amino)pentan-2-yl)-5-(pyridin-2- yl)isoxazole-3-carboxamide | 442.2 | >1 μM |
| 129 | 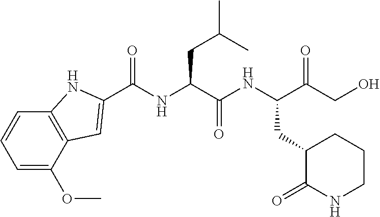 [SMILES: COc1cccc2[nH]c(C(=O)N[C@@H](CC(C)C)C(=O)N[C@@H](C[C@@H]3CCCNC3=O)C(=O)CO)cc12] | N-((S)-1-(((S)-4-hydroxy-3-oxo-1- ((S)-2-oxopiperidin-3-yl)butan-2- yl)amino)-4-methyl-1-oxopentan-2- yl)-4-methoxy-1H-indole-2- carboxamide | 487.6638 | * |
| 130 | 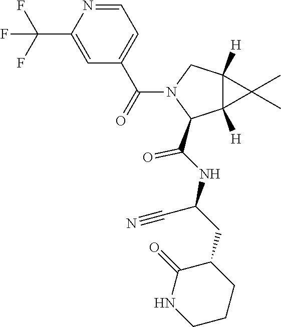 [SMILES: CC1(C)[C@@H]2[C@@H](C(=O)N[C@H](C#N)C[C@@H]3CCCNC3=O)N(C(=O)c3ccnc(C(F)(F)F)c3)C[C@@H]21] | (1R,2S,5S)-N-((S)-1-cyano-2-((S)-2- oxopiperidin-3-yl)ethyl)-6,6-dimethyl- 3-(2-(trifluoromethyl)isonicotinoyl)-3- azabicyclo[3.1.0]hexane-2- carboxamide | 478.34 | >1 μM |
| 131 | 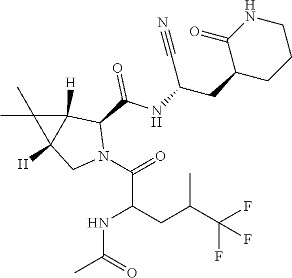 [SMILES: CC(=O)NC(CC(C)C(F)(F)F)C(=O)N1C[C@H]2[C@@H]([C@H]1C(=O)N[C@H](C#N)C[C@@H]1CCCNC1=O)C2(C)C] | (1R,2S,5S)-3-(2-acetamido-5,5,5- trifluoro-4-methylpentanoyl)-N-((S)-1- cyano-2-((S)-2-oxopiperidin-3- yl)ethyl)-6,6-dimethyl-3- azabicyclo[3.1.0]hexane-2- carboxamide | 514.4313 | * |
| 132 | 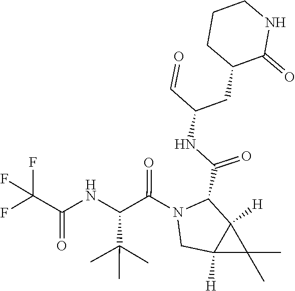 [SMILES: CC(C)(C)[C@H](NC(=O)C(F)(F)F)C(=O)N1C[C@H]2[C@@H]([C@H]1C(=O)N[C@H](C=O)C[C@@H]1CCCNC1=O)C2(C)C] | (1R,2S,5S)-3-((S)-3,3-dimethyl-2- (2,2,2-trifluoroacetamido)butanoyl)- 6,6-dimethyl-N-((S)-1-oxo-3-((S)-2- oxopiperidin-3-yl)propan-2-yl)-3- azabicyclo[3.1.0]hexane-2- carboxamide | 517.4747 | * |
| 133 | 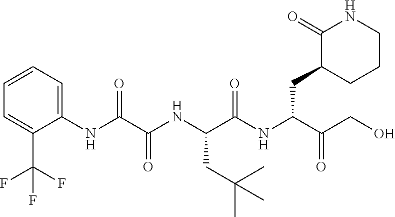 [SMILES: CC(C)(C)C[C@H](NC(=O)C(=O)Nc1ccccc1C(F)(F)F)C(=O)N[C@H](C[C@@H]1CCCNC1=O)C(=O)CO] | N¹-((S)-1-(((R)-4-hydroxy-3-oxo-1- ((S)-2-oxopiperidin-3-yl)butan-2- yl)amino)-4,4-dimethyl-1-oxopentan- 2-yl)-N²-(2- (trifluoromethyl)phenyl)oxalamide | 543.22 | >1 μM |
| 134 | 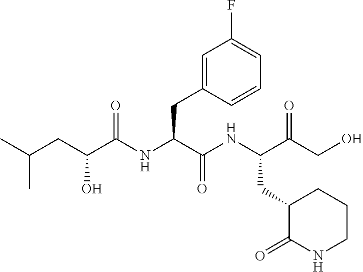 [SMILES: CC(C)C[C@@H](O)C(=O)N[C@@H](Cc1cccc(F)c1)C(=O)N[C@@H](C[C@@H]1CCCNC1=O)C(=O)CO] | (R)-N-((S)-3-(3-fluorophenyl)-1-(((S)- 4-hydroxy-3-oxo-1-((S)-2- oxopiperidin-3-yl)butan-2-yl)amino)- 1-oxopropan-2-yl)-2-hydroxy-4- methylpentanamide | 480.3504 | >1 μM |
| 135 | 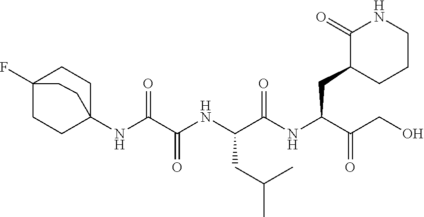 [SMILES: CC(C)C[C@H](NC(=O)C(=O)NC12CCC(F)(CC1)CC2)C(=O)N[C@@H](C[C@@H]1CCCNC1=O)C(=O)CO] | N¹-(4-fluorobicyclo[2.2.2]octan-1-yl)- N²-((S)-1-(((S)-4-hydroxy-3-oxo-1- ((S)-2-oxopiperidin-3-yl)butan-2- yl)amino)-4-methyl-1-oxopentan-2- yl)oxalamide | 511.3798 | >1 μM |
| 136 | 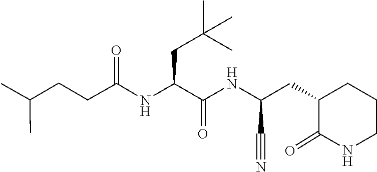 [SMILES: CC(C)CCC(=O)N[C@@H](CC(C)(C)C)C(=O)N[C@H](C#N)C[C@@H]1CCCNC1=O] | (S)-N-((S)-1-cyano-2-((S)-2- oxopiperidin-3-yl)ethyl)-4,4-dimethyl- 2-(4-methylpentanamido)pentanamide | 393.4136 | >1 μM |
| 137 | 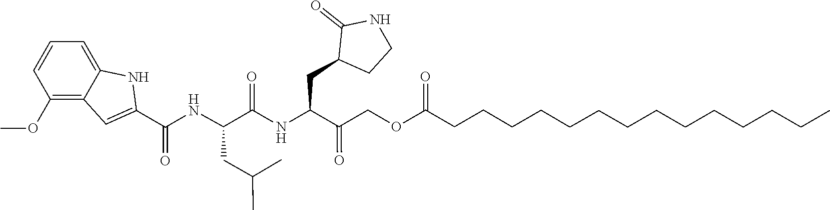 [SMILES: CCCCCCCCCCCCCCC(=O)OCC(=O)[C@H](C[C@@H]1CCNC1=O)NC(=O)[C@H](CC(C)C)NC(=O)c1cc2c(OC)cccc2[nH]1] | (S)-3-((S)-2-(4-methoxy-1H-indole-2- carboxamido)-4-methylpentanamido)- 2-oxo-4-((S)-2-oxopyrrolidin-3- yl)butyl pentadecanoate | 698.2 | ** |
| 138 | 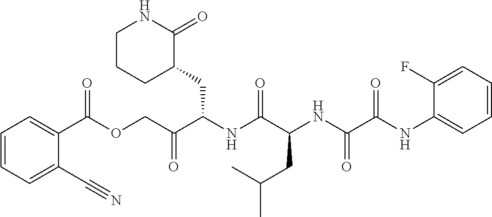 [SMILES: CC(C)C[C@H](NC(=O)C(=O)Nc1ccccc1F)C(=O)N[C@@H](C[C@@H]1CCCNC1=O)C(=O)COC(=O)c1ccccc1C#N] | (S)-3-((S)-2-(2-((2- fluorophenyl)amino)-2- oxoacetamido)-4- methylpentanamido)-2-oxo-4-((S)-2- oxopiperidin-3-yl)butyl 2- cyanobenzoate | 608.53 | *** |

EC50: <0.05 μM: ****
0.05-<0.2 μM: ***
0.2-<0.5 μM: **
0.5-1.0 μM: *

TABLE 2

| Compound | ¹H NMR Data |
|---|---|
| 18 | ¹H NMR (400 MHz, DMSO-d₆) δ ppm 8.57-8.52 (m, 3H), 7.65 (s, 1H), 4.83 |
| | (s, 2H), 4.51-4.30 (m, 2H), 3.55 (d, J = 8.6 Hz, 1H), 3.21-3.05 (m, 2H), 2.36 |
| | (t, J = 7.3 Hz, 2H), 2.29- 2.26 (m, 1H), 2.16-2.08 (m, 1H), 2.05-1.95 (m, 1H), |
| | 1.72-1.45 (m, 11H), 1.38-1.26 (m, 11H), 1.12-1.02 (s, 1H), 0.89-0.85 (m, |
| | 9H). |
| 19 | ¹H NMR (400 MHz, DMSO-d₆) δ ppm 8.57-8.53 (m, 3H), 7.65 (s, 1H), 4.83 |
| | (s, 2H), 4.39-4.34 (m, 2H), 3.32-3.16 (m, 1H), 3.16-3.09 (m, 2H), 2.50-2.49 |
| | (m, 2H), 2.38-2.35 (m, 1H), 2.34-2.33 (m, 1H), 2.33-1.70 (m, 1H), 1.67-1.51 |
| | (m, 14H), 1.29-1.24 (m, 13H), 1.13-1.09 (m, 4H), 0.90-0.84 (m, 5H). |
| 20 | ¹H NMR (400 MHz, DMSO-d₆) δ ppm 10.0(s, 1H), 8.90 (d, J = 8.8 Hz, 1H), |
| | 8.55 (d, J = 8.0 Hz, 1H), 7.63 (s, 1H), 7.41 (d, J = 8.0 Hz, 1H), 7.31-7.11 (m, |
| | 7H), 5.09 (t, J = 6.0 Hz, 1H), 4.66-4.64 (m, 1H), 4.49 (m, 1H), 4.25-4.09 (m, |
| | 2H), 3.16-3.02 (m, 4H), 2.32-2.29 (m, 1H), 2.15 (s, 4H), 1.93 (m, 1H), 1.67- |
| | 1.63 (m, 2H). |
| 21 | (400 MHz, DMSO-d₆) δ ppm 10.29 (s, 1H), 8.90 (d, J = 8.5 Hz, 1H), 8.49 (d, |
| | J = 8.0 Hz, 1H), 7.69 (td, J = 7.8, 1.9 Hz, 1H), 7.46 (s, 1H), 7.35-7.19 (m, |
| | 3H), 5.09 (t, J = 5.9 Hz, 1H), 4.49 (ddd, J= 11.5, 7.9, 3.8 Hz, 1H), 4.43- |
| | 4.34 (m, 1H), 4.23 (dd, J = 18.7, 6.1Hz, 1H), 4.13 (dd, J = 18.8, 5.8 Hz, 1H), |
| | 3.14-3.07 (m, 2H), 3.02-2.87 (m, 1H), 2.24-2.05 (m, 2H), 1.89-1.79 |
| | (m, 1H), 1.78-1.48 (m, 5H), 1.39-1.28 (m, 1H), 0.91 (d, J = 6.3 Hz, 3H), |
| | 0.88 (d, J = 6.0 Hz, 3H). |
| 27 | (500 MHz, DMSO-d₆) δ ppm 10.11 (s, 1H), 8.80 (d, J = 8.6 Hz, 1H), 8.50 (d, |
| | J = 8.0 Hz, 1H), 7.49 (dd, J = 7.8, 0.8 Hz, 1H), 7.26 (d, J = 7.4 Hz, 1H), 7.22 |
| | (td, J = 7.7, 1.3 Hz, 1H), 7.15 (td, J = 7.4, 1.4 Hz, 1H), 5.09 (t, J = 6.0 Hz, |
| | 1H), 4.50 (ddd, J = 11.7, 7.9, 3.9 Hz, 1H), 4.44-4.36 (m, 2H), 4.23 (dd, J = |
| | 18.8, 6.1 Hz, 1H), 4.14 (dd, J = 18.7, 5.9 Hz, 1H), 3.16-3.03 (m, 2H), 2.21 |
| | (s, 3H), 2.20-2.15 (m, 1H), 2.11 (ddd, J = 13.6, 11.5, 4.2 Hz, 1H), 1.89- |
| | 1.80 (m, 1H), 1.78-1.64 (m, 3H), 1.63-1.49 (m, 3H), 1.40-1.30 (m, 1H), |
| | 0.92 (d, J = 6.4 Hz, 3H), 0.90 (d, J = 6.2 Hz, 3H). |
| 34 | ¹H NMR (400 MHz, DMSO-d₆) δ ppm 10.25 (d, J = 6.0 Hz, 1H), 8.96-8.81 |
| | (m, 1H), 8.49-8.31 (m, 1H), 7.75-7.68 (m, 1H), 7.47-7.46 (m, 1H), 7.34-7.21 |
| | (m, 3H), 5.13-4.94 (m, 1H), 4.39-4.12 (m, 4H), 3.20-3.95 (m, 2H), 2.25-2.15 |
| | (m, 1H), 3.88 (s, 3H), 1.86-1.12 (m, 12H), 0.93-0.89 (m, 6H). |
| 38 | (400 MHz, DMSO-d₆) δ ppm 10.19-10.05 (m, 2H), 9.42 (s, 1H), 8.82 (d, J = |
| | 8.6 Hz, 1H), 8.74 (d, J = 8.9 Hz, 0H), 8.56 (d, J = 7.5 Hz, 1H), 7.73 (d, J = 9.2 |
| | Hz, 1H), 7.64 (s, 1H), 7.54-7.43 (m, 4H), 7.38-7.07 (m, 7H), 5.80-5.69 |
| | (m, 1H), 4.72-4.65 (m, 1H), 4.50-4.37 (m, 2H), 4.25-4.18 (m, 1H), 3.20- |
| | 3.03 (m, 5H), 2.25-2.02 (m, 10H), 1.95-1.80 (m, 1H), 1.79-1.72 (m, |
| | 1H), 1.71-1.47 (m, 10H), 1.40 (t, J = 11.5 Hz, 0H), 0.95-0.84 (m, 14H). |
| 40 | (500 MHz, CDC₃) δ ppm 9.50 (s, 1H), 9.44 (s, 1H), 8.64 (d, J = 5.2 Hz, 1H), |
| | 8.35 (t, J = 7.8 Hz, 1H), 7.88 (d, J = 8.6 Hz, 1H), 7.20-7.09 (m, 4H), 5.68 (s, |
| | 1H), 4.66-4.57 (m, 1H), 4.34-4.26 (m, 1H), 3.45-3.34 (m, 2H), 2.55- |
| | 2.38 (m, 2H), 1.99-1.93 (m, 2H), 1.93-1.80 (m, 3H), 1.77-1.62 (m, 5H), |
| | 1.44-1.33 (m, 1H), 1.30-1.11 (m, 4H), 1.08-0.91 (m, 2H). |
| 58 | ¹H NMR (400 MHz, DMSO-d₆) δ ppm 10.3 (s, 1H), 9.00 (d, J = 8.4 Hz, 1H), |
| | 8.87 (d, J = 7.6 Hz, 1H), 7.72-7.69 (m, 2H), 7.34-7.23 (m, 3H), 5. (q, J = 6.8 |
| | Hz, 1H), 4.37-4.35 (m, 1H), 3.17-3.12 (m, 2H), 2.50-2.34 (m, 1H), 2.18-2.11 |
| | (m, 2H), 1.82-1.53 (m, 5H), 0.92-0.87 (m, 6H). |
| 111 | ¹H NMR (400 MHz, DMSO-d₆) δ ppm 9.59 (s, 1H), 8.61 (d, J = 9.1 Hz, 1H), |
| | 8.37 (d, J = 8.1 Hz, 1H), 7.46 (s, 1H), 5.08 (t, J = 6.0 Hz, 1H), 4.53-4.28 (m, |
| | 3H), 4.28-4.05 (m, 2H), 3.13-3.06 (m, 2H), 2.64-2.58 (m, 2H), 2.37 (d, J |
| | = 2.2 Hz, 6H), 2.20-1.96 (m, 2H), 1.95-1.68 (m, 5H), 1.66-1.56 (m, 1H), |
| | 1.57-1.45 (m, 2H), 1.41-1.28 (m, 2H), 1.12 (s, 3H). |

1. A compound of Formula (I): 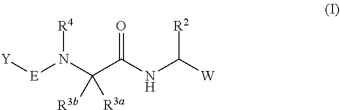
wherein
Y is 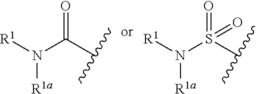
wherein
R¹ and R^{1a} are independently selected from the group consisting of H, C₁-C₈-alkyl, C₃-C₁₀-cycloalkyl, —(C₃-C₁₀-cycloalkyl)-(C₆-C₁₀-aryl), C₆-C₁₀-aryl, 5- to 10-membered heteroaryl (wherein 1-4 heteroaryl members are independently selected from N, O, and S), —(C₁-C₆-alkyl)(C₆-C₁₀-aryl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S)), and —(C₁-C₆-alkyl)(5- to 10-membered heteroaryl (wherein 1-4 heteroaryl members are independently selected from N, O, and S));
or R¹ and R^{1a} together with the nitrogen atom to which they are bound form a 4-10 membered mono or bicyclic fused, bridged, or spiro-fused ring wherein the ring members are selected from C, N, O, and S;
wherein R¹ and R^{1a} are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₁-C₆-haloalkoxy, C₃-C₈-cycloalkyl (optionally substituted with 1-3 substituents independently selected from halo and NH₂), CN, and CONR⁷R⁸;
or Y is selected from the group consisting of C₁-C₆-alkyl (optionally interrupted by one or more heteroatoms selected from —NH—, O, and S), C₁-C₆-haloalkyl, —(C₁-C₆-alkyl)-(C₃-C₁₀-cycloalkyl), 5- to 10-membered heteroaryl (wherein 1-4 heteroaryl members are independently selected from N, O, and S), —C(O)—C₆-C₁₀-aryl, —C(O)-(5- to 10-membered heteroaryl (wherein 1-4 heteroaryl members are independently selected from N, O, and S)), —C(O)—O—C₆-C₁₀-aryl, —C(O)—C₆-C₁₀-aryl, —C(O)—NH—C₆-C₁₀-aryl, —C(O)—C₁-C₆-alkyl-O—C₆-C₁₀-aryl, —C(O)—O—(C₁-C₆-alkyl)(C₆-C₁₀-aryl), —(C₂-C₆-alkenyl)(C₆-C₁₀-aryl), -(5- to 10-membered heteroaryl (wherein 1-4 heteroaryl members are independently selected from N, O, and S))—(C₆-C₁₀-aryl), —C(O)—O—(C₁-C₆-alkyl), —C(O)—C₁-C₆-alkyl-(5- to 10-membered heteroaryl (wherein 1-4 heteroaryl members are independently selected from N, O, and S)), —(C₁-C₆-alkyl)(C₆-C₁₀-aryl), —C(O)-(3- to 6-membered heterocycloalkyl (wherein 1-4 ring members are independently selected from N, O, and S)), —C(O)—O-(3- to 6-membered heterocycloalkyl (wherein 1-4 ring members are independently selected from N, O, and S)), —C(O)—C₁-C₆-alkyl optionally substituted with halo, —C(O)—C₃-C₁₀-cycloalkyl, and —C(O)—NH-(5- to 10-membered heteroaryl (wherein 1-4 heteroaryl members are independently selected from N, O, and S));
wherein Y is optionally substituted with one to three substituents selected from the group consisting of halo, C₁-C₆-alkyl, C₁-C₆-haloalkyl, C₁-C₆-alkoxy, C₃-C₈-cycloalkyl (optionally substituted with 1-3 substituents independently selected from halo and NH₂), NH₂, C₁-C₆-alkyl optionally substituted with NH₂, 3- to 6-membered heterocycloalkyl (wherein 1-4 ring members are independently selected from N, O, and S), 5- to 10-membered heteroaryl (wherein 1-4 heteroaryl members are independently selected from N, O, and S), CN, COR⁷, CONR⁷R⁸, OR⁷, SR⁷, NR⁷C(O)O—(C₁-C₆-alkyl)(C₆-C₁₀-aryl), NR⁷C(O)R⁸, and SO₂R⁷;
R⁷ and R⁸ are independently selected from H, C₁-C₆-alkyl, C₁-C₆-haloalkyl, and C₃-C₈-cycloalkyl;
E is a bond, —C(O)—, or —NHC(R^{9a})(R^{9b})C(O)—;
R^{9a} and R^{9b} are independently selected from the group consisting of H, C₁-C₆-alkyl, C₃-C₈-cycloalkyl, —C₁-C₆-alkyl-O—C₁-C₆-alkyl, —C(O)C₁-C₆-alkyl (optionally substituted with one to three of C₁-C₆-alkyl, C₃-C₈-cycloalkyl, halo, C₁-C₆-haloalkyl), 3- to 6-membered heterocycloalkyl (wherein 1-4 ring members are independently selected from N, O, and S), and —(C₁-C₆-alkyl)-(3- to 6-membered heterocycloalkyl (wherein 1-4 ring members are independently selected from N, O, and S)),
R^{9a} and R^{9b} are optionally and independently substituted with 1 to 5 substituents independently selected from the group consisting of halo, OH, NH₂, C₁-C₆-alkyl, C₁-C₆-haloalkyl, C₃-C₈-cycloalkyl (optionally substituted with 1-3 substituents independently selected from halo and NH₂);
W is selected from the group consisting of CN, C(O)H, —C(O)CH₂OH, —C(O)CH₂OC(O)R⁵, and —C(O)CH₂OC(O)C(O)R⁵,
wherein R⁵ is selected from the group consisting of H, C₁-C₂₀-alkyl, C₆-C₁₀-aryl optionally substituted with CN, —(C₁-C₆-alkyl)-(C₆-C₁₀-aryl), C₁-C₆-alkoxy, —(C₃-C₈-cycloalkyl)-(3- to 6-membered heterocycloalkyl (wherein 1-4 ring members are independently selected from N, O, and S)), and -(3- to 6-membered heterocycloalkyl (wherein 1-4 ring members are independently selected from N, O, and S))—(C₃-C₈-cycloalkyl);
R² is selected from the group consisting of 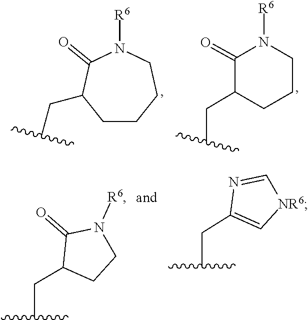
R⁶ is H or C₁-C₆-alkyl;
R^{3a}, R^{3b}, and R⁴ are selected independently from the group consisting of H, C₁-C₆-alkyl, C₃-C₁₀-cycloalkyl, —(C₁-C₆-alkyl)(C₃-C₁₀-cycloalkyl), —(C₁-C₆-alkyl)(C₆-C₁₀-aryl), —(C₁-C₆-alkyl)(3- to 6-membered heterocycloalkyl (wherein 1-4 ring members are independently selected from N, O, and S), C₂-C₈-alkenyl, and —(C₁-C₆-alkyl)(5- to 10-membered heteroaryl) (wherein 1-4 heteroaryl members are independently selected from N, O, and S),
wherein R^{3a}, R^{3b}, and R⁴ are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₃-C₈-cycloalkyl (optionally substituted with 1-3 substituents independently selected from halo and NH₂), CN, and CONR⁷R⁸;
or R^{3a} and R⁴, R^{3b} and R⁴, or R^{3a} and R^{3b} together with the atoms to which they are bound form a 3-10 membered mono- or bicyclic ring that, if bicyclic, is optionally fused, bridged, or spiro-fused, wherein the mono- or bicyclic ring is optionally substituted with one to three substituents selected from halo, OH, C₂-C₆-alkenyl, C₁-C₆-haloalkyl, C₁-C₆-alkoxy, C₃-C₈-cycloalkyl (optionally and independently substituted with 1-3 substituents selected from halo and NH₂), C₁-C₆-alkyl optionally substituted with NH₂, 3- to 6-membered heterocycloalkyl (wherein 1-4 ring members are independently selected from N, O, and S), CN, and CONR⁷R⁸;
or a pharmaceutically acceptable salt thereof.

2. A compound according to claim 1,
wherein
Y is 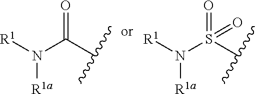
wherein
R¹ and R^{1a} are independently selected from the group consisting of H, C₁-C₈-alkyl, C₃-C₁₀-cycloalkyl, C₆-C₁₀-aryl, 5- to 10-membered heteroaryl (wherein 1-4 heteroaryl members are independently selected from N, O, and S), —(C₁-C₆-alkyl)(C₆-C₁₀-aryl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S)), and —(C₁-C₆-alkyl)(5- to 10-membered heteroaryl (wherein 1-4 heteroaryl members are independently selected from N, O, and S));
or R¹ and R^{1a} together with the nitrogen atom to which they are bound form a 4-10 membered mono or bicyclic fused, bridged, or spiro-fused ring wherein the ring members are selected from C, N, O, and S;
wherein R¹ and R^{1a} are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₁-C₆-haloalkoxy, C₃-C₈-cycloalkyl (optionally substituted with 1-3 substituents independently selected from halo and NH₂), CN, and CONR⁷R⁸;
or Y is selected from the group consisting of 5- to 10-membered heteroaryl (wherein 1-4 heteroaryl members are independently selected from N, O, and S), —C(O)—C₆-C₁₀-aryl, —C(O)-(5- to 10-membered heteroaryl (wherein 1-4 heteroaryl members are independently selected from N, O, and S)), —C(O)—O—C₆-C₁₀-aryl, —C(O)—NH—C₆-C₁₀-aryl, —C(O)—C₁-C₆-alkyl-O—C₆-C₁₀-aryl, —C(O)—C₁-C₆-alkyl-(5- to 10-membered heteroaryl (wherein 1-4 heteroaryl members are independently selected from N, O, and S)), —(C₁-C₆-alkyl)(C₆-C₁₀-aryl), —C(O)-(3- to 6-membered heterocycloalkyl (wherein 1-4 ring members are independently selected from N, O, and S)), —C(O)—O-(3- to 6-membered heterocycloalkyl (wherein 1-4 ring members are independently selected from N, O, and S)), —C(O)—C₁-C₆-alkyl optionally substituted with halo, —C(O)—C₃-C₁₀-cycloalkyl, and —C(O)—NH-(5- to 10-membered heteroaryl (wherein 1-4 heteroaryl members are independently selected from N, O, and S));
wherein Y is optionally substituted with one to three substituents selected from the group consisting of halo, C₁-C₆-alkyl, C₁-C₆-haloalkyl, C₁-C₆-alkoxy, C₃-C₈-cycloalkyl (optionally substituted with 1-3 substituents independently selected from halo and NH₂), NH₂, C₁-C₆-alkyl optionally substituted with NH₂, 3- to 6-membered heterocycloalkyl (wherein 1-4 ring members are independently selected from N, O, and S), CN, COR⁷, CONR⁷R⁸, OR⁷, SR⁷, and SO₂R⁷;
R⁷ and R⁸ are independently selected from H, C₁-C₆-alkyl, and C₃-C₈-cycloalkyl;
E is a bond, —C(O)—, or —NHC(R^{9a})(R^{9b})C(O)—;
R^{9a} and R^{9b} are independently selected from the group consisting of H, C₁-C₆-alkyl, C₃-C₈-cycloalkyl, —C₁-C₆-alkyl-O—C₁-C₆-alkyl, —C(O)C₁-C₆-alkyl (optionally substituted with one to three of C₁-C₆-alkyl, C₃-C₈-cycloalkyl, halo, and C₁-C₆-haloalkyl), 3- to 6-membered heterocycloalkyl (wherein 1-4 ring members are independently selected from N, O, and S),
R^{9a} and R^{9b} are optionally and independently substituted with 1 to 5 substituents independently selected from the group consisting of halo, OH, NH₂, C₁-C₆-alkyl, C₁-C₆-haloalkyl, C₃-C₈-cycloalkyl (optionally substituted with 1-3 substituents independently selected from halo and NH₂);
W is selected from the group consisting of CN, C(O)H, —C(O)CH₂OH, —C(O)CH₂OC(O)R⁵, and —C(O)CH₂OC(O)C(O)R⁵,
wherein R⁵ is selected from the group consisting of H, C₁-C₂₀-alkyl, C₆-C₁₀-aryl, —(C₃-C₈-cycloalkyl)-(3- to 6-membered heterocycloalkyl (wherein 1-4 ring members are independently selected from N, O, and S)), and -(3- to 6-membered heterocycloalkyl (wherein 1-4 ring members are independently selected from N, O, and S))—(C₃-C₈-cycloalkyl);
R² is selected from the group consisting of 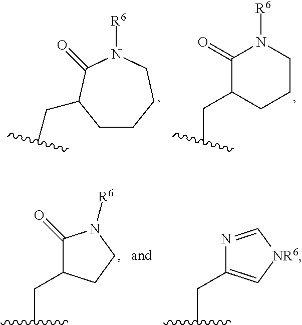
R⁶ is H or C₁-C₆-alkyl;
R^{3a}, R^{3b}, and R⁴ are selected independently from the group consisting of H, C₁-C₆-alkyl, C₃-C₁₀-cycloalkyl, —(C₁-C₆-alkyl)(C₃-C₁₀-cycloalkyl), —(C₁-C₆-alkyl)(C₆-C₁₀-aryl), —(C₁-C₆-alkyl)(3- to 6-membered heterocycloalkyl (wherein 1-4 ring members are independently selected from N, O, and S), and —(C₁-C₆-alkyl)(5- to 10-membered heteroaryl) (wherein 1-4 heteroaryl members are independently selected from N, O, and S), wherein R^{3a}, R^{3b}, and R⁴ are optionally and independently substituted with 1 to 5 substituents independently selected from the group consisting of halo, OH, NH₂, C₁-C₆-alkyl optionally substituted with NH₂, C₁-C₆-haloalkyl, C₁-C₆-alkoxy, C₃-C₈-cycloalkyl (optionally substituted with 1-3 substituents independently selected from halo and NH₂), CN, and CONR⁷R⁸;
or R^{3a} and R⁴, R^{3b} and R⁴, or R^{3a} and R^{3b} together with the atoms to which they are bound form a 3-10 membered mono- or bicyclic ring that, if bicyclic, is optionally fused, bridged, or spiro-fused, wherein the mono- or bicyclic ring is optionally substituted with one to three substituents selected from halo, OH, C₂-C₆-alkenyl, C₁-C₆-haloalkyl, C₁-C₆-alkoxy, C₃-C₈-cycloalkyl (optionally and independently substituted with 1-3 substituents selected from halo and NH₂), C₁-C₆-alkyl optionally substituted with NH₂, 3- to 6-membered heterocycloalkyl (wherein 1-4 ring members are independently selected from N, O, and S), CN, and CONR⁷R⁸;
or a pharmaceutically acceptable salt thereof.

3. The compound or pharmaceutically acceptable salt thereof according to claim 1, wherein W is C(O)H.

4. The compound or pharmaceutically acceptable salt thereof according to claim 1, wherein W is CN.

5. The compound or pharmaceutically acceptable salt thereof according to claim 1, wherein W is selected from the group consisting of C(O)CH₂OH, —C(O)CH₂OC(O)R⁵, and —C(O)CH₂OC(O)C(O)R⁵.

6. The compound or pharmaceutically acceptable salt thereof according to claim 1, wherein W is C(O)CH₂OH.

7. The compound or pharmaceutically acceptable salt thereof according to claim 1, wherein R² is

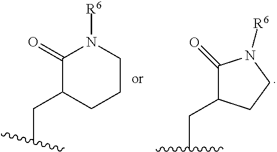

8. The compound or pharmaceutically acceptable salt thereof according to claim 1, wherein R² is

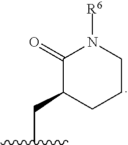

9. The compound or pharmaceutically acceptable salt thereof according to claim 1, wherein R² is

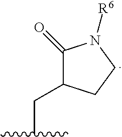

10. The compound or pharmaceutically acceptable salt thereof according to claim 1, wherein R⁶ is H.

11. The compound or pharmaceutically acceptable salt thereof according to claim 1, wherein R^{3a} is H and R^{3b} is selected from the group consisting of optionally substituted C₁-C₆-alkyl and —(C₁-C₆-alkyl)(C₃-C₁₀-cycloalkyl).

12. The compound or pharmaceutically acceptable salt thereof according to claim 1, wherein R^{3a} is H and R^{3b} is optionally substituted C₁-C₆-alkyl.

13. The compound or pharmaceutically acceptable salt thereof according to claim 1, wherein R^{3a} is H and R^{3b} is or

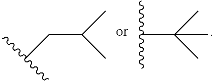

14. The compound or pharmaceutically acceptable salt thereof according to claim 1, wherein R^{3a} is H and R^{3b} is

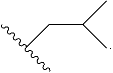 [SMILES: *CC(C)C]

15. The compound or pharmaceutically acceptable salt thereof according to claim 1, where in the moiety 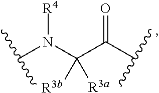
R^{3a} is H, and R^{3b} and R⁴ together with the atoms to which they are bound form an optionally substituted 3-10 membered mono- or bicyclic ring that, if bicyclic, is optionally fused, bridged, or spiro-fused.

16. The compound or pharmaceutically acceptable salt thereof according to claim 15, wherein the moiety 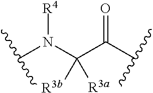
is selected from the group consisting of

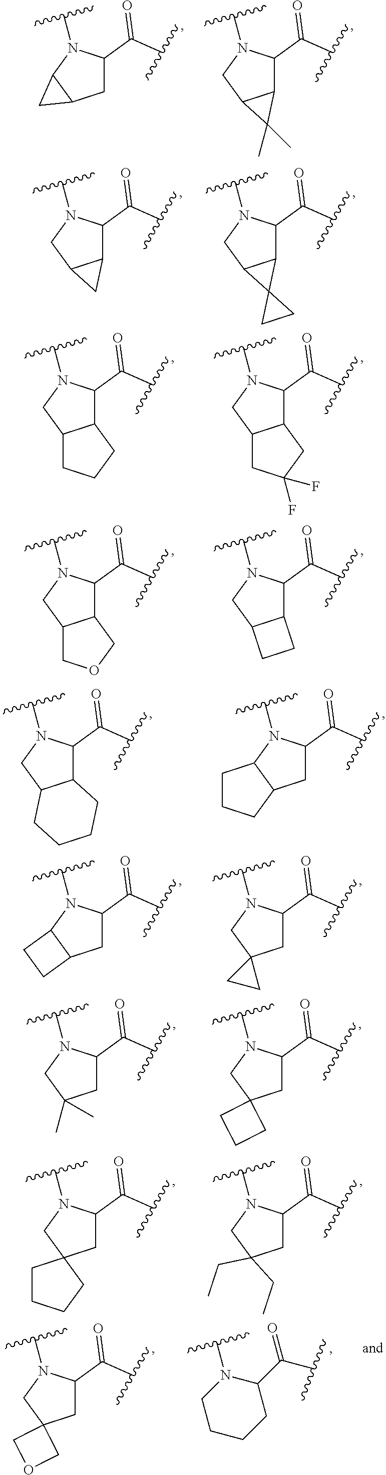

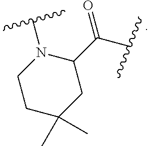 [SMILES: *C(=O)C1CC(C)(C)CCN1*]

17. The compound or pharmaceutically acceptable salt thereof according to claim 1, wherein E is a bond.

18. The compound or pharmaceutically acceptable salt thereof according to claim 1, wherein E is —C(O)—.

19. The compound or pharmaceutically acceptable salt thereof according to claim 1, wherein Y is

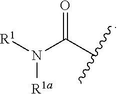

20. The compound or pharmaceutically acceptable salt thereof according to claim 1, wherein R¹ is H.

21. The compound or pharmaceutically acceptable salt thereof according to claim 1, wherein Y is selected from the group consisting of:

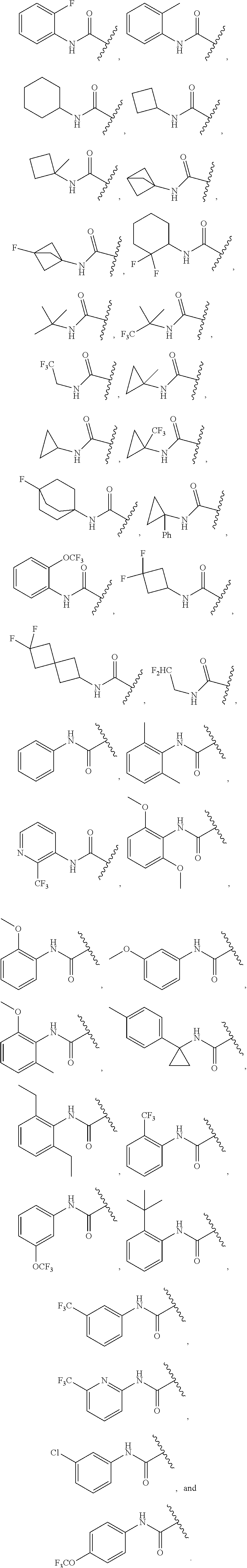

22. The compound or pharmaceutically acceptable salt thereof according to claim 1, wherein
Y is 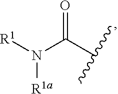
wherein R¹ is H and R^{1a} is C₁-C₈-alkyl, C₃-C₁₀-cycloalkyl, or C₆-C₁₀-aryl, wherein R^{1a} is optionally substituted with 1 to 5 substituents independently selected from the group consisting of halo, C₁-C₆-alkyl, C₁-C₆-haloalkyl, and C₁-C₆-haloalkoxy;
or Y is C₁-C₆-alkyl, —C(O)—C₁-C₆-alkyl, —C(O)—C₁-C₆-alkyl-O—C₆-C₁₀-aryl, or -(5- to 10-membered heteroaryl (wherein 1-4 heteroaryl members are independently selected from N, O, and S))—(C₆-C₁₀-aryl), wherein Y is optionally substituted with one to three substituents selected from the group consisting of halo, C₁-C₆-alkyl, and C₁-C₆-haloalkyl;
E is a bond, —C(O)—, or —NHCH(C₁-C₆-alkyl)C(O)—;
W is CN, C(O)H, —C(O)CH₂OH, or —C(O)CH₂OC(O)(C₁-C₂₀-alkyl);
R² is 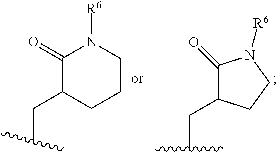
R^{3a}, R^{3b}, and R⁴ are selected independently from the group consisting of H, C₁-C₆-alkyl, and —(C₁-C₆-alkyl)(C₃-C₁₀-cycloalkyl), wherein the cycloalkyl is optionally substituted with one to three C₁-C₆-alkyl;
or the moiety 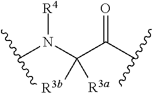
is selected from the group consisting of

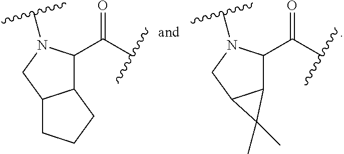 [SMILES: *C(=O)C1C2CCCC2CN1* ; *C(=O)C1C2C(CN1*)C2(C)C]

23. The compound or pharmaceutically acceptable salt thereof according to claim 22, wherein
Y is 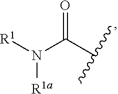
wherein R¹ is H and R^{1a} is C₁-C₈-alkyl or C₆-C₁₀-aryl, wherein R^{1a} is optionally substituted with 1 to 5 substituents independently selected from the group consisting of halo, C₁-C₆-haloalkyl, and C₁-C₆-haloalkoxy;
E is —C(O)—;
W is —C(O)CH₂OH;
R² is 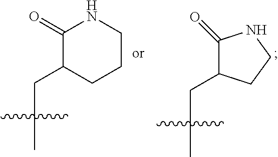 [SMILES: *CC1CCCNC1=O ; *CC1CCNC1=O]
R^{3a} is H;
R^{3b} is C₁-C₆-alkyl or —(C₁-C₆-alkyl)(C₃-C₁₀-cycloalkyl); and
R⁴ is H.

24. The compound or pharmaceutically acceptable salt thereof according to claim 1, wherein the compound is selected from the group consisting of:
N—((S)-1-(((S)-4-hydroxy-3-oxo-1-((S)-2-oxopyrrolidin-3-yl)butan-2-yl)amino)-4-methyl-1-oxopentan-2-yl)-4-methoxy-1H-indole-2-carboxamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-phenyloxalamide;
N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl pentadecanoate;
(S)-3-((S)-2-(2-((2-fluorophenyl)amino)-2-oxoacetamido)-4-methylpentanamido)-2-oxo-4-((S)-2-oxopyrrolidin-3-yl)butyl 4-(piperidin-1-yl)cyclohexane-1-carboxylate;
(S)-3-((S)-2-(2-((2-fluorophenyl)amino)-2-oxoacetamido)-4-methylpentanamido)-2-oxo-4-((S)-2-oxopyrrolidin-3-yl)butyl 1-cyclohexylpiperidine-4-carboxylate;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2-methoxyphenyl)oxalamide;
N¹—((S)-3-cyclohexyl-1-(((S)-4-hydroxy-3-oxo-1-((S)-2-oxopyrrolidin-3-yl)butan-2-yl)amino)-1-oxopropan-2-yl)-N²-(o-tolyl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(3-methoxyphenyl)oxalamide;
(S)-2-(3-(2-fluorophenyl)ureido)-N—((S)-4-hydroxy-3-oxo-1-((S)-2-oxopyrrolidin-3-yl)butan-2-yl)-4-methylpentanamide;
(S)-3-((S)-2-(2-((2,6-dimethylphenyl)amino)-2-oxoacetamido)-4-methylpentanamido)-2-oxo-4-((S)-2-oxopyrrolidin-3-yl)butyl 2-oxo-2-phenylacetate;
N¹-(2,6-dimethylphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2,6-dimethylphenyl)-N²-((2S)-1-((4-hydroxy-3-oxo-1-((S)-2-oxopyrrolidin-3-yl)butan-2-yl)amino)-4-methyl-1-oxopentan-2-yl)oxalamide;
N¹-cyclohexyl-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2,6-dimethoxyphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2-methoxy-6-methylphenyl)oxalamide;
N¹-(2,6-diethylphenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
(S)-3-((S)-2-(2-(cyclohexylamino)-2-oxoacetamido)-4-methylpentanamido)-2-oxo-4-((S)-2-oxopyrrolidin-3-yl)butyl heptanoate;
(S)-3-((S)-3-cyclohexyl-2-(2-(cyclohexylamino)-2-oxoacetamido)propanamido)-2-oxo-4-((S)-2-oxopyrrolidin-3-yl)butyl heptanoate;
N¹—((S)-3-(2-fluorophenyl)-1-(((S)-4-hydroxy-3-oxo-1-((S)-2-oxopyrrolidin-3-yl)butan-2-yl)amino)-1-oxopropan-2-yl)-N²-(o-tolyl)oxalamide;
N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹-(3-fluorobicyclo[1.1.1]pentan-1-yl)-N²—((S)-1-(((S)-4-hydroxy-3-oxo-1-((S)-2-oxopyrrolidin-3-yl)butan-2-yl)amino)-4,4-dimethyl-1-oxopentan-2-yl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(1-(p-tolyl)cyclopropyl)oxalamide;
N¹—((S)-3-cyclohexyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-3-cyclohexyl-1-(((R)-4-hydroxy-3-oxo-1-((S)-2-oxopiperidin-3-yl)butan-2-yl)amino)-1-oxopropan-2-yl)-N²-(2-fluorophenyl)oxalamide;
N¹—((S)-1-(((S)-4-hydroxy-3-oxo-1-((S)-2-oxopyrrolidin-3-yl)butan-2-yl)amino)-4-methyl-1-oxopentan-2-yl)-N²-(2,2,2-trifluoroethyl)oxalamide;
N¹—((S)-1-(((S)-4-hydroxy-3-oxo-1-((S)-2-oxopiperidin-3-yl)butan-2-yl)amino)-4-methyl-1-oxopentan-2-yl)-N²-(o-tolyl)oxalamide;
N¹-(2,6-diethylphenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-1-(((S)-4-hydroxy-3-oxo-1-((S)-2-oxopiperidin-3-yl)butan-2-yl)amino)-4-methyl-1-oxopentan-2-yl)-N²-(1-methylcyclopropyl)oxalamide;
(1S,3aR,6aS)-2-(2-((2-fluorophenyl)amino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide;
(1S,3aR,6aS)-2-(2-(tert-butylamino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide;
(1S,3aR,6aS)-2-(2-(tert-butylamino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
(1S,3aR,6aS)-2-(2-((2-fluorophenyl)amino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
N¹-(2-fluorophenyl)-N²—((S)-1-(((S)-4-hydroxy-3-oxo-1-((S)-2-oxoazepan-3-yl)butan-2-yl)amino)-4-methyl-1-oxopentan-2-yl)oxalamide;
4-methoxy-N—((S)-4-methyl-1-oxo-1-(((S)-1-oxo-3-((S)-2-oxopyrrolidin-3-yl)propan-2-yl)amino)pentan-2-yl)-1H-indole-2-carboxamide;
(1R,2S,5S)-6,6-dimethyl-N—((S)-1-oxo-3-((S)-2-oxopyrrolidin-3-yl)propan-2-yl)-3-(2-(4-(trifluoromethoxy)phenoxy)acetyl)-3-azabicyclo[3.1.0]hexane-2-carboxamide;
N¹—((S)-4-methyl-1-oxo-1-(((S)-1-oxo-3-((S)-2-oxopyrrolidin-3-yl)propan-2-yl)amino)pentan-2-yl)-N²-phenyloxalamide;
N¹—((S)-4-methyl-1-oxo-1-(((S)-1-oxo-3-((S)-2-oxopyrrolidin-3-yl)propan-2-yl)amino)pentan-2-yl)-N²-(o-tolyl)oxalamide;
N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide;
N¹—((S)-3-cyclohexyl-1-oxo-1-(((S)-1-oxo-3-((S)-2-oxopyrrolidin-3-yl)propan-2-yl)amino)propan-2-yl)-N²-(2-fluorophenyl)oxalamide;
N¹-(2-fluorophenyl)-N²—((S)-4-methyl-1-oxo-1-(((S)-1-oxo-3-((S)-2-oxopiperidin-3-yl)propan-2-yl)amino)pentan-2-yl)oxalamide;
N¹-cyclohexyl-N²—((S)-4-methyl-1-oxo-1-(((S)-1-oxo-3-((S)-2-oxopyrrolidin-3-yl)propan-2-yl)amino)pentan-2-yl)oxalamide;
N¹—((S)-4-methyl-1-oxo-1-(((S)-1-oxo-3-((S)-2-oxopyrrolidin-3-yl)propan-2-yl)amino)pentan-2-yl)-N²-(1-methylcyclopropyl)oxalamide;
(1S,3aR,6aS)-2-(2-((2-fluorophenyl)amino)-2-oxoacetyl)-N—((S)-1-oxo-3-((S)-2-oxopyrrolidin-3-yl)propan-2-yl)octahydrocyclopenta[c]pyrrole-1-carboxamide;
(1S,3aR,6aS)-2-(2-(2,4-dichlorophenoxy)acetyl)-N—((S)-1-oxo-3-((S)-2-oxopiperidin-3-yl)propan-2-yl)octahydrocyclopenta[c]pyrrole-1-carboxamide;
(1R,2S,5S)-3-(2-(tert-butylamino)-2-oxoacetyl)-6,6-dimethyl-N—((S)-1-oxo-3-((S)-2-oxopiperidin-3-yl)propan-2-yl)-3-azabicyclo[3.1.0]hexane-2-carboxamide;
(1S,3aR,6aS)-2-(2-oxo-2-((1,1,1-trifluoro-2-methylpropan-2-yl)amino)acetyl)-N—((S)-1-oxo-3-((S)-2-oxopiperidin-3-yl)propan-2-yl)octahydrocyclopenta[c]pyrrole-1-carboxamide;
(1R,2S,5S)-3-(2-(cyclohexylamino)-2-oxoacetyl)-6,6-dimethyl-N—((S)-1-oxo-3-((S)-2-oxopiperidin-3-yl)propan-2-yl)-3-azabicyclo[3.1.0]hexane-2-carboxamide;
(1S,3aR,6aS)-2-((S)-3,3-dimethyl-2-(2,2,2-trifluoroacetamido)butanoyl)-N—((S)-1-oxo-3-((S)-2-oxopiperidin-3-yl)propan-2-yl)octahydrocyclopenta[c]pyrrole-1-carboxamide;
(1S,3aR,6aS)-2-((R)-3-fluoro-3-methyl-2-(2,2,2-trifluoroacetamido)butanoyl)-N—((S)-1-oxo-3-((S)-2-oxopiperidin-3-yl)propan-2-yl)octahydrocyclopenta[c]pyrrole-1-carboxamide;
(1S,3aR,6aS)-2-((S)-2-(3,3-difluorocyclobutyl)-2-(2,2,2-trifluoroacetamido)acetyl)-N—((S)-1-oxo-3-((S)-2-oxopiperidin-3-yl)propan-2-yl)octahydrocyclopenta[c]pyrrole-1-carboxamide;
N—((S)-3,3-dimethyl-1-oxo-1-((1S,3aR,6aS)-1-(((S)-1-oxo-3-((S)-2-oxopiperidin-3-yl)propan-2-yl)carbamoyl)hexahydrocyclopenta[c]pyrrol-2(1H)-yl)butan-2-yl)-5-(trifluoromethyl)isoxazole-3-carboxamide;
(1S,3aR,6aS)-2-(O-(difluoromethyl)-N-(2,2,2-trifluoroacetyl)-L-threonyl)-N—((S)-1-oxo-3-((S)-2-oxopiperidin-3-yl)propan-2-yl)octahydrocyclopenta[c]pyrrole-1-carboxamide;
(1S,3aR,6aS)-2-((S)-2-(1-methylcyclopropyl)-2-(2,2,2-trifluoroacetamido)acetyl)-N—((S)-1-oxo-3-((S)-2-oxopiperidin-3-yl)propan-2-yl)octahydrocyclopenta[c]pyrrole-1-carboxamide;
(1R,2S,5S)-3-((S)-3-methoxy-3-methyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide;
(1R,2S,5S)-3-((R)-3-fluoro-3-methyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide;
(1R,2S,5S)—N—((S)-1-cyano-2-((S)-2-oxopyrrolidin-3-yl)ethyl)-3-((S)-4,4-dimethyl-2-(2,2,2-trifluoroacetamido)pentanoyl)-6,6-dimethyl-3-azabicyclo[3.1.0]hexane-2-carboxamide;
N¹—((S)-1-(((S)-1-cyano-2-((S)-2-oxopyrrolidin-3-yl)ethyl)amino)-4-methyl-1-oxopentan-2-yl)-N²-(2-fluorophenyl)oxalamide;
(1R,2S,5S)—N—((S)-1-cyano-2-((S)-2-oxopyrrolidin-3-yl)ethyl)-3-(2-((2-fluorophenyl)amino)-2-oxoacetyl)-6,6-dimethyl-3-azabicyclo[3.1.0]hexane-2-carboxamide;
(1S,3aR,6aS)—N—((S)-1-cyano-2-((S)-2-oxopiperidin-3-yl)ethyl)-2-(2-((2-fluorophenyl)amino)-2-oxoacetyl)octahydrocyclopenta[c]pyrrole-1-carboxamide;
(1S,3aR,6aS)—N—((S)-1-cyano-2-((S)-2-oxopiperidin-3-yl)ethyl)-2-(2-(2,4-dichlorophenoxy)acetyl)octahydrocyclopenta[c]pyrrole-1-carboxamide;
(1S,3aR,6aS)—N—((S)-1-cyano-2-((S)-2-oxopyrrolidin-3-yl)ethyl)-2-(2-((2-fluorophenyl)amino)-2-oxoacetyl)octahydrocyclopenta[c]pyrrole-1-carboxamide;
(1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide;
(S)—N—((S)-1-cyano-2-((S)-2-oxopyrrolidin-3-yl)ethyl)-5-(2-((2-fluorophenyl)amino)-2-oxoacetyl)-5-azaspiro[2.4]heptane-6-carboxamide;
(1R,2S,5S)—N—((S)-1-cyano-2-((S)-2-oxopyrrolidin-3-yl)ethyl)-6,6-dimethyl-3-(2-oxo-2-((1-(trifluoromethyl)cyclopropyl)amino)acetyl)-3-azabicyclo[3.1.0]hexane-2-carboxamide;
(1R,2S,5S)—N—((S)-1-cyano-2-((S)-2-oxopiperidin-3-yl)ethyl)-3-((S)-3-methoxy-3-methyl-2-(2,2,2-trifluoroacetamido)butanoyl)-6,6-dimethyl-3-azabicyclo[3.1.0]hexane-2-carboxamide;
(1R,2S,5S)—N—((S)-1-cyano-2-((S)-2-oxopiperidin-3-yl)ethyl)-3-((R)-3-fluoro-3-methyl-2-(2,2,2-trifluoroacetamido)butanoyl)-6,6-dimethyl-3-azabicyclo[3.1.0]hexane-2-carboxamide;
(1R,2S,5S)—N—((S)-1-cyano-2-((S)-2-oxopiperidin-3-yl)ethyl)-6,6-dimethyl-3-((S)-2-(tetrahydro-2H-pyran-4-yl)-2-(2,2,2-trifluoroacetamido)acetyl)-3-azabicyclo[3.1.0]hexane-2-carboxamide;
(1R,2S,5S)—N—((S)-1-cyano-2-((S)-2-oxopiperidin-3-yl)ethyl)-3-((S)-2-(4,4-difluorocyclohexyl)-2-(2,2,2-trifluoroacetamido)acetyl)-6,6-dimethyl-3-azabicyclo[3.1.0]hexane-2-carboxamide;
(1S,3aR,6aS)—N—((S)-1-cyano-2-((S)-2-oxopiperidin-3-yl)ethyl)-2-((S)-3,3-dimethyl-2-(2,2,2-trifluoroacetamido)butanoyl)octahydrocyclopenta[c]pyrrole-1-carboxamide;
(1S,3aR,6aS)—N—((S)-1-cyano-2-((S)-2-oxopiperidin-3-yl)ethyl)-2-((R)-3-fluoro-3-methyl-2-(2,2,2-trifluoroacetamido)butanoyl)octahydrocyclopenta[c]pyrrole-1-carboxamide;
(3aS,4S,6aR)—N—((S)-1-cyano-2-((S)-2-oxopiperidin-3-yl)ethyl)-5-((S)-3,3-dimethyl-2-(2,2,2-trifluoroacetamido)butanoyl)hexahydro-1H-furo[3,4-c]pyrrole-4-carboxamide;
(1S,3aR,6aS)—N—((S)-1-cyano-2-((S)-2-oxopiperidin-3-yl)ethyl)-2-((S)-3,3-dimethyl-2-(2,2,2-trifluoroacetamido)butanoyl)-5,5-difluorooctahydrocyclopenta[c]pyrrole-1-carboxamide;
(1S,3aR,6aS)—N—((S)-1-cyano-2-((S)-2-oxopiperidin-3-yl)ethyl)-2-((2S)-3,3-dimethyl-2-(tetrahydrofuran-2-carboxamido)butanoyl)octahydrocyclopenta[c]pyrrole-1-carboxamide;
N—((S)-1-((1S,3aR,6aS)-1-(((S)-1-cyano-2-((S)-2-oxopiperidin-3-yl)ethyl)carbamoyl)hexahydrocyclopenta[c]pyrrol-2(1H)-yl)-3,3-dimethyl-1-oxobutan-2-yl)-5-(trifluoromethyl)isoxazole-3-carboxamide;
(1S,3aR,6aS)—N—((S)-1-cyano-2-((S)-2-oxopiperidin-3-yl)ethyl)-2-(O-(difluoromethyl)-N-(2,2,2-trifluoroacetyl)-L-threonyl)octahydrocyclopenta[c]pyrrole-1-carboxamide;
(1S,3aR,6aS)—N—((S)-1-cyano-2-((S)-2-oxopiperidin-3-yl)ethyl)-2-(N-(2,2,2-trifluoroacetyl)-O-(trifluoromethyl)-L-threonyl)octahydrocyclopenta[c]pyrrole-1-carboxamide;
(1S,3aR,6aS)—N—((S)-1-cyano-2-((S)-2-oxopiperidin-3-yl)ethyl)-2-(N-(2,2,2-trifluoroacetyl)-O-(trifluoromethyl)-L-seryl)octahydrocyclopenta[c]pyrrole-1-carboxamide;
(1S,3aR,6aS)—N—((S)-1-cyano-2-((S)-2-oxopiperidin-3-yl)ethyl)-2-(O-(difluoromethyl)-N-(2,2,2-trifluoroacetyl)-L-seryl)octahydrocyclopenta[c]pyrrole-1-carboxamide;
(1S,3aR,6aS)—N—((S)-1-cyano-2-((S)-2-oxopiperidin-3-yl)ethyl)-2-((S)-4,4,4-trifluoro-2-(2,2,2-trifluoroacetamido)-3-(trifluoromethyl)butanoyl)octahydrocyclopenta[c]pyrrole-1-carboxamide;
(1S,3aR,6aS)-2-((S)-2-acetamido-4,4,4-trifluoro-3-(trifluoromethyl)butanoyl)-N—((S)-1-cyano-2-((S)-2-oxopiperidin-3-yl)ethyl)octahydrocyclopenta[c]pyrrole-1-carboxamide;
(1S,3aR,6aS)—N—((S)-1-cyano-2-((S)-2-oxopiperidin-3-yl)ethyl)-2-((S)-3-(3,3-difluoroazetidin-1-yl)-2-(2,2,2-trifluoroacetamido)propanoyl)octahydrocyclopenta[c]pyrrole-1-carboxamide;
(1S,3aR,6aS)—N-((1S)-1-cyano-2-(2-oxopiperidin-3-yl)ethyl)-2-((1S,2R)-2-(2,2,2-trifluoroacetamido)-[1,1′-bi(cyclopropane)]-2-carbonyl)octahydrocyclopenta[c]pyrrole-1-carboxamide;
(1S,3aR,6aS)—N—((S)-1-cyano-2-((S)-2-oxopiperidin-3-yl)ethyl)-2-((S)-2-(1-methylcyclopropyl)-2-(2,2,2-trifluoroacetamido)acetyl)octahydrocyclopenta[c]pyrrole-1-carboxamide;
1,1,1-trifluoro-2-methylpropan-2-yl ((S)-1-((1S,3aR,6aS)-1-(((S)-1-cyano-2-((S)-2-oxopiperidin-3-yl)ethyl)carbamoyl)hexahydrocyclopenta[c]pyrrol-2(1H)-yl)-3,3-dimethyl-1-oxobutan-2-yl)carbamate;
(1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-3-azaspiro[bicyclo[3.1.0]hexane-6,1′-cyclopropane]-2-carboxamide;
N¹-(2-fluorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(o-tolyl)oxalamide;
N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide;
(1S,3aR,6aS)—N—((S)-4-hydroxy-3-oxo-1-((S)-2-oxopiperidin-3-yl)butan-2-yl)-2-(2-oxo-2-(o-tolylamino)acetyl)octahydrocyclopenta[c]pyrrole-1-carboxamide;
(1S,3aR,6aS)—N—((R)-4-hydroxy-3-oxo-1-((S)-2-oxopiperidin-3-yl)butan-2-yl)-2-(2-oxo-2-(o-tolylamino)acetyl)octahydrocyclopenta[c]pyrrole-1-carboxamide;
(S)-5-(2-((2-fluorophenyl)amino)-2-oxoacetyl)-N—((S)-4-hydroxy-3-oxo-1-((S)-2-oxopyrrolidin-3-yl)butan-2-yl)-5-azaspiro[2.4]heptane-6-carboxamide;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(3-(trifluoromethoxy)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
N¹-(3-fluorobicyclo[1.1.1]pentan-1-yl)-N²—((S)-1-(((S)-4-hydroxy-3-oxo-1-((S)-2-oxopyrrolidin-3-yl)butan-2-yl)amino)-3-(1-methylcyclobutyl)-1-oxopropan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethoxy)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(3-(trifluoromethyl)phenyl)oxalamide;
N¹-(2-(tert-butyl)phenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(4-(trifluoromethoxy)phenyl)oxalamide;
N¹-(3-chlorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl 3-methylbutanoate;
(S)-3-((S)-2-(2-((2-fluorophenyl)amino)-2-oxoacetamido)-4-methylpentanamido)-2-oxo-4-((S)-2-oxopiperidin-3-yl)butyl 3-methylbutanoate;
(S)-3-((S)-2-(2-((2-fluorophenyl)amino)-2-oxoacetamido)-4-methylpentanamido)-2-oxo-4-((S)-2-oxopiperidin-3-yl)butyl acetate;
(S)-3-((S)-2-(2-((2-fluorophenyl)amino)-2-oxoacetamido)-4-methylpentanamido)-2-oxo-4-((S)-2-oxopyrrolidin-3-yl)butyl acetate;
(S)-3-((S)-2-(2-((2-fluorophenyl)amino)-2-oxoacetamido)-4-methylpentanamido)-2-oxo-4-((S)-2-oxopiperidin-3-yl)butyl isopropyl carbonate;
(S)-3-((S)-2-(2-((2-fluorophenyl)amino)-2-oxoacetamido)-4-methylpentanamido)-2-oxo-4-((S)-2-oxopyrrolidin-3-yl)butyl isopropyl carbonate;
(S)-2-((S)-2-(2-((4-fluorobicyclo[2.2.2]octan-1-yl)amino)-2-oxoacetamido)-4-methylpentanamido)-3-((S)-2-oxopiperidin-3-yl)propanoic acid;
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(4-fluorobicyclo[2.2.2]octan-1-yl)oxalamide;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(6-(trifluoromethyl)pyridin-2-yl)oxalamide;
N¹-(tert-butyl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
N¹-(tert-butyl)-N²—((S)-3-cyclopropyl-1-(((S)-4-hydroxy-3-oxo-1-((S)-2-oxopiperidin-3-yl)butan-2-yl)amino)-1-oxopropan-2-yl)oxalamide;
N¹-(tert-butyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)pyridin-3-yl)oxalamide;
(S)—N—((S)-1-cyano-2-((S)-2-oxopyrrolidin-3-yl)ethyl)-5-(4-methylpentanoyl)-5-azaspiro[2.4]heptane-6-carboxamide;
(S)-3-((S)-2-(1H-indole-2-carboxamido)-4-methylpentanamido)-2-oxo-4-((S)-2-oxopyrrolidin-3-yl)butyl heptanoate;
(S)-3-((S)-2-(4-methoxy-1H-indole-2-carboxamido)-4-methylpentanamido)-2-oxo-4-((S)-2-oxopyrrolidin-3-yl)butyl heptanoate;
(S)-3-((S)-2-((((3-chlorobenzyl)oxy)carbonyl)amino)-4-methylpentanamido)-2-oxo-4-((S)-2-oxopyrrolidin-3-yl)butyl isobutyrate;
(S)-3-((S)-2-((((3-chlorobenzyl)oxy)carbonyl)amino)-4-methylpentanamido)-2-oxo-4-((S)-2-oxopyrrolidin-3-yl)butyl 2-phenylacetate;
3-chlorobenzyl ((S)-1-(((S)-4-hydroxy-3-oxo-1-((S)-2-oxopyrrolidin-3-yl)butan-2-yl)amino)-4-methyl-1-oxopentan-2-yl)carbamate;
(S)-3-((S)-2-(4-methoxy-1H-indole-2-carboxamido)-4-methylpentanamido)-2-oxo-4-((S)-2-oxopyrrolidin-3-yl)butyl 1-cyclohexylpiperidine-4-carboxylate;
(S)-3-((S)-2-(4-methoxy-1H-indole-2-carboxamido)-4-methylpentanamido)-2-oxo-4-((S)-2-oxopyrrolidin-3-yl)butyl 4-(piperidin-1-yl)cyclohexane-1-carboxylate;
N—((S)-3-cyclopropyl-1-oxo-1-(((S)-1-oxo-3-((S)-2-oxopyrrolidin-3-yl)propan-2-yl)amino)propan-2-yl)-5-(2-fluorophenyl)isoxazole-3-carboxamide;
5-(2-fluorophenyl)-N—((S)-4-methyl-1-oxo-1-(((S)-1-oxo-3-((S)-2-oxopiperidin-3-yl)propan-2-yl)amino)pentan-2-yl)isoxazole-3-carboxamide;
N—((S)-4-methyl-1-oxo-1-(((S)-1-oxo-3-((S)-2-oxopiperidin-3-yl)propan-2-yl)amino)pentan-2-yl)-5-(pyridin-2-yl)isoxazole-3-carboxamide;
N—((S)-4-methyl-1-oxo-1-(((S)-1-oxo-3-((S)-2-oxopyrrolidin-3-yl)propan-2-yl)amino)pentan-2-yl)-5-(pyridin-2-yl)isoxazole-3-carboxamide;
N—((S)-1-(((S)-4-hydroxy-3-oxo-1-((S)-2-oxopiperidin-3-yl)butan-2-yl)amino)-4-methyl-1-oxopentan-2-yl)-4-methoxy-1H-indole-2-carboxamide;
(1R,2S,5S)—N—((S)-1-cyano-2-((S)-2-oxopiperidin-3-yl)ethyl)-6,6-dimethyl-3-(2-(trifluoromethyl)isonicotinoyl)-3-azabicyclo[3.1.0]hexane-2-carboxamide;
(1R,2S,5S)-3-(2-acetamido-5,5,5-trifluoro-4-methylpentanoyl)-N—((S)-1-cyano-2-((S)-2-oxopiperidin-3-yl)ethyl)-6,6-dimethyl-3-azabicyclo[3.1.0]hexane-2-carboxamide;
(1R,2S,5S)-3-((S)-3,3-dimethyl-2-(2,2,2-trifluoroacetamido)butanoyl)-6,6-dimethyl-N—((S)-1-oxo-3-((S)-2-oxopiperidin-3-yl)propan-2-yl)-3-azabicyclo[3.1.0]hexane-2-carboxamide;
N¹—((S)-1-(((R)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
(R)—N—((S)-3-(3-fluorophenyl)-1-(((S)-4-hydroxy-3-oxo-1-((S)-2-oxopiperidin-3-yl)butan-2-yl)amino)-1-oxopropan-2-yl)-2-hydroxy-4-methylpentanamide;
N¹-(4-fluorobicyclo[2.2.2]octan-1-yl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
(S)—N—((S)-1-cyano-2-((S)-2-oxopiperidin-3-yl)ethyl)-4,4-dimethyl-2-(4-methylpentanamido)pentanamide;
(S)-3-((S)-2-(4-methoxy-1H-indole-2-carboxamido)-4-methylpentanamido)-2-oxo-4-((S)-2-oxopyrrolidin-3-yl)butyl pentadecanoate; and
(S)-3-((S)-2-(2-((2-fluorophenyl)amino)-2-oxoacetamido)-4-methylpentanamido)-2-oxo-4-((S)-2-oxopiperidin-3-yl)butyl 2-cyanobenzoate.

25. The compound or pharmaceutically acceptable salt thereof according to claim 1, wherein the compound is selected from the group consisting of:
N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-1-(((S)-4-hydroxy-3-oxo-1-((S)-2-oxopiperidin-3-yl)butan-2-yl)amino)-4-methyl-1-oxopentan-2-yl)-N²-(o-tolyl)oxalamide;
(1S,3aR,6aS)-2-(2-(tert-butylamino)-2-oxoacetyl)-N—((S)-4-hydroxy-3-oxo-1-((S)-2-oxopyrrolidin-3-yl)butan-2-yl)octahydrocyclopenta[c]pyrrole-1-carboxamide;
(1S,3aR,6aS)-2-(2-((2-fluorophenyl)amino)-2-oxoacetyl)-N—((S)-4-hydroxy-3-oxo-1-((S)-2-oxopiperidin-3-yl)butan-2-yl)octahydrocyclopenta[c]pyrrole-1-carboxamide;
N¹—((S)-4-methyl-1-oxo-1-(((S)-1-oxo-3-((S)-2-oxopyrrolidin-3-yl)propan-2-yl)amino)pentan-2-yl)-N²-(o-tolyl)oxalamide;
N¹-(2-fluorophenyl)-N²—((S)-4-methyl-1-oxo-1-(((S)-1-oxo-3-((S)-2-oxopyrrolidin-3-yl)propan-2-yl)amino)pentan-2-yl)oxalamide;
N¹—((S)-3-cyclohexyl-1-oxo-1-(((S)-1-oxo-3-((S)-2-oxopyrrolidin-3-yl)propan-2-yl)amino)propan-2-yl)-N²-(2-fluorophenyl)oxalamide;
N¹-cyclohexyl-N²—((S)-4-methyl-1-oxo-1-(((S)-1-oxo-3-((S)-2-oxopyrrolidin-3-yl)propan-2-yl)amino)pentan-2-yl)oxalamide;
(1S,3aR,6aS)-2-(2-(2,4-dichlorophenoxy)acetyl)-N—((S)-1-oxo-3-((S)-2-oxopiperidin-3-yl)propan-2-yl)octahydrocyclopenta[c]pyrrole-1-carboxamide;
(1R,2S,5S)-3-(2-(cyclohexylamino)-2-oxoacetyl)-6,6-dimethyl-N—((S)-1-oxo-3-((S)-2-oxopiperidin-3-yl)propan-2-yl)-3-azabicyclo[3.1.0]hexane-2-carboxamide;
N¹—((S)-1-(((S)-1-cyano-2-((S)-2-oxopyrrolidin-3-yl)ethyl)amino)-4-methyl-1-oxopentan-2-yl)-N²-(2-fluorophenyl)oxalamide;
(1R,2S,5S)—N—((S)-1-cyano-2-((S)-2-oxopiperidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide;
N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
N—((S)-3-cyclopropyl-1-oxo-1-(((S)-1-oxo-3-((S)-2-oxopyrrolidin-3-yl)propan-2-yl)amino)propan-2-yl)-5-(2-fluorophenyl)isoxazole-3-carboxamide;
(1R,2S,5S)—N—((S)-1-cyano-2-((S)-2-oxopyrrolidin-3-yl)ethyl)-3-((S)-4,4-dimethyl-2-(2,2,2-trifluoroacetamido)pentanoyl)-6,6-dimethyl-3-azabicyclo[3.1.0]hexane-2-carboxamide;
N¹-(2-fluorophenyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide;
(S)-3-((S)-2-(2-((2-fluorophenyl)amino)-2-oxoacetamido)-4-methylpentanamido)-2-oxo-4-((S)-2-oxopyrrolidin-3-yl)butyl acetate;
(S)-3-((S)-2-(2-((2-fluorophenyl)amino)-2-oxoacetamido)-4-methylpentanamido)-2-oxo-4-((S)-2-oxopiperidin-3-yl)butyl acetate;
N¹-(3-fluorobicyclo[1.1.1]pentan-1-yl)-N²—((S)-1-(((S)-4-hydroxy-3-oxo-1-((S)-2-oxopiperidin-3-yl)butan-2-yl)amino)-3-(1-methylcyclobutyl)-1-oxopropan-2-yl)oxalamide;
(S)-3-((S)-2-(2-((2-fluorophenyl)amino)-2-oxoacetamido)-4-methylpentanamido)-2-oxo-4-((S)-2-oxopiperidin-3-yl)butyl isopropyl carbonate;
(S)-3-((S)-2-(2-((2-fluorophenyl)amino)-2-oxoacetamido)-4-methylpentanamido)-2-oxo-4-((S)-2-oxopyrrolidin-3-yl)butyl isopropyl carbonate;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethoxy)phenyl)oxalamide; and
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide.

26. The compound or pharmaceutically acceptable salt thereof according to claim 1, wherein the compound is selected from the group consisting of:
N¹-(2-fluorophenyl)-N²—((S)-1-(((S)-4-hydroxy-3-oxo-1-((S)-2-oxopyrrolidin-3-yl)butan-2-yl)amino)-4-methyl-1-oxopentan-2-yl)oxalamide;
N¹-(2-fluorophenyl)-N²—((S)-1-(((S)-4-hydroxy-3-oxo-1-((S)-2-oxopiperidin-3-yl)butan-2-yl)amino)-4-methyl-1-oxopentan-2-yl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethyl)phenyl)oxalamide;
N¹—((S)-1-(((S)-4-hydroxy-3-oxo-1-((S)-2-oxopiperidin-3-yl)butan-2-yl)amino)-4,4-dimethyl-1-oxopentan-2-yl)-N²-(2-(trifluoromethoxy)phenyl)oxalamide;
N¹-(tert-butyl)-N²—((S)-1-(((S)-4-hydroxy-3-oxo-1-((S)-2-oxopiperidin-3-yl)butan-2-yl)amino)-4,4-dimethyl-1-oxopentan-2-yl)oxalamide; and
N¹—((S)-3-cyclopropyl-1-(((S)-4-hydroxy-3-oxo-1-((S)-2-oxopiperidin-3-yl)butan-2-yl)amino)-1-oxopropan-2-yl)-N²-(2-fluorophenyl)oxalamide.

27. A pharmaceutical composition comprising a compound or pharmaceutically acceptable salt thereof according to claim 1 and a pharmaceutically acceptable carrier.

28. A method for inhibiting the main protease (M^{pro}) of severe acute respiratory syndrome Coronavirus-2 (SARS-CoV-2), comprising contacting M^{pro} with a compound or pharmaceutically acceptable thereof according to claim 1.

29. A method for treating COVID-19 in a subject suffering therefrom, or for preventing COVID-19 in a subject, comprising administering to the subject a compound or pharmaceutically acceptable thereof according to claim 1.
